(19) United States
(12) Patent Application Publication
(10) Pub. No.: US 2024/0316169 A1
(43) Pub. Date: Sep. 26, 2024

(54) UTILIZATION OF ANTIBODIES TO SHAPE ANTIBODY RESPONSES TO AN ANTIGEN

(71) Applicant: Regeneron Pharmaceuticals, Inc., Tarrytown, NY (US)

(72) Inventors: Andrew J. Murphy, Croton-on-Hudson, NY (US); Christos KYRATSOUS, Irvington, NY (US); Alina BAUM, Tarry town, NY (US); Christopher PETRO, Tarrytown, NY (US)

(73) Assignee: Regeneron Pharmaceuticals, Inc., Tarrytown, NY (US)

(21) Appl. No.: 18/576,623

(22) PCT Filed: Jul. 1, 2022
(86) PCT No.: PCT/US22/35968

§ 371 Date: Jan. 4, 2024

Related U.S. Application Data
(60) Provisional application No. 63/218,486, filed on Jul. 5, 2021.

(51) Int. Cl. A61K 39/00; A61K 39/145; A61K 39/215; A61K 39/42; A61P 37/04

(52) U.S. Cl. CPC A61K 39/0008; A61K 39/145; A61K 39/215; A61K 39/42; A61P 37/04; A61K 2039/53; A61K 2039/545; C12N 2770/20023; C12N 2770/20052

(57) ABSTRACT

Described herein are methods and compositions for directing an antibody response in a subject away from one or more first epitopes of an antigen (e.g., immunodominant epitopes of a vaccine antigen) and towards one or more second epitopes of the antigen by administering one or more antibodies targeting the one or more first epitopes of the antigen.

### CROSS-REFERENCE TO RELATED APPLICATIONS

This application claims priority to U.S. Provisional Application No. 63/218,486, filed Jul. 5, 2021, the disclosure of which is herein incorporated by reference in its entirety.

### FIELD

Described herein are methods and compositions for directing an antibody response in a subject away from one or more first epitopes of an antigen (e.g., immunodominant epitopes of a vaccine antigen) and towards one or more second epitopes of the antigen by administering one or more antibodies targeting the one or more first epitopes of the antigen.

### BACKGROUND

Pathogenic organisms such as viruses and bacteria have evolved elaborate strategies to defeat the host immune response. Such strategies often hamper efforts to develop successful vaccines against many pathogenic organisms. For example, a vaccine that elicits an immune response against surface-exposed antigens of a pathogenic organism may be extremely effective against certain strains, but poorly effective against variant strains, due to frequent alteration in the surface-exposed antigens. A separate problem in vaccine design is that some epitopes elicit an undesirable immune response. Therefore, vaccine strategies that can steer immune response towards desired antigen epitopes and away from undesirable epitopes are needed to improve effectiveness of current vaccines.

### SUMMARY

As specified in the Background section above, there is a great need for development of methods that drive an antibody response toward desired antigen epitopes and away from undesirable epitopes. The present disclosure addresses this and other needs by providing methods and compositions for directing the antibody response away from one or more undesirable epitopes of an antigen.

In one aspect, the invention provides a method for redirecting an antibody response in a subject from one or more first epitopes of an antigen towards one or more second epitopes of the antigen, the method comprising administering to the subject (i) the antigen or a nucleic acid molecule encoding the antigen and (ii) one or more antibodies targeting the one or more first epitopes of the antigen or one or more nucleic acid molecules encoding the one or more antibodies, wherein the antigen or a nucleic acid molecule encoding the antigen and the one or more antibodies or one or more nucleic acid molecules encoding the one or more antibodies are administered to the subject in amounts effective for generating antibodies to one or more second epitopes of the antigen.

In another aspect, the invention provides a method for shielding one or more first epitopes of an antigen from recognition by the immune system of a subject, the method comprising administering to the subject (i) the antigen or a nucleic acid molecule encoding the antigen and (ii) one or more antibodies targeting the one or more first epitopes of the antigen or one or more nucleic acid molecules encoding the one or more antibodies, wherein the one or more antibodies or one or more nucleic acid molecules encoding the one or more antibodies are administered to the subject in an amount effective to shield one or more first epitopes of the antigen from recognition by the immune system of the subject.

In another aspect, the invention provides a method for generating one or more antibodies targeting a second epitope of an antigen, the method comprising administering to a subject (i) the antigen or a nucleic acid molecule encoding the antigen and (ii) one or more antibodies targeting one or more first epitopes of the antigen or one or more nucleic acid molecules encoding the one or more antibodies, wherein the antigen or a nucleic acid molecule encoding the antigen and the one or more antibodies or one or more nucleic acid molecules encoding the one or more antibodies are administered to the subject in amounts effective for generating antibodies to one or more second epitopes of the antigen.

In some embodiments, the above-described method(s) further comprise isolating from the subject one or more antibodies which target the antigen or isolating cells producing antibodies which target the antigen.

In some embodiments, the isolating comprises binding of the antibodies or cells producing the antibodies to the antigen, wherein the antigen comprises a detectable label.

In some embodiments, the cells producing antibodies are B cells.

In some embodiments, the above-described methods further comprise generating a monoclonal antibody (mAb) based on the antibody isolated from the subject or an antigen-binding fragment thereof.

In some embodiments, the monoclonal antibody (mAb) is a human antibody.

In some embodiments, the monoclonal antibody (mAb) is a humanized antibody.

In another aspect, the invention provides a method for increasing efficacy of a vaccine in a subject in need thereof, wherein the vaccine comprises an antigen or a nucleic acid molecule encoding the antigen, the method comprising administering to the subject (i) the vaccine and (ii) one or more antibodies or one or more nucleic acid molecules encoding the one or more antibodies targeting one or more first epitopes of the antigen, wherein the vaccine and the one or more antibodies or one or more nucleic acid molecules encoding the one or more antibodies are administered to the subject in amounts effective for increasing efficacy of the vaccine.

In some embodiments, the vaccine is administered to the subject in a prime-boost regimen, and wherein the prime-boost regimen comprises administering the one or more antibodies or one or more nucleic acid molecules encoding the one or more antibodies to the subject after administering a prime dose of the vaccine to the subject but before administering a boost dose of the vaccine to the subject.

In some embodiments, the one or more antibodies or one or more nucleic acid molecules encoding the one or more antibodies are administered to the subject before administering the antigen or the nucleic acid molecule encoding the antigen.

In some embodiments, the one or more antibodies or one or more nucleic acid molecules encoding the one or more antibodies are administered to the subject up to three weeks before administering the antigen or the nucleic acid molecule encoding the antigen.

In some embodiments, the one or more antibodies or one or more nucleic acid molecules encoding the one or more antibodies are administered to the subject up to three days before administering the antigen or the nucleic acid molecule encoding the antigen.

In some embodiments, the one or more antibodies or one or more nucleic acid molecules encoding the one or more antibodies are administered to the subject after administering the antigen or the nucleic acid molecule encoding the antigen.

In some embodiments, the one or more antibodies or one or more nucleic acid molecules encoding the one or more antibodies are administered to the subject up to three weeks after administering the antigen or the nucleic acid molecule encoding the antigen.

In some embodiments, the one or more antibodies or one or more nucleic acid molecules encoding the one or more antibodies are administered to the subject during administering the antigen or the nucleic acid molecule encoding the antigen.

In some embodiments, (i) the one or more antibodies or one or more nucleic acid molecules encoding the one or more antibodies and (ii) the antigen or the nucleic acid molecule encoding the antigen are administered as different formulations.

In some embodiments, (i) the one or more antibodies or one or more nucleic acid molecules encoding the one or more antibodies and (ii) the antigen or the nucleic acid molecule encoding the antigen are administered in the same formulation.

In some embodiments, the method comprises administering to the subject a nucleic acid molecule encoding (i) the one or more antibodies and (ii) the antigen.

In some embodiments, the nucleic acid molecule is an RNA molecule

In some embodiments, the RNA molecule is an mRNA molecule.

In some embodiments, the nucleic acid molecule is a DNA molecule.

In some embodiments, the nucleic acid molecule is chemically modified.

In some embodiments, the nucleic acid molecule comprises at least one regulatory element operably linked to a nucleotide sequence encoding the antigen and/or a nucleotide sequence encoding the one or more antibodies.

In some embodiments, the regulatory element is a promoter.

In some embodiments, the nucleic acid molecule is comprised within a vector.

In some embodiments, the vector is a viral vector.

In some embodiments, the viral vector is a retroviral vector, an adenoviral vector, an adeno-associated virus vector, an alphaviral vector, a herpes virus vector, a baculovirus vector, or a vaccinia virus vector.

In some embodiments, the retroviral vector is a lentiviral vector.

In some embodiments, the vector is a non-viral vector.

In some embodiments, the non-viral vector is a minicircle plasmid, a Sleeping Beauty transposon, a piggyBac transposon, or a single- or double-stranded DNA molecule that is used as a template for homology directed repair (HDR) based gene editing.

In some embodiments, the one or more first epitopes are immunodominant epitopes.

In some embodiments, the immunodominant epitopes are less conserved than other epitopes of the antigen between different strains or species of a pathogen from which the antigen is derived.

In some embodiments, the antigen is a protein antigen.

In some embodiments, the antigen is a non-protein antigen.

In some embodiments, the antigen is derived from a Class I pathogen.

In some embodiments, the antigen is derived from a Class II pathogen.

In some embodiments, the pathogen is a virus.

In some embodiments, the virus is a coronavirus.

In some embodiments, the coronavirus is SARS-CoV-2.

In some embodiments, the antigen is SARS-CoV-2 spike glycoprotein and the one or more first epitopes are neutralizing epitopes comprised within receptor binding domain (RBD) of the SARS-CoV-2 spike glycoprotein.

In some embodiments, the virus is an influenza virus.

In some embodiments, the antigen is influenza hemagglutinin (HA), and the one or more first epitopes are comprised within sialic-acid, receptor binding site (RBS) on the HA head.

In some embodiments, the antigen is an endogenous molecule of the subject.

In some embodiments, the antigen is targeted by an immune response in an autoimmune disease.

In some embodiments, the one or more antibodies are monoclonal antibodies (mAbs).

In some embodiments, the subject is a mammal.

In some embodiments, the subject is a human.

In some embodiments, the subject is an experimental animal.

In some embodiments, the subject is a mouse.

In another aspect, the invention provides a nucleic acid molecule encoding an antigen and one or more antibodies targeting one or more first epitopes of the antigen.

In some embodiments, the nucleic acid molecule is an RNA molecule

In some embodiments, the RNA molecule is an mRNA molecule.

In some embodiments, the nucleic acid molecule is a DNA molecule.

In some embodiments, the nucleic acid molecule is chemically modified.

In some embodiments, the nucleic acid molecule comprises at least one regulatory element operably linked to a nucleotide sequence encoding the antigen and/or a nucleotide sequence encoding the one or more antibodies.

In some embodiments, the regulatory element is a promoter.

In another aspect, the invention provides a vector comprising the nucleic acid molecule encoding an antigen and one or more antibodies targeting one or more first epitopes of the antigen.

In some embodiments, the vector is a viral vector.

In some embodiments, the viral vector is a retroviral vector, an adenoviral vector, an adeno-associated virus vector, an alphaviral vector, a herpes virus vector, a baculovirus vector, or a vaccinia virus vector.

In some embodiments, the retroviral vector is a lentiviral vector.

In some embodiments, the vector is a non-viral vector.

In some embodiments, the non-viral vector is a minicircle plasmid, a Sleeping Beauty transposon, a piggyBac transposon, or a single or double stranded DNA molecule that is used as a template for homology directed repair (HDR) based gene editing.

In another aspect, the invention provides an isolated host cell comprising a nucleic acid molecule disclosed herein, or a vector disclosed herein. In some embodiments, the host cell is a mammalian cell.

In another aspect, the invention provides a lipid nanoparticle comprising a nucleic acid disclosed herein or a vector disclosed herein.

In another aspect, the invention provides a formulation comprising a nucleic acid molecule disclosed herein, a vector disclosed herein, or a lipid nanoparticle disclosed herein.

In another aspect, the invention provides a formulation comprising an antigen or a nucleic acid molecule encoding the antigen, and one or more antibodies targeting one or more first epitopes of the antigen or one or more nucleic acid molecules encoding the one or more antibodies.

In another aspect, the invention provides a formulation comprising two or more monoclonal antibodies (mAbs) targeting one or more first epitopes of an antigen.

In another aspect, the invention provides a formulation comprising two or more monoclonal antibodies (mAbs) targeting a combination of first epitopes and second epitopes of an antigen.

In some embodiments, the first epitopes are immunodominant epitopes.

In some embodiments, the immunodominant epitopes are less conserved than other epitopes of the antigen between different strains or species of a pathogen from which the antigen is derived.

In some embodiments, the antigen is a protein antigen.

In some embodiments, the antigen is a non-protein antigen.

In some embodiments, the antigen is derived from a Class I pathogen.

In some embodiments, the antigen is derived from a Class II pathogen.

In some embodiments, the pathogen is a virus.

In some embodiments, the virus is a coronavirus.

In some embodiments, the coronavirus is SARS-CoV-2.

In some embodiments, the antigen is SARS-CoV-2 spike glycoprotein and the first epitopes are neutralizing epitopes comprised within receptor binding domain (RBD) of the SARS-CoV-2 spike glycoprotein.

In some embodiments, the virus is an influenza virus.

In some embodiments, the antigen is influenza hemagglutinin (HA), and the one or more first epitopes are comprised within sialic-acid, receptor binding site (RBS) on the HA head.

In some embodiments, the antigen is a molecule targeted by an immune response in an autoimmune disease.

In another aspect, the invention provides a kit comprising (i) an antigen or a nucleic acid molecule encoding the antigen, and (ii) one or more antibodies targeting one or more first epitopes of the antigen or one or more nucleic acid molecules encoding the one or more antibodies.

In one aspect, the invention provides a method for redirecting an antibody response in a subject from one or more undesirable epitopes of an antigen towards other epitopes of said antigen, said method comprising administering to the subject an effective amount of one or more antibodies targeting said one or more undesirable epitopes, wherein said one or more antibodies are administered to the subject before or during administering said antigen or a nucleic acid encoding said antigen. In some embodiments, said one or more antibodies are administered before (e.g., about 3 days before) administering said antigen or a nucleic acid encoding said antigen to the subject. In some embodiments, the method further comprises isolating from the subject antibodies which recognize other antigen epitopes that are not undesirable epitopes and optionally further comprises generating monoclonal antibodies (mAbs) based on the antibodies isolated from the subject. For non-limiting examples of methods for isolating and characterizing antibodies see, e.g., U.S. Pat. Nos. 8,062,640; 7,582,298; and 10,752,698 incorporated herein by reference in their entirety.

In another aspect, the invention provides a method for increasing efficacy of a vaccine in a subject, wherein the vaccine comprises an antigen or a nucleic acid encoding said antigen, said method comprising administering to the subject an effective amount of one or more antibodies targeting one or more undesirable epitopes of said antigen, wherein said one or more antibodies are administered to the subject before or during administering said vaccine. In some embodiments, said one or more antibodies are administered before (e.g., about 3 days before) administering said vaccine to the subject. In some embodiments, said vaccine is administered in a prime-boost regimen, and said one or more antibodies are administered after administering prime but before (e.g., about 3 days before) administering boost of said vaccine to the subject.

In some embodiments of any of the above methods of the invention, said one or more undesirable epitopes are immunodominant epitopes. In some embodiments, said immunodominant epitopes are less conserved than other epitopes of said antigen between different strains or species of a pathogen from which said antigen is derived.

In some embodiments of any of the above methods of the invention, the antigen is a protein antigen.

In some embodiments of any of the above methods of the invention, the antigen is derived from a Class I pathogen.

In some embodiments of any of the above methods of the invention, the antigen is derived from a Class II pathogen. In some embodiments, said pathogen is a virus. In some embodiments, said virus is a coronavirus. In some embodiments, said coronavirus is SARS-CoV-2. In some embodiments, said antigen is SARS-CoV-2 spike glycoprotein and said one or more undesirable epitopes are neutralizing epitopes comprised within receptor binding domain (RBD) of said SARS-CoV-2 spike glycoprotein.

In a further aspect, the invention provides a method for shielding one or more undesirable epitopes of an antigen from recognition by the immune system in a subject, said method comprising administering to the subject an effective amount of one or more antibodies targeting said one or more undesirable epitopes. In some embodiments, said antigen is an endogenous molecule (e.g., protein) of a subject. In some embodiments, said antigen is targeted by an immune response in an autoimmune disease.

In some embodiments of any of the above methods of the invention, said one or more antibodies are monoclonal antibodies (mAbs).

In another aspect, the invention provides a composition comprising two or more monoclonal antibodies (mAbs) targeting undesirable epitopes of an antigen. In another aspect, the invention provides a composition comprising two or more monoclonal antibodies (mAbs) targeting a combination of desirable and undesirable epitopes of an antigen. In some embodiments, said undesirable epitopes are immunodominant epitopes. In some embodiments, said immunodominant epitopes are less conserved than other epitopes of said antigen between different strains or species of a pathogen from which said antigen is derived. In some embodiments, the antigen is a protein antigen. In some embodiments, the antigen is derived from a Class I pathogen. In some embodiments, the antigen is derived from a Class II pathogen. In some embodiments, said pathogen is a virus. In some embodiments, said virus is a coronavirus. In some embodiments, said coronavirus is SARS-CoV-2. In some embodiments, said antigen is SARS-CoV-2 spike glycoprotein and said undesirable epitopes are neutralizing epitopes comprised within receptor binding domain (RBD) of said SARS-CoV-2 spike glycoprotein. In some embodiments, said antigen is a molecule (e.g., protein) targeted by an immune response in an autoimmune disease.

These and other aspects described herein will be apparent to those of ordinary skill in the art in the following description, claims and drawings.

### BRIEF DESCRIPTION OF THE DRAWINGS

FIG. 1 is a schematic of antibody blockade during antigen immunization and resulting antibody responses as described herein.

FIG. 2 is a schematic of a study design to assess antibody responses with or without anti-αSARS-CoV-2 (alpha severe acute respiratory syndrome coronavirus 2) receptor-binding domain (RBD) monoclonal antibody (mAb) treatment during SARS-CoV-2 spike or RBD immunization.

FIGS. 3A-3E show Immunoglobulin G (IgG) binding levels at day 42 (three weeks post-boost) across all SARS-CoV-2 spike regions from mice pre-treated with anti-αSARS-CoV-2 RBD mAbs (E10933 and E10987) before the priming immunization (day-3, empty circle symbols) of SARS-CoV-2 spike trimer, RBD, or phosphate buffered saline (PBS) or before the booster immunization (day 18, filled circle symbols). A subset of mice received no mAb treatment (empty square symbols). Panels depict the antigen-specific IgG responses to certain spike regions: RBD (FIG. 3A), spike trimer (FIG. 3B), S1 (FIG. 3C), N Protein N-Terminal Domain (NTD) (FIG. 3D), and S2 (FIG. 3E). Numbers depict mean fluorescent intensity (MFI) IgG levels for each group.

FIG. 4 depicts SARS-CoV-2 spike pseudoviral neutralization titer (pVNT50) responses at day 42 from SARS-CoV-2 spike trimer, RBD, or PBS prime, boosted immunized mice. Mice were pre-treated with anti-αSARS-CoV-2 RBD mAbs (E10933 and E10987) before the priming immunization (day-3, empty circle symbols) of SARS-CoV-2 spike trimer, RBD, or PBS before the booster immunization (day 18, filled circle symbols). A subset of mice received no mAb treatment (empty square symbols). Numbers depict mean pVNT50 titers for each group.

FIGS. 5A-5B illustrate correlation analysis of anti-αSARS-CoV-2 RBD antibody levels to pVNT50 titers at day 42 from SARS-CoV-2 spike trimer (FIG. 5A) and RBD (FIG. 5B) prime, boosted immunized mice. Mice were pre-treated with anti-αSARS-CoV-2 RBD mAb (E10933 and E10987) before the priming immunization (day-3, empty circle symbols) of SARS-CoV-2 spike trimer, RBD, or PBS or before the booster immunization (day 18, filled circle symbols). A subset of mice received no mAb treatment (empty square symbols).

FIGS. 6A-6H show specific binding responses of anti-SARS-CoV-2 neutralizing mAbs to various SARS-CoV-2 Variants of Concern (VOC) spike proteins (wild type, FIG. 6A; Omicron BA.1, FIG. 6B; Omicron BA.2, FIG. 6C; Omicron BA.3, FIG. 6D; Alpha, FIG. 6E; Beta, FIG. 6F; Delta, FIG. 6G; and, Gamma, FIG. 6H). Numbers depict mean MFI IgG levels for each mAb.

FIG. 7 depicts a study design to assess E10933 and E10987 dose titration on skewing antibody responses to SARS-CoV-2 spike immunization.

FIGS. 8A-8B display SARS-CoV-2 spike pseudoviral neutralization titers (pVNT50) (FIG. 8A), and IgG binding levels to RBD (FIG. 8B) at day 42 (three weeks post-boost) from mice pre-treated with anti-αSARS-CoV-2 RBD mAb from 10 mg/kg to 0.0001 mg/kg (E10933 and E10987, square symbols), isotype control mAb at 10 mg/kg (E1932, black symbols) or PBS (open symbols) before the priming immunization (day-3) with SARS-CoV-2 spike trimer. All mice received a booster at D21 with the same vaccination formulation. Numbers depict mean pVNT50s or mean MFI IgG levels for each group.

FIG. 9 shows an immunization scheme described herein.

FIGS. 10A-10B depicts serum titers against SARS-CoV-2 spike RBD of VelocImmune (VI) mice with or without pre-dosed human anti-SARS-CoV-2 antibodies. FIG. 10A shows titers against SARS-CoV-2 spike protein (RBD).mmH with hIgG depletion. FIG. 10B shows titers against SARS-CoV-2 spike protein (RBD).mmH without hIgG depletion. Mice were pre-treated with anti-SARS-CoV-2 spike mAbs prior to immunization while a control cohort that did not receive mAbs. (1) Control, no mAb treatment (saline); pre-treatment with (2) E15160+E14315; (3) E15160; (4) E14315; or (5) E10933+E10987. Antibody titers were assayed with (a) or without (b) depletion of dosed human mAbs antibodies to the RBD protein.

FIG. 11 shows mouse anti-human antibody (MAHA) titers from mice pre-treated with SARS-CoV-2 spike mAb. (1) Control, no mAb treatment (saline); pre-treatment with (2) E15160+E14315; (3) E15160; (4) E14315; or (5) E10933+E10987. Antibody titers were assayed on plates coated with respective anti-SARS-CoV-2 human mAbs.

FIG. 12 shows anti-SARS-CoV-2 Spike specific hIgG levels (μg/ml) in antisera from mice pre-treated with SARS-CoV-2 Spike mAb. (1) Control, no mAb treatment (saline); pre-treatment with (2) E15160+E14315; (3) E15160; (4) E14315; or (5) E10933+E10987. Antibody titers were assayed on plates coated with respective anti-SARS-CoV-2 human mAbs. *BDL (below detection limit) data are not shown in scatter plot.

FIGS. 13A-13D show percentage inhibition on binding to surface captured SARS-CoV-2 RBD protein of individual mAb derived from different pre-treatment immunization arms by RBD pre-complexed E10933 (FIG. 13A), E10987 (FIG. 13B), E14315 (FIG. 13C), or E15160 (FIG. 13D). Value on the top of each graph are the percentage of the total mAbs derived from each pre-treatment arm that were blocked >50% by RBD pre-complexed E10933 mAb-1 (FIG. 13A), E10987 mAb-1 (FIG. 13B), E10987 mAb-1 (FIG. 13C) and E15160 mAb-1 (FIG. 13D).

FIG. 14 shows an example of a study design to modulate influenza hemagglutinin (HA) antibody responses in which mice are pre-dosed with mAb1 which has specificity to sialic-acid, receptor binding site (RBS) on the HA head or mAb 2 which binds the HA head outside of the RBS. Mice are subsequently immunized with an HA trimeric protein of H3 serotype from A/Perth/16/2009 (H3N2). At end of study hemagglutinin inhibition serum titers (HAI) from immunized mice are assessed (i.e., serum antibodies that bind to the RBS on HA from influenza and inhibit agglutination of red blood cells). Mice dosed with mAb 1 or combination of mAb 1 and mAb 2 are expected to not elicit HAI serum titers due to mAb 1 blocking the RBS site during immunization and thus inhibiting antibodies specific to that site. Additionally, sera from these mice will be assessed for anti-HA IgG binding titers across different Influenza HA serotypes to determine cross-reactivity. Mice dosed with combination of mAb 1 and mAb 2 may block B-cell immunity to the HA head, directing immunity down to the stem portion of HA which is more conserved across HA serotypes and sites for broadly neutralizing antibodies.

### DETAILED DESCRIPTION

An immune response against surface-exposed antigens is typically most effective against an infection. At the same time, because of this immune response, such surface exposed antigens are under constant evolutionary pressure to evolve and evade the immune system. Thus, a vaccine that elicits an immune response against a specific strain of pathogen may be extremely effective against that strain, but poorly effective against variant strains. To account for the evolution of virulent strains, a vaccine may have to target multiple antigens, target new antigens as the pathogen evolves, or target conserved antigens.

A separate problem in vaccine design is that some epitopes elicit an undesirable immune response. For example, inducing non-neutralizing antibodies can enhance Fc-mediated infection of macrophages, which is the mechanism behind Dengue shock syndrome. Another problem is the induction of an immune response that cross reacts with host antigens. This phenomenon can be seen in Guillain-Barré syndrome, which is associated with Campylobacter infection, but is also associated with influenza infection. Guillain-Barré syndrome was a reported side-effect of the 1976 swine flu vaccination program. Accordingly, the selection of epitopes for vaccines is far from routine.

The vaccine-induced polyclonal antibody response can often be targeted to a few immunodominant epitopes or epitopes associated with suboptimal antibody properties, such as the immunodominant “head” epitope of the influenza hemagglutinin (HA) antigen.

The present disclosure provides methods and compositions for directing an antibody response in a subject from one or more first epitopes of an antigen (e.g., immunodominant epitopes of a vaccine antigen which are less conserved between different strains or species of a pathogen from which the antigen is derived) and towards one or more second epitopes of the antigen by administering one or more antibodies (e.g., monoclonal antibodies (mAbs)) targeting said one or more first epitopes. Without wishing to be bound by any specific theory, the antibodies likely block the exposure of the undesirable epitopes to B cell receptors (BCRs) and subsequent generation or amplification of antibodies targeting those epitopes. This antibody-mediated epitope blockade can therefore steer the immune responses to alternative, exposed (non-antibody blocked) epitopes, and thus shape the resulting antibody response to desired antigen epitopes.

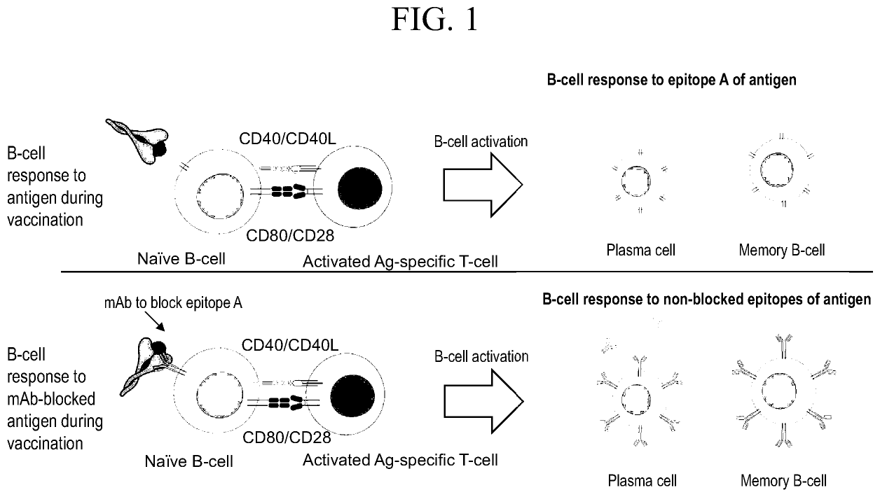

A non-limiting embodiment of the above-described disclosure is displayed in FIG. 1. The upper panel of FIG. 1 shows a typical B-cell response being generated to an antigen during vaccination based on immunodominant epitopes, which are inherent to the antigen. Naïve B-cells that have B-cell receptors (BCRs) to these immunodominant epitopes can quickly bind to the epitopes and are subsequently activated by T-cells. Activation of the BCRs establishes an effector and memory B-cell and antibody response to that epitope that may dominate the host's immune response. In the context of the present disclosure, the lower panel demonstrates that inclusion of antigen-specific mAbs that bind to certain epitopes will block those epitopes from BCR recognition, allowing for other naïve B-cells with BCRs outside of the blocked epitope to bind and subsequently to get activated. This would allow the host to establish B-cell and antibody immunity outside the blocked epitope.

### Definitions

Unless defined otherwise, all technical and scientific terms used herein have the same meaning as commonly understood by one of ordinary skill in the art to which this disclosure belongs.

Singular forms “a”, “an”, and “the” include plural references unless the context clearly dictates otherwise. Thus, for example, a reference to “a method” includes one or more methods, and/or steps of the type described herein and/or which will become apparent to those persons skilled in the art upon reading this disclosure.

The term “about” or “approximately” includes being within a statistically meaningful range of a value. Such a range can be within an order of magnitude, preferably within 50%, more preferably within 20%, still more preferably within 10%, and even more preferably within 5% of a given value or range. The allowable variation encompassed by the term “about” or “approximately” depends on the particular system under study, and can be readily appreciated by one of ordinary skill in the art.

The terms “comprise(s),” “include(s),” “having,” “has,” and “contain(s),” are intended to be open-ended transitional phrases, terms, or words that do not preclude the possibility of additional acts or structures.

The term “antigen” as used herein refers to a substance such as a protein, polypeptide, peptide, polysaccharide, glycoprotein, glycolipid, nucleotide, portions thereof, or combinations thereof, which elicits an immune response, e.g., elicits an immune response when present in a subject (for example, when present in a human or mammalian subject).

“Antibody” as used herein encompasses polyclonal and monoclonal antibodies and refers to immunoglobulin molecules of classes IgA (e.g., IgA1 or IgA2), IgD, IgE, IgG (e.g., IgG1, IgG2, IgG3 and IgG4) or IgM, or fragments, or derivatives thereof, including without limitation Fab, F(ab′)2, Fd, single chain antibodies, diabodies, bispecific antibodies, bifunctional antibodies, humanized antibodies, and various derivatives thereof.

The terms “antigen-binding portion” or “antigen-binding fragment” of an antibody or antigen-binding protein, and the like, as used herein, include any naturally occurring, enzymatically obtainable, synthetic, or genetically engineered polypeptide or glycoprotein that specifically binds an antigen to form a complex. Non-limiting examples of antigen-binding fragments include: (i) Fab fragments; (ii) F(ab′)2 fragments; (iii) Fd fragments; (iv) Fv fragments; (v) single-chain Fv (scFv) molecules; (vi) dAb fragments; and (vii) minimal recognition units consisting of the amino acid residues that mimic the hypervariable region of an antibody (e.g., an isolated complementarity determining region (CDR) such as a CDR3 peptide), or a constrained FR3-CDR3-FR4 peptide. Other engineered molecules, such as domain-specific antibodies, single domain antibodies, domain-deleted antibodies, chimeric antibodies, CDR-grafted antibodies, diabodies, triabodies, tetrabodies, minibodies, nanobodies (e.g., monovalent nanobodies, bivalent nanobodies, etc.), small modular immunopharmaceuticals (SMIPs), and shark variable IgNAR domains, are also encompassed within the expression “antigen-binding fragment,” as used herein.

An antigen-binding fragment of an antibody will, in some embodiments of the disclosure, comprise at least one variable domain. The variable domain may be of any size or amino acid composition and will generally comprise at least one CDR, which is adjacent to or in frame with one or more framework sequences. In antigen-binding fragments having a VH domain associated with a VL domain, the VH and VL domains may be situated relative to one another in any suitable arrangement. For example, the variable region may be dimeric and contain VH-VH, VH-VL or VL-VL dimers. Alternatively, the antigen-binding fragment of an antibody may contain a monomeric VH or VL domain. In certain embodiments, an antigen-binding fragment of an antibody may contain at least one variable domain covalently linked to at least one constant domain. Non-limiting, exemplary configurations of variable and constant domains that may be found within an antigen-binding fragment of an antibody of the present disclosure include: (i) VH-CH1; (ii) VH-CH2; (iii) VH-CH3; (iv) VH-CH1-CH2; (v) VH-CH1-CH2-CH3; (vi) VH-CH2-CH3; (vii) VH-CL; (viii) VL-CH1; (ix) VL-CH2; (x) VL-CH3; (xi) VL-CH1-CH2; (xii) VL-CH1-CH2-CH3; (xiii) VL-CH2-CH3; and (xiv) VL-CL. In any configuration of variable and constant domains, including any of the exemplary configurations listed above, the variable and constant domains may be either directly linked to one another or may be linked by a full or partial hinge or linker region. A hinge region may consist of at least 2 (e.g., 5, 10, 15, 20, 40, 60 or more) amino acids, which result in a flexible or semi-flexible linkage between adjacent variable and/or constant domains in a single polypeptide molecule. Moreover, an antigen-binding fragment of an antibody of the present disclosure may comprise a homodimer or heterodimer (or other multimer) of any of the variable and constant domain configurations listed above in non-covalent association with one another and/or with one or more monomeric VH or VL domain (e.g., by disulfide bond(s)).

In certain embodiments of the disclosure, the antibodies are human antibodies. The term “human antibody” is intended to include antibodies having variable and constant regions derived from human germline immunoglobulin sequences. The human antibodies may include amino acid residues not encoded by human germline immunoglobulin sequences (e.g., mutations introduced by random or site-specific mutagenesis in vitro or by somatic mutation in vivo), for example in the CDRs and in particular CDR3. However, the term “human antibody”, as used herein, is not intended to include antibodies in which CDR sequences derived from the germline of another mammalian species, such as a mouse, have been grafted onto human framework sequences.

The antibodies discussed herein may, in some embodiments, be recombinant human antibodies. The term “recombinant human antibody” is intended to include all human antibodies that are prepared, expressed, created or isolated by recombinant means, such as antibodies expressed using a recombinant expression vector transfected into a host cell, antibodies isolated from a recombinant, combinatorial human antibody library, antibodies isolated from an animal (e.g., a mouse) that is transgenic for human immunoglobulin genes (see e.g., Taylor et al. (1992) Nucl. Acids Res. 20:6287-6295) or antibodies prepared, expressed, created or isolated by any other means that involves splicing of human immunoglobulin gene sequences to other DNA sequences. Such recombinant human antibodies have variable and constant regions derived from human germline immunoglobulin sequences. In certain embodiments, however, such recombinant human antibodies are subjected to in vitro mutagenesis (or, when an animal transgenic for human Ig sequences is used, in vivo somatic mutagenesis) and thus the amino acid sequences of the VH and VL regions of the recombinant antibodies are sequences that, while derived from and related to human germline VH and VL sequences, may not naturally exist within the human antibody germline repertoire in vivo.

In the context of the present disclosure, the term “neutralizing antibody” or “nAb” refers to an antibody, or antigen-binding fragment that binds to a pathogen (e.g., a virus) and interferes with its ability to infect a cell. Non-limiting examples of neutralizing antibodies include antibodies that bind to a viral particle and inhibit successful transduction, e.g., one or more steps selected from binding, entry, trafficking to the nucleus, and transcription of the viral genome. Some neutralizing antibodies may block a virus at the post-entry step. In the context of specific embodiments of the present disclosure, a “neutralizing” or anti-spike glycoprotein antigen-binding protein, e.g., antibody or antigen-binding fragment, may refer to a molecule that inhibits an activity of spike glycoprotein, e.g., inhibits the ability of spike glycoprotein to bind to a receptor such as ACE2, to be cleaved by a protease such as TMPRSS2, or to mediate viral entry into a host cell or viral reproduction in a host cell.

“Antibody-producing cells” and “cells expressing antibodies” disclosed herein can encompass cells in which the antibodies expressed are bound to or anchored in the cell membrane, i.e., cell surface antibodies, as well as cells that secrete antibody.

The term “immune response” refers to a response of a cell of the immune system (e.g., a B-cell, T-cell, macrophage or polymorphonucleocyte) to a stimulus such as an antigen (e.g., a viral antigen). Active immune responses can involve differentiation and proliferation of immunocompetent cells, which leads to synthesis of antibodies or the development of cell-mediated reactivity, or both. An active immune response can be mounted by the host after exposure to an antigen (e.g., by infection or by vaccination). Active immune response can be contrasted with passive immunity, which can be acquired through the transfer of substances such as, e.g., an antibody, transfer factor, thymic graft, and/or cytokines from an actively immunized host to a non-immune host.

As used herein in connection with a viral infection and vaccination, the terms “protective immune response” or “protective immunity” refer to an immune response that confers some benefit to the subject in that it prevents or reduces the infection or prevents or reduces the development of a disease associated with the infection.

The terms “immunogenic composition”, “vaccine composition”, or “vaccine”, which are used interchangeably, refer to a composition comprising at least one immunogenic and/or antigenic component that induces an immune response in a subject (e.g., humoral and/or cellular response). In certain embodiments, the immune response is a protective immune response. A vaccine may be administered for the prevention or treatment of a disease, such as an infectious disease. A vaccine composition may include, for example, live or killed infectious agents, recombinant infectious agents (e.g., recombinant viral particles, virus-like particles, nanoparticles, liposomes, or cells expressing immunogenic and/or antigenic components), antigenic proteins or peptides, nucleic acids, etc. Vaccines may be administered with an adjuvant to boost the immune response.

The term “epitope” refers to an antigenic determinant that interacts with a specific antigen-binding site of an antigen-binding protein, e.g., a variable region of an antibody molecule, known as a paratope.

The term “immunodominant epitope” refers to an epitope within an antigen that selectively provokes an immune response in a host to the effective or functional exclusion, which may be partial or complete, of other epitopes of that antigen.

The term “Class I pathogens” refers to pathogens which have one or more of the following properties: (1) infect narrow age range; (2) host exhibits spontaneous recovery; (3) host generates long lasting protective immunity; (4) pathogen is genetically stable with limited antigenic variation; (5) immune responses are directed to multiple epitopes.

The term “Class II pathogens” refers to pathogens which have one or more of the following properties: (1) pathogen infects wide age range; (2) pathogens frequently persist as latent infections; (3) no or low long-lasting protective immunity; (4) priming with wild-type antigens offer little protection or strain-specific protection; (5) pathogen exhibits high mutation rate and tolerates high degree of variation in epitope regions; (6) immune responses are limited to a smaller number of genetically variable and strain-specific epitopes and suggest early cross-reactive recall.

The terms “derivative” and “variant” are used herein interchangeably to refer to an entity that has significant structural identity with a reference entity but differs structurally from the reference entity in the presence or level of one or more chemical moieties as compared with the reference entity. In many embodiments, a derivative also differs functionally from its reference entity. In general, whether a particular entity is properly considered to be a “derivative” of a reference entity is based on its degree of structural identity with the reference entity. As will be appreciated by those skilled in the art, any biological or chemical reference entity has certain characteristic structural elements. A derivative, by definition, is a distinct entity that shares one or more such characteristic structural elements. To give but a few examples, a small molecule may have a characteristic core structural element (e.g., a macrocycle core) and/or one or more characteristic pendent moieties so that a derivative of the small molecule is one that shares the core structural element and the characteristic pendent moieties but differs in other pendent moieties and/or in types of bonds present (single vs double, E vs Z, etc.) within the core. A derivative nucleic acid may have a characteristic sequence element comprised of a plurality of nucleotide residues having designated positions relative to one another in linear or three-dimensional space. In some embodiments, the nucleic acid sequence of a derivative may be 50%, 60%, 70%, 80%, 81%, 82%, 83%, 84%, 85%, 86%, 87%, 88%, 89%, 90%, 91%, 92%, 93%, 94%, 95%, 96%, 97%, 98%, 99%, 99.5%, or more identical over the full length of the reference sequence or a fragment thereof. A derivative peptide or polypeptide may have a characteristic sequence element comprised of a plurality of amino acids having designated positions relative to one another in linear or three-dimensional space and/or contributing to a particular biological function. Derivative peptides and polypeptides include peptides and polypeptides that differ in amino acid sequence from the reference peptide or polypeptide by the insertion, deletion, and/or substitution of one or more amino acids, but retain at least one biological activity of such reference peptide or polypeptide (e.g., the ability to mediate cell infection by a virus, the ability to mediate membrane fusion, the ability to be bound by a specific antibody or to promote an immune response, etc.). In some non-limiting embodiments, a derivative peptide or polypeptide shows the sequence identity over the full length with the reference peptide or polypeptide (or a fragment thereof) that is at least 50%, 60%, 70%, 80%, 81%, 82%, 83%, 84%, 85%, 86%, 87%, 88%, 89%, 90%, 91%, 92%, 93%, 94%, 95%, 96%, 97%, 98%, 99%, 99.5%, or more. Alternatively, or in addition, a derivative peptide or polypeptide may differ from a reference peptide or polypeptide as a result of one or more and/or one or more differences in chemical moieties attached to the polypeptide backbone (e.g., in glycosylation, phosphorylation, acetylation, myristoylation, palmitoylation, oxidation, formylation, amidation, polyglutamylation, ADP-ribosylation, pegylation, biotinylation, etc.). In some embodiments, a derivative peptide or polypeptide lacks one or more of the biological activities of the reference polypeptide or has a reduced or increased level of one or more biological activities as compared with the reference polypeptide. Derivatives of a particular peptide or polypeptide may be found in nature or may be synthetically or recombinantly produced. As used herein, the term “derivative” or “variant” also encompassed various fusion proteins and conjugates, including fusions or conjugates with detection tags (e.g., HA tag, histidine tag, biotin, fusions with fluorescent or luminescent domains, etc.), dimerization/multimerization sequences, Fc, signaling sequences, etc.

The term “coronavirus” as used herein refers to any virus of the subfamily Coronavirinae within the family Coronaviridae, within the order Nidovirales. Non-limiting examples a coronavirus include SARS-CoV-2, MERS-COV, and SARS-CoV.

The term “CoV-S” or “S protein” or “spike protein” or “spike glycoprotein” or “S glycoprotein”, and the like, refers to the spike protein of a coronavirus and includes protein variants of the spike protein. A spike protein disclosed herein cam be specific S proteins such as SARS-CoV-2 S protein, MERS-COV S protein, and SARS-CoV S protein. In the context of the present disclosure, a spike protein may be isolated from different SARS-CoV-2 isolates, as well as recombinant SARS-CoV-2 spike protein or a fragment thereof.

The term “coronavirus infection” or “CoV infection” or “SARS-CoV-2 infection” as used herein, refers to infection with a coronavirus such as SARS-CoV-2, MERS-COV, or SARS-CoV. The term includes coronavirus respiratory tract infections, often in the lower respiratory tract. Symptoms can include high fever, dry cough, shortness of breath, pneumonia, gastro-intestinal symptoms such as diarrhea, organ failure (kidney failure and renal dysfunction), septic shock, and death in severe cases.

The term “encoding” can refer to encoding from either the (+) or (−) sense strand of the polynucleotide for expression in the virus particle.

The terms “protein” and “polypeptide”, used interchangeably herein, encompass all kinds of naturally occurring and synthetic proteins, including protein fragments of all lengths, fusion proteins and modified proteins, including without limitation, glycoproteins, as well as all other types of modified proteins (e.g., proteins resulting from phosphorylation, acetylation, myristoylation, palmitoylation, glycosylation, oxidation, formylation, amidation, polyglutamylation, ADP-ribosylation, PEGylation, biotinylation, etc.). Small polypeptides of less than 100 amino acids, preferably less than 50 amino acids, may be referred to as “peptides”.

The terms “polynucleotide” and “nucleic acid”, used interchangeably herein, include polymeric forms of nucleotides of any length, including ribonucleotides (RNA), deoxyribonucleotides (DNA), or analogs or modified versions thereof. They include single-, double-, and multi-stranded DNA or RNA, genomic DNA, complementary DNA (cDNA), DNA-RNA hybrids, and polymers comprising purine bases, pyrimidine bases, or other natural, chemically modified, biochemically modified, non-natural, or derivatized nucleotide bases.

The term “operably linked” or the like refers to a juxtaposition wherein the components described are in a relationship permitting them to function in their intended manner. For example, a control sequence “operably linked” to a coding sequence is ligated in such a way that expression of the coding sequence is achieved under conditions compatible with the control sequences. “Operably linked” sequences include both expression control sequences that are contiguous with a gene of interest and expression control sequences that act in trans or at a distance to control a gene of interest (or sequence of interest). The term “expression control sequence” includes polynucleotide sequences, which are necessary to affect the expression and processing of coding sequences to which they are ligated. “Expression control sequences” include appropriate transcription initiation, termination, promoter and enhancer sequences; efficient RNA processing signals such as splicing and polyadenylation signals; sequences that stabilize cytoplasmic mRNA; sequences that enhance translation efficiency (i.e., Kozak consensus sequence); sequences that enhance polypeptide stability; and when desired, sequences that enhance polypeptide secretion. The nature of such control sequences differs depending upon the host organism. For example, in prokaryotes, such control sequences generally include promoter, ribosomal binding site and transcription termination sequence, while in eukaryotes typically such control sequences include promoters and transcription termination sequence. The term “control sequences” is intended to include components whose presence is essential for expression and processing and can also include additional components whose presence is advantageous, for example, leader sequences and fusion partner sequences.

The term “isolated” refers to a homogenous population of molecules (such as polynucleotides or polypeptides) which have been substantially separated and/or purified away from other components of the system the molecules are produced in, such as a recombinant cell, as well as a protein that has been subjected to at least one purification or isolation step. “Isolated” refers to a molecule that is substantially free of other cellular material and/or chemicals and encompasses molecules that are isolated to a higher purity, such as to 80%, 81%, 82%, 83%, 84%, 85%, 86%, 87%, 88%, 89%, 90%, 91%, 92%, 93%, 94%, 95%, 96%, 97%, 98%, 99% or 100% purity.

The term “effective” applied to dose or amount refers to that quantity of a compound (e.g., a recombinant virus) or composition (e.g., pharmaceutical, vaccine or immunogenic and/or antigenic composition) that is sufficient to result in a desired activity upon administration to a subject in need thereof. Note that when a combination of active ingredients is administered, the effective amount of the combination may or may not include amounts of each ingredient that would have been effective if administered individually. The exact amount required will vary from subject to subject, depending on the species, age, and general condition of the subject, the severity of the condition being treated, the particular drug or drugs employed, the mode of administration, and the like.

The term “administration” and the like refers to and includes the administration of a composition to a subject or system (e.g., to a cell, organ, tissue, organism, or relevant component or set of components thereof). The skilled artisan will appreciate that route of administration may vary depending, for example, on the subject or system to which the composition is being administered, the nature of the composition, the purpose of the administration, etc. For example, in certain embodiments, administration to an animal subject (e.g., to a human or a rodent) may be bronchial (including by bronchial instillation), buccal, enteral, interdermal, intra-arterial, intradermal, intragastric, intramedullary, intramuscular, intranasal, intraperitoneal, intrathecal, intravenous, intraventricular, mucosal, nasal, oral, rectal, subcutaneous, sublingual, topical, tracheal (including by intratracheal instillation), transdermal, vaginal and/or vitreal. In some embodiments, administration may involve intermittent dosing. In some embodiments, administration may involve continuous dosing (e.g., perfusion) for at least a selected period of time.

In the context of the present disclosure insofar as it relates to any of the disease conditions recited herein, the terms “treat”, “treatment”, and the like mean to relieve or alleviate at least one symptom associated with such condition, or to slow or reverse the progression of such condition. Within the meaning of the present disclosure, the term “treat” also denotes to arrest, delay the onset (i.e., the period prior to clinical manifestation of a disease) and/or reduce the risk of developing or worsening a disease. The terms “treat”, “treatment”, and the like regarding a state, disorder or condition may also include (1) preventing or delaying the appearance of at least one clinical or sub-clinical symptom of the state, disorder or condition developing in a subject that may be afflicted with or predisposed to the state, disorder or condition but does not yet experience or display clinical or subclinical symptoms of the state, disorder or condition; or (2) inhibiting the state, disorder or condition, i.e., arresting, reducing or delaying the development of the disease or a relapse thereof (in case of maintenance treatment) or at least one clinical or sub-clinical symptom thereof; or (3) relieving the disease, i.e., causing regression of the state, disorder or condition or at least one of its clinical or sub-clinical symptoms. For example, in the context of SARS-CoV-2 infection, non-limiting examples of the symptoms of the COVID-19 disease, include, without limitation, fever, cough, shortness of breath, pneumonia, acute respiratory distress syndrome (ARDS), acute lung syndrome, loss of sense of smell, loss of sense of taste, sore throat, nasal discharge, gastro-intestinal symptoms (e.g., diarrhea), organ failure (e.g., kidney failure and renal dysfunction), septic shock, and death. When used in connection with a disease caused by a viral infection (e.g., SARS-CoV-2 infection, influenza infection), the terms “prevent”, “preventing” or “prevention” refer to prevention of spread of infection in a subject exposed to the virus, e.g., prevention of the virus from entering the subject's cells.

The terms “individual” or “subject” or “patient” or “animal” refers to humans, veterinary animals (e.g., cats, dogs, cows, horses, sheep, pigs, etc.) and experimental animal models of diseases (e.g., mice, rats, rabbits, ferrets, monkeys, etc.). In some embodiments, the subject is a human. In some embodiments, the subject may be in need of prevention and/or treatment of a disease or disorder such as viral infection or cancer. The subject may have a viral infection, e.g., a SARS-CoV-2 infection or an influenza infection or be predisposed to developing an infection. Subjects predisposed to developing an infection, or subjects who may be at elevated risk for contracting an infection (e.g., of coronavirus or influenza virus), include subjects with compromised immune systems because of autoimmune disease, subjects receiving immunosuppressive therapy (for example, following organ transplant), subjects afflicted with human immunodeficiency syndrome (HIV) or acquired immune deficiency syndrome (AIDS), subjects with forms of anemia that deplete or destroy white blood cells, subjects receiving radiation or chemotherapy, or subjects afflicted with an inflammatory disorder. Additionally, subjects of very young (e.g., 5 years of age or younger) or old age (e.g., 65 years of age or older) may be at increased risk. Moreover, a subject may be at risk of contracting a viral infection due to proximity to an outbreak of the disease, e.g., subject resides in a densely populated city or in close proximity to subjects having confirmed or suspected infections of a virus, or choice of employment, e.g., hospital worker, pharmaceutical researcher, traveler to infected area, or frequent flier. In some embodiments, the subject is an experimental animal (e.g., mouse, rat, rabbit, ferret, monkey, etc.). In some embodiments, the methods described herein are applied to an experimental animal (e.g., mouse, rat, rabbit, ferret, monkey, etc.) to generate therapeutic antibodies targeting one or more desirable epitope(s) of an antigen.

The phrase “pharmaceutically acceptable”, as used in connection with compositions described herein, refers to molecular entities and other ingredients of such compositions that are physiologically tolerable and do not typically produce untoward reactions when administered to a subject (e.g., a human). Preferably, the term “pharmaceutically acceptable” means approved by a regulatory agency of the Federal or a state government or listed in the U.S. Pharmacopeia or other generally recognized pharmacopeia for use in mammals, and more particularly in humans.

The practice of the present disclosure employs, unless otherwise indicated, conventional techniques of statistical analysis, molecular biology (including recombinant techniques), virology, microbiology, cell biology, chemistry and biochemistry, which are within the skill of the art. Such tools and techniques are described in detail in e.g., Sambrook, Fritsch & Maniatis, Molecular Cloning: A Laboratory Manual, Second Edition. Cold Spring Harbor, NY: Cold Spring Harbor Laboratory Press, 1989 (herein “Sambrook et al., 1989”); DNA Cloning: A Practical Approach, Volumes I and II (D. N. Glover ed. 1985); Oligonucleotide Synthesis (M. J. Gait ed. 1984); Nucleic Acid Hybridization [B. D. Hames & S. J. Higgins eds. (1985)]; Transcription And Translation [B. D. Hames & S. J. Higgins, eds. (1984)]; Animal Cell Culture [R. I. Freshney, ed. (1986)]; Immobilized Cells And Enzymes [IRL Press, (1986)]; B. Perbal, A Practical Guide To Molecular Cloning (1984); Ausubel, F. M. et al. (eds.). Current Protocols in Molecular Biology. John Wiley & Sons, Inc., 1994. These techniques include site directed mutagenesis as described in Kunkel, Proc. Natl. Acad. Sci. USA 82: 488-492 (1985), U.S. Pat. No. 5,071,743, Fukuoka et al., Biochem. Biophys. Res. Commun. 263: 357-360 (1999); Kim and Maas, BioTech. 28: 196-198 (2000); Parikh and Guengerich, BioTech. 24: 4 28-431 (1998); Ray and Nickoloff, BioTech. 13: 342-346 (1992); Wang et al., BioTech. 19: 556-559 (1995); Wang and Malcolm, BioTech. 26: 680-682 (1999); Xu and Gong, BioTech. 26: 639-641 (1999), U.S. Pat. Nos. 5,789,166 and 5,932,419, Hogrefe, Strategies 14. 3: 74-75 (2001), U.S. Pat. Nos. 5,702,931, 5,780,270, and 6,242,222, Angag and Schutz, Biotech. 30: 486-488 (2001), Wang and Wilkinson, Biotech. 29: 976-978 (2000), Kang et al., Biotech. 20: 44-46 (1996), Ogel and McPherson, Protein Engineer. 5: 467-468 (1992), Kirsch and Joly, Nucl. Acids. Res. 26: 1848-1850 (1998), Rhem and Hancock, J. Bacteriol. 178: 3346-3349 (1996), Boles and Miogsa, Curr. Genet. 28: 197-198 (1995), Barrenttino et al., Nuc. Acids. Res. 22: 541-542 (1993), Tessier and Thomas, Meths. Molec. Biol. 57: 229-237, and Pons et al., Meth. Molec. Biol. 67: 209-218.

### Antigens and Epitopes

An antigen as used in the present disclosure can be a substance such as a protein, polypeptide, peptide, polysaccharide, glycoprotein, glycolipid, nucleotide, portions thereof, or combinations thereof, which elicits an immune response, e.g., elicits an immune response when present in a subject (for example, when present in a human or mammalian subject). As a non-limiting example, when present within a cell or subject, an antigen may cause the immune system to produce an immune response to the antigen, for example by triggering the production of antibodies against the antigen, e.g., binding and/or neutralizing antibodies can trigger B cell and/or T cell responses specific to the antigen, and ultimately can cause protective or prophylactic response against subsequent encounter with the antigen or with a pathogen with which the antigen is associated.

In some embodiments, the antigen is a protein antigen. In some embodiments, the antigen disclosed herein may comprise a full-length protein, for example, a full-length viral protein, or may comprise a fragment (e.g., a polypeptide or peptide fragment, subunit or domain of a protein, e.g., a viral protein or subunit domain).

In some embodiments, the antigen is a non-protein antigen.

In some embodiments, the antigen is an endogenous molecule of the subject. In some embodiments, the antigen is targeted by an immune response in an autoimmune disease.

In some embodiments, the antigen is associated with infectious diseases, autoimmune diseases, tumor cells, and/or cells within the tumor microenvironment, extracellular matrix, or specific tissues.

Non-limiting examples of infectious-associated antigens include those derived from Coronoviridae (e.g., coronaviruses); Orthomyxoviridae (e.g., influenza viruses); Flaviridae (e.g., dengue viruses, encephalitis viruses, yellow fever viruses); Adenoviridae (most adenoviruses); Papovaviridae (papilloma viruses, polyoma viruses); Arena viridae (hemorrhagic fever viruses); Calciviridae (e.g., strains that cause gastroenteritis); Filoviridae (e.g., ebola viruses); Herpesviridae (herpes simplex virus (HSV) 1 and 2, varicella zoster virus, cytomegalovirus (CMV), herpes virus); Iridoviridae (e.g., African swine fever virus); Paramyxoviridae (e.g., parainfluenza viruses, mumps virus, measles virus, respiratory syncytial virus); Hepadnaviridae (Hepatitis B virus; HBsAg); Reoviridae (e.g., reoviruses, orbiviurses and rotaviruses); Retroviridae (e.g., human immunodeficiency viruses, such as HIV-1 (also referred to as HTLV-III, LAV or HTLV-III/LAV, or HIV-III); and other isolates, such as HIV-LP), Norwalk and related viruses, and astroviruses; Birnaviridae; Bungaviridae (e.g., Hantaan viruses, bunga viruses, phleboviruses and Nairo viruses); Parvovirida (parvoviruses); Picornaviridae (e.g., polio viruses, hepatitis A virus; Togaviridae (e.g., equine encephalitis viruses, rubella viruses); enteroviruses, human Coxsackie viruses, rhinoviruses, echoviruses); Poxviridae (variola viruses, vaccinia viruses, pox viruses); Papillomaviridae (e.g., papillomavirus); Rhabdoviradae (e.g., vesicular stomatitis viruses, rabies viruses); and unclassified viruses (e.g., the agents of non-A, non-B hepatitis (i.e. Hepatitis C) the agent of delta hepatitis, the agents of Spongiform encephalopathies).

Additional viral antigens may be derived from a strain of virus selected from: Varicella-zoster virus; Epstein-barr virus; Human cytomegalovirus; Human herpes virus, type 8; BK virus; JC virus; Smallpox; polio virus; Hepatitis B virus; Human bocavirus; Parvovirus B19; Human astrovirus; Norwalk virus; coxsackievirus; hepatitis A virus; poliovirus; rhinovirus; Severe acute respiratory syndrome virus; Hepatitis C virus; Yellow Fever virus; Dengue virus; West Nile virus; Rubella virus; Hepatitis E virus; Human Immunodeficiency virus (HIV); Influenza virus; Guanarito virus; Junin virus; Lassa virus; Machupo virus; Sabiá virus; Crimean-Congo hemorrhagic fever virus; Ebola virus; Marburg virus; Measles virus; Mumps virus; Parainfluenza virus; Respiratory syncytial virus; Human metapneumovirus; Hendra virus; Nipah virus; Rabies virus; Hepatitis D; Rotavirus; Orbivirus; Coltivirus; Banna virus; Human Enterovirus; Hanta virus; West Nile virus; Middle East Respiratory Syndrome Corona Virus; Japanese encephalitis virus; Vesicular exanthernavirus; and Eastern equine encephalitis.

Additional infectious antigens include bacterial antigens, fungal antigens, parasite antigens, or prion antigens, or the like. Non-limiting examples of infectious bacteria include but are not limited to: Streptococcus (viridans group), Streptococcus agalactiae (Group B Streptococcus), Streptococcus bovis, Streptococcus faecalis, Streptococcus pneumoniae, Streptococcus pyogenes (Group A Streptococcus), Bacteroides sp., Borelia burgdorferi, Chlamydia., Clostridium perfringers, Clostridium tetani, Enterobacter aerogenes, Enterococcus faecium, Enterococcus sp., Erysipelothrix rhusiopathiae, Neisseria meningitidis, Actinomyces israelli, Fusobacterium nucleatum, Treponema pallidium, and Treponema pertenue, pathogenic Campylobacter sp., Rickettsia, Staphylococcus aureus, Streptobacillus monihformis, Streptococcus (anaerobic sps.), Haemophilus influenzae, Helicobacter pyloris, Klebsiella pneumoniae, Legionella pneumophilia, Leptospira, Corynebacterium diphtheriae, Corynebacterium sp., Listeria monocytogenes, Mycobacteria sps. (e.g., M tuberculosis, M avium, M gordonae, M intracellulare, M kansaii), Neisseria gonorrhoeae, Bacillus antracis, Pseudomonas aeruginosa or Pasteurella multocida. Infectious fungi include, for example, Coccidioides immitis, Blastomyces dernatitidis, Cryptococcus neoformans, Histoplasma capsulatuin, Chlamydia trachomatis and Candida albicans. Addional infectious organisms (i.e., protists) include Plasmodium e.g., Plasmodium ovale, Plasmodium falciparum, Plasmodium malariae, Plasmodium vivax, Toxoplasma gondii and Shistosoma.

In some embodiments, the antigen is associated with an autoimmune disease or disorder. An antigen associated with an autoimmune disease or disorder may be derived, for example, from cell receptors and/or cells which produce “self”-directed antibodies. In some embodiments, the antigen is associated with an autoimmune disease or disorder such as, e.g., vasculitis, Wegener's granulomatosis, Hashimoto's thyroiditis, psoriasis Graves' disease, Guillain-Barré syndrome, chronic inflammatory demyelinating polyneuropathy Crohn's disease, ulcerative colitis, Rheumatoid arthritis (RA), multiple sclerosis (MS), Sjögren's syndrome, sarcoidosis, Systemic lupus erythematosus, Type 1 diabetes mellitus, insulin dependent diabetes mellitus (IDDM), autoimmune thyroiditis, reactive arthritis, Myasthenia gravis, ankylosing spondylitis, scleroderma, polymyositis, or dermatomyositis.

Non-limiting examples of autoimmune antigens include platelet antigen, islet cell antigen, myelin protein antigen, Rheumatoid factor, anticitrullinated protein, glucose-6-phosphate isomerase, receptors such as lipocortin 1, neutrophil nuclear proteins such as lactoferrin and 25-35 kD nuclear protein, Sm antigens, e.g., in snRNPs, granular proteins such as bactericidal permeability increasing protein (BPI), elastase, fibrin, vimentin, filaggrin, fibrinogen, collagen I and II peptides, plasminogen, alpha-enolase, translation initiation factor 4G1, perinuclear factor, keratin, Sa (cytoskeletal protein vimentin), citrullinated proteins and peptides such as CCP-1, CCP-2 (cyclical citrullinated peptides), circulating serum proteins such as RFs (IgG, IgM), components of articular cartilage such as collagen II, IX, and XI, nuclear components such as RA33/hnRNP A2, ferritin, stress proteins such as HSP-65,-70,-90, BiP, inflammatory/immune factors such as B7-H1, IL-1 alpha, and IL-8, enzymes such as alpha-enolase, calpastatin, dipeptidyl peptidase, eukaryotic translation elongation factor 1 alpha 1 aldolase-A, osteopontin, cathepsin G, myeloperoxidase, proteinase 3 antigen, rheumatoid factor, histones, nucleic acids such as, RNA, dsDNA, ssDNA, and ribonuclear particles, ribosomal P proteins, myelin protein, cardiolipin, vimentin, Sm antigens (including, e.g., SmD's and SmB′/B), U1RNP, A2/B1 hnRNP, Ro (SSA), and La (SSB) antigens.

In some embodiments, the antigen is an endogenous molecule of a subject. In some embodiments, the antigen is targeted by an immune response in an autoimmune disease disclosed herein.

In some embodiments, the antigen can be a tumor antigen. In some embodiments, the tumor antigen is associated with ovarian cancer, cervical cancer glioblastoma, bladder cancer, head and neck cancer, liver cancer, pancreatic cancer, prostate cancer, renal cell carcinoma or hematologic malignancy.

Non-limiting examples of tumor antigens include 5T4, 707-AP, AFP, ART-4, B7-H3, B7H4, BAGE, BCMA, Bcrabl, CA125, CAMEL, CAP-1, CASP-8, CD 30, CD133, CD19, CD20, CD22, CD25, CD33, CD4, CD52, CD56, CD70, CD79, CD80, CDC27/m, CDK4/m, CEA, Claudin 18.2, CLL1, cMET, CT, Cyp-B, DAM, EGFR, EGFRvIII, ELF2M, EMMPRIN, EpCam, EpCAM, EpCAM, ErbB3, ETV6-AML1, FGFR1, FGFR3, FOLR1, FSHR, G250, GAGE, GD2, GnT-V, Gp100, GPC-3, GPRC5D, HAGE, HAST-2, HER-2/neu, HLA-A* 0201-R170I, HPV-E7, HSP70-2M, hTERT (or hTRT), iCE, IGF-1R, IL13Rα2, IL-2R, IL-5, KIAA0205, LAGE, LDLR/FUT, MAGE, MART-1/Melan-A, MART-2/Ski, MC1R, Mesothelin, MET, MN/C IX-antigen, MUC1, MUC16, MUM-1, MUM-2, MUM-3, myosin/m, NA88-A, Nectin-4, SLITRK6, NY-ESO1, NY-Eso-1, NY-Eso-B, p190 minor bcr-abl, PAP, PDGFRα, Pm1/RARα, PRAME, proteinase-3, PSA, PSM, PSMA, RAGE, ROBO1, RU1, RU2, SAGE, SART-1, SART-3, SLAM F7, survivin, TEL/AML1, TGFβ, TPI/m, TRP-1, TRP-2, TRP-2/INT2, VEGF, WT1, α5β1-integrin, and β-catenin/m.

In some embodiments, the antigen may be derived from a Class I pathogen. A Class I pathogens disclosed herein may have one or more of the following properties: (1) infect narrow age range; (2) host exhibits spontaneous recovery; (3) host generates long lasting protective immunity; (4) pathogen is genetically stable with limited antigenic variation; (5) immune responses are directed to multiple epitopes. Non-limiting examples of Class I pathogens include viruses such as, e.g., measles, mumps rubella, diphtheria, Canine distemper, rabies, and poliovirus. See, e.g., Tobin et al., Vaccine, 2008, 26:6189-6199.

In some embodiments, the antigen may be derived from a Class II pathogen. A Class II pathogens disclosed herein have one or more of the following properties: (1) pathogen infects wide age range; (2) pathogens frequently persist as latent infections; (3) no or low long-lasting protective immunity; (4) priming with wild-type antigens offer little protection or strain-specific protection; (5) pathogen exhibits high mutation rate and tolerates high degree of variation in epitope regions; (6) immune responses are limited to a smaller number of genetically variable and strain-specific epitopes and suggest early cross-reactive recall. Non-limiting examples of Class II pathogens include, e.g., coronaviruses such as SARS-CoV-2, influenza virus, human immunodeficiency virus type 1 (HIV-1), caprine arthritis encephalitis virus (CAEV), human rhinovirus (HRV), Foot-and-Mouth Disease virus (FMDV), Hepatitis C virus, non-typeable Haemophilus influenza viruses, malaria parasites, Mycoplasma, Trypanosomes, Schistosomes, Leishmania, Anaplasma, Enteroviruses, Astroviruses, Rhinoviruses, Norwalk viruses, toxigenic/pathogenic E. coli, Neisseria, and Streptomyces. See, e.g., Tobin et al., Vaccine, 2008, 26:6189-6199.

In some embodiments, when the antigen is derived from a pathogen disclosed herein, the pathogen can be a virus.

In some embodiments, the pathogen may be a virus.

In some embodiments, the virus may be an influenza virus. In some embodiments, the virus is a strain of Influenza A or Influenza B or combinations thereof. In some embodiments, the strain of Influenza A or Influenza B is associated with birds, pigs, horses, dogs, humans or non-human primates. In some embodiments, the antigenic polypeptide is a hemagglutinin protein or fragment thereof. In some embodiments, the hemagglutinin protein is H1, H2, H3, H4, H5, H6, H7, H8, H9, H10, H11, H12, H13, H14, H15, H16, H17, H18, or a fragment thereof. In some embodiments, the hemagglutinin protein does not comprise a head domain (HA1). In some embodiments, the hemagglutinin protein comprises a portion of the head domain (HA1). In some embodiments, the hemagglutinin protein does not comprise a cytoplasmic domain. In some embodiments, the hemagglutinin protein comprises a portion of the cytoplasmic domain. In some embodiments, the hemagglutinin protein is truncated. In some embodiments, the truncated hemagglutinin protein comprises a portion of the transmembrane domain. In some embodiments, the amino acid sequence of the hemagglutinin protein or fragment thereof comprises at least 90%, 91%, 92%, 93%, 94%, 95%, 96%, 97%98%, or 99% identify with any of the known amino acid sequences for influenza antigens.

In some embodiments the influenza virus may be an Influenza A virus such as but not limited to A/Perth/16/2009(H3N2). In certain embodiments, the antigen is influenza hemagglutinin (HA), and the one or more first epitopes are comprised within sialic-acid, receptor binding site (RBS) on the HA head. In certain embodiments, the antigen is HA trimeric protein of H3 serotype from A/Perth/16/2009. In some embodiments, the one or more epitopes is E4123 of influenza hemagglutinin (HA). In some embodiments, E4123 may be comprised within the sialic-acid, receptor binding site (RBS) on the HA head. In some embodiments of the disclosure, the antigen is influenza hemagglutinin (HA) comprising the sequence of SEQ ID NO: 19, or a fragment or derivative thereof that has at least 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.5% or more amino acid sequence identity to SEQ ID NO: 19.

In some embodiments, the virus may be a coronavirus. Without wishing to be bound by theory, coronavirus virions are spherical with diameters of approximately 125 nm. The most prominent feature of coronaviruses is the club-shape spike projections emanating from the surface of the virion. These spikes are a defining feature of the virion and give them the appearance of a solar corona, prompting the name, coronaviruses. Within the envelope of the virion is the nucleocapsid. Coronaviruses have helically symmetrical nucleocapsids, which is uncommon among positive-sense RNA viruses, but far more common for negative-sense RNA viruses. SARS-CoV-2, MERS-COV, and SARS-CoV belong to the coronavirus family. The initial attachment of the virion to the host cell is initiated by interactions between the S protein and its receptor. The sites of receptor binding domains (RBD) within the S1 region of a coronavirus S protein vary depending on the virus, with some having the RBD at the C-terminus of S1. The S-protein/receptor interaction is the primary determinant for a coronavirus to infect a host species and also governs the tissue tropism of the virus. Many coronaviruses utilize peptidases as their cellular receptor. Following receptor binding, the virus must next gain access to the host cell cytosol. This is generally accomplished by acid-dependent proteolytic cleavage of S protein by a cathepsin, TMPRRS2 or another protease, followed by fusion of the viral and cellular membranes.

A coronavirus disclosed herein can be any virus of the subfamily Coronavirinae within the family Coronaviridae, within the order Nidovirales. Based on the phylogenetic relationships and genomic structures, this subfamily consists of four genera: Alphacoronavirus, Betacoronavirus, Gammacoronavirus and Deltacoronavirus. Without wishing to be bound by theory, the alphacoronaviruses and betacoronaviruses may infect mammals. The gammacoronaviruses and deltacoronaviruses may infect birds, but some of them can also infect mammals. Alphacoronaviruses and betacoronaviruses may cause, e.g., respiratory illness in humans and gastroenteritis in animals. In some embodiments, the antibodies or antigen-binding fragments disclosed herein can bind to and/or neutralize an alphacoronavirus, a betacoronavirus, a gammacoronavirus, and/or a deltacoronavirus. In certain embodiments, this binding and/or neutralization can be specific for a particular genus of coronavirus or for a particular subgroup of a genus. The three highly pathogenic viruses, SARS-CoV-2, SARS-CoV and MERS-COV, may cause severe respiratory syndrome in humans. The other four human coronaviruses, HCoV-NL63, HCoV-229E, HCoV-OC43 and HKU1, induce only mild upper respiratory diseases in immunocompetent hosts, although some of them can cause severe infections in infants, young children and elderly individuals. Additional non-limiting examples of commercially important coronaviruses include transmissible gastroenteritis coronavirus (TGEV), porcine respiratory coronavirus, canine coronavirus, feline enteric coronavirus, feline infectious peritonitis virus, rabbit coronavirus, murine hepatitis virus, sialodacryoadenitis virus, porcine hemagglutinating encephalomyelitis virus, bovine coronavirus, avian infectious bronchitis virus, and turkey coronavirus. Reviewed in Cui et al., Nature Reviews Microbiology, 2019, 17:181-192; Fung et al., Annu. Rev. Microbiol., 2019, 73:529-557.

In some embodiments, the coronavirus is SARS-CoV-2.

Coronavirus entry into host cells is mediated by the transmembrane spike (S) glycoprotein (interchangeably referred to as “spike glycoprotein”, “S glycoprotein”, “S protein” or “spike protein”, and the like) which is the main target of anti-viral neutralizing antibodies and is the focus of therapeutic and vaccine design. S glycoprotein is a 1273 amino acid type I membrane glycoprotein which assembles into trimers that constitute the spikes or peplomers on the surface of the enveloped coronavirus particle. S glycoprotein comprises two functional subunits responsible for binding to the host cell receptor (S1 subunit) and fusion of the viral and cellular membranes (S2 subunit). For many coronaviruses, including SARS-CoV-1 and SARS-CoV-2, S glycoprotein is cleaved at the boundary between the S1 and S2 subunits, which remain non-covalently bound in the prefusion conformation. The distal S1 subunit comprises the receptor-binding domain(s) (RBD) and contributes to stabilization of the prefusion state of the membrane-anchored S2 subunit that contains the fusion machinery. S is further cleaved by host proteases at the S2′ site located immediately upstream of the fusion peptide. This cleavage has been proposed to activate the protein for membrane fusion via conformational changes. Walls et al., Cell, published online Mar. 9, 2020; available at doi.org/10.1016/j.cell.2020.02.058, which is incorporated herein by reference in its entirety.

In some embodiments, the antigen disclosed here is a SARS-CoV-2 spike glycoprotein and the one or more first epitopes are neutralizing epitopes comprised within the receptor binding domain (RBD) of the SARS-CoV-2 spike glycoprotein.

S proteins disclosed herein include protein variants of spike protein isolated from different SARS-CoV-2 isolates as well as recombinant SARS-CoV-2 spike protein or a fragment thereof. In certain embodiments, the S protein may comprise an S protein of a SARS-CoV-2 variant, such as an alpha variant (e.g., B.1.1.7), a beta variant (e.g., B. 1.351, B. 1.351.2, or B. 1.351.3), a gamma variant (e.g., P.1, or P.1.1 or P.1.2), a delta variant (e.g., B.1.617.2, or AY.1, or AY.2, or AY.3) or an omicron variant (e.g., B.1.1.529), including but not limited to BA.1, BA.2, BA.3, BA.4, BA.5 and descendent lineages. It also includes BA.1/BA.2 circulating recombinant forms such as XE.

SARS-CoV-1 and SARS-CoV-2 can interact directly with angiotensin-converting enzyme 2 (ACE2) to enter target cells and may also employ the cellular serine protease, transmembrane protease, serine 2 (TMPRSS2) for S protein priming (Hoffmann et al., Cell, 2020, 181:1-10; available at doi.org/10.1016/j.cell.2020.02.052). SARS-CoV-S and SARS-CoV-2-S share about 76% amino acid identity. The receptor binding domain (RBD) in the S glycoprotein is the most variable part of the coronavirus genome. Six RBD amino acids have been shown to be critical for binding to ACE2 receptors and for determining the host range of SARS-CoV-like viruses. They are Y442, L472, N479, D480, T487 and Y4911 in SARS-CoV, which correspond to L455, F486, Q493, S494, N501 and Y505 in SARS-CoV-2 (Andersen et al., Nature Medicine, 2020; available at doi.org/10.1038/s41591-020-0820-9). SARS-CoV-1 subunits/domains and corresponding amino acid residues for SARS-CoV-1 (SEQ ID NO: 11) and SARS-CoV-2 (SEQ ID NO: 1), as well as percent identity match across the subunits/domains for SARS-CoV-1 versus SARS-CoV-2, as determined by sequence alignment (CLUSTAL O(1.2.4) multiple sequence alignment), is displayed in Table 1.

TABLE 1
Amino acid residues of subunits/domains
for SARS-CoV-1 and SARS-CoV-2

| | Residues SARS- | Residues SARS- | |
|---|---|---|---|
| SARS-CoV-1 | CoV-1 | CoV-2 | % |
| subunits/domains | (SEQ ID NO: 11) | (SEQ ID NO: 1) | Identity |
| Full protein | 1-1255 | | 75.9 |
| Signal peptide | 1-13 | | 53.9 |
| Extracellular | 14-1195 | | |
| Transmembrane | 1196-1216 | | |
| Cytoplasmic | 1217-1255 | | 97.4 |
| S1 | 14-667 | 14-684 | 63.6 |
| S2 | 668-1255 | | 90 |
| S2′ | 798-1255 | | 93 |
| Cleavage site | 667-668 | | 100 |
| Cleavage site | 797-798 | | 100 |
| Receptor-binding | 306-527 | 319-541 | 73.1 |
| domain RBD) | | | |

In certain embodiments of the disclosure, the S glycoprotein antigen may be a full-length SARS-CoV-2 S glycoprotein (comprising or consisting of SEQ ID NO: 1) or a fragment or derivative thereof that has at least 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.5% or more amino acid sequence identity to SEQ ID NO: 1.

The wild-type coronavirus S glycoprotein comprises an S1 subunit that facilitates binding of the coronavirus to cell surface proteins. Without wishing to be bound by theory, the S1 subunit of the wildtype S glycoprotein controls which cells are infected by the coronavirus. The wild-type S glycoprotein also comprises a S2 subunit, which is a transmembrane subunit that facilitates viral and cellular membrane fusion. In the various aspects and embodiments described herein, a fragment or derivative of SARS-CoV-2 S glycoprotein can comprise the S1 subunit of the SARS-CoV-2 S glycoprotein or a fragment or derivative that has at least 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.5% or more amino acid sequence identity to the S1 subunit of the SARS-CoV-2 S glycoprotein. In some embodiments described herein, a fragment or derivative of SARS-CoV-2 S glycoprotein can comprise a sequence that has at least 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.5% or more amino acid sequence identity to amino acids 14-684 of SEQ ID NO: 1. In the various aspects and embodiments described herein, a fragment or derivative of SARS-CoV-2 S glycoprotein can comprise the S2 subunit of the SARS-CoV-2 S glycoprotein or a fragment or derivative that has at least 91%, 92%, 93%, 94%, 95%, 96%, 97%, 98%, 99%, 99.5% or more amino acid sequence identity to the S2 subunit of the SARS-CoV-2 S glycoprotein.

The wild-type coronavirus S glycoprotein comprises a receptor binding domain (RBD) that facilitates binding of the coronavirus to its receptor on the host cell. The RBD of the SARS-CoV-2 spike (S) glycoprotein is described, e.g., in Anderson et al., Nature Medicine, 2020 (available at doi.org/10.1038/s41591-020-0820-9). In the various aspects and embodiments described herein, a fragment or derivative of SARS-CoV-2 S glycoprotein can comprise the RBD of the SARS-CoV-2 S glycoprotein, or a fragment or derivative that has at least 74%, 75%, 76%, 77%, 78%, 80%, 81%, 82%, 83%, 84%, 85%, 86%, 87%, 88%, 89%, 90%, 91%, 92%, 93%, 94%, 95%, 96%, 97%, 98%, 99%, 99.5% or more amino acid sequence identity to the RBD of the SARS-CoV-2 S glycoprotein. In some embodiments described herein, a fragment or derivative of SARS-CoV-2 S glycoprotein can comprise a sequence that has at least 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.5% or more amino acid sequence identity to amino acids 319-541 of SEQ ID NO: 1.

In certain embodiments, the SARS-CoV-2 S glycoprotein derivative, or fragment thereof, may comprise or consist of an insertion, deletion, and/or substitution of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, or 39 residues of the SARS-CoV-2 S glycoprotein. Non-limiting examples of amino acids for potential deletion include, e.g., a tyrosine at position (145), an asparagine at position (679), a serine at position (680), proline at position (681), an arginine at position (682), an arginine at position (683), an alanine at position (684), and/or an arginine at position (685), positions as denoted in SEQ ID NO: 1, or the equivalent amino acid residue in a mutant SARS-CoV-2 S glycoprotein sequence. Non-limiting examples of amino acids for potential substitution include, e.g., a leucine changed to a phenylalanine at position (5) a tyrosine changed to an asparagine at position (28), a threonine changed to an isoleucine at position (29), a histidine changed to a tyrosine at position (49), a leucine changed to a phenylalanine at position (54), an asparagine changed to a lysine at position (74), a glutamic acid changed to an aspartic acid at position (96), an aspartic acid changed to an asparagine at position (111), a phenylalanine changed to a leucine at position (157), a glycine changed to a valine at position (181), a serine changed to a tryptophan at position (221), a serine changed to an arginine at position (247), an alanine changed to a threonine at position (348), an arginine changed to an isoleucine at position (408), a glycine changed to a serine at position (476), a valine changed to an alanine at position (483), a histidine changed to a glutamine at position (519), an alanine changed to a serine at position (520), an aspartic acid changed to an asparagine at position (614), an aspartic acid changed to a glycine at position (614), an asparagine changed to an isoleucine at position (679), a serine change to a leucine at position (680), an arginine changed to a glycine at position (682), an arginine changed to a serine at position (683), an arginine changed to a glutamine at position (685), an arginine changed to a serine at position (685), a phenylalanine changed to a cysteine at position (797), an alanine changed to a valine at position (930), an aspartic acid changed to a tyrosine at position (936), an alanine changed to a valine at position (1078), an aspartic acid changed to a histidine at position (1168), and/or an aspartic acid changed to a histidine at position (1259), positions as denoted in SEQ ID NO: 1, or the equivalent amino acid residue in a mutant SARS-CoV-2 S glycoprotein sequence. See Becerra-Flores and Cardozo, “SARS-CoV-2 viral spike G614 mutation exhibits higher case fatality rate,” The International Journal of Clinical Practice, published online May 6, 2020; Eaaswarkhanth et al., “Could the D614G substitution in the SARS-CoV-2 spike (S) protein be associated with higher COVID-19 mortality?” International Journal of Infectious Diseases, 96: July 2020, Pages 459-460; Tang et al., “The SARS-CoV-2 Spike Protein D614G Mutation Shows Increasing Dominance and May Confer a Structural Advantage to the Furin Cleavage Domain,” Preprints 2020, 2020050407 (doi: 10.20944/preprints202005.0407.v1); Hansen et. al., “Studies in humanized mice and convalescent humans yield a SARS-CoV-2 antibody cocktail” Science, published online Jun. 15, 2020; Lokman et al., “Exploring the genomic and proteomic variations of SARS-CoV-2 spike glycoprotein: A computational biology approach”, Infection, Genetics and Evolution: Journal of Molecular Epidemiology and Evolutionary Genetics in Infectious s Diseases, 2020 June; 84:104389. DOI: 10.1016/j.meegid.2020.104389, each of which incorporated herein by reference in their entirety for all intended purposes. In certain embodiments, the SARS-CoV-2 S glycoprotein derivative, or fragments thereof, differs in amino acid sequence from the reference peptide or polypeptide (e.g., wild-type SARS-CoV-2 spike protein) by changing a serine to an arginine at position (247), an aspartic acid to an asparagine at position (614), and/or an arginine to a glutamine at position (685), positions as denoted in SEQ ID NO: 1, or the equivalent amino acid residue in a mutant SARS-CoV-2 S glycoprotein sequence. In certain embodiments, the SARS-CoV-2 S glycoprotein derivative, or fragments thereof, differs in amino acid sequence from the reference peptide or polypeptide by changing a serine to an arginine at position (247). In certain embodiments, the SARS-CoV-2 S glycoprotein derivative, or fragments thereof, differs in amino acid sequence from the reference peptide or polypeptide by changing an aspartic acid to an asparagine at position (614). In certain embodiments, the SARS-CoV-2 S glycoprotein derivative, or fragments thereof, differs in amino acid sequence from the reference peptide or polypeptide by changing an arginine to a glutamine at position (685). In certain embodiments, the SARS-CoV-2 S glycoprotein derivative, or fragments thereof, differs in amino acid sequence from the reference peptide or polypeptide by changing a serine to an arginine at position (247) and an aspartic acid to an asparagine at position (614). In certain embodiments, the SARS-CoV-2 S glycoprotein derivative, or fragments thereof, differs in amino acid sequence from the reference peptide or polypeptide by changing a serine to an arginine at position (247) and an arginine to a glutamine at position (685). In certain embodiments, the SARS-CoV-2 S glycoprotein derivative, or fragments thereof, differs in amino acid sequence from the reference peptide or polypeptide by changing an aspartic acid to an asparagine at position (614) and an arginine to a glutamine at position (685). In certain embodiments, the SARS-CoV-2 S glycoprotein derivative, or fragments thereof, differs in amino acid sequence from the reference peptide or polypeptide by changing a serine to an arginine at position (247), an aspartic acid to an asparagine at position (614), and an arginine to a glutamine at position (685). In certain embodiments, the SARS-CoV-2 S glycoprotein derivative, or fragments thereof, result in a more lytic phenotype. In certain embodiments, the SARS-CoV-2 S glycoprotein derivative, or fragments thereof, differs in amino acid sequence from the reference peptide or polypeptide (e.g., wild-type SARS-CoV-2 spike protein) by changing an asparagine to a tyrosine at position (501), and/or a glutamic acid to a lysine at position (484), and/or an aspartic acid to a glycine at position (614), and/or deletion of residues 69-70, positions as denoted in SEQ ID NO: 1, or the equivalent amino acid residue in a mutant SARS-CoV-2 S glycoprotein sequence. In certain embodiments, the SARS-CoV-2 S glycoprotein derivative, or fragments thereof, differs in amino acid sequence from the reference peptide or polypeptide by changing an asparagine to a tyrosine at position (501). In certain embodiments, the SARS-CoV-2 S glycoprotein derivative, or fragments thereof, differs in amino acid sequence from the reference peptide or polypeptide by changing a glutamic acid to a lysine at position (484). In certain embodiments, the SARS-CoV-2 S glycoprotein derivative, or fragments thereof, differs in amino acid sequence from the reference peptide or polypeptide by changing an aspartic acid to a glycine at position (614). In certain embodiments, the SARS-CoV-2 S glycoprotein derivative, or fragments thereof, differs in amino acid sequence from the reference peptide or polypeptide by deletion of residues 69-70. In certain embodiments, the SARS-CoV-2 S glycoprotein derivative, or fragments thereof, differs in amino acid sequence from the reference peptide or polypeptide by changing an asparagine to a tyrosine at position (501) and a glutamic acid to a lysine at position (484). In certain embodiments, the SARS-CoV-2 S glycoprotein derivative, or fragments thereof, differs in amino acid sequence from the reference peptide or polypeptide by changing an asparagine to a tyrosine at position (501) and an aspartic acid to a glycine at position (614). In certain embodiments, the SARS-CoV-2 S glycoprotein derivative, or fragments thereof, differs in amino acid sequence from the reference peptide or polypeptide by changing an asparagine to a tyrosine at position (501) and deletion of residues 69-70. In certain embodiments, the SARS-CoV-2 S glycoprotein derivative, or fragments thereof, differs in amino acid sequence from the reference peptide or polypeptide by changing a glutamic acid to a lysine at position (484) and an aspartic acid to a glycine at position (614). In certain embodiments, the SARS-CoV-2 S glycoprotein derivative, or fragments thereof, differs in amino acid sequence from the reference peptide or polypeptide by changing a glutamic acid to a lysine at position (484) and deletion of residues 69-70. In certain embodiments, the SARS-CoV-2 S glycoprotein derivative, or fragments thereof, differs in amino acid sequence from the reference peptide or polypeptide by changing an aspartic acid to a glycine at position (614) and deletion of residues 69-70. In certain embodiments, the SARS-CoV-2 S glycoprotein derivative, or fragments thereof, differs in amino acid sequence from the reference peptide or polypeptide by changing an asparagine to a tyrosine at position (501), a glutamic acid to a lysine at position (484), and an aspartic acid to a glycine at position (614). In certain embodiments, the SARS-CoV-2 S glycoprotein derivative, or fragments thereof, differs in amino acid sequence from the reference peptide or polypeptide by changing an asparagine to a tyrosine at position (501), changing a glutamic acid to a lysine at position (484), and deletion of residues 69-70. In certain embodiments, the SARS-CoV-2 S glycoprotein derivative, or fragments thereof, differs in amino acid sequence from the reference peptide or polypeptide by changing an asparagine to a tyrosine at position (501), changing an aspartic acid to a glycine at position (614), and deletion of residues 69-70. In certain embodiments, the SARS-CoV-2 S glycoprotein derivative, or fragments thereof, differs in amino acid sequence from the reference peptide or polypeptide by changing a glutamic acid to a lysine at position (484), changing an aspartic acid to a glycine at position (614), and deletion of residues 69-70. In certain embodiments, the SARS-CoV-2 S glycoprotein derivative, or fragments thereof, differs in amino acid sequence from the reference peptide or polypeptide by changing an asparagine to a tyrosine at position (501), changing a glutamic acid to a lysine at position (484), changing an aspartic acid to a glycine at position (614) and deletion of residues 69-70. In certain embodiments, the SARS-CoV-2 S glycoprotein derivative, or fragments thereof, differs in amino acid sequence from the reference peptide or polypeptide (e.g., wild-type SARS-CoV-2 spike protein) by inactivating the furin cleavage site within the spike protein. In certain embodiments, the SARS-CoV-2 S glycoprotein derivative, or fragments thereof, differs in amino acid sequence from the reference peptide or polypeptide (e.g., wild-type SARS-CoV-2 spike protein) by changing Q⁶⁷⁷TNSPRRARSV⁶⁸⁷ (SEQ ID NO: 12), as denoted in SEQ ID NO: 1, or the equivalent amino acid residue in a mutant SARS-CoV-2 S glycoprotein sequence, to QTILRSV (SEQ ID NO: 13) or to QTNSPGSASSV (SEQ ID NO: 14). In certain embodiments, the SARS-CoV-2 S glycoprotein derivative, or fragments thereof, result in a monobasic furin cleavage site in the S1/S2 interface (QTILRSV, SEQ ID NO: 13) or deletion of the furin cleavage site (QTNSPGSASSV, SEQ ID NO: 14) phenotype. In certain embodiments, the alteration to the furin cleavage site can lead to a spike stabilized pseudoparticles. See Hansen et. al., “Studies in humanized mice and convalescent humans yield a SARS-CoV-2 antibody cocktail” Science, published online Jun. 15, 2020, incorporated herein by reference in its entirety for all intended purposes.

In certain embodiments, the SARS-CoV-2 S glycoprotein fragment or derivative lacks one or more C-terminal residues of the full-length SARS-CoV-2 S glycoprotein. For example, the SARS-CoV-2 S glycoprotein fragment may lack 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39 of the C-terminal residues of the SARS-CoV-2 S glycoprotein. In certain embodiments, the SARS-CoV-2 S glycoprotein fragment or derivative lacks the 19 C-terminal residues of the SARS-CoV-2 S glycoprotein. The SARS-CoV-2 S glycoprotein fragment or derivative may comprise the amino acid sequence of SEQ ID NO: 2, or a sequence at least 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.5% or more identical to the amino acid sequence of SEQ ID NO: 2. In some embodiments described herein, a fragment or derivative of SARS-CoV-2 S glycoprotein can comprise a sequence that has at least 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.5% or more amino acid sequence identity to amino acids 14-684 of SEQ ID NO: 2. In some embodiments described herein, a fragment or derivative of SARS-CoV-2 S glycoprotein can comprise a sequence that has at least 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.5% or more amino acid sequence identity to amino acids 319-541 of SEQ ID NO: 2.

Non-limiting examples of amino acid residue positions for insertions, deletions and/or substitutions for SARS-CoV-2 variant lineages B.1.1.7, (20I/501Y.V1 or VOC 202012/01), B.1.351 (20H/501Y.V2), P.1 (B1.1.28.1 or 20J/501.V3, 484K.V2), B.1.429 (CAL.20C, CA VUI), B.1.2 20C-US, B1.1.17, 20E (EU1), 20A.EU2, N439K-D614G, Mink Cluster 5 variant are displayed in Table 2:

TABLE 2
Amino acid residue positions for insertions, deletions
and/or substitutions for SARS-CoV-2 variant lineages

| | | | |
|---|---|---|---|
| Mutations | Location | Phenotypes | References |

COVID VARIANT: B.1.1.7 lineage, (20I/501Y.V1 or VOC 202012/01)
Origin: UK
hCoV-19/England/SHEF-10C8326/2021

| | | | |
|---|---|---|---|
| N501Y | RBD | One of the six key | Horby P, Huntley C, Davies N, Edmunds |
| | | amino acids interacting | J, Ferguson N, Medley G, et al. |
| | | with ACE-2 receptor. | NERVTAG paper on COVID-19 variant |
| | | Associated with | of concern B.1.1.7 (2021) |
| | | increased | [gov.uk/government/publications/nervtag- |
| | | transmissibility (more | paper-on-covid-19-variant-ofconcern- |
| | | efficient/rapid | b117] |
| | | transmissibility). | Accession number: SAMN17373206 |
| 69-70 | | Potential | Wu K, Werner A P, Moliva J I, et |
| deletion | | conformational change | al. mRNA-1273 vaccine induces |
| | | in spike protein. | neutralizing antibodies against spike |
| | | Reduced sensitivity to | mutants from global SARS-CoV-2 |
| | | neutralizing antibodies. | variants. [Preprint Posted Jan. 25, |
| | | Associated with | 2021] |
| | | increased | GenBank: MW487270.1 |
| | | transmissibility (more | |
| | | efficient/rapid | |
| | | transmissibility). | |
| P681H | Near | Associated with | Xie X, Zou J, Fontes-Garfias C R, et al. |
| | S1/S2 | increased | Neutralization of N501Y mutant SARS- |
| | furin | transmissibility (more | CoV-2 by BNT162b2 vaccine-elicited |
| | cleavage | efficient/rapid | sera. [Preprint Posted Jan. 7, 2021] |
| | site | transmissibility). | Greaney A J, Loes A N, Crawford K H D, et |
| | | | al. Comprehensive mapping of mutations |
| | | | to the SARS-CoV-2 receptor-binding |
| | | | domain that affect recognition by |
| | | | polyclonal human serum antibodies. |
| | | | [Preprint Posted Jan. 4, 2021] |
| | | | Severe acute respiratory syndrome |
| | | | coronavirus 2 isolate SARS-CoV- |
| | | | 2/human/USA/NYI.B1-7.01-21/2021, |
| | | | complete genome |
| Y144 del | | | Weisblum Y, Schmidt F, Zhang F, et al. |
| | | | Escape from neutralizing antibodies by |
| | | | SARS-CoV-2 spike protein variants |
| | | | [eLife 2020; 9: e61312] |
| A570D | | | |
| T716I | | | |
| S982A | | | |
| D1118H | | | |

COVID VARIANT: B.1.351 (20H/501Y.V2)
Origin: South Africa

| | | | |
|---|---|---|---|
| K417N | RBD | Resistant to neutralizing | Weisblum Y, Schmidt F, Zhang F, et al. |
| | | antibodies. | Escape from neutralizing antibodies by |
| | | | SARS-CoV-2 spike protein variants |
| | | | [eLife 2020; 9: e61312] |
| | | | hCoV-19/Belgium/AZDelta05413- |
| | | | 2105R/2021 |
| E484K | RBD | Resistant to neutralizing | Resende P C, Bezerra J F, de Vasconcelos |
| | | antibodies. E484K may | R H T, at al. Spike E484K mutation in the |
| | | affect neutralization by | first SARS-CoV-2 reinfection case |
| | | some polyclonal and | confirmed in Brazil, 2020external icon. |
| | | mAb, potentially by | [Posted on virological.org on Jan. 10, |
| | | disrupting the | 2021] |
| | | immunodominant B cell | |
| | | epitope, and is thought | |
| | | to be the mutation that | |
| | | drives immune escape. | |
| N501Y | RBD | Resistant to neutralizing | |
| | | antibodies, increased | |
| | | transmissibility. | |
| D614G | | | |
| A701V | | | |
| L18F | NTD | | |
| D80A | NTD | | |
| D215G | NTD | | |
| L242-244 | NTD | | |
| del | | | |
| R246I | NTD | Disrupts N5-loop (large, | |
| | | solvent exposed loop in | |
| | | NTD) and displaces the | |
| | | loop | |

COVID VARIANT: P.1 lineage (B1.1.28.1 or 20J/501.V3, 484K.V2)
Origin: Brazil

| | | | |
|---|---|---|---|
| K417T | RBD | altered transmissibility | Resende P C, Bezerra J F, de Vasconcelos |
| E484K | RBD | and antigenic profile, | R H T, at al. Spike E484K mutation in the |
| N501Y | RBD | which may affect ability | first SARS-CoV-2 reinfection case |
| L18F | NTD | of Ab generated through | confirmed in Brazil, 2020external icon. |
| T20N | NTD | previous natural | [Posted on virological.org on Jan. 10, |
| P26S | | infection or vaccination | 2021] |
| D138Y | | to recognize and | hCoV-19/Brazil/RR-1087/2021 |
| R190S | | neutralize virus | |
| D614G | | | |
| H655Y | | | |
| T1027I | | | |

COVID VARIANT: B.1.429 (CAL.20C, CA VUI)
Origin: California

S131
W152C
L452R
D614G

COVID VARIANT: B.1.2 lineage 20C-US

| | | |
|---|---|---|
| Q677H | Adjacent | Adrian A. Pater et al., Emergence and |
| | to furin | Evolution of a Prevalent New SARS- |
| | cleavage | CoV-2 Variant in the United States |
| | site | [doi.org/10.1101/2021.01.11.426287] |
| Other | | |
| mutations | | |
| in ORFs | | |

COVID VARIANT: B1.1.17

Weisblum Y, Schmidt F, Zhang F, et al.
Escape from neutralizing antibodies by
SARS-CoV-2 spike protein variants
[eLife 2020; 9: e61312]

COVID VARIANT: 20E (EU1)

A22V
D614G

COVID VARIANT: 20A.EU2

S477N
D614G

COVID VARIANT: N439K-D614G

N439K
D614G

COVID VARIANT: Mink Cluster 5 variant

| | | |
|---|---|---|
| H69 del | | |
| V70 del | | |
| Y453F | RBD | Increased binding |
| | | affinity for mink Ace2 |
| D614G | | |
| I692V | | |
| M1229I | | |

In certain embodiments, the SARS-CoV-2 S glycoprotein fragment or derivative thereof comprises a D614G mutation. The SARS-CoV-2 S glycoprotein fragment or derivative which may comprise a D614G mutation may comprise the amino acid sequence of SEQ ID NO: 3, or a sequence at least 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.5% or more identical to the amino acid sequence of SEQ ID NO: 3.

In certain embodiments, the SARS-CoV-2 S glycoprotein fragment or derivative thereof may be any various SARS-CoV-2 S glycoprotein described in Table 3 disclosed herein, or any fragment or derivative thereof. The SARS-Cov-2 S glycoprotein may be, for example, WT spike trimer, Omicron BA.1, Omicron BA.2, Omicron BA.3, Alpha, Beta, Delta, or Gamma, or a fragment of derivative thereof.

In certain embodiments, the SARS-CoV-2 S glycoprotein fragment or derivative thereof comprises a R682G, R683S, R685S, K986P, and/or a V987P mutation(s). The SARS-CoV-2 S glycoprotein fragment or derivative which may comprise a R682G, R683S, R685S, K986P, and/or a V987P mutation(s) may comprise the amino acid sequence of SEQ ID NO: 5, or a sequence at least 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.5% or more identical to the amino acid sequence of SEQ ID NO: 5.

In some embodiments, the SARS-CoV-2 S glycoprotein fragment or derivative thereof comprises may comprise the amino acid sequence of SEQ ID NO: 6, or a sequence at least 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.5% or more identical to the amino acid sequence of SEQ ID NO: 6.

In certain embodiments of the disclosure, the SARS-CoV-2 S glycoprotein or a fragment or derivative thereof can comprise a consensus sequence derived from two or more different strains, mutants or variants of SARS-CoV-2. In other embodiments, the methods of the disclosure use a mixture of SARS-CoV-2 S glycoproteins (or fragments or derivatives thereof) from two or more different strains, mutants or variants of SARS-CoV-2.

In certain embodiments, the antigen(s) disclosed herein, e.g., SARS-CoV-2 S glycoprotein or a fragment or a derivative thereof, may comprise a detectable label. In certain embodiments, the antigen(s) may comprise a reporter molecule. The detectable label or reporter molecule can be a radioisotope, such as ³H, ¹⁴C, ³²P, ³⁵S, or ¹²⁵I; a fluorescent or chemiluminescent moiety such as fluorescein isothiocyanate, or rhodamine; or an enzyme such as alkaline phosphatase, β-galactosidase, horseradish peroxidase, or luciferase. In some embodiments, the detectable label or reporter molecule can be a his-tag, or a polyhistidine tag. In some embodiments, the detectable label or reporter molecule can be a C-terminal mFc tag, myc-myc-histidine tag, or a myc-myc-hexahistidine tag. By way of a non-limiting example, a SARS-CoV-2 glycoprotein disclosed herein may comprise an Fc tag, e.g., a mouse Fc tag (mFc). In some embodiments, a SARS-CoV-2 S glycoprotein fragment or derivative thereof comprising a mFC may comprise the amino acid sequence of SEQ ID NO: 4, or a sequence at least 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.5% or more identical to the amino acid sequence of SEQ ID NO: 4. By way of another non-limiting example, a SARS-CoV-2 glycoprotein disclosed herein may comprise a myc-myc-hexahistidine tag. In some embodiments, a SARS-CoV-2 S glycoprotein fragment or derivative thereof comprising a myc-myc-hexahistidine tag may comprise the amino acid sequence of SEQ ID NO: 5, or a sequence at least 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.5% or more identical to the amino acid sequence of SEQ ID NO: 5.

In certain aspects and embodiments of the present disclosure, methods disclosed herein may comprise a SARS-CoV-1 S glycoprotein or a fragment or derivative thereof. By way of a non-limiting example, a SARS-CoV-1 S glycoprotein or a fragment or derivative thereof may comprise the amino acid sequence of SEQ ID NO: 11, or a sequence at least 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.5% or more identical to the amino acid sequence of SEQ ID NO: 11.

An antigen disclosed herein can be distinct from an epitope which may comprise a substructure of an antigen, e.g., a polypeptide or carbohydrate structure, which may be recognized by an antigen binding site. In particular, an epitope disclosed herein may comprise an antigenic determinant that interacts with a specific antigen-binding site of an antigen-binding protein, e.g., a variable region of an antibody molecule, known as a paratope. A single antigen disclosed hererein may have more than one epitope. Thus, different antibodies may bind to different areas on an antigen and may have different biological effects. An epitope disclosed herein may also comprise a site on an antigen to which B cells and/or T cells respond. An epitope may also include a region of an antigen that is bound by an antibody.

Epitopes may be defined as structural or functional. Functional epitopes are generally a subset of the structural epitopes and have those residues that directly contribute to the affinity of the interaction. Epitopes may be linear or conformational, that is, composed of non-linear amino acids. In certain embodiments, epitopes may include determinants that are chemically active surface groupings of molecules such as amino acids, sugar side chains, phosphoryl groups, or sulfonyl groups, and, in certain embodiments, may have specific three-dimensional structural characteristics, and/or specific charge characteristics.

Epitopes can include B cell epitopes and T cell epitopes. B-cell epitopes are peptide sequences which are required for recognition by specific antibody producing B-cells. B cell epitopes refer to a specific region of the antigen that is recognized by an antibody. The portion of an antibody that binds to the epitope is called a paratope. An epitope may be a conformational epitope or a linear epitope, based on the structure and interaction with the paratope. A linear, or continuous, epitope is defined by the primary amino acid sequence of a particular region of a protein. The sequences that interact with the antibody are situated next to each other sequentially on the protein, and the epitope can usually be mimicked by a single peptide. Conformational epitopes are epitopes that are defined by the conformational structure of the native protein. These epitopes may be continuous or discontinuous, i.e., components of the epitope can be situated on disparate parts of the protein, which are brought close to each other in the folded native protein structure.

T-cell epitopes are peptide sequences which, in association with proteins on APC, are required for recognition by specific T-cells. T cell epitopes are processed intracellularly and presented on the surface of APCs, where they are bound to MHC molecules including MHC class II and MHC class I. The peptide epitope may be any length that is reasonable for an epitope. In some embodiments, the peptide epitope is 9-30 amino acids. For example, the length may be 9-22, 9-29, 9-28, 9-27, 9-26, 9-25, 9-24, 9-23, 9-21, 9-20, 9-19, 9-18, 10-22, 10-21, 10-20, 11-22, 22-21, 11-20, 12-22, 12-21, 12-20, 13-22, 13-21, 13-20, 14-19, 15-18, or 16-17 amino acids.

Methods for determining the epitope of an antigen-binding protein, e.g., antibody or fragment or polypeptide, include alanine scanning mutational analysis, peptide blot analysis (Reineke (2004) Methods Mol. Biol. 248: 443-63), peptide cleavage analysis, crystallographic studies and NMR analysis. In addition, methods such as epitope excision, epitope extraction and chemical modification of antigens can be employed (Tomer (2000) Prot. Sci. 9: 487-496). Another method that can be used to identify the amino acids within a polypeptide with which an antigen-binding protein (e.g., antibody or fragment or polypeptide) interacts is hydrogen/deuterium exchange detected by mass spectrometry. In general terms, the hydrogen/deuterium exchange method involves deuterium-labeling the protein of interest, followed by binding the antigen-binding protein, e.g., antibody or fragment or polypeptide, to the deuterium-labeled protein. Next, the protein/antigen-binding protein complex is transferred to water and exchangeable protons within amino acids that are protected by the antibody complex undergo deuterium-to-hydrogen back-exchange at a slower rate than exchangeable protons within amino acids that are not part of the interface. As a result, amino acids that form part of the protein/antigen-binding protein interface may retain deuterium and therefore exhibit relatively higher mass compared to amino acids not included in the interface. After dissociation of the antigen-binding protein (e.g., antibody or fragment or polypeptide), the target protein is subjected to protease cleavage and mass spectrometry analysis, thereby revealing the deuterium-labeled residues which correspond to the specific amino acids with which the antigen-binding protein interacts.

In certain embodiments, the epitope disclosed herein may comprise an immunodominant epitope. An immunodominant epitope may comprise an epitope within an antigen that selectively provokes an immune response in a host to the effective or functional exclusion, which may be partial or complete, of other epitopes of that antigen. In some embodiments, the one or more first epitopes disclosed herein are immunodominant epitopes. In some embodiments, the immunodominant epitopes are less conserved than other epitopes of the antigen between different strains or species of a pathogen from which the antigen is derived.

Non-limiting examples of epitopes include epitopes that are targeted by the anti-SARS-CoV-2 S glycoprotein antibodies E10933, E10987, E14315, and E15160 as described herein. In some embodiments, the one or more first epitopes may comprise one or more epitopes that are targeted by the anti-SARS-CoV-2 S glycoprotein antibodies E10933, E10987, E14315, or E15160 as described herein. Non-limiting examples of an epitope that can be targeted by an antibody against SARS CoV-2 are described in U.S. Pat. No. 10,787,501, which is incorporated herein by reference in its entirety for all purposes.

In some embodiments, the one or more first epitopes comprises a sequence that is contained within the RBD domain of a SARS-CoV-2 S glycoprotein such as those disclosed herein.

In some embodiments, the one or more first epitopes comprises a sequence that is contained within amino acids 319-541 of SEQ ID NO: 1, or a sequence has at least 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or more amino acid sequence identity to such a sequence contained within SEQ ID NO: 319-541 of SEQ ID NO: 1.

In some embodiments, the one or more first epitopes comprises a sequence that is contained within amino acids 319-541 of SEQ ID NO: 2, or a sequence has at least 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or more amino acid sequence identity to such a sequence contained within SEQ ID NO: 319-541 of SEQ ID NO: 2.

In some embodiments, the one or more first epitopes of the SARS-CoV-2 spike glycoprotein antigen disclosed herein may be a neutralizing epitope(s), e.g., comprised within the receptor binding domain (RBD) of the SARS-CoV-2 spike glycoprotein. In some embodiments, the neutralizing epitopes may be targeted by antibodies, e.g., neutralizing antibodies, disclosed herein.

In some embodiments, the one or more first epitopes comprise the epitope targeted by anti-influenza hemagglutinin (HA) antibody E4123 as described herein. In some embodiments, the epitope may be comprised within the sialic-acid, receptor binding site (RBS) on the HA head.

In some embodiments, the one or more first epitopes of the influenza hemagglutinin (HA) antigen disclosed herein may be a neutralizing epitope(s), e.g., comprised within the sialic-acid, receptor binding site (RBS) on the HA head. In some embodiments, the neutralizing epitopes may be targeted by antibodies, e.g., neutralizing antibodies, disclosed herein.

### Antibodies

In certain embodiments, an antibody disclosed herein may comprise immunoglobulin molecules comprising four polypeptide chains, two heavy chains (HCs) and two light chains (LCs) inter-connected by disulfide bonds (i.e., “full antibody molecules”), as well as multimers thereof (e.g., IgM). Each heavy chain may comprise a heavy chain variable region (“HCVR” or “VH”) and a heavy chain constant region (comprised of domains CH1, CH2 and CH3). Each light chain may comprise a light chain variable region (“LCVR or “VL”) and a light chain constant region (CL). The VH and VL regions may be further subdivided into regions of hypervariability, termed complementarity determining regions (CDR), interspersed with regions that are more conserved, termed framework regions (FR). Each VH and VL may comprise three CDRs and four FRs, arranged from amino-terminus to carboxy-terminus in the following order: FR1, CDR1, FR2, CDR2, FR3, CDR3, FR4. Heavy chain CDRs can also be referred to as HCDRs or CDR-Hs, and numbered as described above (e.g., HCDR1, HCDR2, and HCDR3 or CDR-H1, CDR-H2, and CDR-H3). Likewise, light chain CDRs can be referred to as LCDRs or CDR-Ls, and numbered LCDR1, LCDR2, and LCDR3, or CDR-L1, CDR-L2, and CDR-L3. In certain embodiments of the disclosure, the FRs of the antibody (or antigen binding fragment thereof) are identical to the human germline sequences or are naturally or artificially modified.

In certain embodiments, the present disclosure includes monoclonal antibodies and antigen-binding fragments thereof. A monoclonal antibody disclosed herein can comprise a population of substantially homogeneous antibodies, i.e., the antibody molecules comprising the population are identical in amino acid sequence except for possible naturally occurring mutations that may be present in minor amounts. In certain embodiments, compositions disclosed herein may comprise, two or more monoclonal antibodies (mAbs) targeting one or more first epitopes, e.g., immunodominant epitopes of an antigen. The immunodominant epitopes may be less conserved than other epitopes of the antigen between different strains or species of a pathogen from which the antigen is derived.

In some embodiments of the disclosure, an antibody or antigen-binding fragment disclosed herein may comprise a heavy chain constant domain, e.g., of the type IgA (e.g., IgA1 or IgA2), IgD, IgE, IgG (e.g., IgG1, IgG2, IgG3 and IgG4) or IgM. In some embodiments, antibody or antigen-binding fragment thereof may comprise a light chain constant domain, e.g., of the type kappa or lambda.

In some embodiments, the antibody may comprise a human antibody or antigen-binding fragment thereof. A human antigen-binding protein, such as an antibody, as used herein, includes antibodies having variable and constant regions derived from human germline immunoglobulin sequences whether in a human cell or grafted into a non-human cell, e.g., a mouse cell. The human mAbs of the disclosure may include amino acid residues not encoded by human germline immunoglobulin sequences (e.g., mutations introduced by random or site-specific mutagenesis in vitro or by somatic mutation in vivo).

In certain embodiments, the present disclosure includes chimeric antibodies and antigen-binding fragments thereof. A chimeric antibody disclosed herein may comprise an antibody having the variable domain from a first antibody and the constant domain from a second antibody, where the first and second antibodies are from different species

The present disclosure further includes hybrid antigen-binding proteins, e.g., antibodies and antigen-binding fragments thereof, and methods of use thereof. A hybrid antibody of the disclosure may comprise is an antibody having the variable domain from a first antibody and the constant domain from a second antibody, wherein the first and second antibodies are from different animals, or wherein the variable domain, but not the constant region, is from a first animal. For example, a variable domain can be taken from an antibody isolated from a human and expressed with a fixed constant region not isolated from that antibody. Hybrid antibodies are synthetic and non-naturally occurring because the variable and constant regions they contain are not isolated from a single natural source.

The present disclosure further includes recombinant antibodies or antigen-binding fragments thereof. In some embodiments, the recombinant antibody of the disclosure may comprise molecules created, expressed, isolated or obtained by technologies or methods known in the art as recombinant DNA technology which include, e.g., DNA splicing and transgenic expression. The term includes antibodies expressed in a non-human mammal (including transgenic non-human mammals, e.g., transgenic mice), or a cell (e.g., CHO cells) expression system, or a non-human cell expression system, or isolated from a recombinant combinatorial human antibody library. In some embodiments, a recombinant antibody shares a sequence with an antibody isolated from an organism (e.g., a mouse or a human), but has been expressed via recombinant DNA technology. Such antibodies may have post-translational modifications (e.g., glycosylation) that differ from the antibody as isolated from the organism.

In certain embodiments, an antibody or antigen binding fragment thereof disclosed herein may target one or more first epitope of a SARS-CoV-2 antigen disclosed herein. In some embodiments, the antigen is SARS-CoV-2 spike glycoprotein and the first epitopes are neutralizing epitopes comprised within the receptor binding domain (RBD) of the SARS-CoV-2 spike glycoprotein. Without wishing to be bound by theory, the RBD domain of coronaviruses constantly switches between a standing-up and lying-down position (Yuan 2017 and Gui 2017), suggesting that some neutralizing antibody targeting may be context dependent. As proteolytic activation of spike is also required for membrane fusion and virus entry into cells the S1/S2 cleavage boundary may also be a target for neutralizing antibodies.

In some embodiments, the antibody and/or antigen binding fragment thereof may be selected from anti-SARS-CoV-2 S glycoprotein antibodies E10933, E10987, E14315, and E15160 as described herein, or antigen binding fragment thereof, or a combination thereof. In some embodiments, the antibody is a monoclonal antibody (mAb).

In some embodiments, the mAb described herein may be mAb E10933. In some embodiments, the mAb described herein may be mAb E10987. In some embodiments, the mAb described herein may be mAb E14315. In some embodiments, the mAb described herein may be mAb E15160.

The antibody and antigen-binding fragments thereof of the present disclosure, in some embodiments of the disclosure, include a heavy chain having at least 70% (e.g., 80%, 85%, 90%, 91%, 92%, 93%, 94%, 95%, 96%, 97%, 98%, 99%, or greater) amino acid sequence identity to the HC amino acid sequence set in SEQ ID NO: 20; and/or a light chain having at least 70% (e.g., 80%, 85%, 90%, 91%, 92%, 93%, 94%, 95%, 96%, 97%, 98%, 99%, or greater) amino acid sequence identity to the LC amino acid sequence set forth in SEQ ID NO: 21. In some embodiments, the heavy chain variable (VH) region of the HC disclosed herein can comprise a sequence that has at least 70% (e.g., 80%, 85%, 90%, 91%, 92%, 93%, 94%, 95%, 96%, 97%, 98%, 99%, or greater) amino acid sequence identity to amino acids 1-120 of SEQ ID NO: 20. In some embodiments, the light chain variable (VL) region of the LC disclosed herein can comprise a sequence that has at least 70% (e.g., 80%, 85%, 90%, 91%, 92%, 93%, 94%, 95%, 96%, 97%, 98%, 99%, or greater) amino acid sequence identity to amino acids 1-111 of SEQ ID NO: 21.

The antibody and antigen-binding fragments thereof of the present disclosure, in some embodiments of the disclosure, include a heavy chain having at least 70% (e.g., 80%, 85%, 90%, 91%, 92%, 93%, 94%, 95%, 96%, 97%, 98%, 99%, or greater) amino acid sequence identity to the HC amino acid sequence set in SEQ ID NO: 22; and/or a light chain having at least 70% (e.g., 80%, 85%, 90%, 91%, 92%, 93%, 94%, 95%, 96%, 97%, 98%, 99%, or greater) amino acid sequence identity to the LC amino acid sequence set forth in SEQ ID NO: 23. In some embodiments, the heavy chain variable (VH) region of the HC disclosed herein can comprise a sequence that has at least 70% (e.g., 80%, 85%, 90%, 91%, 92%, 93%, 94%, 95%, 96%, 97%, 98%, 99%, or greater) amino acid sequence identity to amino acids 1-120 of SEQ ID NO: 22. In some embodiments, the light chain variable (VL) region of the LC disclosed herein can comprise a sequence that has at least 70% (e.g., 80%, 85%, 90%, 91%, 92%, 93%, 94%, 95%, 96%, 97%, 98%, 99%, or greater) amino acid sequence identity to amino acids 1-107 of SEQ ID NO: 23.

The antibody and antigen-binding fragments thereof of the present disclosure, in some embodiments of the disclosure, include a heavy chain having at least 70% (e.g., 80%, 85%, 90%, 91%, 92%, 93%, 94%, 95%, 96%, 97%, 98%, 99%, or greater) amino acid sequence identity to the HC amino acid sequence set in SEQ ID NO: 24; and/or a light chain having at least 70% (e.g., 80%, 85%, 90%, 91%, 92%, 93%, 94%, 95%, 96%, 97%, 98%, 99%, or greater) amino acid sequence identity to the LC amino acid sequence set forth in SEQ ID NO: 25. In some embodiments, the heavy chain variable (VH) region of the HC disclosed herein can comprise a sequence that has at least 70% (e.g., 80%, 85%, 90%, 91%, 92%, 93%, 94%, 95%, 96%, 97%, 98%, 99%, or greater) amino acid sequence identity to amino acids 1-121 of SEQ ID NO: 24. In some embodiments, the light chain variable (VL) region of the LC disclosed herein can comprise a sequence that has at least 70% (e.g., 80%, 85%, 90%, 91%, 92%, 93%, 94%, 95%, 96%, 97%, 98%, 99%, or greater) amino acid sequence identity to amino acids 1-108 of SEQ ID NO: 25.

The antibody and antigen-binding fragments thereof of the present disclosure, in some embodiments of the disclosure, include a heavy chain having at least 70% (e.g., 80%, 85%, 90%, 91%, 92%, 93%, 94%, 95%, 96%, 97%, 98%, 99%, or greater) amino acid sequence identity to the HC amino acid sequence set in SEQ ID NO: 26; and/or a light chain having at least 70% (e.g., 80%, 85%, 90%, 91%, 92%, 93%, 94%, 95%, 96%, 97%, 98%, 99%, or greater) amino acid sequence identity to the LC amino acid sequence set forth in SEQ ID NO: 27. In some embodiments, the heavy chain variable (VH) region of the HC disclosed herein can comprise a sequence that has at least 70% (e.g., 80%, 85%, 90%, 91%, 92%, 93%, 94%, 95%, 96%, 97%, 98%, 99%, or greater) amino acid sequence identity to amino acids 1-123 of SEQ ID NO: 26. In some embodiments, the light chain variable (VL) region of the LC disclosed herein can comprise a sequence that has at least 70% (e.g., 80%, 85%, 90%, 91%, 92%, 93%, 94%, 95%, 96%, 97%, 98%, 99%, or greater) amino acid sequence identity to amino acids 1-107 of SEQ ID NO: 27.

In certain embodiments, an antibody or antigen binding fragment thereof disclosed herein may target one or more first epitope of an influenza antigen disclosed herein. In some embodiments, the antigen is influenza hemagglutinin (HA), and the one or more first epitopes are comprised within sialic-acid, receptor binding site (RBS) on the HA head.

In some embodiments, the antibody and/or antigen binding fragment thereof may be the anti-influenza hemagglutinin (HA) antibody E4123 described herein. In some embodiments, the antibody and/or antigen binding fragment may target an epitope comprised within the sialic-acid, receptor binding site (RBS) on the HA head.

A variant antibody or antigen-binding fragments thereof may include a polypeptide comprising an amino acid sequence that is set forth herein except for one or more (e.g., 1, 2, 3, 4, 5, 6, 7, 8, 9 or 10) mutations such as, for example, missense mutations (e.g., conservative substitutions), non-sense mutations, deletions, or insertions.

### Nucleic Acid Molecules

In some embodiments, the antigens or antibodies disclosed herein may be administered to a subject as one or more nucleic acid molecule encoding the antigens and/or antibodies. Accordingly, in some embodiments, the present disclosure provides a nucleic acid molecule encoding an antigen disclosed herein. In some embodiments, the present disclosure provides a nucleic acid molecule encoding one or more antibodies or antibody fragments described herein targeting one or more first epitopes of the antigen. In some embodiments, the present disclosure provides one or more nucleic acid molecules encoding each of the antigen and one or more antibodies or antibody fragments targeting one or more first epitopes of the antigen. In some embodiments, the nucleic acid molecules encoding the antigen and one or more antibodies or antibody fragments are co-administered.

In some embodiments, the present disclosure provides a nucleic acid molecule encoding an antigen and one or more antibodies targeting one or more epitopes of the antigen that is encoded by the disclosed nucleic acid molecule.

In some embodiments, the nucleic acid molecules described herein are DNA molecules.

In some embodiments, the nucleic acid molecules described herein are RNA molecules. In a specific embodiment, the nucleic acid molecules are messenger RNA (mRNA) molecules. When the mRNA molecule(s) encoding an antigen is delivered to a cell, the mRNA may be processed into a polypeptide by the intracellular machinery which can then process the polypeptide into antigenic fragments capable of stimulating an immune response against the infectious disease or cancer.

The nucleic acid molecules according to the present disclosure can be single-stranded or double-stranded, linear or circular, or in particular in the form of mRNA.

The nucleic acid molecules described herein include one or more open reading frames encoding the antigen and/or the one or more antibodies targeting one or more epitopes of the antigen. As used herein, the term “open reading frame”, abbreviated as “ORF”, refers to a segment or region of a nucleic acid molecule that encodes a polypeptide. The ORF comprises a continuous stretch of non-overlapping, in-frame codons, beginning with the initiation codon and ending with a stop codon, and is translated by the ribosome.

A nucleic acid molecule of the present disclosure may be mono-, bi- or multicistronic, coding for an antigen and/or one or more antibodies described herein. In some embodiment, a nucleic acid molecule of the present disclosure may contain at least two coding regions, one of which coding for an antigen and the other(s) coding for one or more antibodies targeting one or more epitopes of the antigen. The one or more antibodies, or one or more epitopes, may be identical or distinct. As a non-limiting example, a nucleic acid molecule of the present disclosure may contain three coding regions, one coding for an antigen, one coding for one antibody targeting one epitope of the antigen, and the other one coding for another antibody targeting another epitope of the antigen. In other embodiments, a nucleic acid molecule of the present disclosure may code for an antigen and one or more antibodies within the same coding region.

In some embodiments, nucleic acid molecules of the present disclosure may include one or more internal ribosomal entry site (IRES). An IRES can function as the sole ribosome binding site, but it can also serve to provide a nucleic acid molecule according to the present disclosure which codes for an antigen and/or one or more antibodies to be translated by the ribosomes independently of one another (“multicistronic construct”). Such a nucleic acid molecule can code, for example, a complete sequence of an antibody, by linking the corresponding coding regions of the heavy and light chain with one another with an IRES sequence. However, the heavy and light chain to be encoded by a nucleic acid molecule of the present disclosure may also be located in one single “cistron”. In some embodiments, the light chain sequence is 3′ to the heavy chain sequence. In some embodiments, the light chain sequence is 5′ to the heavy chain sequence. An IRES sequences described herein may be employed in particular for simultaneous and uniform expression of the light and the heavy chains of the antibody coded by the nucleic acid molecule according to the present disclosure. Non-limiting examples of IRES sequences which can be used in the present disclosure include those derived from classical swine fever viruses (CSFV), cricket paralysis viruses (CrPV), encephalomyocarditis viruses (ECMV), picornaviruses (e.g., foot and mouth disease viruses (FMDV)), pest viruses (CFFV), polio viruses (PV), hepatitis C viruses (HCV), murine leukoma virus (MLV), simian immunodeficiency viruses (SIV), or super IRES sequences.

In some embodiments, nucleic acid molecules of the present disclosure may encode one or more self-cleaving peptides. A “self-cleaving peptide” or a “self-cleaving sequence” encoding a self-cleaving domain is a peptide or coding sequence, respectively, that induces ribosomal skipping during protein translation, resulting in a break. Examples of protease cleavage sites are the cleavage sites of potyvirus NIa proteases (e.g. tobacco etch virus protease), potyvirus HC proteases, potyvirus P1 (P35) proteases, byovirus NIa proteases, byovirus RNA-2-encoded proteases, aphthovirus L proteases, enterovirus 2A proteases, rhinovirus 2A proteases, picorna 3C proteases, comovirus 24K proteases, nepovirus 24K proteases, RTSV (rice tungro spherical virus) 3C-like protease, PYVF (parsnip yellow fleck virus) 3C-like protease, thrombin, factor Xa and enterokinase. Due to its high cleavage stringency, TEV (tobacco etch virus) protease cleavage sites are particularly preferred. In some embodiments, the isolated nucleic acid includes a self-cleaving peptidyl sequence encoding a self-cleaving peptidyl domain between the heavy chain sequence and the light chain sequence. Preferred self-cleaving peptides include those derived from potyvirus and cardiovirus 2A peptides. In some embodiments, self-cleaving peptides are selected from 2A peptides derived from FMDV (foot-and-mouth disease virus), equine rhinitis A virus, Thosea asigna virus and porcine teschovirus.

In some embodiments, self-cleaving peptidyl linker sequences used herein is a 2A sequence. In some embodiments, the self-cleaving peptidyl linker sequence is a T2A sequence or a P2A sequence. In some embodiments, the self-cleaving peptidyl linker sequence is a foot-and-mouth disease virus sequence. In some embodiments, the self-cleaving peptidyl linker sequence is PVKQLLNFDLLKLAGDVESNPGP (SEQ ID NO: 15). In some embodiments, the self-cleaving peptidyl linker sequence is an equine rhinitis A virus sequence. In some embodiments, the self-cleaving peptidyl linker sequence is QCTNYALLKLAGDVESNPGP (SEQ ID NO: 16). In embodiments, the self-cleaving peptidyl linker sequence is a porcine teschovirus 1 sequence. In embodiments, the self-cleaving peptidyl linker sequence is ATNFSLLKQAGDVEENPGP (SEQ ID NO: 17). In some embodiments, the self-cleaving peptidyl linker sequence is Thosea asigna virus sequence. In some embodiments, the self-cleaving peptidyl linker sequence is EGRGSLLTCGDVESNPGP (SEQ ID NO: 18).

In some embodiments, a nucleic acid molecule of the present disclosure comprises a nucleotide sequence encoding a polypeptide sequence that is a SARS-CoV-2 S glycoprotein or a variant and/or fragment thereof.

In some embodiments, a nucleic acid molecule of the present disclosure comprises a nucleotide sequence encoding a polypeptide sequence that is at least 70%, at least 75%, at least 80%, at least 85%, at least 90%, at least 95%, at least 96%, at least 97%, at least 98%, at least 99%, or 100% identity to the polypeptide sequence of any one of SEQ ID NOs: 1-6, or a variant and/or fragment thereof.

In some embodiments, a nucleic acid molecule of the present disclosure comprises one or more nucleotide sequences encoding one or more antibodies selected from the anti-SARS-CoV-2 S glycoprotein mAbs E10933, E10987, E14315, or E15160 as described herein. In some embodiments, a nucleic acid molecule of the present disclosure comprises one or more nucleotide sequences encoding two antibodies selected from the anti-SARS-CoV-2 S glycoprotein mAbs E10933, E10987, E14315, or E15160 as described herein. In some embodiments, a nucleic acid molecule of the present disclosure comprises one or more nucleotide sequences encoding three antibodies selected from the anti-SARS-CoV-2 S glycoprotein mAbs E10933, E10987, E14315, or E15160 as described herein. In some embodiments, a nucleic acid molecule of the present disclosure comprises one or more nucleotide sequences encoding four antibodies selected from the anti-SARS-CoV-2 S glycoprotein mAbs E10933, E10987, E14315, or E15160 as described herein.

In some embodiments, a nucleic acid molecule of the present disclosure comprises one or more nucleotide sequences encoding a heavy chain having at least 70% (e.g., 80%, 85%, 90%, 91%, 92%, 93%, 94%, 95%, 96%, 97%, 98%, 99%, or greater) amino acid sequence identity to the HC amino acid sequence set in SEQ ID NO: 20; and/or a light chain having at least 70% (e.g., 80%, 85%, 90%, 91%, 92%, 93%, 94%, 95%, 96%, 97%, 98%, 99%, or greater) amino acid sequence identity to the LC amino acid sequence set forth in SEQ ID NO: 21. In some embodiments, a nucleic acid molecule of the present disclosure comprises one or more nucleotide sequences encoding a heavy chain variable (VH) region of the HC comprising a sequence that has at least 70% (e.g., 80%, 85%, 90%, 91%, 92%, 93%, 94%, 95%, 96%, 97%, 98%, 99%, or greater) amino acid sequence identity to amino acids 1-120 of SEQ ID NO: 20; and/or a light chain variable (VL) region of the LC comprising a sequence that has at least 70% (e.g., 80%, 85%, 90%, 91%, 92%, 93%, 94%, 95%, 96%, 97%, 98%, 99%, or greater) amino acid sequence identity to amino acids 1-111 of SEQ ID NO: 21.

In some embodiments, a nucleic acid molecule of the present disclosure comprises one or more nucleotide sequences encoding a heavy chain having at least 70% (e.g., 80%, 85%, 90%, 91%, 92%, 93%, 94%, 95%, 96%, 97%, 98%, 99%, or greater) amino acid sequence identity to the HC amino acid sequence set in SEQ ID NO: 22; and/or a light chain having at least 70% (e.g., 80%, 85%, 90%, 91%, 92%, 93%, 94%, 95%, 96%, 97%, 98%, 99%, or greater) amino acid sequence identity to the LC amino acid sequence set forth in SEQ ID NO: 23. In some embodiments, a nucleic acid molecule of the present disclosure comprises one or more nucleotide sequences encoding a heavy chain variable (VH) region of the HC comprising a sequence that has at least 70% (e.g., 80%, 85%, 90%, 91%, 92%, 93%, 94%, 95%, 96%, 97%, 98%, 99%, or greater) amino acid sequence identity to amino acids 1-120 of SEQ ID NO: 22; and/or a light chain variable (VL) region of the LC comprising a sequence that has at least 70% (e.g., 80%, 85%, 90%, 91%, 92%, 93%, 94%, 95%, 96%, 97%, 98%, 99%, or greater) amino acid sequence identity to amino acids 1-107 of SEQ ID NO: 23.

In some embodiments, a nucleic acid molecule of the present disclosure comprises one or more nucleotide sequences encoding a heavy chain having at least 70% (e.g., 80%, 85%, 90%, 91%, 92%, 93%, 94%, 95%, 96%, 97%, 98%, 99%, or greater) amino acid sequence identity to the HC amino acid sequence set in SEQ ID NO: 24; and/or a light chain having at least 70% (e.g., 80%, 85%, 90%, 91%, 92%, 93%, 94%, 95%, 96%, 97%, 98%, 99%, or greater) amino acid sequence identity to the LC amino acid sequence set forth in SEQ ID NO: 25. In some embodiments, a nucleic acid molecule of the present disclosure comprises one or more nucleotide sequences encoding a heavy chain variable (VH) region of the HC comprising a sequence that has at least 70% (e.g., 80%, 85%, 90%, 91%, 92%, 93%, 94%, 95%, 96%, 97%, 98%, 99%, or greater) amino acid sequence identity to amino acids 1-121 of SEQ ID NO: 24; and/or a light chain variable (VL) region of the LC comprising a sequence that has at least 70% (e.g., 80%, 85%, 90%, 91%, 92%, 93%, 94%, 95%, 96%, 97%, 98%, 99%, or greater) amino acid sequence identity to amino acids 1-108 of SEQ ID NO: 25.

In some embodiments, a nucleic acid molecule of the present disclosure comprises one or more nucleotide sequences encoding a heavy chain having at least 70% (e.g., 80%, 85%, 90%, 91%, 92%, 93%, 94%, 95%, 96%, 97%, 98%, 99%, or greater) amino acid sequence identity to the HC amino acid sequence set in SEQ ID NO: 26; and/or a light chain having at least 70% (e.g., 80%, 85%, 90%, 91%, 92%, 93%, 94%, 95%, 96%, 97%, 98%, 99%, or greater) amino acid sequence identity to the LC amino acid sequence set forth in SEQ ID NO: 27. In some embodiments, a nucleic acid molecule of the present disclosure comprises one or more nucleotide sequences encoding a heavy chain variable (VH) region of the HC comprising a sequence that has at least 70% (e.g., 80%, 85%, 90%, 91%, 92%, 93%, 94%, 95%, 96%, 97%, 98%, 99%, or greater) amino acid sequence identity to amino acids 1-123 of SEQ ID NO: 26; and/or a light chain variable (VL) region of the LC comprising a sequence that has at least 70% (e.g., 80%, 85%, 90%, 91%, 92%, 93%, 94%, 95%, 96%, 97%, 98%, 99%, or greater) amino acid sequence identity to amino acids 1-107 of SEQ ID NO: 27.

In some embodiments, a nucleic acid molecule of the present disclosure comprises a nucleotide sequence encoding a polypeptide sequences that is at least 70%, at least 75%, at least 80%, at least 85%, at least 90%, at least 95%, at least 96%, at least 97%, at least 98%, at least 99%, or 100% identity to the polypeptide sequence of any one of SEQ ID NOs: 1-6, or a variant and/or fragment thereof; and one or more nucleotide sequences encoding one or more (e.g., 2, 3, 4) antibodies selected from the anti-SARS-CoV-2 S glycoprotein mAbs E10933, E10987, E14315, or E15160 as described herein.

In some embodiments, a nucleic acid molecule of the present disclosure comprises a nucleotide sequence encoding a polypeptide sequence that is an influenza hemagglutinin, or a variant and/or fragment thereof. In some embodiments, a nucleic acid molecule of the present disclosure comprises a nucleotide sequence encoding a polypeptide sequence that is at least 70%, at least 75%, at least 80%, at least 85%, at least 90%, at least 95%, at least 96%, at least 97%, at least 98%, at least 99%, or 100% identity to the polypeptide sequence of any one of SEQ ID NO: 19, or a variant and/or fragment thereof.

In some embodiments, the nucleotide sequence that encodes an antigen and/or one or more antibodies described herein is operatively linked to a promoter for expression. A “promoter” is a regulatory region of DNA usually comprising a TATA box capable of directing RNA polymerase II to initiate RNA synthesis at the appropriate transcription initiation site for a particular polynucleotide sequence. A promoter may additionally comprise other regions which influence the transcription initiation rate. As used herein, the term “promoter” encompasses enhancers. The promoter sequences disclosed herein modulate transcription of an operably linked polynucleotide. A promoter can be active in one or more of the cell types disclosed herein (e.g., a eukaryotic cell, a non-human mammalian cell, a human cell, a rodent cell, a pluripotent cell, a one-cell stage embryo, a differentiated cell, or a combination thereof). A promoter can be, for example, a constitutively active promoter, a conditional promoter, an inducible promoter, a temporally restricted promoter (e.g., a developmentally regulated promoter), or a spatially restricted promoter (e.g., a cell-specific or tissue-specific promoter).

Examples of constitutive promoters include, but are not limited to, cytomegalovirus (CMV) promoter, EF1a, SV40, PGK1 (human or mouse), Ubc, human beta actin, CAG, Ac5, Polyhedrin, TEF1, GDS, CaMV35S, Ubi, H1, and U6 promoters.

Inducible promoters can include, for example, chemically regulated promoters and physically-regulated promoters. Chemically regulated promoters include, for example, alcohol-regulated promoters (e.g., an alcohol dehydrogenase (alcA) gene promoter), tetracycline-regulated promoters (e.g., a tetracycline-responsive promoter, a tetracycline operator sequence (tetO), a tet-On promoter, or a tet-Off promoter), steroid regulated promoters (e.g., a rat glucocorticoid receptor, a promoter of an estrogen receptor, or a promoter of an ecdysone receptor), or metal-regulated promoters (e.g., a metalloprotein promoter). Physically regulated promoters include, for example temperature-regulated promoters (e.g., a heat shock promoter such as Hsp70- and Hsp90-derived promoters) and light-regulated promoters (e.g., a light-inducible promoter or a light-repressible promoter). Other inducible promoters include lac, sp6, and an T7 promotor.

Tissue-specific promoters can be, for example, neuron-specific promoters, glia-specific promoters, muscle cell-specific promoters, heart cell-specific promoters, kidney cell-specific promoters, bone cell-specific promoters, endothelial cell-specific promoters, or immune cell-specific promoters (e.g., a B cell promoter or a T cell promoter).

Developmentally regulated promoters include, for example, promoters active only during an embryonic stage of development, or only in an adult cell.

Other non-limiting examples of promoters useful in the nucleic acid molecules of the present disclosure include a CB7/CAG promoter and associated upstream regulatory sequences, EF-1 alpha promoter, mU1a promoter, UB6 promoter, chicken beta-actin (CBA) promoter, and liver-specific promoters, such as TBG (Thyroxine-binding Globulin) promoter, APOA2 promoter, SERPINA1 (hAAT) promoter, ApoE.hAAT, or muscle-specific promoters, such as a human desmin promoter, CK8 promoter or Pitx3 promoter, inducible promoters, such as a hypoxia-inducible promoter or a rapamycin-inducible promoter, or a combination thereof.

In some embodiments, nucleic acid molecules of the present disclosure may include one promoter. In some embodiments, nucleic acid molecules of the present disclosure may include more than one (e.g., 2, 3, 4, or more) promoter.

In some embodiments, nucleic acid molecules of the present disclosure may encode a signal peptide fused to an antigen and/or one or more antibodies described herein. Such signal peptides are sequences which conventionally comprise a length of from 15 to 60 amino acids and are preferably localized on the N-terminus of the coded protein. Signal peptides are typically needed for the translocation across the membrane on the secretory pathway and, thus, universally control the entry of most proteins both in eukaryotes and prokaryotes to the secretory pathway. In eukaryotes, the signal peptide of a nascent precursor protein directs the ribosome to the rough endoplasmic reticulum (ER) membrane and initiates the transport of the growing peptide chain across it for processing. ER processing produces mature proteins, wherein the signal peptide is cleaved from precursor proteins, typically by an ER-resident signal peptidase of the host cell, or they remain uncleaved and function as a membrane anchor. A signal peptide may also facilitate the targeting of the protein to the cell membrane.

In some embodiments, a signal peptide may have a length of 15-60 amino acids. For example, a signal peptide may have a length of 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60 amino acids. In some embodiments, a signal peptide has a length of 20-60, 25-60, 30-60, 35-60, 40-60, 45-60, 50-60, 55-60, 15-55, 20-55, 25-55, 30-55, 35-55, 40-55, 45-55, 50-55, 15-50, 20-50, 25-50, 30-50, 35-50, 40-50, 45-50, 15-45, 20-45, 25-45, 30-45, 35-45, 40-45, 15-40, 20-40, 25-40, 30-40, 35-40, 15-35, 20-35, 25-35, 30-35, 15-30, 20-30, 25-30, 15-25, 20-25, or 15-20 amino acids.

Signal peptides can be derived from heterologous genes (which regulate expression of genes other than the antigens of interest in nature) or from the same genes encoding the antigens of interest. Examples of signal sequences which can be used according to the present disclosure are include, but are not limited to, signal sequences of conventional and non-conventional MHC molecules, cytokines, calreticulin and calnexin, Erp57, immunoglobulins, the invariant chain, Lamp1, tapasin, and all further membrane-located, endosomally-lysosomally or endoplasmic reticulum-associated proteins.

In some embodiments, a nucleic acid molecule of the present disclosure is not chemically modified and comprises the standard ribonucleotides. In some embodiments, nucleotides and nucleosides of the present disclosure comprise standard nucleoside residues such as those present in transcribed RNA (e.g., A, G, C, or U). In some embodiments, nucleotides and nucleosides of the present disclosure comprise standard deoxyribonucleosides such as those present in DNA (e.g., dA, dG, dC, or dT). The skilled artisan will appreciate that, except where otherwise noted, polynucleotide sequences set forth in the instant application will recite “T”s in a representative DNA sequence but where the sequence represents RNA, the “T”s would be substituted for “U”s.

In some embodiments the nucleic acid molecule is chemically modified. Chemical modification of a nucleic acid molecule can facilitate certain desirable properties of the molecule of the disclosure, for example, influencing the type of immune response to the molecule. For example, appropriate chemical modification of mRNAs can reduce unwanted innate immune responses against mRNA components and/or can facilitate desirable levels of protein expression of the antigen or antigens of interest.

In some embodiments, nucleic acid molecules of the present disclosure comprise a chemically modified nucleobase. Modified nucleic acids may be or may include, for example, deoxyribonucleic acids (DNAs), ribonucleic acids (RNAs), e.g. mRNAs, DNA-RNA hybrids, threose nucleic acids (TNAs), glycol nucleic acids (GNAs), peptide nucleic acids (PNAs), locked nucleic acids (LNAs, including LNA having a β-D-ribo configuration, α-LNA having an α-L-ribo configuration (a diastereomer of LNA), 2 amino-LNA having a 2′-amino functionalization, and 2′-amino-a-LNA having a 2′-amino functionalization), ethylene nucleic acids (ENA), cyclohexenyl nucleic acids (CeNA) and/or chimeras and/or combinations thereof.

Modified nucleotides can by synthesized by any useful method, such as, for example, chemically, enzymatically, or recombinantly, to include one or more modified or non-natural nucleosides. Polynucleotides can comprise a region or regions of linked nucleosides. Such regions can have variable backbone linkages. The linkages can be standard phosphodiester linkages, in which case the polynucleotides would comprise regions of nucleotides.

The modified nucleic acids disclosed herein can comprise various distinct modifications. In some embodiments, the modified nucleic acids contain one, two, or more (optionally different) nucleoside or nucleotide modifications. In some embodiments, a modified nucleic acid molecule, when introduced to a cell can exhibit one or more desirable properties, e.g., improved protein expression, reduced immunogenicity, or reduced degradation in the cell, as compared to an unmodified nucleic acid molecule.

In some embodiments, the polynucleotides of the present disclosure can have a uniform chemical modification of all or any of the same nucleoside type or a population of modifications produced by mere downward titration of the same starting modification in all or any of the same nucleoside type, or a measured percent of a chemical modification of all any of the same nucleoside type but with random incorporation, such as where all uridines are replaced by a uridine analog, e.g., pseudouridine or 5-methoxyuridine. In another embodiment, the polynucleotides can have a uniform chemical modification of two, three, or four of the same nucleoside type throughout the entire polynucleotide (such as all uridines and all cytosines, etc. are modified in the same way).

Modified nucleotide base pairing encompasses not only the standard adenosine-thymine, adenosine-uracil, or guanosine-cytosine base pairs, but also base pairs formed between nucleotides and/or modified nucleotides comprising non-standard or modified bases, wherein the arrangement of hydrogen bond donors and hydrogen bond acceptors permits hydrogen bonding between a non-standard base and a standard base or between two complementary non-standard base structures. One example of such non-standard base pairing is the base pairing between the modified nucleotide inosine and adenine, cytosine or uracil. Any combination of base/sugar or linker can be incorporated into polynucleotides of the present disclosure.

Modifications of nucleic acids that are useful in the nucleic acid molecules (e.g., mRNA polynucleotides) of the present disclosure include, but are not limited to the following nucleotides, nucleosides, and nucleobases: pseudouridine (ψ); 2-thiouridine (s2U); 4′-thiouridine; 5-methylcytosine; 2-thio-1-methyl-1-deaza-pseudouridine; 2-thio-1-methyl-pseudouridine; 2-thio-5-aza-uridine; 2-thio-dihydropseudouridine; 2-thio-dihydrouridine; 2-thio-pseudouridine; 4-methoxy-2-thio-pseudouridine; 4-methoxy-pseudouridine; 4-thio-1-methyl-pseudouridine; 4-thio-pseudouridine; 5-aza-uridine; dihydropseudouridine; 5-methyluridine; 5-methoxyuridine; 2′-O-methyl uridine; 1-methyl-pseudouridine (m1ψ); 1-ethyl-pseudouridine (e1ψ); 5-methoxy-uridine (mo5U); 5-methyl-cytidine (m5C); α-thio-guanosine; α-thio-adenosine; 5-cyano uridine; 4′-thio uridine 7-deaza-adenine; 1-methyl-adenosine (m1A); 2-methyl-adenine (m2A); N6-methyl-adenosine (m6A); 2, 6-Diaminopurine; 1-methyl-inosine (m1I); wyosine (imG); methylwyosine (mimG); 7-deaza-guanosine; 7-cyano-7-deaza-guanosine (preQO); 7-aminomethyl-7-deaza-guanosine (preQ1); 7-methyl-guanosine (m7G); 1-methyl-guanosine (m1G); 8-oxo-guanosine; 7-methyl-8-oxo-guanosine; 2,8-dimethyladenosine; 2-geranylthiouridine; 2-lysidine; 2-selenouridine; 3-(3-amino-3-carboxypropyl)-5,6-dihydrouridine; 3-(3-amino-3-carboxypropyl)pseudouridine; 3-methylpseudouridine; 5-(carboxyhydroxymethyl)-2′-O-methyluridine methyl ester; 5-aminomethyl-2-geranylthiouridine; 5-aminomethyl-2-selenouridine; 5-aminomethyluridine; 5-carbamoylhydroxymethyluridine; 5-carbamoylmethyl-2-thiouridine; 5-carboxymethyl-2-thiouridine; 5-carboxymethylaminomethyl-2-geranylthiouridine; 5-carboxymethylaminomethyl-2-selenouridine; 5-cyanomethyluridine; 5-hydroxycytidine; 5-methylaminomethyl-2-geranylthiouridine; 7-aminocarboxypropyl-demethylwyosine; 7-aminocarboxypropylwyosine; 7-aminocarboxypropylwyosine methyl ester; 8-methyladenosine; N4, N4-dimethylcytidine; N6-formyladenosine; N6-hydroxymethyladenosine; agmatidine; cyclic N6-threonylcarbamoyladenosine; glutamyl-queuosine; methylated undermodified hydroxywybutosine; N4,N4,2′-O-trimethylcytidine; 5-methylaminomethyl-2-thiouridine; geranylated geranylated 5-carboxymethylaminomethyl-2-thiouridine; 1-methyl-pseudouridine; 1-ethyl-pseudouridine; 1,2′-O-dimethyladenosine; 1-Deazaadenosine triphosphate (TP); 1-methyladenosine; 2 (amino)adenine; 2 (aminopropyl)adenine; 2 (methylthio) N6 (isopentenyl)adenine; 2-(alkyl)adenine; 2-(aminoalkyl)adenine; 2-(aminopropyl)adenine; 2-(halo)adenine; 2-(propyl) adenine; 2′-a-Ethynyladenosine TP; 2′-Amino-2′-deoxy-ATP; 2′-a-Trifluoromethyladenosine TP; 2′-Azido-2′-deoxy-ATP; 2′-b-Ethynyladenosine TP; 2′-b-Trifluoromethyladenosine TP; 2′-Deoxy-2′,2′-difluoroadenosine TP; 2′-Deoxy-2′-a-aminoadenosine TP: 2′-Deoxy-2′-a-azidoadenosine TP; 2′-Deoxy-2′-a-mercaptoadenosine TP; 2′-Deoxy-2′-a-thiomethoxyadenosine TP; 2′-Deoxy-2′-b-aminoadenosine TP; 2′-Deoxy-2′-b-azidoadenosine TP; 2′-Deoxy-2′-b-bromoadenosine TP; 2′-Deoxy-2′-b-chloroadenosine TP; 2′-Deoxy-2′-b-fluoroadenosine TP; 2′-Deoxy-2′-b-iodoadenosine TP; 2′-Deoxy-2′-b-mercaptoadenosine TP; 2′-Deoxy-2′-b-thiomethoxyadenosine TP; 2′Fluoro-N6-Bz-deoxyadenosine TP; 2′-OMe-2-Amino-ATP; 2′-O-methyladenosine; 2′O-methyl-N6-Bz-deoxyadenosine TP; 2′-O-ribosyladenosine (phosphate); 2-aminoadenine; 2-Aminoadenosine TP; 2-Amino-ATP; 2-Azidoadenosine TP; 2-Bromoadenosine TP; 2-Chloroadenosine TP; 2-Fluoroadenosine TP; 2-Iodoadenosine TP; 2-Mercaptoadenosine TP; 2-methoxy-adenine; 2-methyladenosine; 2-methylthio-adenine; 2-methylthio-N6 isopentenyladenosine; 2-methylthio-N6-(cis-hydroxyisopentenyl)adenosine; 2-methylthio-N6-hydroxynorvalyl carbamoyladenosine; 2-methylthio-N6-isopentenyladenosine; 2-methylthio-N6-methyladenosine; 2-methylthio-N6-threonyl carbamoyladenosine; 2-Trifluoromethyladenosine TP; 3-Deaza-3-bromoadenosine TP; 3-Deaza-3-chloroadenosine TP; 3-Deaza-3-fluoroadenosine TP; 3-Deaza-3-iodoadenosine TP; 3-Deazaadenosine TP; 4′-Azidoadenosine TP; 4′-Carbocyclic adenosine TP; 4′-Ethynyladenosine TP; 5′-Homo-adenosine TP; 6 (alkyl)adenine; 6 (methyl)adenine; 6-(alkyl)adenine; 6-(methyl)adenine; 7 (deaza)adenine; 7-deaza-8-aza-adenosine; 7-deaza-adenosine; 7-methyladenine; 8 (alkenyl)adenine; 8 (alkynyl)adenine; 8 (amino)adenine; 8 (thioalkyl)adenine; 8-(alkenyl)adenine; 8-(alkyl)adenine; 8-(alkynyl)adenine; 8-(amino)adenine; 8-(halo)adenine; 8-(hydroxyl)adenine; 8-(thioalkyl)adenine; 8-(thiol)adenine; 8-Aza-ATP; 8-azido-adeno sine; 8-bromo-adenosine TP; 8-Trifluoromethyladenosine TP; 9-Deazaadenosine TP; aza adenine; deaza adenine; Isopentenyladenosine; N1-methyl-adenosine; N6 (methyl)adenine; N6-(cis-hydroxyisopentenyl)adenosine; N6-(isopentyl)adenine; N6, N6 (dimethyl)adenine; N6,2′-O-dimethyladenosine; N6,N6,2′-O-trimethyladenosine; N6,N6-dimethyladenosine; N6-acetyladenosine; N6-cis-hydroxy-isopentenyl-adenosine; N6-glycinylcarbamoyladenosine; N6-hydroxynorvalylcarbamoyladenosine; N6-isopentenyladenosine; N6-methyladenosine; N6-methyl-N6-threonylcarbamoyladenosine; N6-threonyl carbamoyladenosine; 1,2′-O-dimethylguanosine; 1-Me-GTP; 1-methyl-6-thio-guanosine; 1-methylguanosine; 2 (propyl)guanine; 2-(alkyl)guanine; 2′-a-Ethynylguanosine TP; 2′-Amino-2′-deoxy-GTP; 2′-a-Trifluoromethylguanosine TP; 2′-Azido-2′-deoxy-GTP; 2′-b-Ethynylguanosine TP; 2′-b-Trifluoromethylguanosine TP; 2′-Deoxy-2′,2′-difluoroguanosine TP; 2′-Deoxy-2′-a-aminoguanosine TP; 2′-Deoxy-2′-a-azidoguanosine TP; 2′-Deoxy-2′-a-mercaptoguanosine TP; 2′-Deoxy-2′-a-thiomethoxyguanosine TP; 2′-Deoxy-2′-b-aminoguanosine TP; 2′-Deoxy-2′-b-azidoguanosine TP; 2′-Deoxy-2′-b-bromoguanosine TP; 2′-Deoxy-2′-b-chloroguanosine TP; 2′-Deoxy-2′-b-fluoroguanosine TP; 2′-Deoxy-2′-b-iodoguanosine TP; 2′-Deoxy-2′-b-mercaptoguanosine TP; 2′-Deoxy-2′-b-thiomethoxyguanosine TP; 2′Fluoro-N2-isobutyl-guanosine TP; 2′-O-methylguanosine; 2′O-methyl-N2-isobutyl-guanosine TP; 2′-O-ribosylguanosine (phosphate); 4′-Azidoguanosine TP; 4′-Carbocyclic guanosine TP; 4′-Ethynylguanosine TP; 5′-Homo-guanosine TP; 6 (methyl)guanine; 6-(alkyl)guanine; 6-(methyl)guanine; 6-methoxy-guanosine; 6-methyl-guanosine; 6-thio-7-deaza-8-aza-guanosine; 6-thio-7-deaza-guanosine; 6-thio-7-methyl-guanosine; 6-thio-guanosine; 7 (alkyl)guanine; 7 (deaza)guanine; 7 (methyl)guanine; 7-(alkyl)guanine; 7-(deaza)guanine; 7-(methyl)guanine; 7-aminomethyl-7-deazaguanosine; 7-cyano-7-deazaguanosine; 7-deaza-8-aza-guanosine; 7-methylguanosine; 8 (alkyl)guanine; 8 (alkynyl)guanine; 8 (halo)guanine; 8 (thioalkyl)guanine; 8-(alkenyl)guanine; 8-(alkyl)guanine; 8-(alkynyl)guanine; 8-(amino)guanine; 8-(halo)guanine; 8-(hydroxyl)guanine; 8-(thioalkyl)guanine; 8-(thiol)guanine; 8-bromo-guanosine TP; 9-Deazaguanosine TP; Archaeosine; aza guanine; deaza guanine; Methylwyo sine; N (methyl)guanine; N-(methyl)guanine; N1-methyl-guanosine; N2,2′-O-dimethylguanosine; N2,7,2′-O-trimethylguanosine; N2,7-dimethylguanosine; N2,N2,2′-O-trimethylguanosine; N2,N2,7-trimethylguanosine; N2,N2-dimethyl-6-thio-guanosine; N2,N2-dimethylguanosine; N2-isobutyl-guanosine TP; N2-methyl-6-thio-guanosine; N2-methylguanosine; Wyosine; (E)-5-(2-Bromo-vinyl)cytidine TP; 1-methyl-1-deaza-pseudoisocytidine; 1-methyl-pseudoisocytidine; 2-(thio)cytosine; 2,2′-anhydro-cytidine TP hydrochloride; 2,6-diaminopurine; 2′-a-Ethynylcytidine TP; 2′-Amino-2′-deoxy-CTP; 2′-a-Trifluoromethylcytidine TP; 2′-Azido-2′-deoxy-CTP; 2′-b-Ethynylcytidine TP; 2′-b-Trifluoromethylcytidine TP; 2′-Deoxy-2′,2′-difluorocytidine TP; 2′-Deoxy-2′-a-aminocytidine TP; 2′-Deoxy-2′-a-azidocytidine TP; 2′-Deoxy-2′-a-mercaptocytidine TP; 2′-Deoxy-2′-a-thiomethoxycytidine TP; 2′-Deoxy-2′-b-aminocytidine TP; 2′-Deoxy-2′-b-azidocytidine TP; 2′-Deoxy-2′-b-bromocytidine TP; 2′-Deoxy-2′-b-chlorocytidine TP; 2′-Deoxy-2′-b-fluorocytidine TP; 2′-Deoxy-2′-b-iodocytidine TP; 2′-Deoxy-2′-b-mercaptocytidine TP; 2′-Deoxy-2′-b-thiomethoxycytidine TP; 2′Fluor-N4-Bz-cytidine TP; 2′Fluoro-N4-Acetyl-cytidine TP; 2′-O-Methyl-5-(1-propynyl)cytidine TP; 2′-O-methylcytidine; 2′-O-Methyl-N4-Acetyl-cytidine TP; 2′O-methyl-N4-Bz-cytidine TP; 2-aminopurine; 2-methoxy-5-methyl-cytidine; 2-methoxy-cytidine; 2-thio-5-methyl-cytidine; 2-thiocytidine; 3 (deaza) 5 (aza)cytosine; 3 (methyl)cytosine; 3-(alkyl)cytosine; 3-(deaza) 5 (aza)cytosine; 3-(methyl)cytidine; 3′-Ethynylcytidine TP; 3-methylcytidine; 4,2′-O-dimethylcytidine; 4′-Azidocytidine TP; 4′-Carbocyclic cytidine TP; 4′-Ethynylcytidine TP; 4-methoxy-1-methyl-pseudoisocytidine; 4-methoxy-pseudoisocytidine; 4-methylcytidine; 4-thio-1-methyl-1-deaza-pseudoisocytidine; 4-thio-1-methyl-pseudoisocytidine; 4-thio-pseudoisocytidine; 5 (halo)cytosine; 5 (methyl)cytosine; 5 (propynyl)cytosine; 5 (trifluoromethyl)cytosine; 5-(1-Propynyl)ara-cytidine TP; 5-(2-Chloro-phenyl)-2-thiocytidine TP; 5-(4-Amino-phenyl)-2-thiocytidine TP; 5-(alkyl)cytosine; 5-(alkynyl)cytosine; 5-(halo)cytosine; 5-(propynyl)cytosine; 5-(trifluoromethyl)cytosine; 5,2′-O-dimethylcytidine; 5′-Homo-cytidine TP; 5-Aminoallyl-CTP; 5-aza-cytidine; 5-aza-zebularine; 5-bromo-cytidine; 5-Cyanocytidine TP; 5-Ethynylara-cytidine TP; 5-Ethynylcytidine TP; 5-formyl-2′-O-methylcytidine; 5-formylcytidine; 5-hydroxymethylcytidine; 5-iodo-cytidine; 5-Methoxycytidine TP; 5-methylcytidine; 5-methyl-zebularine; 5-propynyl cytosine; 5-Trifluoromethyl-Cytidine TP; 6-(azo)cytosine; 6-aza-cytidine; 7-deaza-2,6-diaminopurine; 7-deaza-8-aza-2,6-diaminopurine; 7-deaza-8-aza-2-aminopurine; 7-deaza-8-aza-adenine, 7-deaza-2-aminopurine; aza cytosine; deaza cytosine; Lysidine; N4 (acetyl)cytosine; N4,2′-O-dimethylcytidine; N4,N4-Dimethyl-2′-OMe-Cytidine TP; N4-acetyl-2′-O-methylcytidine; N4-acetylcytidine; N4-Amino-cytidine TP; N4-Benzoyl-cytidine TP; N4-methylcytidine; Pseudoisocytidine; Pseudo-iso-cytidine; pyrrolo-cytidine; pyrrolo-pseudoisocytidine; Zebularine; a-thio-cytidine; 1-methylinosine; Inosine; 1,2′-O-dimethylinosine; 2′-O-methylinosine; 7-methylinosine; Epoxyqueuosine; galactosyl-queuosine; Mannosylqueuosine; Queuosine; allyamino-thymidine; aza thymidine; deaza thymidine; deoxy-thymidine; 2′-O-methyluridine; 2-thiouridine; 3-methyluridine; 5-carboxymethyluridine; 5-hydroxyuridine; 5-taurinomethyl-2-thiouridine; 5-taurinomethyluridine; Dihydrouridine; Pseudouridine; (3-(3-amino-3-carboxypropyl)uridine; 1-methyl-3-(3-amino-5-carboxypropyl)pseudouridine; 1-methylpseduouridine; 2′-O-methylpseudouridine; 2-thio-2′-O-methyluridine; 3-(3-amino-3-carboxypropyl)uridine; 3,2′-O-dimethyluridine; 3-Methyl-pseudo-Uridine TP; 4-thiouridine; 5-(carboxyhydroxymethyl)uridine; 5-(carboxyhydroxymethyl)uridine methyl ester; 5,2′-O-dimethyluridine; 5,6-dihydro-uridine; 5-aminomethyl-2-thiouridine; 5-carbamoylmethyl-2′-O-methyluridine; 5-5-carbamoylmethyluridine; carboxyhydroxymethyluridine; 5-carboxyhydroxymethyluridine methyl ester; 5-carboxymethylaminomethyl-2′-O-methyluridine; 5-carboxymethylaminomethyl-2-thiouridine; 5-carboxymethylaminomethyluridine; 5-Carbamoylmethyluridine TP; 5-methoxycarbonylmethyl-2′-O-methyluridine; 5-methoxycarbonylmethyl-2-thiouridine; 5-methoxycarbonylmethyluridine; 5-methyluridine), 5-methoxyuridine; 5-methyl-2-thiouridine; 5-methylaminomethyl-2-selenouridine; 5-methylaminomethyl-2-thiouridine; 5-methylaminomethyluridine; 5-Methyldihydrouridine; 5-Oxyacetic acid-Uridine TP; 5-Oxyacetic acid-methyl ester-Uridine TP; N1-methyl-pseudo-uracil; N1-ethyl-pseudo-uracil; uridine 5-oxyacetic acid; uridine 5-oxyacetic acid methyl ester; 3-(3-Amino-3-carboxypropyl)-Uridine TP; 5-(iso-Pentenylaminomethyl)-2-thiouridine TP; 5-(iso-Pentenylaminomethyl)-2′-O-methyluridine TP; 5-(iso-Pentenylaminomethyl)uridine TP; 5-propynyl uracil; a-thio-uridine; 1 (aminoalkylamino-carbonylethylenyl)-2(thio)-pseudouracil; 1 (aminoalkylaminocarbonylethylenyl)-2,4-(dithio)pseudouracil; 1 (aminoalkylaminocarbonylethylenyl)-4 (thio)pseudouracil; 1 (aminoalkylaminocarbonylethylenyl)-pseudouracil; 1 (aminocarbonylethylenyl)-2(thio)-pseudouracil; 1 (aminocarbonylethylenyl)-2,4-(dithio)pseudouracil; 1 (aminocarbonylethylenyl)-4 (thio)pseudouracil; 1 (aminocarbonylethylenyl)-pseudouracil; 1 substituted 2(thio)-pseudouracil; 1 substituted 2,4-(dithio)pseudouracil; 1 substituted 4 (thio)pseudouracil; 1 substituted pseudouracil; 1-(aminoalkylamino-carbonylethylenyl)-2-(thio)-pseudouracil; 1-Methyl-3-(3-amino-3-carboxypropyl) pseudouridine TP; 1-Methyl-3-(3-amino-3-carboxypropyl)pseudo-UTP; 1-Methyl-pseudo-UTP; 1-Ethyl-pseudo-UTP; 2 (thio)pseudouracil; 2′ deoxy uridine; 2′ fluorouridine; 2-(thio)uracil; 2,4-(dithio)psuedouracil; 2′ methyl, 2′amino, 2′azido, 2′fluro-guanosine; 2′-Amino-2′-deoxy-UTP; 2′-Azido-2′-deoxy-UTP; 2′-Azido-deoxyuridine TP; 2′-Deoxy-2′-a-aminouridine TP; 2′-Deoxy-2′-a-azidouridine TP; 2-methylpseudouridine; 3 (3 amino-3 carboxypropyl)uracil; 4 (thio)pseudouracil; 4-(thio) pseudouracil; 4-(thio)uracil; 4-thiouracil; 5 (1,3-diazole-1-alkyl)uracil; 5 (2-aminopropyl)uracil; 5 (aminoalkyl)uracil; 5 (dimethylaminoalkyl)uracil; 5 (guanidiniumalkyl)uracil; 5 (methoxycarbonylmethyl)-2-(thio)uracil; 5 (methoxycarbonyl-methyl)uracil; 5 (methyl) 2 (thio)uracil; 5 (methyl) 2,4 (dithio)uracil; 5 (methyl) 4 (thio)uracil; 5 (methylaminomethyl)-2 (thio)uracil; 5 (methylaminomethyl)-2,4 (dithio)uracil; 5 (methylaminomethyl)-4 (thio)uracil; 5 (propynyl)uracil; 5 (trifluoromethyl)uracil; 5-(2-aminopropyl)uracil; 5-(alkyl)-2-(thio)pseudouracil; 5-(alkyl)-2,4 (dithio)pseudouracil; 5-(alkyl)-4 (thio)pseudouracil; 5-(alkyl)pseudouracil; 5-(alkyl)uracil; 5-(alkynyl)uracil; 5-(allylamino)uracil; 5-(cyanoalkyl)uracil; 5-(dialkylaminoalkyl)uracil; 5-(dimethylaminoalkyl)uracil; 5-(guanidiniumalkyl)uracil; 5-(halo)uracil; 5-(1,3-diazole-1-alkyl)uracil; 5-(methoxy)uracil; 5-(methoxycarbonylmethyl)-2-(thio)uracil; 5-(methoxycarbonyl-methyl)uracil; 5-(methyl) 2(thio)uracil; 5-(methyl) 2,4 (dithio)uracil; 5-(methyl) 4 (thio)uracil; 5-(methyl)-2-(thio)pseudouracil; 5-(methyl)-2,4 (dithio)pseudouracil; 5-(methyl)-4 (thio)pseudouracil; 5-(methyl)pseudouracil; 5-(methylaminomethyl)-2 (thio)uracil; 5-(methylaminomethyl)-2,4(dithio)uracil; 5-(methylaminomethyl)-4-(thio)uracil; 5-(propynyl)uracil; 5-(trifluoromethyl)uracil; 5-aminoallyl-uridine; 5-bromo-uridine; 5-iodo-uridine; 5-uracil; 6 (azo)uracil; 6-(azo)uracil; 6-aza-uridine; allyamino-uracil; aza uracil; deaza uracil; N3 (methyl)uracil; Pseudo-UTP-1-2-ethanoic acid; Pseudouracil; 4-Thio-pseudo-UTP; 1-carboxymethyl-pseudouridine; 1-methyl-1-deaza-pseudouridine; 1-propynyl-uridine; 1-taurinomethyl-1-methyl-uridine; 1-taurinomethyl-4-thio-uridine; 1-taurinomethyl-pseudouridine; 2-methoxy-4-thio-pseudouridine; (+)1-(2-Hydroxypropyl)pseudouridine TP; (2R)-1-(2-Hydroxypropyl)pseudouridine TP; (2S)-1-(2-Hydroxypropyl)pseudouridine TP; (E)-5-(2-Bromo-vinyl)ara-uridine TP; (E)-5-(2-Bromo-vinyl)uridine TP; (Z)-5-(2-Bromo-vinyl)ara-uridine TP; (Z)-5-(2-Bromo-vinyl)uridine TP; 1-(2,2,2-Trifluoroethyl)-pseudo-UTP; 1-(2,2,3,3,3-Pentafluoropropyl)pseudouridine TP; 1-(2,2-Diethoxyethyl)pseudouridine TP; 1-(2,4,6-Trimethylbenzyl)pseudouridine TP; 1-(2,4,6-Trimethyl-benzyl)pseudo-UTP; 1-(2,4,6-Trimethyl-phenyl)pseudo-UTP; 1-(2-Amino-2-carboxyethyl)pseudo-UTP; 1-(2-Amino-ethyl)pseudo-UTP; 1-(2-Hydroxyethyl)pseudouridine TP; 1-(2-Methoxyethyl)pseudouridine TP; 1-(3,4-Bis-trifluoromethoxybenzyl)pseudouridine TP; 1-(3,4-Dimethoxybenzyl)pseudouridine TP; 1-(3-Amino-3-carboxypropyl)pseudo-UTP; 1-(3-Amino-propyl)pseudo-UTP; 1-(3-Cyclopropyl-prop-2-ynyl)pseudouridine TP; 1-(4-Amino-4-carboxybutyl)pseudo-UTP; 1-(4-Amino-benzyl)pseudo-UTP; 1-(4-Amino-butyl)pseudo-UTP; 1-(4-Amino-phenyl)pseudo-UTP; 1-(4-Azidobenzyl)pseudouridine TP; 1-(4-Bromobenzyl)pseudouridine TP; 1-(4-Chlorobenzyl)pseudouridine TP; 1-(4-Fluorobenzyl)pseudouridine TP; 1-(4-Iodobenzyl)pseudouridine TP; 1-(4-Methanesulfonylbenzyl)pseudouridine TP; 1-(4-Methoxybenzyl)pseudouridine TP; 1-(4-Methoxy-benzyl)pseudo-UTP; 1-(4-Methoxy-phenyl)pseudo-UTP; 1-(4-Methylbenzyl)pseudouridine TP; 1-(4-Methyl-benzyl)pseudo-UTP; 1-(4-Nitrobenzyl)pseudouridine TP; 1-(4-Nitro-benzyl)pseudo-UTP; 1(4-Nitro-phenyl)pseudo-UTP; 1-(4-Thiomethoxybenzyl)pseudouridine TP; 1-(4-Trifluoromethoxybenzyl)pseudouridine TP; 1-(4-Trifluoromethylbenzyl)pseudouridine TP; 1-(5-Amino-pentyl)pseudo-UTP; 1-(6-Amino-hexyl)pseudo-UTP; 1,6-Dimethyl-pseudo-UTP; 1-[3-(2-{2-[2-(2-Aminoethoxy)-ethoxy]-ethoxy}-ethoxy)-propionyl]pseudouridine TP; 1-{3-[2-(2-Aminoethoxy)-ethoxy]-propionyl} pseudouridine TP; 1-Acetylpseudouridine TP; 1-Alkyl-6-(1-propynyl)-pseudo-UTP; 1-Alkyl-6-(2-propynyl)-pseudo-UTP; 1-Alkyl-6-allyl-pseudo-UTP; 1-Alkyl-6-ethynyl-pseudo-UTP; 1-Alkyl-6-homoallyl-pseudo-UTP; 1-Alkyl-6-vinyl-pseudo-UTP; 1-Allylpseudouridine TP; 1-Aminomethyl-pseudo-UTP; 1-Benzoylpseudouridine TP; 1-Benzyloxymethylpseudouridine TP; 1-Benzyl-pseudo-UTP; 1-Biotinyl-PEG2-pseudouridine TP; 1-Biotinylpseudouridine TP; 1-Butyl-pseudo-UTP; 1-Cyanomethylpseudouridine TP; 1-Cyclobutylmethyl-pseudo-UTP; 1-Cyclobutyl-pseudo-UTP; 1-Cycloheptylmethyl-pseudo-UTP; 1-Cycloheptyl-pseudo-UTP; 1-Cyclohexylmethyl-pseudo-UTP; 1-Cyclohexyl-pseudo-UTP; 1-Cyclooctylmethyl-pseudo-UTP; 1-Cyclooctyl-pseudo-UTP; 1-Cyclopentylmethyl-pseudo-UTP; 1-Cyclopentyl-pseudo-UTP; 1-Cyclopropylmethyl-pseudo-UTP; 1-Cyclopropyl-pseudo-UTP; 1-Hexyl-pseudo-UTP; 1-Homoallylpseudouridine TP; 1-Hydroxymethylpseudouridine TP; 1-iso-propyl-pseudo-UTP; 1-Me-2-thio-pseudo-UTP; 1-Me-4-thio-pseudo-UTP; 1-Me-alpha-thio-pseudo-UTP; 1-Methanesulfonylmethylpseudouridine TP; 1-Methoxymethylpseudouridine TP; 1-Methyl-6-(2,2,2-Trifluoroethyl)pseudo-UTP; 1-Methyl-6-(4-morpholino)-pseudo-UTP; 1-Methyl-6-(4-thiomorpholino)-pseudo-UTP; 1-Methyl-6-(substituted phenyl)pseudo-UTP; 1-Methyl-6-amino-pseudo-UTP; 1-Methyl-6-azido-pseudo-UTP; 1-Methyl-6-bromo-pseudo-UTP; 1-Methyl-6-butyl-pseudo-UTP; 1-Methyl-6-chloro-pseudo-UTP; 1-Methyl-6-cyano-pseudo-UTP; 1-Methyl-6-dimethylamino-pseudo-UTP; 1-Methyl-6-ethoxy-pseudo-UTP; 1-Methyl-6-ethylcarboxylate-pseudo-UTP; 1-Methyl-6-ethyl-pseudo-UTP; 1-Methyl-6-fluoro-pseudo-UTP; 1-Methyl-6-formyl-pseudo-UTP; 1-Methyl-6-hydroxyamino-pseudo-UTP; 1-Methyl-6-hydroxy-pseudo-UTP; 1-Methyl-6-iodo-pseudo-UTP; 1-Methyl-6-iso-propyl-pseudo-UTP; 1-Methyl-6-methoxy-pseudo-UTP; 1-Methyl-6-methylamino-pseudo-UTP; 1-Methyl-6-phenyl-pseudo-UTP; 1-Methyl-6-propyl-pseudo-UTP; 1-Methyl-6-tert-butyl-pseudo-UTP; 1-Methyl-6-trifluoromethoxy-pseudo-UTP; 1-Methyl-6-trifluoromethyl-pseudo-UTP; 1-Morpholinomethylpseudouridine TP; 1-Pentyl-pseudo-UTP; 1-Phenyl-pseudo-UTP; 1-Pivaloylpseudouridine TP; 1-Propargylpseudouridine TP; 1-Propyl-pseudo-UTP; 1-propynyl-pseudouridine; 1-p-tolyl-pseudo-UTP; 1-tert-Butyl-pseudo-UTP; 1-Thiomethoxymethylpseudouridine TP; 1-Thiomorpholinomethylpseudouridine TP; 1-Trifluoroacetylpseudouridine TP; 1-Trifluoromethyl-pseudo-UTP; 1-Vinylpseudouridine TP; 2,2′-anhydro-uridine TP; 2′-bromo-deoxyuridine TP; 2′-F-5-Methyl-2′-deoxy-UTP; 2′-OMe-5-Me-UTP; 2′-OMe-pseudo-UTP; 2′-a-Ethynyluridine TP; 2′-a-Trifluoromethyluridine TP; 2′-b-Ethynyluridine TP; 2′-b-Trifluoromethyluridine TP; 2′-Deoxy-2′,2′-difluorouridine TP; 2′-Deoxy-2′-a-mercaptouridine TP; 2′-Deoxy-2′-a-thiomethoxyuridine TP; 2′-Deoxy-2′-b-aminouridine TP; 2′-Deoxy-2′-b-azidouridine TP; 2′-Deoxy-2′-b-bromouridine TP; 2′-Deoxy-2′-b-chlorouridine TP; 2′-Deoxy-2′-b-fluorouridine TP; 2′-Deoxy-2′-b-iodouridine TP; 2′-Deoxy-2′-b-mercaptouridine TP; 2′-Deoxy-2′-b-thiomethoxyuridine TP; 2-methoxy-4-thio-uridine; 2-methoxyuridine; 2′-O-Methyl-5-(1-propynyl)uridine TP; 3-Alkyl-pseudo-UTP; 4′-Azidouridine TP; 4′-Carbocyclic uridine TP; 4′-Ethynyluridine TP; 5-(1-Propynyl)ara-uridine TP; 5-(2-Furanyl)uridine TP; 5-Cyanouridine TP; 5-Dimethylaminouridine TP; 5′-Homo-uridine TP; 5-iodo-2′-fluoro-deoxyuridine TP; 5-Phenylethynyluridine TP; 5-Trideuteromethyl-6-deuterouridine TP; 5-Trifluoromethyl-Uridine TP; 5-Vinylarauridine TP; 6-(2,2,2-Trifluoroethyl)-pseudo-UTP; 6-(4-Morpholino)-pseudo-UTP; 6-(4-Thiomorpholino)-pseudo-UTP; 6-(Substituted-Phenyl)-pseudo-UTP; 6-Amino-pseudo-UTP; 6-Azido-pseudo-UTP; 6-Bromo-pseudo-UTP; 6-Butyl-pseudo-UTP; 6-Chloro-pseudo-UTP; 6-Cyano-pseudo-UTP; 6-Dimethylamino-pseudo-UTP; 6-Ethoxy-pseudo-UTP; 6-Ethylcarboxylate-pseudo-UTP; 6-Ethyl-pseudo-UTP; 6-Fluoro-pseudo-UTP; 6-Formyl-pseudo-UTP; 6-Hydroxyamino-pseudo-UTP; 6-Hydroxy-pseudo-UTP; 6-Iodo-pseudo-UTP; 6-iso-Propyl-pseudo-UTP; 6-Methoxy-pseudo-UTP; 6-Methylamino-pseudo-UTP; 6-Methyl-pseudo-UTP; 6-Phenyl-pseudo-UTP; 6-Propyl-pseudo-UTP; 6-tert-Butyl-pseudo-UTP; 6-Trifluoromethoxy-pseudo-UTP; 6-Trifluoromethyl-pseudo-UTP; 2 (amino)purine; 2,4,5-(trimethyl)phenyl; 2′methyl, 2′amino, 2′azido, 2′fluro-cytidine; 2′methyl, 2′amino, 2′azido, 2′fluro-adenine; 2′methyl, 2′amino, 2′azido, 2′fluro-uridine; 2′-amino-2′-deoxyribose; 2,6-(diamino)purine; 1-(aza)-2-(thio)-3-(aza)-phenoxazin-1-yl: 1,3-(diaza)-2-(oxo)-phenthiazin-1-yl; 1,3-(diaza)-2-(oxo)-phenoxazin-1-yl; 1,3,5-(triaza)-2,6-(dioxa)-naphthalene; 2′-azido-2′-deoxyribose; 2′fluoro-2′-deoxyribose; 2′-fluoro-modified bases; 2′-OH-ara-adenosine TP; 2′-OH-ara-cytidine TP; 2′-OH-ara-guanosine TP; 2′-OH-ara-uridine TP; 2′-O-methyl-ribose; 2-amino-6-Chloro-purine; 2-Amino-riboside-TP; 2-aza-inosinyl; 2-oxo-7-aminopyridopyrimidin-3-yl; 2-oxo-pyridopyrimidine-3-yl; 2-pyridinone; 2-thio-zebularine; 3 nitropyrrole; 3-(methyl)-7-(propynyl)isocarbostyrilyl; 3-(methyl)isocarbostyrilyl; 4-(fluoro)-6-(methyl)benzimidazole; 4-(methyl)benzimidazole; 4-(methyl)indolyl; 4,6-(dimethyl)indolyl; 4-demethylwyosine; 5 nitroindole; 5 substituted pyrimidines; 5-(2-carbomethoxyvinyl)uridine TP; 5-(methyl)isocarbostyrilyl; 5-aza-2-thio-zebularine; 5-nitroindole; 6-(aza)pyrimidine; 6-(azo)thymine; 6-(methyl)-7-(aza)indolyl; 6-chloro-purine; 6-phenyl-pyrrolo-pyrimidin-2-on-3-yl; 7-(aminoalkylhydroxy)-1-(aza)-2-(thio)-3-(aza)-phenoxazin-1-yl; 7-(aminoalkylhydroxy)-1-(aza)-2-(thio)-3-(aza)-phenthiazin-1-yl; 7-(aminoalkylhydroxy)-1,3-(diaza)-2-(oxo)-phenoxazin-1-yl; 7-(aminoalkylhydroxy)-1,3-(diaza)-2-(oxo)-phenthiazin-1-yl; 7-(aza)indolyl; 7-(guanidiniumalkylhydroxy)-1-(aza)-2-(thio)-3-(aza)-phenoxazin-1-yl; 7-(guanidiniumalkylhydroxy)-1-(aza)-2-(thio)-3-(aza)-phenoxazinl-yl; 7-(guanidiniumalkylhydroxy)-1-(aza)-2-(thio)-3-(aza)-phenthiazin-1-yl; 7-(guanidiniumalkylhydroxy)-1,3-(diaza)-2-(oxo)-phenoxazin-1-yl; 7-(guanidiniumalkyl-hydroxy)-1,3-(diaza)-2-(oxo)-phenthiazin-1-yl; 7-(propynyl)isocarbostyrilyl; 7-(propynyl)isocarbostyrilyl, propynyl-7-(aza)indolyl; 7-deaza-2-amino-purine; 7-deaza-inosinyl; 7-substituted 1-(aza)-2-(thio)-3-(aza)-phenoxazin-1-yl; 7-substituted 1,3-(diaza)-2-(oxo)-phenoxazin-1-yl; 9-(methyl)-imidizopyridinyl; Alpha-thio-pseudo-UTP; Aminoindolyl; Anthracenyl; bis-ortho-(aminoalkylhydroxy)-6-phenyl-pyrrolo-pyrimidin-2-on-3-yl; bis-ortho-substituted-6-phenyl-pyrrolo-pyrimidin-2-on-3-yl; Difluorotolyl; Formycin A TP; Formycin B TP; Hydroxywybutosine; Hypoxanthine; Imidizopyridinyl; Inosinyl; Isocarbostyrilyl; Isoguanisine; Isowyosine; N2-substituted purines; N6-(19-Amino-pentaoxanonadecyl)adenosine TP; N6-methyl-2-amino-purine; N6-substituted purines; N-alkylated derivative; Napthalenyl; Nitrobenzimidazolyl; Nitroimidazolyl; Nitroindazolyl; Nitropyrazolyl; Nubularine; 06-substituted purines; O-alkylated derivative; ortho-(aminoalkylhydroxy)-6-phenyl-pyrrolo-pyrimidin-2-on-3-yl; ortho-substituted-6-phenyl-pyrrolo-pyrimidin-2-on-3-yl; Oxoformycin TP; para-(aminoalkylhydroxy)-6-phenyl-pyrrolo-pyrimidin-2-on-3-yl; para-substituted-6-phenyl-pyrrolo-pyrimidin-2-on-3-yl; Pentacenyl; Peroxywybutosine; Phenanthracenyl; Phenyl; propynyl-7-(aza)indolyl; Pseudouridine 1-(4-methylbenzenesulfonic acid) TP; Pseudouridine 1-(4-methylbenzoic acid) TP; Pseudouridine TP 1-[3-(2-ethoxy)]propionic acid; Pseudouridine TP 1-[3-{2-(2-[2-(2-ethoxy)-ethoxy]-ethoxy)-ethoxy} ]propionic acid; Pseudouridine TP 1-[3-{2-(2-[2-{2(2-ethoxy)-ethoxy}-ethoxy]-ethoxy)-ethoxy} ]propionic acid; Pseudouridine TP 1-[3-{2-(2-[2-ethoxy]-ethoxy)-ethoxy} ]propionic acid; Pseudouridine TP 1-[3-{2-(2-ethoxy)-ethoxy} ]propionic acid; Pseudouridine TP 1-methylphosphonic acid; Pseudouridine TP 1-methylphosphonic acid diethyl ester; Pseudo-UTP-N1-3-propionic acid; Pseudo-UTP-N1-4-butanoic acid; Pseudo-UTP-N1-5-pentanoic acid; Pseudo-UTP-N1-6-hexanoic acid; Pseudo-UTP-N1-7-heptanoic acid; Pseudo-UTP-N1-methyl-p-benzoic acid; Pseudo-UTP-N1-p-benzoic acid; Pyrenyl; pyridin-4-one ribonucleoside; pyridopyrimidin-3-yl; pyridopyrimidin-3-yl, 2-oxo-7-amino-pyridopyrimidin-3-yl; pyrrolo-pyrimidin-2-on-3-yl; Pyrrolopyrimidinyl; Pyrrolopyrizinyl; Pyrrolosine TP; Qbase; preQObase; preQ1base; Stilbenzyl; substituted 1,2,4-triazoles; Tetracenyl; Tubercidine; undermodified hydroxywybuto sine; Wybutosine; Xanthine; Xanthosine-5′-TP, and a combination thereof.

In some embodiments, the nucleic acid molecules of the present disclosure (e.g., mRNA) can include one of the above-listed modified nucleobases. In some embodiments, the nucleic acid molecules of the present disclosure (e.g., mRNA) can include a combination of at least two (e.g., 2, 3, 4 or more) of the aforementioned modified nucleobases.

In some embodiments, the nucleic acid molecules of the present disclosure (e.g., mRNA) comprise at least one chemically modified nucleobase, sugar, backbone, or any combination thereof. In some embodiments, the at least one chemically modified nucleobase is selected from pseudouracil (ψ), N1-methylpseudouracil (m1ψ), 1-ethylpseudouracil, 2-thiouracil (s2U), 4′-thiouracil, 5-methylcytosine, 5-methyluracil, 5-methoxyuracil, and any combination thereof.

In some embodiments, the nucleic acid molecules can have nucleotides with modified sugar moieties. Exemplary modified sugars include carbocyclic or acyclic sugars, sugars having substituent groups at one or more of the 2′, 3′ or 4′ positions and sugars having substituents in place of one or more hydrogen atoms of the sugar. In some embodiments, the sugar is modified by having a substituent group at the 2′ position. In additional embodiments, the sugar is modified by having a substituent group at the 3′ position. In other embodiments, the sugar is modified by having a substituent group at the 4′ position. Sugar substituent groups on the 2′ position (2′-) may be in the arabino (up) position or ribo (down) position. One example of a 2′-arabino modification is 2′-fluoro. Another example of a 2′-arabino modification is 2′-O-methyl. Other similar modifications may also be made at other positions on the sugar moiety, particularly the 3′ position of the sugar on the 3′ terminal nucleoside or in 2′-5′ linked oligonucleotides and the 5′ position of 5′ terminal nucleotide. In some embodiments, the sugar modification is a 2′-O-alkyl (e.g., 2′-O-methyl, 2′-O-methoxyethyl), 2′-halo (e.g., 2′-fluoro, 2′-chloro, 2′-bromo), and 4′ thio modifications.

Nucleic acid molecules of the present disclosure (e.g., mRNA) can also include backbone modifications, such as one or more phosphorothioate, phosphorodithioate, phosphotriester, boranophosphate, alkylphosphonates, phosphoramidates, phosphordiamidates, thionophosphoramidates, thionoalkylphosphonates, thionoalkylphosphotriesters, or phosphonocarboxylate linkages, where the linkage is the normal 3′-5′ linkage, 2′-5′ linked analog or inverted linkages such as 3′-3′, 5′-5′ and 2′-2′.

In some embodiments at least about 25%, at least about 30%, at least about 40%, at least about 50%, at least about 60%, at least about 70%, at least about 80%, at least about 90%, at least about 95%, at least about 99%, or 100% of the guanines, adenines, uracils or thymines are chemically modified.

Naturally-occurring eukaryotic mRNA molecules usually contain stabilizing elements, including, but not limited to untranslated regions (UTR) at their 5′-end (5′ UTR) and/or at their 3′-end (3′ UTR), in addition to other structural features, such as a 5′-cap structure or a 3′-poly(A) tail. Both the 5′ UTR and the 3′ UTR are typically transcribed from the genomic DNA and are elements of the premature mRNA. Characteristic structural features of mature mRNA, such as the 5′-cap and the 3′-poly(A) tail are usually added to the transcribed (premature) mRNA during mRNA processing.

In some embodiments, nucleic acid molecules of the present disclosure (e.g., mRNA) contain a 5′ and/or 3′ flanking region. Examples of elements that can be included in the 5′ and/or 3′ flanking region include, but are not limited to, untranslated regions (UTRs), Kozak sequences, an oligo(dT) sequence, detectable tags, and multiple cloning sites. Any portion of the flanking regions can be sequence-optimized and any can independently contain one or more different modifications as described herein, before and/or after sequence optimization.

In some embodiments, a 5′ UTR and/or a 3′ UTR region can be provided as flanking regions. Untranslated regions (UTRs) are nucleic acid sections of a polynucleotide before a start codon (5′ UTR) and after a stop codon (3′ UTR) that are not translated. Multiple 5′ or 3′ UTRs can be included in the flanking regions and can be the same or of different sequences.

A UTR can be homologous or heterologous to the coding region in a polynucleotide. In some embodiments, the UTR is homologous to the nucleotide sequence encoding the antigen and/or antibodies. In some embodiments, the UTR is heterologous to the nucleotide sequence encoding the antigen and/or antibodies. In some embodiments, the polynucleotide comprises two or more 5′ UTRs or functional fragments thereof, each of which have the same or different nucleotide sequences. In some embodiments, the polynucleotide comprises two or more 3′ UTRs or functional fragments thereof, each of which have the same or different nucleotide sequences.

In some embodiments, the 5′ UTR and the 3′ UTR can be heterologous. In some embodiments, the 5′ UTR can be derived from a different species than the 3′ UTR. In some embodiments, the 3′ UTR can be derived from a different species than the 5′ UTR.

Exemplary UTRs of the application include, but are not limited to, one or more 5′ UTR and/or 3′ UTR derived from the gene sequence of: an albumin (e.g., human albumin); an actin (e.g., human α or β actin); an ATP synthase (e.g., ATP5A1 or the β subunit of mitochondrial H+-ATP synthase); calreticulin (Calr); a globin, such as an α- or β-globin (e.g., a Xenopus, mouse, rabbit, or human globin); a glucose transporter (e.g., hGLUT1 (human glucose transporter 1)); a glyceraldehyde-3-phosphate dehydrogenase (GAPDH); a strong Kozak translational initiation signal; a human cytochrome b-245 α polypeptide (CYBA); a collagen (e.g., collagen type I, alpha 2 (Col1A2), collagen type I, alpha 1 (Col1A1), collagen type VI, alpha 2 (Col6A2), collagen type VI, alpha 1 (Col6A1)); a hydroxysteroid (17-β) dehydrogenase (HSD17B4); a virus (e.g., a tobacco etch virus (TEV), a Venezuelan equine encephalitis virus (VEEV), a Dengue virus, a cytomegalovirus (CMV) (e.g., CMV immediate early 1 (IE1)), a hepatitis virus (e.g., hepatitis B virus), a sindbis virus, or a PAV barley yellow dwarf virus (BYDV-PAV)); a heat shock protein (e.g., hsp70); a translation initiation factor (e.g., eIF4G); a tubulin; a histone; a citric acid cycle enzyme; a nucleobindin (e.g., Nucb1); a topoisomerase (e.g., a TOP gene lacking the 5′ TOP motif (the oligopyrimidine tract)); a ribosomal protein Large 32 (L32); a growth hormone (e.g., bovine (bGH) or human (hGH)); an elongation factor (e.g., elongation factor 1 al (EEF1A1)); a manganese superoxide dismutase (MnSOD); a myocyte enhancer factor 2A (MEF2A); a β-F1-ATPase, a creatine kinase, a myoglobin, a granulocyte-colony stimulating factor (G-CSF); a ribophorin (e.g., ribophorin I (RPNI)); a low density lipoprotein receptor-related protein (e.g., LRP1); a cardiotrophin-like cytokine factor (e.g., Nnt1); a procollagen-lysine, 2-oxoglutarate 5-dioxygenase 1 (Plod1); a ribosomal protein (e.g., human or mouse ribosomal protein, such as rps9); and functional fragments thereof and any combination thereof.

In some embodiments, the 5′ UTR may be a 5′ UTR derived from: β-globin; a strong Kozak translational initiation signal; a cytochrome b-245 α polypeptide (CYBA); a DEN; a HSD17B4; a 5′ proximal open reading frame of rubella virus (RV) RNA encoding nonstructural proteins; a Hsp70; an eIF4G; a GLUT1; a TEV; a TEEV; functional fragments thereof and any combination thereof.

In some embodiments, the 3′ UTR may be a 3′ UTR derived from a f3-globin; a CYBA; an albumin; a growth hormone (GH); an HBV; α-globin; a DEN; a BYDV-PAV; EEF1A1; a MnSOD; a β subunit of mitochondrial H(+)-ATP synthase (β-mRNA); a GLUT1; a MEF2A; a β-F1-ATPase; a VEEV; functional fragments thereof and combinations thereof.

In some embodiments, polynucleotide sequences of the present discourse may be engineered to incorporate UTR elements typically found in abundantly expressed genes of specific target organs. For example, introduction of 5′ UTR of liver-expressed mRNA, such as albumin, serum amyloid A, alpha fetoprotein, Apolipoprotein A/B/E, erythropoietin, transferrin, or Factor VIII, can enhance expression of polynucleotides in hepatic cell lines or liver. Likewise, use of 5′ UTR from other tissue-specific mRNA to improve expression in that tissue is possible for muscle (e.g., Herculin, MyoD, Myosin, Myoglobin, Myogenin), for endothelial cells (e.g., CD36, Tie-1), for myeloid cells (e.g., C/EBP, AML1, G-CSF, GM-CSF, CD11b, MSR, Fr-1, i-NOS), for leukocytes (e.g., CD45, CD18), for adipose tissue (e.g., CD36, GLUT4, ACRP30, adiponectin) and for lung epithelial cells (e.g., SP-A/B/C/D).

In some embodiments, UTRs are selected from a family of transcripts whose proteins share a common function, structure, feature or property. For example, an encoded polypeptide can belong to a family of proteins (i.e., that share at least one function, structure, feature, localization, origin, or expression pattern), which are expressed in a particular cell, tissue or at some time during development. The UTRs from any of the genes or mRNA can be swapped for any other UTR of the same or different family of proteins to create a new polynucleotide.

Additionally, one or more synthetic UTRs can be used.

In some embodiments, the polynucleotide comprises multiple UTRs, e.g., a double, a triple or a quadruple 5′ UTR or 3′ UTR. For example, a double UTR comprises two copies of the same UTR either in series or substantially in series.

Other non-UTR sequences can be incorporated into the polynucleotides of the disclosure. For example, introns or portions of intron sequences can be incorporated into the polynucleotides of the disclosure. Incorporation of intronic sequences can increase protein production as well as polynucleotide expression levels. In some embodiments, the polynucleotide of the disclosure comprises an internal ribosome entry site (IRES) such as those described herein instead of or in addition to a UTR.

In some embodiments, the UTR can also include at least one translational enhancer elements. As a non-limiting example, the translational enhancer element can be located between the transcription promoter and the start codon. In some embodiments, the 5′ UTR comprises a translational enhancer element. In some embodiments, the 3′ UTR comprises a translational enhancer element. In some embodiments, the polynucleotide of the disclosure comprises one or multiple copies of a translational enhancer element. The translational enhancer element in a translational enhancer polynucleotide can be organized in one or more sequence segments.

In some embodiments, a polynucleotide (e.g., mRNA) of the present disclosure may comprise a 5′ cap structure. 5′-capping of polynucleotides may be completed concomitantly during the in vitro transcription reaction using the following chemical RNA cap analogs to generate the 5′-guanosine cap structure according to manufacturer protocols: 3′-O-Me-m7G(5′)ppp(5′) G [the ARCA cap]; G(5′)ppp(5′)A; G(5′)ppp(5′)G; m7G(5′)ppp(5′)A; or m7G(5′)ppp(5′)G. 5′-capping of modified RNA may be completed post-transcriptionally using a Vaccinia Virus Capping Enzyme to generate the “Cap 0” structure: m7G(5′)ppp(5′)G. Cap 1 structure may be generated using both Vaccinia Virus Capping Enzyme and a 2′-O-methyl-transferase to generate: m7G(5′)ppp(5′)G-2′-O-methyl. Cap 2 structure may be generated from the Cap 1 structure followed by the 2′-O-methylation of the 5′-antepenultimate nucleotide using a 2′-O-methyl-transferase. Cap 3 structure may be generated from the Cap 2 structure followed by the 2′-O-methylation of the 5′-preantepenultimate nucleotide using a 2′-O-methyl-transferase. Enzymes may be derived from a recombinant source.

In some embodiments a polynucleotide (e.g., mRNA) of the present disclosure has a 5′ terminal cap that comprises a Cap0, Cap1, ARCA, inosine, N1-methyl-guanosine, 2′-fluoro-guanosine, 7-deaza-guano sine, 8-oxo-guanosine, 2-amino-guanosine, LNA-guanosine, 2-azidoguanosine, Cap2, Cap4, 5′ methylG cap, or an analog thereof.

In some embodiments, a polynucleotide (e.g., mRNA) of the present disclosure may comprise a 3′-poly(A) region. The 3′-poly(A) region can be an essential element for the stability of the individual mRNA and may also enhance the expression level of the encoded protein. The 3′-poly(A) region is typically a stretch of adenine nucleotides added to the 3′-end of the transcribed mRNA. It can, in some cases, comprise up to about 400 adenine nucleotides. In some embodiments, the poly-(A) region may have about 10 to about 200, about 20 to about 180, about 50 to about 160, about 70 to about 140, or about 80 to about 120 nucleotides in length.

In some embodiments, a polynucleotide (e.g., mRNA) of the disclosure includes a stabilizing element. Stabilizing elements may include, e.g., a histone stem-loop. The histone stem-loop is generally derived from histone genes and includes an intramolecular base pairing of two neighbored partially or entirely reverse complementary sequences separated by a spacer, consisting of a short sequence, which forms the loop of the structure. The unpaired loop region typically cannot base pair with either of the stem loop elements. It occurs more often in RNA, as is a key component of many RNA secondary structures but may also be present in single-stranded DNA. Stability of the stem-loop structure generally depends on the length, number of mismatches or bulges, and base composition of the paired region. In some embodiments, wobble base pairing (non-Watson-Crick base pairing) may be present. In some embodiments, the histone stem-loop sequence comprises a length of 15 to 45 nucleotides. In some embodiments, the histone stem-loop sequence comprises a length of 15 to 30 nucleotides, 20 to 35 nucleotides, 25 to 40 nucleotides, or 30 to 45 nucleotides.

In some embodiments, a polynucleotide (e.g., mRNA) of the disclosure has one or more AU-rich sequences removed. These sequences, also referred to as “AURES”, are destabilizing sequences found in the 3′ UTR. The AURES may be removed from the polynucleotide (e.g., mRNA) of the disclosure.

In some embodiments, the nucleotide sequence encoding an antigen and/or antibodies of the disclosure is codon optimized. Codon optimization takes advantage of the degeneracy of codons, as exhibited by the multiplicity of three-base pair codon combinations that specify an amino acid, and generally includes a process of modifying a nucleic acid sequence for enhanced expression in particular host cells (e.g., packaging cells) and/or target cells by replacing at least one codon of the native sequence with a codon that is more frequently or most frequently used in the genes of the host cells and/or target cells while maintaining the native amino acid sequence. For example, a nucleic acid encoding an antigen protein can be modified to substitute codons having a higher frequency of usage in a given prokaryotic or eukaryotic cell, including a human cell, a non-human cell, a mammalian cell, a rodent cell, a mouse cell, a rat cell, a hamster cell, or any other host and/or target cell, as compared to the naturally occurring nucleic acid sequence. Codon usage tables are readily available, for example, at the “Codon Usage Database.” These tables can be adapted in a number of ways. Computer algorithms for codon optimization of a particular sequence for expression in a particular host and/or target are also available (see, e.g., Gene Forge).

In some embodiments, a polynucleotide (e.g., mRNA) of the disclosure may be codon-optimized such that the levels of G/C are enhanced. The G/C-content of nucleic acid molecules (e.g., mRNA) may influence the stability of the RNA. RNA having an increased amount of guanine (G) and/or cytosine (C) residues may be functionally more stable than mRNA containing a large amount of adenine (A) and thymine (T) or uracil (U) nucleotides.

In some embodiments, a nucleic acid molecule according to the present disclosure has a length of from 50 to 15,000 nucleotides, e.g., a length of from 50 to 13,000 nucleotides, from 100 to 12,000 nucleotides, from 200 to 10,000 nucleotides, from 300 to 9,000 nucleotides, from 400 to 8,000 nucleotides, from 450 to 8,000 nucleotides, from 500 to 7,000 nucleotides, from 600 to 6,000 nucleotides, from 700 to 5,000 nucleotides, or from 800 to 4,500 nucleotides. In some embodiments, a nucleic acid molecule according to the present disclosure has a length of about 300 nucleotides, about 400 nucleotides, about 500 nucleotides, about 600 nucleotides, about 700 nucleotides, about 800 nucleotides, about 900 nucleotides, about 1000 nucleotides, about 1100 nucleotides, about 1200 nucleotides, about 1300 nucleotides, about 1400 nucleotides, about 1500 nucleotides, about 1600 nucleotides, about 1700 nucleotides, about 1800 nucleotides, about 1900 nucleotides, about 2000 nucleotides, about 2400 nucleotides, about 2500 nucleotides, about 2700 nucleotides, about 3000 nucleotides, about 3500 nucleotides, about 4000 nucleotides, about 4500 nucleotides, about 5000 nucleotides, about 5500 nucleotides, about 6000 nucleotides, about 6500 nucleotides, about 7000 nucleotides, about 7500 nucleotides, about 8000 nucleotides, about 8500 nucleotides, about 9000 nucleotides, about 9500 nucleotides, about 10000 nucleotides, or about 12000 nucleotides.

When transfected into mammalian host cells, the modified nucleic acid molecule (e.g., mRNA) may have a stability of between 12-18 hours, or greater than 18 hours, e.g., 24, 36, 48, 60, 72, or greater than 72 hours and are capable of being expressed by the mammalian host cells.

In some embodiments, nucleic acid molecules of the disclosure are chemically synthesized and/or purified. As a non-limiting example, nucleic acids the present disclosure may be manufactured in whole or in part using solid phase techniques. Solid-phase chemical synthesis of nucleic acids is an automated method wherein molecules are immobilized on a solid support and synthesized step by step in a reactant solution. Solid-phase synthesis is useful in site-specific introduction of chemical modifications in the nucleic acid sequences. Alternatively, the synthesis of nucleic acids of the present disclosure by the sequential addition of monomer building blocks may be carried out in a liquid phase. As a further alternative, a combination of synthetic methods may be used. For example, the use of solid-phase or liquid-phase chemical synthesis in combination with enzymatic ligation may be used to generate long chain nucleic acids.

### Vectors

In some embodiment, the nucleic acid molecule described herein (e.g., nucleic acid molecule encoding an antigen and/or one or more antibodies targeting one or more epitopes of the antigen) is comprised within a vector. The vector can be a viral vector or non-viral vector.

In some embodiments, the vector is a viral vector. Non-limiting examples of viral vectors include adenovirus, adeno-associated virus (AAV, e.g., AAV8, AAV9, AAVrh10, AAVS3), lentivirus, helper-dependent adenovirus, herpes simplex virus, poxvirus, hemagglutinin virus of Japan (HVJ), alphavirus (e.g., semliki forest virus (SFV), sindbis virus (SIN)), vaccinia virus, baculovirus vectors, and retrovirus vectors (e.g., murine leukemia virus (MLV), human immunodeficiency virus (HIV)).

In some embodiments, the viral vectors described herein are recombinant viral vectors. In some embodiments, the viral vectors described herein are altered such that they are replication-deficient in humans. In some embodiments, the viral vectors are hybrid vectors, e.g., an AAV vector placed into a “helpless” adenoviral vector. In some embodiments, viral vectors comprise a viral capsid from a first virus and viral envelope proteins from a second virus, e.g., VSV-G protein from vesicular stomatitus virus (VSV).

In some embodiments, the viral vectors described herein are AAV based viral vectors. In some embodiments, the AAV-based vectors described herein do not encode the AAV rep gene (required for replication) and/or the AAV cap gene (required for synthesis of the capsid proteins) (the rep and cap proteins may be provided by the packaging cells in trans). Multiple AAV serotypes have been identified. In some embodiments, AAV based vectors described herein comprise capsid components from one or more of AAV1, AAV2, AAV3, AAV4, AAV5, AAV6, AAV7, AAV8, AAV9, AAV10, AAV11, AAV12, AAV13, AAV14, AAV15, AAV16, AAVS3, AAV.rh8, AAV.rhIO, AAV.rh20, AAV.rh39, AAV.rh46, AAV.rh73, AAV.Rh74, AAV.RHM4-1, AAV.hu37, AAV.Anc80, AAV.Anc80L65, AAV.7m8, AAV.PHP.B, AAV.PHP.eB, AAV2.5, AAV2tYF, AAV3B, AAV.LK03, AAV.HSC1, AAV.HSC2, AAV.HSC3, AAV.HSC4, AAV.HSC5, AAV.HSC6, AAV.HSC7, AAV.HSC8, AAV.HSC9, AAV.HSC10, AAV.HSC11, AAV.HSC12, AAV.HSC13, AAV.HSC14, AAV.HSC15, or AAV.HSC16 or other rAAV particles, or combinations of two or more thereof. In some embodiments, AAV-based vectors provided herein comprise components from one or more serotypes of AAV. In some embodiments, AAV-based vectors described herein comprise components from one or more serotypes of AAV with tropism to desired tissues (e.g., liver, muscle, heart, kidney, neuron).

In some embodiments, the viral vectors described herein are lentivirus-based viral vectors. In some embodiments, lentiviral vectors described herein are derived from human lentiviruses. In some embodiments, lentiviral vectors described herein are derived from non-human lentiviruses. In some embodiments, lentiviral vectors described herein are packaged into a lentiviral capsid. In some embodiments, lentiviral vectors described herein comprise one or more of the following elements: long terminal repeats, a primer binding site, a polypurine tract, att sites, and an encapsidation site.

In some embodiments, the viral vectors described herein are HIV-based viral vectors. In some embodiments, HIV-based vectors described herein comprise at least two polynucleotides, wherein the gag and pol genes are from an HIV genome and the env gene is from another virus.

In some embodiments, the viral vectors described herein are herpes simplex virus-based viral vectors. In some embodiments, herpes simplex virus-based vectors described herein are modified such that they do not comprise one or more immediately early (IE) genes, rendering them non-cytotoxic.

In some embodiments, the viral vectors provided herein are MLV based viral vectors. In some embodiments, MLV-based vectors provided herein comprise up to 8 kb of heterologous DNA in place of the viral genes.

In some embodiments, the viral vectors provided herein are alphavirus-based viral vectors. In some embodiments, alphavirus vectors provided herein are recombinant, replication defective alphaviruses. In some embodiments, alphavirus replicons in the alphavirus vectors provided herein are targeted to specific cell types by displaying a functional heterologous ligand on their virion surface.

In some embodiments, the vector is a non-viral vector. Non-limiting examples of non-viral vectors include a plasmid (e.g., minicircle plasmid), a Sleeping Beauty transposon, a piggyBac transposon, or a single- or double-stranded DNA molecule that is used as a template for homology directed repair (HDR) based gene editing.

### Nanoparticles

In some embodiments, polypeptide (e.g., antigens, antibodies), nucleic acid molecule(s) encoding the antigens and/or one or more antibodies, or vectors comprising the nucleic acid molecule(s) described herein may be formulated in a carrier. The term “carrier” denotes an organic or inorganic ingredient, natural or synthetic, with which the nucleic acid molecule(s) is combined to facilitate administration.

In some embodiments, the carrier is a lipid nanoparticle (LNP), a polymeric nanoparticle, an inorganic nanoparticle, a lipid carrier such as a lipidoid, a liposome, a lipoplex, a peptide carrier, a nanoparticle mimic, a nanotube, or a conjugate.

Nanoparticle compositions are typically sized on the order of micrometers or smaller and can include a lipid bilayer. Nanoparticle compositions include, for example, lipid nanoparticles (LNPs), liposomes, and lipoplexes. In some embodiments, nanoparticle compositions are vesicles including one or more lipid bilayers. In some embodiments, a nanoparticle composition includes two or more concentric bilayers separated by aqueous compartments. Lipid bilayers can be functionalized and/or crosslinked to one another. Lipid bilayers can include one or more ligands, proteins, or channels.

In some embodiments, the nucleic acid molecule(s) is formulated in a lipid nanoparticle (LNP). The use of LNPs enables the effective delivery of chemically modified or unmodified mRNA vaccines. Both modified and unmodified LNP formulated mRNA vaccines are superior to conventional vaccines by a significant degree. Accordingly, lipid nanoparticles (LNPs) comprising the nucleic acid molecule(s), or the vectors of the present disclosure are provided.

In some embodiments, a lipid nanoparticle may comprise lipids such as a phospholipid, an ionizable lipid (such as an ionizable cationic lipid), or a structural lipid.

The LNPs disclosed herein can comprise one or more phospholipids, for example, one or more saturated or (poly)unsaturated phospholipids or a combination thereof. Phospholipids typically comprise a phospholipid moiety and one or more fatty acid moieties. A phospholipid moiety may be, e.g., phosphatidyl choline, phosphatidyl ethanolamine, phosphatidic acid, phosphatidyl glycerol, phosphatidyl serine, 2-lysophosphatidyl choline, or a sphingomyelin. A fatty acid moiety may be, e.g., alpha-linolenic acid, arachidic acid, arachidonic acid, erucic acid, eicosapentaenoic acid, behenic acid, docosapentaenoic acid, docosahexaenoic acid, lauric acid, myristic acid, myristoleic acid, phytanoic acid, palmitic acid, palmitoleic acid, stearic acid, oleic acid, or linoleic acid.

Phospholipids also include, but are not limited to, glycerophospholipids such as phosphatidylcholines, phosphatidylethanolamines, phosphatidylserines, phosphatidylinositols, phosphatidy glycerols, phosphatidic acids, and phosphosphingolipid, such as sphingomyelin. Non-limiting examples of phospholipid that can be used in the preparation of the composition of the present disclosure include dioleoyl phosphatidylcholine (DOPC), 1,2-dipalmitoyl-sn-glycero-3-phosphocholine (DPPC), Dioleoyl Phosphatidylethanolamine (DOPE), 1,2-dipalmitoyl-sn-glycero-3-succinate (DGS), or a combination thereof. Lecithin, a natural mixture of phospholipids typically derived from chicken eggs, sheep's wool, soybean and other vegetable sources, may also be used,

Non-natural phospholipid species including natural species with modifications and substitutions including branching, oxidation, cyclization, and alkynes can also be used. For example, a phospholipid can be functionalized with or cross-linked to one or more alkynes (e.g., an alkenyl group in which one or more double bonds is replaced with a triple bond). Under appropriate reaction conditions, an alkyne group can undergo a copper-catalyzed cycloaddition upon exposure to an azide. Such reactions can be useful in functionalizing a lipid bilayer of a nanoparticle composition to facilitate membrane permeation or cellular recognition or in conjugating a nanoparticle composition to a useful component such as a targeting or imaging moiety (e.g., a probe).

The LNPs disclosed herein can comprise one or more ionizable lipids. Examples of ionizable lipids that can be used in the LNPs of the present disclosure include 1,2-dioleyloxy-N,N-dimethylaminopropane (DODMA), (13Z,165Z)-N,N-dimethyl-3-nonydocosa-13-16-dien-1-amine (L608), 2-({8-[(3β)-cholest-5-en-3-yloxy]octyl}oxy)-N,N-dimethyl-3-[(9Z,12Z)-octadeca-9,12-dien-1-yloxy]propan-1-amine (Octyl-CLinDMA), 1,2-dilinoleyloxy-N,N-dimethylaminopropane (DLin-DMA), 2,2-dilinoleyl-4-dimethylaminomethyl-[1,3]-dioxolane (DLin-K-DMA), heptatriaconta-6,9,28,31-tetraen-19-yl 4-(dimethylamino)butanoate (DLin-MC3-DMA), 2,2-dilinoleyl-4-(2-dimethylaminoethyl)[1,3]-dioxolane (DLin-KC2-DMA), 3-(didodecylamino)-N1,N1,4-tridodecyl-1-piperazineethanamine (KL10), N1-[2-(didodecylamino)ethyl]-N1,N4,N4-tridodecyl-1,4-piperazinediethanamine (KL22), 14,25-ditridecyl-15,18,21,24-tetraaza-octatriacontane (KL25), (2R)-2-({8-[(3β)-cholest-5-en-3-yloxy]octyl}oxy)-N,N-dimethyl-3-[(9Z, 12Z)-octadeca-9,12-dien-1-yloxy]propan-1-amine (Octyl-CLinDMA (2R)), and (2S)-2-({8-[(3β)-cholest-5-en-3-yloxy]octyl}oxy)-N,N-dimethyl-3-[(9Z,12Z)-octadeca-9,12-dien-1-yloxy]propan-1-amine (Octyl-CLinDMA (2S)). Additionally, an ionizable amino lipid can also be a lipid including a cyclic amine group.

The LNPs disclosed herein can comprise one or more structural lipids. As used herein, the term “structural lipid” refers to sterols and also to lipids containing sterol moieties. Incorporation of structural lipids in the lipid nanoparticle may help mitigate aggregation of other lipids in the particle.

Structural lipids can include, but are not limited to, alpha-tocopherol, brassicasterol, cholesterol, campesterol, ergosterol, fecosterol, hopanoids, phytosterols, sitosterol, stigmasterol, steroids, tomatidine, tomatine, ursolic acid, and derivatives or mixtures thereof. In some embodiments, the structural lipid is a sterol. In some embodiments, the structural lipid is a steroid. In some embodiments, the structural lipid is cholesterol. In some embodiments, the structural lipid is a cholesterol derivative. Cholesterol derivatives suitable for use in the present disclosure include cholesterol β-D-glucoside, cholesterol 3-sulfate sodium salt, positively charged cholesterol such as DC-cholesterol and other cholesterol like molecules such as Campesterol, Ergosterol, Betulin, Lupeol, β-Sitosterol, α, β-Amyrin and bile acids.

In further embodiments, LNPs disclosed herein can comprise one or more polyethylene glycol (PEG)-modified lipids or PEGylated lipids. Non-limiting examples of PEG-modified lipids include PEG-modified phosphatidylethanolamine and phosphatidic acid, PEG-ceramide conjugates (e.g., PEG-CerC14 or PEG-CerC20), PEG-modified dialkylamines and PEG-modified 1,2-diacyloxypropan-3-amines. For example, a PEG lipid can be PEG-DMG, PEG-DLPE, PEG-c-DOMG, PEG-DMPE, PEG-DPPC, or a PEG-DSPE lipid.

In some embodiments, the PEG-modified lipid includes, but not limited to 1,2-dimyristoyl-sn-glycerol methoxypolyethylene glycol (PEG-DMG), 1,2-distearoyl-sn-glycero-3-phosphoethanolamine-N-[amino(polyethylene glycol)] (PEG-DSPE), PEG-dipalmetoleyl, PEG-dioleyl, PEG-distearyl, PEG-diacylglycamide (PEG-DAG), PEG-disteryl glycerol (PEG-DSG), PEG-dipalmitoyl phosphatidylethanolamine (PEG-DPPE), or PEG-1,2-dimyristyloxlpropyl-3-amine (PEG-c-DMA).

In some embodiments, the lipid moiety of the PEG-lipids includes those having lengths of from about C14 to about C22, preferably from about C14 to about C16. In some embodiments, a PEG moiety, e.g., a mPEG-NH2, has a size of about 1000, 2000, 5000, 10,000, 15,000 or 20,000 Da.

The LNPs of the present disclosure can include one or more additional components, such as carbohydrates, polymers, permeability enhancer molecules, surface altering agents (e.g., surfactants).

Carbohydrates can include, for example, simple sugars (e.g., glucose) and polysaccharides (e.g., glycogen and derivatives and analogs thereof).

A polymer can be included in and/or used to encapsulate or partially encapsulate a pharmaceutical composition disclosed herein (e.g., a pharmaceutical composition in lipid nanoparticle form). A polymer can be biodegradable and/or biocompatible. Examples of polymers include, but are not limited to, polyamines, polyacetylenes, polyacrylates, polyamides, polycarbamates, polycarbonates, polyethylenes, polyethers, polyesters, polyureas, polystyrenes, polyimides, polysulfones, polyurethanes, polyethyleneimines, polyisocyanates, polymethacrylates, polyacrylonitriles, and polyarylates.

In some embodiments, the ratio between the lipid composition and the polynucleotide can range from about 5:1 to about 60:1 (wt/wt). For example, the ratio between the lipid composition and the polynucleotide (e.g., mRNA) can be about 5:1, 6:1, 7:1, 8:1, 9:1, 10:1, 11:1, 12:1, 13:1, 14:1, 15:1, 16:1, 17:1, 18:1, 19:1, 20:1, 21:1, 22:1, 23:1, 24:1, 25:1, 26:1, 27:1, 28: 1, 29: 1, 30:1, 31: 1, 32:1, 33:1, 34: 1, 35:1, 36: 1, 37: 1, 38:1, 39:1, 40:1, 41:1, 42: 1, 43: 1, 44: 1, 45:1, 46: 1, 47: 1, 48: 1, 49: 1, 50:1, 51:1, 52:1, 53:1, 54:1, 55:1, 56:1, 57:1, 58:1, 59:1 or 60:1 (wt/wt). In some embodiments, the lipid nanoparticles described herein can comprise polynucleotides (e.g., mRNA) in a lipid:polynucleotide weight ratio of about 5:1 to about 10:1, from about 5:1 to about 15:1, from about 5:1 to about 20:1, from about 5:1 to about 25:1, from about 5:1 to about 30:1, from about 5:1 to about 35:1, from about 5:1 to about 40:1, from about 5:1 to about 45:1, from about 5:1 to about 50:1, from about 5:1 to about 55:1, from about 5:1 to about 60:1, from about 10:1 to about 15:1, from about 10:1 to about 20:1, from about 10:1 to about 25:1, from about 10:1 to about 30:1, from about 10:1 to about 35:1, from about 10:1 to about 40:1, from about 10:1 to about 45:1, from about 10:1 to about 50:1, from about 10:1 to about 55:1, from about 10:1 to about 60:1, from about 15:1 to about 20: 1, from about 15:1 to about 25:1,from about 15:1 to about 30:1, from about 15:1 to about 35:1, from about 15:1 to about 40:1, from about 15:1 to about 45:1, from about 15:1 to about 50:1, from about 15:1 to about 55:1, or from about 15:1 to about 60:1.

In one embodiment, the LNPs described herein can comprise the polynucleotide (e.g., mRNA) in a concentration from about 0.01 mg/ml to 2 mg/ml such as, but not limited to, 0.01 mg/mL, 0.02 mg/mL, 0.03 mg/mL, 0.04 mg/mL 0.05 mg/mL, 0.06 mg/mL, 0.07 mg/mL, 0.08 mg/mL, 0.09 mg/mL, 0.1 mg/ml, 0.2 mg/ml, 0.3 mg/ml, 0.4 mg/ml, 0.5 mg/ml, 0.6 mg/ml, 0.7 mg/ml, 0.8 mg/ml, 0.9 mg/ml, 1.0 mg/ml, 1.1 mg/ml, 1.2 mg/ml, 1.3 mg/ml, 1.4 mg/ml, 1.5 mg/ml, 1.6 mg/ml, 1.7 mg/ml, 1.8 mg/ml, 1.9 mg/ml, 2.0 mg/ml or greater than 2.0 mg/ml. In some embodiments, lipid nanoparticles described herein can comprise the polynucleotide (e.g., mRNA) in a concentration of about 0.01-0.1 mg/mL, 0.05-0.2 mg/mL, 0.1-0.3 mg/mL, 0.2-0.4 mg/mL, 0.3-0.6 mg/mL, 0.4-0.8 mg/mL, 0.5-1 mg/mL, 0.8-1.2 mg/mL, 1-1.5 mg/mL, or 1-2 mg/mL.

Nanoparticle compositions can be characterized by a variety of methods. For example, microscopy, e.g., transmission electron microscopy or scanning electron microscopy, can be used to examine the morphology and size distribution of a nanoparticle composition. Dynamic light scattering or potentiometry (e.g., potentiometric titrations) can be used to measure zeta potentials and determine particle sizes. Instruments such as the Zetasizer Nano ZS (Malvern Instruments Ltd, Malvern, Worcestershire, UK) can also be used to measure multiple characteristics of a nanoparticle composition, e.g., particle size, polydispersity index, and zeta potential.

In some embodiments, LNPs of the present disclosure have a diameter from about 10 to about 1000 nm such as, but not limited to, about 100 nm, about 150 nm, about 200 nm, about 250 nm, about 300 nm, about 350 nm, about 400 nm, about 450 nm, about 500 nm, about 550 nm, about 600 nm, about 650 nm, about 700 nm, about 750 nm, about 800 nm, about 850 nm, about 900 nm, about 950 nm or about 1000 nm. In some embodiments, LNPs of the present disclosure have a diameter of about 10 to about 20 nm, about 10 to about 30 nm, about 10 to about 40 nm, about 10 to about 50 nm, about 10 to about 60 nm, about 10 to about 70 nm, about 10 to about 80 nm, about 10 to about 90 nm, about 20 to about 30 nm, about 20 to about 40 nm, about 20 to about 50 nm, about 20 to about 60 nm, about 20 to about 70 nm, about 20 to about 80 nm, about 20 to about 90 nm, about 20 to about 100 nm, about 30 to about 40 nm, about 30 to about 50 nm, about 30 to about 60 nm, about 30 to about 70 nm, about 30 to about 80 nm, about 30 to about 90 nm, about 30 to about 100 nm, about 40 to about 50 nm, about 40 to about 60 nm, about 40 to about 70 nm, about 40 to about 80 nm, about 40 to about 90 nm, about 40 to about 100 nm, about 50 to about 60 nm, about 50 to about 70 nm, about 50 to about 80 nm, about 50 to about 90 nm, about 50 to about 100 nm, about 60 to about 70 nm, about 60 to about 80 nm, about 60 to about 90 nm, about 60 to about 100 nm, about 70 to about 80 nm, about 70 to about 90 nm, about 70 to about 100 nm, about 80 to about 90 nm, about 80 to about 100 nm, about 90 to about 100 nm, about 100 to about 150 nm, about 100 to about 200 nm, about 100 to about 300 nm, about 200 to about 400 nm, about 200 to about 300 nm, about 200 to about 500 nm, about 300 to about 400 nm, about 400 to about 600 nm, about 500 to about 800 nm, about 600 to about 900 nm, about 700 to about 1000 nm, about 800 to about 1000 nm.

A polydispersity index (PDI) is a measure of the size distribution of the lipid vesicle particles. The PDI can be calculated by determining the mean particle size of the lipid vesicle particles and the standard deviation from that size. There are techniques and instruments available for measuring the PDI of lipid vesicle particles. For example, DLS is a well-established technique for measuring the particle size and size distribution of particles in the submicron size range, with available technology to measure particle sizes of less than 1 nm (LS Instruments, CH; Malvern Instruments, UK). A small (e.g., less than 0.3) polydispersity index generally indicates a narrow particle size distribution. For a perfectly uniform sample, the PDI would be 0.0. In some embodiments, PDI of a lipid vesicle particle prepared according to the methods described herein prior to dehydration is between about 0.1 to about 0.7. In some embodiments, PDI of a lipid vesicle particle prepared according to the methods described herein prior to dehydration is about 0.1 to about 0.2, about 0.1 to about 0.3, about 0.1 to about 0.4, about 0.2 to about 0.5, about 0.3 to about 0.6, about 0.4 to about 0.7, or about 0.5 to 0.7. In some embodiments, PDI of a lipid vesicle particle described herein is about 0.1, about 0.15, about 0.2, about 0.3, about 0.4, about 0.5, about 0.6, or about 0.7.

In addition to LNPs, polypeptides or polynucleotides described herein may be formulated in other carriers. Examples of other suitable carriers include, but are not limited to, liposomes, lipoids and lipoplexes, particulate or polymeric nanoparticles, inorganic nanoparticles, peptide carriers, nanoparticle mimics, nanotubes, conjugates, immune stimulating complexes (ISCOM), virus-like particles (VLPs), self-assembling proteins, or emulsion delivery systems such as cationic submicron oil-in-water emulsions.

Liposomes are amphiphilic lipids which can form bilayers in an aqueous environment to encapsulate an aqueous core. The polypeptide or polynucleotide (e.g., mRNA) may be incorporated into the aqueous core. These lipids can have an anionic, cationic or zwitterionic hydrophilic head group. Liposomes can be formed from a single lipid or from a mixture of lipids. A mixture may comprise (1) a mixture of anionic lipids; (2) a mixture of cationic lipids; (3) a mixture of zwitterionic lipids; (4) a mixture of anionic lipids and cationic lipids; (5) a mixture of anionic lipids and zwitterionic lipids; (6) a mixture of zwitterionic lipids and cationic lipids; or (7) a mixture of anionic lipids, cationic lipids and zwitterionic lipids. Similarly, a mixture may comprise both saturated and unsaturated lipids. Exemplary phospholipids include, but are not limited to, phosphatidylcholines, phosphatidylserines, and phosphatidylethanolamines, phosphatidylglycerols. Cationic lipids include, but are not limited to, 1,2-distearyloxy-N,N-dimethyl-3-aminopropane (DSDMA), dioleoyl trimethylammonium propane (DOTAP), 1,2-dioleyloxy-N,Ndimethyl-3-aminopropane (DODMA), 1,2-dilinoleyloxy-N,N-dimethyl-3-aminopropane (DLinDMA), 1,2-dilinolenyloxy-N,N-dimethyl-3-aminopropane (DLenDMA). Zwitterionic lipids include, but are not limited to, acyl zwitterionic lipids and ether zwitterionic lipids. Examples of useful zwitterionic lipids include dodecylphosphocholine, DPPC, and DOPC.

Polymeric microparticles or nanoparticles can also be used to encapsulate or adsorb a polypeptide or polynucleotide (e.g., mRNA). The particles may be substantially non-toxic and biodegradable. The particles useful for delivering a polynucleotide (e.g., mRNA) may have an optimal size and zeta potential. For example, the microparticles may have a diameter in the range of 0.02 μm to 8 μm. In the instances when the composition has a population of micro- or nanoparticles with different diameters, at least 80%, 85%, 90%, or 95% of those particles ideally have diameters in the range of 0.03-7 μm. The particles may also have a zeta potential of between 40-100 mV, in order to provide maximal adsorption of the polynucleotide (e.g., mRNA) to the particles.

Non-toxic and biodegradable polymers include, but are not limited to, poly(ahydroxy acids), polyhydroxy butyric acids, polylactones (including polycaprolactones), polydioxanones, polyvalerolactone, polyorthoesters, polyanhydrides, polycyanoacrylates, tyrosine-derived polycarbonates, polyvinyl-pyrrolidinones or polyester-amides, one or more natural polymers such as a polysaccharide, for example pullulan, alginate, inulin, and chitosan, and combinations thereof. In some embodiments, the particles are formed from poly(ahydroxy acids), such as a poly(lactides) (PLA), poly(g-glutamic acid) (g-PGA), poly(ethylene glycol) (PEG), polystyrene, copolymers of lactide and glycolide such as a poly(D,L-lactide-co-glycolide) (PLG), and copolymers of D,L-lactide and caprolactone. Useful PLG polymers can include those having a lactide/glycolide molar ratio ranging, for example, from 20:80 to 80:20 e.g., 25:75, 40:60, 45:55, 55:45, 60:40, 75:25. Useful PLG polymers include those having a molecular weight between, for example, 5,000-200,000 Da e.g., between 10,000-100,000, 20,000-70,000, 40,000-50,000 Da.

The polymeric nanoparticle may also form hydrogel nanoparticles, hydrophilic three-dimensional polymer networks with favorable properties including flexible mesh size, large surface area for multivalent conjugation, high water content, and high loading capacity for antigens. Polymers such as Poly(L-lactic acid) (PLA), PLGA, PEG, and polysaccharides are suitable for forming hydrogel nanoparticles.

For example, the inorganic nanoparticles may be calcium phosphate nanoparticles, silicon nanoparticles or gold nanoparticles. Inorganic nanoparticles typically have a rigid structure and comprise a shell in which a polypeptide or polynucleotide is encapsulated or a core to which the polypeptide or polynucleotide may be covalently attached. The core may comprise one or more atoms such as gold (Au), silver (Ag), copper (Cu) atoms, Au/Ag, Au/Cu, Au/Ag/Cu, Au/Pt, Au/Pd or Au/Ag/Cu/Pd or calcium phosphate (CaP).

Other molecules suitable for complexing with the polypeptides or polynucleotides of the disclosure include cationic molecules, such as, polyamidoamine, dendritic polylysine, polyethylene irinine or polypropylene imine, polylysine, chitosan, DNA-gelatin coarcervates, DEAE dextran, dendrimers, or polyethylenimine (PEI).

In some embodiments, polypeptides or polynucleotides of the present disclosure can be conjugated to nanoparticles. Nanoparticles that may be used for conjugation with antigens and/or antibodies of the present disclosure include but not are limited to chitosan-shelled nanoparticles, carbon nanotubes, PEGylated liposomes, poly(d,l-lactide-co-glycolide)/montmorillonite (PLGA/MMT) nanoparticles, poly(lactide-co-glycolide) (PLGA) nanoparticles, poly-(malic acid)-based nanoparticles, and other inorganic nanoparticles (e.g., nanoparticles made of magnesium-aluminium layered double hydroxides with disuccinimidyl carbonate (DSC), and TiO₂ nanoparticles). Nanoparticles can be developed and conjugated to an antigens and/or antibodies contained in a composition for targeting virus-infected cells.

Oil-in-water emulsions may also be used for delivering a polypeptide or polynucleotide (e.g., mRNA) to a subject. Examples of oils useful for making the emulsions include animal (e.g., fish) oil or vegetable oil (e.g., nuts, grains and seeds). The oil may be biodegradable and biocompatible. Exemplary oils include, but are not limited to, tocopherols and squalene, a shark liver oil which is a branched, unsaturated terpenoid and combinations thereof. Terpenoids are branched chain oils that are synthesized biochemically in 5-carbon isoprene units.

The aqueous component of the emulsion can be water or can be water in which additional components have been added. For example, it may include salts to form a buffer e.g., citrate or phosphate salts, such as sodium salts. Exemplary buffers include a borate buffer, a citrate buffer, a histidine buffer a phosphate buffer, a Tris buffer, or a succinate buffer.

In some embodiments, the oil-in water emulsions include one or more cationic molecules. For example, a cationic lipid can be included in the emulsion to provide a positively charged droplet surface to which negatively-charged polynucleotide (e.g., mRNA) can attach. Exemplary cationic lipids include, but are not limited to: 1,2-dioleoyloxy-3-(trimethylammonio)propane (DOTAP), 1,2-Dimyristoyl-3-Trimethyl-AmmoniumPropane (DMTAP), 3′-[N-(N′,N′-Dimethylaminoethane)-carbamoyl]Cholesterol (DC Cholesterol), dimethyldioctadecyl-ammonium (DDA e.g., the bromide), dipalmitoyl(C16:0)trimethyl ammonium propane (DPTAP), distearoyltrimethylammonium propane (DSTAP). Other useful cationic lipids include benzalkonium chloride (BAK), benzethonium chloride, cholesterol hemisuccinate choline ester, lipopolyamines (e.g., dioctadecylamidoglycylspermine (DOGS), dipalmitoyl phosphatidylethanol-amidospermine (DPPES)), cetramide, cetylpyridinium chloride (CPC), cetyl trimethylammonium chloride (CTAC), cationic derivatives of cholesterol (e.g., cholesteryl-3.beta.-oxysuccinamidoethylenetrimethylammonium salt, cholesteryl-3.beta.-oxysuccinamidoethylene-dimethylamine, cholesteryl-3.beta.-carboxyamidoethylenetrimethylammonium salt, and cholesteryl-3.beta.-carboxyamidoethylenedimethylamine), N,N′,N′-polyoxyethylene (10)-N-tallow-1,3-diaminopropane, dodecyltrimethylammonium bromide, hexadecyltrimethyl-ammonium bromide, mixed alkyl-trimethyl-ammonium bromide, benzyldimethyldodecylammonium chloride, benzyldimethylhexadecyl-ammonium chloride, benzyltrimethylammonium methoxide, cetyldimethylethylammonium bromide, dimethyldioctadecyl ammonium bromide (DDAB), methylbenzethonium chloride, decamethonium chloride, methyl mixed trialkyl ammonium chloride, methyl trioctylammonium chloride), N,N-dimethyl-N-[2 (2-methyl-4-(1,1,3,3tetramethylbutyl)-phenoxy]-ethoxy)ethyl]-benzenemetha-naminium chloride (DEBDA), cholesteryl (4′-trimethylammonio) butanoate), N-alkyl pyridinium salts (e.g., cetylpyridinium bromide and cetylpyridinium chloride), N-alkylpiperidinium salts, dicationic bolaform electrolytes (C12Me6; C12BU6), dialkylglycetylphosphorylcholine, lysolecithin, L-alpha.dioleoylphosphatidylethanolamine, lipopoly-L (or D)-lysine (LPLL, LPDL), poly(L (or D)-lysine conjugated to N-glutarylphosphatidylethanolamine, dialkyldimethylammonium salts, [1-(2,3-dioleyloxy)-propyl]-N,N,N,trimethylammonium chloride, 1,2-diacyl-3-(trimethylammonio) propane (acyl group can be dimyristoyl, dipalmitoyl, distearoyl, or dioleoyl), 1,2-diacyl-3 (dimethylammonio)propane (acyl group can be dimyristoyl, dipalmitoyl, distearoyl, or dioleoyl), 1,2-dioleoyl-3-(4′-trimethyl-ammonio)butanoyl-sn-glycerol, 1,2-dioleoyl 3-succinyl-sn-glycerol choline ester, didodecyl glutamate ester with pendant amino group (C GluPhCnN), and ditetradecyl glutamate ester with pendant amino group (C14GluCnN+).

In some embodiments, in addition to the oil and cationic lipid, an emulsion can also include a non-ionic surfactant and/or a zwitterionic surfactant. Examples of useful surfactants include, but are not limited to: the polyoxyethylene sorbitan esters surfactants, e.g., polysorbate 20 and polysorbate 80; copolymers of ethylene oxide, propylene oxide, and/or butylene oxide, linear block copolymers; phospholipids, e.g., phosphatidylcholine; polyoxyethylene fatty ethers derived from lauryl, cetyl, stearyl and oleyl alcohols; polyoxyethylene-9-lauryl ether; octoxynols; (octylphenoxy)polyethoxyethanol; and sorbitan esters.

### Methods of the Disclosure

In one aspect, the present disclosure provides a method for redirecting an antibody response in a subject from one or more first epitopes of an antigen towards one or more second epitopes of said antigen. In certain embodiments, the method comprises administering to the subject (i) the antigen or a nucleic acid molecule encoding the antigen and (ii) one or more antibodies targeting the one or more first epitopes of the antigen or one or more nucleic acid molecules encoding the one or more antibodies, wherein the antigen or a nucleic acid molecule encoding the antigen and the one or more antibodies or one or more nucleic acid molecules encoding the one or more antibodies are administered to the subject in amounts effective for generating antibodies to one or more second epitopes of the antigen.

In another aspect, the present disclosure provides a method for shielding one or more first epitopes of an antigen from recognition by the immune system of a subject. In certain embodiments, the method comprises administering to the subject (i) the antigen or a nucleic acid molecule encoding the antigen and (ii) one or more antibodies targeting the one or more first epitopes of the antigen or one or more nucleic acid molecules encoding the one or more antibodies, wherein the one or more antibodies or one or more nucleic acid molecules encoding the one or more antibodies are administered to the subject in an amount effective to shield one or more first epitopes of the antigen from recognition by the immune system of the subject.

In yet another aspect, the present disclosure provides a method for generating one or more antibodies targeting a second epitope of an antigen. In certain embodiments, the method comprises administering to a subject (i) the antigen or a nucleic acid molecule encoding the antigen and (ii) one or more antibodies targeting one or more first epitopes of the antigen or one or more nucleic acid molecules encoding the one or more antibodies, wherein the antigen or a nucleic acid molecule encoding the antigen and the one or more antibodies or one or more nucleic acid molecules encoding the one or more antibodies are administered to the subject in amounts effective for generating antibodies to one or more second epitopes of the antigen.

In still yet another aspect, the present disclosure provides a method for increasing efficacy of a vaccine in a subject in need thereof, wherein the vaccine comprises an antigen or a nucleic acid molecule encoding the antigen. In certain embodiments the method comprises administering to the subject (i) the vaccine and (ii) one or more antibodies or one or more nucleic acid molecules encoding the one or more antibodies targeting one or more first epitopes of the antigen, wherein the vaccine and the one or more antibodies or one or more nucleic acid molecules encoding the one or more antibodies are administered to the subject in amounts effective for increasing efficacy of the vaccine.

In certain embodiments, the subject is a mammal. In certain embodiments, the subject is human. In certain embodiments, the subject is an experimental animal such as, but not limited to, a mouse, a rat, a rabbit, a dog, a cat, or a primate (e.g., a non-human primate).

In certain aspects and embodiments of the present disclosure, methods disclosed herein may comprise administering to a subject an effective amount of one or more antibodies targeting the one or more first epitopes of the antigen or one or more nucleic acid molecules encoding the one or more antibodies, wherein the one or more antibodies or one or more nucleic acid molecules encoding the one or more antibodies are administered to the subject before or during administering the antigen or a nucleic acid molecule encoding the antigen.

In some embodiments, the one or more antigens and/or antibodies disclosed herein may be administered at an amount effective to achieve a concentration of the one or more antibodies in a bodily fluid of the subject greater than or equal to 1000 mg/L-0.01 mg/L. In some embodiments, the one or more antigens and/or antibodies may be administered at an amount effective to achieve a concentration of the one or more antibodies in a bodily fluid of the subject greater than or equal to, for example, 990 mg/L-10 mg/L, 980 mg/L-20 mg/L, 970 mg/L-30 mg/L, 960 mg/L-40 mg/L, 950 mg/L-50 mg/L, 940 mg/L-60 mg/L, 930 mg/L-70 mg/L, 920 mg/L-80 mg/L, 910 mg/L-90 mg/L, 900 mg/L-100 mg/L, 890 mg/L-110 mg/L, 880 mg/L-120 mg/L, 870 mg/L-130 mg/L, 860 mg/L-140 mg/L, 850 mg/L-150 mg/L, 840 mg/L-160 mg/L, 830 mg/L-170 mg/L, 820 mg/L-180 mg/L, 810 mg/L-190 mg/L, 800 mg/L-200 mg/L, 790 mg/L-210 mg/L, 780 mg/L-220 mg/L, 770 mg/L-230 mg/L, 760 mg/L-240 mg/L, 750 mg/L-250 mg/L, 740 mg/L-260 mg/L, 730 mg/L-270 mg/L, 720 mg/L-280 mg/L, 710 mg/L-290 mg/L, 700 mg/L-300 mg/L, 690 mg/L-310 mg/L, 680 mg/L-320 mg/L, 670 mg/L-330 mg/L, 660 mg/L-340 mg/L, 650 mg/L-350 mg/L, 640 mg/L-360 mg/L, 630 mg/L-370 mg/L, 620 mg/L-380 mg/L, 610 mg/L-390 mg/L, 600 mg/L-400 mg/L, 590 mg/L-410 mg/L, 580 mg/L-420 mg/L, 570 mg/L-430 mg/L, 560 mg/L-440 mg/L, 550 mg/L-450 mg/L, 540 mg/L-460 mg/L, 530 mg/L-470 mg/L, 520 mg/L-480 mg/L, or 510 mg/L-490 mg/L, or more. In some embodiments, the one or more antigens and/or antibodies may be administered at an amount effective to achieve a concentration of the one or more antibodies in a bodily fluid of the subject greater than or equal to, for example, 0.01 mg/L, 0.02 mg/L, 0.03 mg/L, 0.04 mg/L, 0.05 mg/L, 0.06 mg/L, 0.07 mg/L, 0.08 mg/L, 0.09 mg/L, 0.1 mg/L, 0.3 mg/L, 0.5 mg/L, 0.7 mg/L, 0.9 mg/L, 1 mg/L, 2 mg/L, 3 mg/L, 4 mg/L, 5 mg/L, 6 mg/L, 7 mg/L, 8 mg/L, 9 mg/L, 10 mg/L, 30 mg/L, 50 mg/L, 70 mg/L, 90 mg/L, 110 mg/L, 130 mg/L, 150 mg/L, 170 mg/L, 190 mg/L, 210 mg/L, 230 mg/L, 250 mg/L, 270 mg/L, 290 mg/L, 310 mg/L, 330 mg/L, 350 mg/L, 370 mg/L, 390 mg/L, 410 mg/L, 430 mg/L, 450 mg/L, 470 mg/L, 490 mg/L, 510 mg/L, 530 mg/L, 550 mg/L, 570 mg/L, 590 mg/L, 610 mg/L, 630 mg/L, 650 mg/L, 670 mg/L, 690 mg/L, 710 mg/L, 730 mg/L, 750 mg/L, 770 mg/L, 790 mg/L, 810 mg/L, 830 mg/L, 850 mg/L, 870 mg/L, 890 mg/L, 910 mg/L, 930 mg/L, 950 mg/L, 970 mg/L, 990 mg/L, 1000 mg/L, or more. In some embodiments, the bodily fluid is whole blood, plasma, serum, saliva, or urine.

In some embodiments, the one or more antigens and/or antibodies disclosed herein may be administered, for example, without limitation, as a protein, protein fragment, and/or protein fusion.

In some embodiments, the antigens and/or antibody or plurality thereof disclosed herein may be administered as a nucleic acid molecule (e.g., DNA and/or RNA molecule) that contains the antigen and/or antibody of interest and expressing the antigen and/or antibody of interest using the host cellular expression machinery to express the antigen and/or antibody polypeptide in vivo. Nucleic acid molecule encoding the one or more antigens and/or antibodies disclosed herein are further described in the sections above.

In certain embodiments, (i) the one or more antibodies or one or more nucleic acid molecules encoding the one or more antibodies and (ii) the antigen or the nucleic acid molecule encoding the antigen are administered as different formulations.

In certain embodiments, (i) the one or more antibodies or one or more nucleic acid molecules encoding the one or more antibodies and (ii) the antigen or the nucleic acid molecule encoding the antigen are administered in the same formulation. When the one or more antibodies or one or more nucleic acid molecules encoding the one or more antibodies and the antigen or the nucleic acid molecule encoding the antigen are administered in the same formulation, the method may comprise administering to the subject a nucleic acid molecule encoding (i) the one or more antibodies and (ii) the antigen. In some embodiments, the nucleic acid molecule is an RNA molecule (e.g., an mRNA molecule). In some embodiments, the nucleic acid molecule is a DNA molecule. In some embodiments, the nucleic acid molecule is chemically modified. The chemical modifications may comprise any number of chemical modifications disclosed herein. In some embodiments, the nucleic acid molecule disclosed herein may be comprised within a vector disclosed herein.

In some embodiments, the one or more antigens or nucleic acid molecules encoding the one or more antigens may be administered as a vaccine. Accordingly, a vaccine comprising one or more antigens or a nucleic acid molecules encoding the antigen(s) disclosed herein is provided herein.

The one or more antigens and/or antibodies, or related nucleic acid molecules encoding same, disclosed herein may be adapted for administration by any appropriate route such as, e.g., parenteral (including subcutaneous, intramuscular, or intravenous), enteral (including oral or rectal), inhalation, or intranasal routes.

Such compositions may be prepared, for example, by mixing the active ingredient with the carrier(s) or excipient(s) under sterile conditions.

Accordingly, provided herein are also formulations comprising the antigens and/or antibodies, or antigen- and/or antibody-based molecules (such as vaccines, complexes, fusion proteins, or conjugates comprising the antigen and/or antibodies, and related nucleic acid molecules, vectors, cells, or binding moieties disclosed herein) of the present disclosure.

In some embodiments, provided herein is a formulation comprising an antigen or a nucleic acid molecule encoding the antigen, and one or more antibodies targeting one or more first epitopes of the antigen or one or more nucleic acid molecules encoding the one or more antibodies.

In some embodiments, provided herein is a formulation comprising two or more monoclonal antibodies (mAbs) targeting one or more first epitopes of an antigen.

Compositions based on the antigens and/or antibodies, or antigen- and/or antibody-based molecules (such as vaccines, complexes, fusion proteins, or conjugates comprising the antigen and/or antibodies, and related nucleic acid molecules, vectors, cells, or binding moieties disclosed herein) can be formulated in any conventional manner using one or more physiologically acceptable carriers and/or excipients. antigens and/or antibodies, or antigen- and/or antibody-based molecules (such as vaccines, complexes, fusion proteins, or conjugates comprising the antigen and/or antibodies, and related nucleic acid molecules, vectors, cells, or binding moieties disclosed herein) can be formulated for administration by, for example, injection, inhalation, or insulation (either through the mouth or the nose) or by oral, buccal, parenteral or rectal administration, or by administration directly to an organ or tissue.

The antigens and/or antibodies, or antigen- and/or antibody-based molecules can be formulated for a variety of modes of administration, including systemic, topical, or localized administration. For systemic administration, injection is preferred, including intramuscular, intravenous, intraperitoneal, and subcutaneous. For the purposes of injection, the pharmaceutical compositions can be formulated in liquid solutions, preferably in physiologically compatible buffers, such as Hank's solution or Ringer's solution. In addition, the compositions may be formulated in solid form and redissolved or suspended immediately prior to use. Lyophilized forms of the pharmaceutical composition are also suitable.

In some embodiments, the compositions comprising antigens and/or antibodies, or antigen- and/or antibody-based molecules of the present disclosure may be lyophilized. As a non-limiting example, the obtained lyophilizate can be reconstituted into a hydrous composition by adding a hydrous solvent. In some embodiments, the hydrous composition may be able to be directly administered parenterally to a subject. Therefore, a further embodiment of the present disclosure is a hydrous pharmaceutical composition, obtainable via reconstitution of the lyophilizate with a hydrous solvent.

In some embodiments, the compositions disclosed herein may comprise a lyophilized formulation. As a non-limiting example, the lyophilization formulation may comprise antigens and/or antibodies, or antigen- and/or antibody-based molecules of the disclosure, mannitol, and/or TWEEN 80®. As another non-limiting example, the lyophilization formulation may comprise the antigens and/or antibodies, or antigen- and/or antibody-based molecules disclosed herein, mannitol and poloxamer 188. In some embodiments, the pharmaceutical composition may comprise a lyophilization formulation comprising a reconstituted-liquid composition.

In some embodiments, compositions of the present disclosure may provide a formulation with an enhanced solubility and/or moistening of the lyophilizate over previously known compositions. As a non-limiting example, enhanced solubility and/or moistening of the lyophilizate may be achieved using an appropriate composition of excipients. In this way, compositions of the present disclosure comprising antigens and/or antibodies, or antigen- and/or antibody-based molecules disclosed herein may be developed to show a desired shelf stability at (e.g., at −20° C., +5° C., or +25° C.) and can be easily resolubilized such that the lyophilizate can be completely dissolved through the use of a buffer or other excipients from seconds up to two or more minutes, with or without the use of an of ultrasonic homogenizer. As a non-limiting example, the pH-value of the resulting solution may be between pH 2.7 and pH 9. Furthermore, the compositions can be easily provided to a subject via any appropriate delivery route disclosed herein, e.g., parenteral (including subcutaneous, intramuscular, or intravenous), enteral (including oral or rectal), inhalation, or intranasal routes.

Non-limiting examples of delivery routes that may be useful for administering the antigens and/or antibodies, or antigen- and/or antibody-based molecules include, auricular (in or by way of the ear), biliary perfusion, buccal (directed toward the cheek), cardiac perfusion, caudal block, conjunctival, cutaneous, dental (to a tooth or teeth), dental intracoronal, diagnostic, electro-osmosis, endocervical, endosinusial, endotracheal, enema, enteral (into the intestine), epicutaneous (application onto the skin), epidural (into the dura mater), extra-amniotic administration, extracorporeal, eye drops (onto the conjunctiva), gastroenteral, hemodialysis, infiltration, insufflation (snorting), interstitial, intra-abdominal, intra-amniotic, intra-arterial (into an artery), intra-articular, intrabiliary, Intrabronchial, intrabursal, intracardiac (into the heart), intracartilaginous (within a cartilage), intracaudal (within the cauda equine), intracavernous injection (into a pathologic cavity), intracavitary (into the base of the penis), intracerebral (into the cerebrum), intracerebroventricular (into the cerebral ventricles), intracisternal (within the cisterna magna cerebellomedularis), intracorneal (within the cornea), intracoronary (within the coronary arteries), intracorporus cavernosum (within the dilatable spaces of the corporus cavernosa of the penis), intradermal (into the skin itself), intradiscal (within a disc), intraductal (within a duct of a gland), intraduodenal (within the duodenum), intradural (within or beneath the dura), intraepidermal (to the epidermis), intraesophageal (to the esophagus), intragastric (within the stomach), intragingival (within the gingivae), intraileal (within the distal portion of the small intestine), intralesional (within or introduced directly to a localized lesion), intraluminal (within a lumen of a tube), intralymphatic (within the lymph), intramedullary (within the marrow cavity of a bone), intrameningeal (within the meninges), intramuscular (into a muscle), intramyocardial (within the myocardium), intraocular (within the eye), intraosseous infusion (into the bone marrow), intraovarian (within the ovary), intraparenchymal (into brain tissue), intrapericardial (within the pericardium), intraperitoneal (infusion or injection into the peritoneum), intrapleural (within the pleura), intraprostatic (within the prostate gland), intrapulmonary (within the lungs or its bronchi), intrasinal (within the nasal or periorbital sinuses), intraspinal (within the vertebral column), intrasynovial (within the synovial cavity of a joint), intratendinous (within a tendon), intratesticular (within the testicle), intrathecal (into the spinal canal), intrathecal (within the cerebrospinal fluid at any level of the cerebrospinal axis), intrathoracic (within the thorax), intratubular (within the tubules of an organ), intratumor (within a tumor), intratympanic (within the aurus media), intrauterine, intravaginal administration, intravascular (within a vessel or vessels), intravenous (into a vein), intravenous bolus, intravenous drip, intraventricular (within a ventricle), intravesical infusion, intravitreal (through the eye), iontophoresis (by means of electric current where ions of soluble salts migrate into the tissues of the body), irrigation (to bathe or flush open wounds or body cavities), laryngeal (directly upon the larynx), nasal administration (through the nose), nasogastric (through the nose and into the stomach), nerve block, occlusive dressing technique (topical route administration which is then covered by a dressing which occludes the area), ophthalmic (to the external eye), or in ear drops, oral (by way of the mouth), oropharyngeal (directly to the mouth and pharynx), parenteral, percutaneous, periarticular, peridural, perineural, periodontal, photopheresis, rectal, respiratory (within the respiratory tract by inhaling orally or nasally for local or systemic effect), retrobulbar (behind the pons or behind the eyeball), soft tissue, spinal, subarachnoid, subconjunctival, subcutaneous (under the skin), sublabial, sublingual, submucosal, topical, transdermal, transdermal (diffusion through the intact skin for systemic distribution), transmucosal (diffusion through a mucous membrane), transplacental (through or across the placenta), transtracheal (through the wall of the trachea), transtympanic (across or through the tympanic cavity), transvaginal, ureteral (to the ureter), urethral (to the urethra) and vaginal.

For oral administration, the pharmaceutical compositions may take the form of, for example, tablets or capsules prepared by conventional means with pharmaceutically acceptable excipients such as binding agents (e.g. pregelatinized maize starch, polyvinylpyrrolidone or hydroxypropyl methylcellulose); fillers (e.g. lactose, microcrystalline cellulose or calcium hydrogen phosphate); lubricants (e.g. magnesium stearate, talc or silica); disintegrants (e.g. potato starch or sodium starch glycolate); or wetting agents (e.g. sodium lauryl sulfate). The tablets can also be coated by methods well known in the art. Liquid preparations for oral administration may take the form of, for example, solutions, syrups or suspensions, or they may be presented as a dry product for constitution with water or other suitable vehicle before use. Such liquid preparations may be prepared by conventional means with pharmaceutically acceptable additives such as suspending agents (e.g., sorbitol syrup, cellulose derivatives or hydrogenated edible fats); emulsifying agents (e.g., lecithin or acacia); non-aqueous vehicles (e.g., ationd oil, oily esters, ethyl alcohol or fractionated vegetable oils); and preservatives (e.g., methyl or propyl-p-hydroxybenzoates or sorbic acid). The preparations can also contain buffer salts, flavoring, coloring and sweetening agents as appropriate.

The compositions can be formulated for parenteral administration by injection, e.g., by bolus injection or continuous infusion. Formulations for injection can be presented in a unit dosage form, e.g., in ampoules or in multi-dose containers, with an optionally added preservative. The compositions can further be formulated as suspensions, solutions or emulsions in oily or aqueous vehicles, and may contain other agents including suspending, stabilizing and/or dispersing agents.

Additionally, the compositions can also be formulated as a depot preparation. These long-acting formulations can be administered by implantation (e.g., subcutaneously or intramuscularly) or by intramuscular injection. Thus, for example, the antigens and/or antibodies, or antigen- and/or antibody-based molecules may be formulated with suitable polymeric or hydrophobic materials (e.g., as an emulsion in an acceptable oil) or ion exchange resins, or as sparingly soluble derivatives, for example, as a sparingly soluble salt. Other suitable delivery systems include microspheres, which offer the possibility of local noninvasive delivery of drugs over an extended period. This technology can include microspheres having a precapillary size, which can be injected, e.g., via a coronary catheter into any selected part of an organ without causing inflammation or ischemia. The administered therapeutic may then be slowly released from the microspheres and absorbed by the surrounding cells present in the selected tissue.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, bile salts, and fusidic acid derivatives. In addition, detergents may be used to facilitate permeation. Transmucosal administration can occur using nasal sprays or suppositories. For topical administration, the antigens and/or antibodies, or antigen- and/or antibody-based molecules described herein can be formulated into ointments, salves, gels, or creams.

Forms of the antigens and/or antibodies, or antigen- and/or antibody-based molecules suitable for injectable use can include sterile aqueous solutions or dispersions; formulations including sesame oil, peanut oil or aqueous propylene glycol; and sterile powders for the extemporaneous preparation of sterile injectable solutions or dispersions. In all cases, the form must be sterile and must be fluid. It must be stable under the conditions of manufacture and certain storage parameters (e.g., refrigeration and freezing) and must be preserved against the contaminating action of microorganisms, such as bacteria and fungi.

Antigens and/or antibodies, or antigen- and/or antibody-based molecules can be formulated into a composition in a neutral or salt form. Salts, include the acid addition salts (formed with the free amino groups of the protein) which may be formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like.

A carrier can also be a solvent or dispersion medium containing, for example, water, ethanol, polyol (for example, glycerol, propylene glycol, and liquid polyethylene glycol, and the like), suitable mixtures thereof, and vegetable oils. The proper fluidity can be maintained, for example, using a coating, such as lecithin, by the maintenance of the required particle size in the case of dispersion and by the use of surfactants. The prevention of the action of microorganisms can be brought about by various antibacterial and antifungal agents known in the art. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

Sterile injectable solutions can be prepared by incorporating the active compounds or constructs in the required amount in the appropriate solvent with various of the other ingredients enumerated above, as required, followed by filtered sterilization.

Upon formulation, solutions can be administered in a manner compatible with the dosage formulation and in such amount as is effective. The formulations are easily administered in a variety of dosage forms, such as the type of injectable solutions described above, but slow-release capsules or microparticles and microspheres and the like can also be employed.

For parenteral administration in an aqueous solution, for example, the solution should be suitably buffered if necessary and the liquid diluent first rendered isotonic with sufficient saline or glucose. These aqueous solutions are especially suitable for intravenous, intratumorally, intramuscular, subcutaneous and intraperitoneal administration. By way of a non-limiting example, one dosage could be dissolved in 1 ml of isotonic NaCl solution and either added to 1000 ml of hypodermoclysis fluid or injected at the proposed site of infusion.

The individual responsible for administration will, in any event, determine the appropriate dose for the subject. For example, a subject may be administered an antigens and/or antibodies, or antigen- and/or antibody-based molecules (such as vaccines, complexes, fusion proteins, or conjugates comprising the antigen and/or antibodies, and related nucleic acid molecules, vectors, cells, or binding moieties disclosed herein) herein on a daily or weekly basis for a time period or on a monthly, bi-yearly or yearly basis.

In some embodiments, the one or more antibodies and/or antibody-based molecules (e.g., nucleic acid molecules encoding the one or more antibodies) disclosed herein may be administered to a subject before administering the one or more antigens and/or antigen-based molecules (e.g., nucleic acid molecules encoding the one or more antigens, and/or vaccines comprising the one or more antigens or nucleic acid molecules encoding the one or more antigens) disclosed herein.

In some embodiments, the one or more antibodies or one or more nucleic acid molecules encoding the one or more antibodies may be administered to the subject up to 2 weeks, 3 weeks, or 4 weeks or more before administering the antigen or the nucleic acid molecule encoding the antigen. In some embodiments, the one or more antibodies or one or more nucleic acid molecules encoding the one or more antibodies may be administered to the subject up to 1, 2, 3, 4, 5, or 6 months or more before administering the antigen or the nucleic acid molecule encoding the antigen.

In some embodiments, the one or more antibodies or one or more nucleic acid molecules encoding the one or more antibodies may be administered to the subject up to 3 weeks before administering the antigen or the nucleic acid molecule encoding the antigen.

In some embodiments, the one or more antibodies or one or more nucleic acid molecules encoding the one or more antibodies may be administered to the subject up to 1 week before administering the antigen or the nucleic acid molecule encoding the antigen.

In some embodiments, the one or more antibodies or one or more nucleic acid molecules encoding the one or more antibodies may be administered to the subject up to 1, 2, 3, 4, 5, 6, or 7 days or more before administering the antigen or the nucleic acid molecule encoding the antigen.

In some embodiments, the one or more antibodies or one or more nucleic acid molecules encoding the one or more antibodies may be administered to the subject up to 3 days before administering the antigen or the nucleic acid molecule encoding the antigen.

In some embodiments, the one or more antibodies or one or more nucleic acid molecules encoding the one or more antibodies may be administered to the subject up to 30 minutes, 1 hour, 2 hours, 3 hours, 4 hours, 5 hours, 6 hours, 7 hours, 8 hours, 12 hours, 16 hours, or 24 hours or more before administering antigen or the nucleic acid molecule encoding the antigen.

In some embodiments, the one or more antibodies and/or antibody-based molecules (e.g., nucleic acid molecules encoding the one or more antibodies) disclosed herein may be administered to a subject after administering the one or more antigens and/or antigen-based molecules (e.g., nucleic acid molecules encoding the one or more antigens, and/or vaccines comprising the one or more antigens or nucleic acid molecules encoding the one or more antigens) disclosed herein.

In some embodiments, the one or more antibodies or one or more nucleic acid molecules encoding the one or more antibodies may be administered to the subject after administering the antigen or the nucleic acid molecule encoding the antigen.

In some embodiments, the one or more antibodies or one or more nucleic acid molecules encoding the one or more antibodies may be administered to the subject up to 2 weeks, 3 weeks, or 4 weeks or more after administering the antigen or the nucleic acid molecule encoding the antigen. In some embodiments, the one or more antibodies or one or more nucleic acid molecules encoding the one or more antibodies may be administered to the subject up to 1, 2, 3, 4, 5, or 6 months or more after administering the antigen or the nucleic acid molecule encoding the antigen.

In some embodiments, the one or more antibodies or one or more nucleic acid molecules encoding the one or more antibodies may be administered to the subject up to 3 weeks after administering the antigen or the nucleic acid molecule encoding the antigen.

In some embodiments, the one or more antibodies or one or more nucleic acid molecules encoding the one or more antibodies may be administered to the subject up to 1 week after administering the antigen or the nucleic acid molecule encoding the antigen.

In some embodiments, the one or more antibodies or one or more nucleic acid molecules encoding the one or more antibodies may be administered to the subject up to 1, 2, 3, 4, 5, 6, or 7 days or more after administering the antigen or the nucleic acid molecule encoding the antigen.

In some embodiments, the one or more antibodies or one or more nucleic acid molecules encoding the one or more antibodies may be administered to the subject up to 3 days after administering the antigen or the nucleic acid molecule encoding the antigen.

In some embodiments, the one or more antibodies or one or more nucleic acid molecules encoding the one or more antibodies may be administered to the subject up to 30 minutes, 1 hour, 2 hours, 3 hours, 4 hours, 5 hours, 6 hours, 7 hours, 8 hours, 12 hours, 16 hours, or 24 hours or more after administering the antigen or the nucleic acid molecule encoding the antigen.

In some embodiments, the one or more antibodies and/or antibody-based molecules (e.g., nucleic acid molecules encoding the one or more antibodies) disclosed herein may be administered to a subject during administering of the one or more antigens and/or antigen-based molecules (e.g., nucleic acid molecules encoding the one or more antigens, and/or vaccines comprising the one or more antigens or nucleic acid molecules encoding the one or more antigens) disclosed herein.

In certain embodiments, the one or more antibodies or one or more nucleic acid molecules encoding the one or more antibodies are administered to the subject during administering of the antigen or the nucleic acid molecule encoding the antigen.

In addition to the compositions formulated for parenteral administration, such as intravenous, intratumorally, intradermal or intramuscular injection, other forms include, e.g., tablets or other solids for oral administration; liposomal formulations; time release capsules; biodegradable and any other form currently used.

One may also use intranasal or inhalable solutions or sprays, aerosols or inhalants. Nasal solutions can be aqueous solutions designed to be administered to the nasal passages in drops or sprays. Nasal solutions can be prepared so that they are similar in many respects to nasal secretions. Thus, the aqueous nasal solutions usually are isotonic and slightly buffered to maintain a pH of 5.5 to 7.5. In addition, antimicrobial preservatives, similar to those used in ophthalmic preparations, and appropriate drug stabilizers, if required, may be included in the formulation. Various commercial nasal preparations are known and can include, for example, antibiotics and antihistamines and are used for asthma prophylaxis.

Oral formulations can include excipients as, for example, pharmaceutical grades of mannitol, lactose, starch, magnesium stearate, sodium saccharine, cellulose, magnesium carbonate and the like. These compositions take the form of solutions, suspensions, tablets, pills, capsules, sustained release formulations or powders. In certain defined embodiments, oral compositions will include an inert diluent or assimilable edible carrier, or they may be enclosed in hard or soft-shell gelatin capsule, or they may be compressed into tablets, or they may be incorporated directly with the food of the diet. For oral administration, the compositions may be incorporated with excipients and used in the form of ingestible tablets, buccal tablets, troches, capsules, elixirs, suspensions, syrups, wafers, and the like.

The tablets, troches, pills, capsules and the like may also contain the following: a binder, as gum tragacanth, acacia, cornstarch, or gelatin; excipients, such as dicalcium phosphate; a disintegrating agent, such as corn starch, potato starch, alginic acid and the like; a lubricant, such as magnesium stearate; and a sweetening agent, such as sucrose, lactose or saccharin may be added or a flavoring agent, such as peppermint, oil of wintergreen, or cherry flavoring. When the dosage unit form is a capsule, it may contain, in addition to materials of the above type, a liquid carrier. Various other materials may be present as coatings or to otherwise modify the physical form of the dosage unit. For instance, tablets, pills, or capsules may be coated with shellac, sugar, or both. A syrup of elixir may contain the active compounds sucrose as a sweetening agent methyl and propylparabens as preservatives, a dye and flavoring, such as cherry or orange flavor.

Dose ranges and frequency of administration can vary depending on the nature of the composition as well as parameters of a specific subject and the route of administration used. A dose can also depend on the subject in which it is being administered. For example, a lower dose may be required if the subject is juvenile, and a higher dose may be required if the subject is an adult human subject. In certain embodiments, a more accurate dose can depend on the weight of the subject. In certain embodiments, a more accurate dose can depend on the age of the subject. A suitable, non-limiting example of a dosage of a composition disclosed herein may vary depending upon the age and the size of a subject to be administered, target disease, the purpose of the treatment, conditions, route of administration, and the like. Non-limiting examples of dosages include, e.g., 0.01 to about 20 mg/kg body weight, more preferably about 0.02 to about 7, about 0.03 to about 5, or about 0.05 to about 3 mg/kg body weight. The frequency and the duration of the treatment can be adjusted. In certain embodiments, the initial dose may be followed by administration of a second or a plurality of subsequent doses in an amount that can be approximately the same or less than that of the initial dose, wherein the subsequent doses are separated by at least 1 day to 3 days; at least one week, at least 2 weeks; at least 3 weeks; at least 4 weeks; at least 5 weeks; at least 6 weeks; at least 7 weeks; at least 8 weeks; at least 9 weeks; at least 10 weeks; at least 12 weeks; or at least 14 weeks.

Compositions may include administration to a subject intravenously, intratumorally, intradermally, intraarterially, intraperitoneally, intralesionally, intracranially, intraarticularly, intraprostaticaly, intrapleurally, intratracheally, intranasally, intravitreally, intravaginally, intrarectally, topically, intratumorally, intramuscularly, intrathecally, subcutaneously, subconjunctival, intravesicularlly, mucosally, intrapericardially, intraumbilically, intraocularly, orally, locally, by inhalation, by injection, by infusion, by continuous infusion, by localized perfusion, via a catheter, via a lavage, in a cream, or in a lipid composition.

Compositions as disclosed herein can also include adjuvants such as aluminum salts and other mineral adjuvants, tensoactive agents, bacterial derivatives, vehicles and cytokines. Adjuvants can also have antagonizing immunomodulating properties. For example, adjuvants can stimulate Th1 or Th2 immunity. Compositions and methods as disclosed herein can also include adjuvant therapy.

The antigen(s) and/or nucleic acid molecules encoding the antigen(s) of the disclosure may be provided in the form of a vaccine composition. As an example, the vaccine composition may be useful for the treatment or prevention of a coronavirus and/or an influenza infection, and/or coronavirus-induced and or influenza-induced diseases or disorders. Non-limiting examples of coronavirus vaccines include Comirnaty, Spikevax, Vaxzevria, Nuvaxovid, and Vidprevtyn. Non-limiting examples of influenza vaccines include Afluria, Fluarix, Flublok, Flulaval, Fluvirin, and Fluzone. Without wishing to be bound by theory, vaccines may take several forms (see, e.g., Schlom, J Natl Cancer Inst. 2012; 104(8):599-613; Salgaller, Cancer Res. 1996; 56(20):4749-57 and Marchand, Int J Cancer. 1999; 80(2):219-30). The vaccine composition may include additional antigens or antigen-based molecules such that the antigens or antigen-based molecules of the disclosure is one of a mixture of antigen-based molecules. Adjuvants may be added to the vaccine composition to augment the immune response. In particular for antigen-containing vaccines compositions of the disclosure, pharmaceutically acceptable adjuvants include, but are not limited to, aluminum salts, Amplivax, AS 15, Aquila's QS21 stimulon, AsA404 (DMXAA), beta-glucan, BCG, CP-870,893, CpG7909, CyaA, dSLIM, GM-CSF, IC30, IC31, Imiquimod, ImuFact EVIP321, IS Patch, ISS, 1018 ISS, ISCOMATRIX, Juvlmmune, LipoVac, MF59, monophosphoryl lipid A, Montanide IMS 1312, Montanide ISA 206, Montanide ISA 50V, Montanide ISA-51, OK-432, OM-174, OM-197-MP-EC, ONTAK, poly-ICLC, PepTel®, Pam3Cys, PLGA microparticles, resiquimod, SRL172, Virosomes and other Virus-like particles, YF-17D, VEGF trap, R848, and/or vadimezan.

Alternatively, the vaccine composition may take the form of an antigen-presenting cell (APC) displaying the antigen of the disclosure, e.g., in complex with an MHC. In some embodiments, the APC is an immune cell for example, without limitation, a dendritic cell or a B cell. The antigen may be pulsed onto the surface of the cell (Thurner, J Exp Med. 1999; 190(11):1669-78), or nucleic acid encoding for the antigen of the disclosure may be introduced into dendritic cells or B cells (e.g., by electroporation. Van Tendeloo, Blood. 2001; 98(1):49-56).

In some embodiments, the vaccine disclosed herein may be administered to a subject in a prime-boost regimen. In such an embodiments, the one or more antibodies or one or more nucleic acid molecules encoding the one or more antibodies may be administered to the subject after administering a prime dose of the vaccine but before administering a boost dose of the vaccine to the subject.

In some embodiments, the one or more antibodies or one or more nucleic acid molecules encoding the one or more antibodies may be administered to the subject up to 2 weeks, 3 weeks, or 4 weeks or more after administering the prime dose of the vaccine or the nucleic acid molecule encoding the prime dose of the vaccine. In some embodiments, the one or more antibodies or one or more nucleic acid molecules encoding the one or more antibodies may be administered to the subject up to 1, 2, 3, 4, 5, or 6 months or more after administering the prime dose of the vaccine or the nucleic acid molecule encoding the prime dose of the vaccine.

In some embodiments, the one or more antibodies or one or more nucleic acid molecules encoding the one or more antibodies may be administered to the subject up to 1 week after administering the prime dose of the vaccine or the nucleic acid molecule encoding the prime dose of the vaccine.

In some embodiments, the one or more antibodies or one or more nucleic acid molecules encoding the one or more antibodies may be administered to the subject up to 3 weeks after administering the prime dose of the vaccine or the nucleic acid molecule encoding the prime dose of the vaccine.

In some embodiments, the one or more antibodies or one or more nucleic acid molecules encoding the one or more antibodies may be administered to the subject up to 1, 2, 3, 4, 5, 6, or 7 days or more after administering the prime dose of the vaccine or the nucleic acid molecule encoding the antigen.

In some embodiments, the one or more antibodies or one or more nucleic acid molecules encoding the one or more antibodies may be administered to the subject up to 3 days after administering the prime dose of the vaccine or the nucleic acid molecule encoding the prime dose of the vaccine.

In some embodiments, the one or more antibodies or one or more nucleic acid molecules encoding the one or more antibodies may be administered to the subject up to 30 minutes, 1 hour, 2 hours, 3 hours, 4 hours, 5 hours, 6 hours, 7 hours, 8 hours, 12 hours, 16 hours, or 24 hours or more after administering the prime dose of the vaccine or the nucleic acid molecule encoding the prime dose of the vaccine.

In some embodiments, the one or more antibodies or one or more nucleic acid molecules encoding the one or more antibodies may be administered to the subject up to 2 weeks, 3 weeks, or 4 weeks or more before administering the boost dose of the vaccine or the nucleic acid molecule encoding the boost dose of the vaccine. In some embodiments, the one or more antibodies or one or more nucleic acid molecules encoding the one or more antibodies may be administered to the subject up to 1, 2, 3, 4, 5, or 6 months or more before administering the boost dose of the vaccine or the nucleic acid molecule encoding the boost dose of the vaccine.

In some embodiments, the one or more antibodies or one or more nucleic acid molecules encoding the one or more antibodies may be administered to the subject up to 1 week before administering the boost dose of the vaccine or the nucleic acid molecule encoding the boost dose of the vaccine.

In some embodiments, the one or more antibodies or one or more nucleic acid molecules encoding the one or more antibodies may be administered to the subject up to 1, 2, 3, 4, 5, 6, or 7 days or more before administering the boost dose of the vaccine or the nucleic acid molecule encoding the boost dose of the vaccine.

In some embodiments, the one or more antibodies or one or more nucleic acid molecules encoding the one or more antibodies may be administered to the subject up to 3 days before administering the boost dose of the vaccine or the nucleic acid molecule encoding the boost dose of the vaccine.

In some embodiments, the one or more antibodies or one or more nucleic acid molecules encoding the one or more antibodies may be administered to the subject up to 30 minutes, 1 hour, 2 hours, 3 hours, 4 hours, 5 hours, 6 hours, 7 hours, 8 hours, 12 hours, 16 hours, or 24 hours or more before administering the boost of the vaccine or the nucleic acid molecule encoding the boost of the vaccine.

The compositions of the disclosure may be administered directly into the subject, into an organ or systemically i.d., i.m., s.c., i.p. and i.v., or applied ex vivo to cells derived from the subject or a human cell line which are subsequently administered to the subject or used in vitro to select a subpopulation of immune cells derived from the subject, which are then re-administered to the subject. If the nucleic acid is administered to cells in vitro, it may be useful for the cells to be transfected so as to co-express immune-stimulating cytokines, such as interleukin-2. The antigens and/or antibodies, or antigen- and/or antibody-based molecules may be substantially pure or combined with an immune-stimulating adjuvant or used in combination with immune-stimulatory cytokines, or be administered with a suitable delivery system, e.g., liposomes, viral particles, virus-like particles (VLPs). The antigens and/or antibodies, or antigen- and/or antibody-based molecules may also be conjugated to a suitable carrier such as keyhole limpet haemocyanin (KLH) or mannan (see, e.g., WO 95/18145 and Longenecker et al., 1993).

Methods for introducing antigens and/or antibodies of the present disclosure into a cell or subject can include, for example, vector delivery, particle-mediated delivery, exosome-mediated delivery, lipid nanoparticle (LNP)-mediated delivery, cell-penetrating-peptide-mediated delivery, or implantable-device-mediated delivery. In some embodiments, a nucleic acid or protein can be introduced into a cell or subject in a carrier such as a poly(lactic acid) (PLA) microsphere, a poly(D,L-lactic-coglycolic-acid) (PLGA) microsphere, a liposome, a micelle, an inverse micelle, a lipid cochleate, or a lipid microtubule.

### Generation and Isolation of Antibodies

In some embodiments, the present disclosure provides methods comprising isolating from a subject (e.g., a human or a mouse) one or more antibodies which target an antigen disclosed herein and/or isolating cells producing antibodies which target the antigen disclosed herein. In a related aspect, the methods described herein may comprise isolating from a subject one or more antibodies which target the one or more second epitopes of the antigen and/or isolating cells producing antibodies which target the one or more second epitopes of the antigen. In some embodiments, the one or more antibodies are monoclonal antibodies.

In some embodiments, the isolating comprises binding of the antibodies or cells producing the antibodies described herein to the antigen. In certain embodiments, the antibodies and/or the antigen(s) may comprise a detectable label. In certain embodiments, the antibodies and/or the antigen(s) may comprise a reporter molecule. The detectable label or reporter molecule can be a radioisotope, such as ³H, ¹⁴C, ³²P, ³⁵S, or ¹²⁵I; a fluorescent or chemiluminescent moiety such as fluorescein isothiocyanate, or rhodamine; or an enzyme such as alkaline phosphatase, β-galactosidase, horseradish peroxidase, or luciferase. In some embodiments, the detectable label or reporter molecule can be a his-tag, or a polyhistidine tag. In some embodiments, the detectable label or reporter molecule can be a C-terminal mFc tag, a myc-myc-histidine tag, or a myc-myc-hexahistidine tag. Specific exemplary assays that can be used to detect or measure spike glycoprotein in a sample include neutralization assays, enzyme-linked immunosorbent assay (ELISA), radioimmunoassay (RIA), and fluorescence-activated cell sorting (FACS).

In some embodiments, the above-described methods may further comprise generating a monoclonal antibody (mAb) based on the antibody isolated from the subject or an antigen-binding fragment thereof. In some embodiments, the monoclonal antibody (mAb) is a human antibody. In some embodiments, the monoclonal antibody is a humanized antibody.

Antibody-producing cells, otherwise called cells expressing antibodies, disclosed herein can encompass cells in which the antibodies expressed are bound to or anchored in the cell membrane, i.e., cell surface antibodies, as well as cells that secrete antibody. Antibody-producing cells may be derived from the starting primary antibody-producing cells, or the primary antibody-producing cells selected by the methods of the disclosure. As such, cell lines, plasma cells, memory B-cells, hybridomas, plasma cell myelomas and recombinant antibody-expressing cells may be derived or isolated from primary antibody-producing cells prior to or following collection of antibody-producing cells expressing high-affinity antibodies. For example, primary antibody-producing cells may be fused to myeloma cells to make hybridomas, or otherwise immortalized, such as infected with a virus (e.g., EBV), or may be differentiated by cell sorting techniques based on protein markers expressed by particular B cell types. For example, selected antibody producing cells expressing high-affinity antibodies may be sorted by FACS based on cell surface B cell markers. In certain aspect, the cells producing antibodies disclosed herein are B cells.

The present disclosure further provides methods in which primary antibody-producing cells expressing an antigen-specific antibody are efficiently selected based on their binding properties in situ, then isolated using techniques for single-cell isolation, such as using fluorescence activated cell sorting (FACS), a high throughput screening method that can sample hundreds of millions of cells in a cell population. The cells expressing desirable high affinity antibodies can be identified and isolated directly from all of the cells producing antibodies (rather than from screening antibody libraries following cloning steps). The antibodies produced by the selected cells can then be cloned and reproduced recombinantly in host cells for direct use, thereby diminishing the number of steps taken while ensuring a higher probability of desirable antibodies.

In some embodiments, the method steps for isolating an antibody disclosed herein may comprise, for example, contacting a population of primary antibody-producing cells with specificity to an antigen of interest with a low concentration of labeled antigen for a time sufficient for the labeled antigen to bind to antibody on the surface of the cells; washing the labeled antigen-bound cells with an appropriate buffer for a period of time from about 15 minutes to about 60 minutes; then isolating the antigen-bound cells. The isolation step may further comprise identifying the antigen-bound cells with an antigen-binding protein comprising a label for identification.

In some embodiments, the present disclosure provides a cell selection method wherein antigen-specific cells are contacted with biotinylated antigen. In such an embodiment, the method may further comprise fluorescently labeled streptavidin. Host cells comprising a nucleic acid molecule encoding the antibody isolated using the methods of the disclosure are also contemplated.

In some embodiments, the present disclosure provides a method to identify and isolate antigen-specific antibody-producing cells that express antibodies exhibiting a high binding affinity for an antigen of interest; the nucleic acids encoding these antibodies can then be cloned into host cells for mass production of the high affinity antibodies.

In some embodiments, a non-human mammal is immunized with an antigen of interest and the animal's immune response to the antigen is monitored using an antigen-specific immunoassay. Once an appropriate immune response has been achieved, antibody-producing cells are collected from the immunized animal. Antibody-producing cells are collected from a number of sources, including but not limited to spleen, lymph node, bone marrow and peripheral blood. For example, following immunization, splenocytes are harvested from an immunized animal. Following removal of red blood cells by lysis, IgG⁺ antigen-positive B cells from the immunized animals are isolated from the cell population using the methods described herein.

In some embodiments, peripheral blood mononuclear cells (PBMCs) are harvested from a human or non-human mammal known to have humoral immunity to an antigen of interest. IgG+, antigen-positive B cells having the highest affinities in the antibody-producing cell population can then be isolated for further processing in accordance with the methods of the disclosure.

To select for the cells that express antibodies exhibiting the highest binding affinity for the antigen of interest, the harvested cells are contacted with a low concentration, for example, from about 0.1 nM to about 25 nM, or from about 1 nM to about 20 nM or from about 2 nM to about 10 nM, of monomeric antigen that is labeled, for a time sufficient for the labeled antigen to bind to antibody on the surface of the immune cells; in some embodiments, exposure of the immune cells to labeled antigen for from about 5 to about 60 minutes is suitable. In some embodiments, the low concentration is less than about 10 nM. In other embodiments, the low concentration of antigen is about 9 nM, about 8 nM, about 7 nM, about 6 nM, about 5 nM, about 4 nM, about 3 nM, about 2 nM, about 1 nM. In still other embodiments, the antigen concentration is 5 nM. In some embodiments, the antigen concentration is less than about 1 nM. In another embodiment, the antigen concentration is 1 nM. In another embodiment, the antigen concentration is less than 1 nM. In other embodiments, the antigen is soluble.

In some embodiments, the label is biotin, e.g., the antigen is biotinylated. Antigen labels, otherwise called detection molecules, enable further detection of the antigen of interest bound to the antibody-producing cells. Detection may be made by immuno-staining with an antibody specific for the label or direct staining with a reagent that binds to the label. Numerous detection kits and techniques are well-known in the art.

Concurrently or successively, the cells may be detected as B cells, in particular IgG+IgM-cells (incubated with anti-B cell marker, anti-IgG or anti-Fc reagents, or the like) in the anticipation of next step single-cell isolation techniques. IgG or B cell detection reagents may be incubated with the cells prior to, during, or following the incubation with antigen of interest. B cell detection reagents are commercially available. (See also Huang, J. et al., 2013 Nature Protocols, 8(10):1907-1915.)

Once unbound antigen has been removed, the selected cells may be enriched for high-affinity antibodies.

Once unbound antigen has been removed, the cells may be contacted with an antigen-binding protein comprising a detectable label, for example, a fluorescent label for the purposes of identifying the antigen-specific cells. In embodiments where the antigen has been biotinylated, a fluorescently labeled streptavidin is used for detection. In the instance where a detectable label is enzymatically activated, the cells are contacted with the appropriate enzyme to detect cells bearing bound antigen.

Using fluorescence-activated cell sorting (FACS) to detect and isolate the enriched high-affinity antibody expressing cells is a highly efficient and sensitive tool for single-cell sorting. Protocols for single cell isolation by flow cytometry are well-known (Huang, J. et al, 2013, supra). To that end, cells that bind fluorescent antigen (or fluorescently labeled streptavidin/biotinylated antigen) are detected and identified as cells that express antibodies that specifically bind the antigen of interest with high affinity and are isolated to individual wells on 96-well, or 384-well plates.

Cells may be sorted and collected by alternative methods known in the art, including but not limited to manual single cell picking, limited dilution and B cell panning of adsorbed antigen, which are all well-known in the art (Rolink, et al., 1996 J Exp Med 183:187-194; Lightwood, D. et al, 2006 J. Immunol. Methods 316(1-2): 133-43. Epub 2006 Sep. 18).

Isolated B cells may be fused with an immortal cell, such as a myeloma cell line, in order to create a hybridoma. Hybridoma techniques are well within the skill of the artisan (Harlow and Lane, 1988, supra). Isolated B cells may be further differentiated or sorted to identify specific B cell types, such as determination by cell surface or gene expression markers.

Once cells are collected, the DNA is prepared from the cells in order to recombinantly produce the antibodies. As mentioned above, B cells may be cultured, fused to myeloma cells or otherwise immortalized, such as infected with a virus (e.g., EBV), in order to make the DNA more abundant, as necessary, prior extracting DNA and cloning antibody genes directly from each sorted B cell. Briefly, genes encoding immunoglobulin variable heavy and light chains (i.e., VH, Ig Vκ and Vλ) are recovered using RT-PCR of mRNA isolated from the selected antibody-producing cells, as performed using conventional techniques, for example, as described by Wang et al. (J. Immunol. Methods 244:217-225) and described herein. Antibody genes are cloned into IgG heavy- and light-chain expression vectors and expressed via transfection of host cells.

For recombinant production of an antibody of the disclosure in a suitable host cell, the nucleic acid encoding the antibody genes are inserted into a replicable vector for further cloning (amplification of the DNA) or for expression (stably or transiently). Many vectors, particularly expression vectors, are available or may be engineered to comprise appropriate regulatory elements. An expression vector in the context of the present disclosure may be any suitable vector, including chromosomal, non-chromosomal, and synthetic nucleic acid vectors (a nucleic acid sequence comprising a suitable set of expression control elements). Examples of such vectors include derivatives of SV40, bacterial plasmids, phage DNA, baculovirus, yeast plasmids, vectors derived from combinations of plasmids and phage DNA, and viral nucleic acid (RNA or DNA) vectors. In one embodiment, an antibody-encoding nucleic acid molecule is comprised in a naked DNA or RNA vector, including, for example, a linear expression element (as described in, for instance, Sykes and Johnston, Nat Biotech 12:355-59 (1997)), a compacted nucleic acid vector (as described in for instance U.S. Pat. No. 6,077,835 and/or WO00/70087), or a plasmid vector such as pBR322, pUC 19/18, or pUC 118/119. Such nucleic acid vectors and the usage thereof are well known in the art (see, for instance, U.S. Pat. Nos. 5,589,466 and 5,973,972).

An expression vector may alternatively be a vector suitable for expression in a yeast system. Any vector suitable for expression in a yeast system may be employed. Suitable vectors include, for example, vectors comprising constitutive or inducible promoters such as yeast alpha factor, alcohol oxidase and PGH.

In certain embodiments, the vector comprises a nucleic acid molecule (or gene) encoding a heavy chain of the antibody and a nucleic acid molecule encoding a light chain of the antibody, wherein the antibody is produced by the B cell selected by a method of the disclosure. The vector utilized includes an expression vector comprising the nucleic acid molecules (genes) described wherein the nucleic acid molecule (gene) is operably linked to an expression control sequence suitable for expression in the host cell.

The choice of vector depends in part on the host cell to be used. Host cells include, but are not limited to, cells of either prokaryotic or eukaryotic (generally mammalian) origin.

In some embodiments, the host cell is a bacterial or yeast cell. In some embodiments, the host cell is a mammalian cell. In other embodiments, the host cell is selected from the group consisting of Chinese hamster ovary (CHO) cells (e.g. CHO K1, DXB-11 CHO, Veggie-CHO, CHOt), COS (e.g. COS-7), stem cell, retinal cells, Vero, CV1, kidney (e.g. HEK293, 293 EBNA, MSR 293, MDCK, HaK, BHK21), HeLa, HepG2, W138, MRC 5, Colo25, HB 8065, HL-60, Jurkat, Daudi, A431 (epidermal), CV-1, U937, 3T3, L cell, C127 cell, SP2/0, NS-0, MMT cell, tumor cell, and a cell line derived from an aforementioned cell.

It will be appreciated that the full-length antibody (heavy chain and light chain comprising variable and constant regions) may be subsequently cloned into an appropriate vector or vectors. Alternatively, the Fab region of an isolated antibody may be cloned into a vector or vectors in line with constant regions of any isotype for the intended purpose. Therefore, any constant region may be utilized in the construction of isolated antibodies, including IgG1, IgG2, IgG3, IgG4, IgM, IgA, IgD, and IgE heavy chain constant regions, or chimeric heavy chain constant regions. Such constant regions can be obtained from any human or animal species depending on the intended use of the antibodies. Also, antibody variable regions or Fab region may be cloned in an appropriate vector(s) for the expression of the protein in other formats, such as ScFv, diabody, etc.

The disclosure provides a mammalian host cell encoding a nucleic acid molecule comprising a high affinity antibody specific for an antigen of interest, wherein a heavy chain variable region and a light chain variable region of the antibody were isolated from a B cell expressing the antibody, and wherein the B cell was selected from a population of cells from an immunized mammal with a low concentration of the antigen in monomeric form.

Binding affinities and kinetic constants of the antibodies derived from cells isolated using the method of the disclosure are determined in accordance with methods known in the art, for example, by surface plasmon resonance. In one embodiment, measurements are conducted at 25° C. on, for example, a Biacore 2000 or similar instrument. Antibodies are captured on an anti-human Fc sensor surface, and soluble monomeric protein is injected over the surface. Kinetic association (kₐ) and dissociation (k_{d}) rate constants are determined by processing and fitting the data to a 1:1 binding model using curve fitting software. Binding dissociation equilibrium constants (K_{D}) and dissociative half-lives (t1/2) are calculated from the kinetic rate constants as: K_{D}(M)=kₐ/k_{d}; and t_{1/2} (min)−(ln 2/(60*k_{d}).

Methods for generating human antibodies in transgenic mice are known in the art. Any such known methods can be used in the context of the present disclosure to make human antibodies that specifically bind to spike glycoprotein. An immunogen comprising any one of the following can be used to generate antibodies to spike glycoprotein. In certain embodiments of the disclosure, the antibodies of the disclosure are obtained from mice immunized with a full length, native spike glycoprotein, or with a live attenuated or inactivated virus, or with DNA encoding the protein or fragment thereof. Alternatively, the spike glycoprotein or a fragment thereof may be produced using standard biochemical techniques and modified and used as immunogen. In one embodiment of the disclosure, the immunogen is a recombinantly produced spike glycoprotein or fragment thereof. In certain embodiments of the disclosure, the immunogen may be a spike polypeptide vaccine. In certain embodiments, one or more booster injections may be administered. In certain embodiments, the immunogen may be a recombinant spike polypeptide expressed in E. coli or in any other eukaryotic or mammalian cells such as Chinese hamster ovary (CHO) cells.

Using VELOCIMMUNE® technology (see, for example, U.S. Pat. No. 6,596,541, Regeneron Pharmaceuticals, VELOCIMMUNE®) or any other known method for generating monoclonal antibodies, high affinity chimeric antibodies to spike glycoprotein can be initially isolated having a human variable region and a mouse constant region. The VELOCIMMUNE® technology involves generation of a transgenic mouse having a genome comprising human heavy and light chain variable regions operably linked to endogenous mouse constant region loci such that the mouse produces an antibody comprising a human variable region and a mouse constant region in response to antigenic stimulation. The DNA encoding the variable regions of the heavy and light chains of the antibody are isolated and operably linked to DNA encoding the human heavy and light chain constant regions. The DNA is then expressed in a cell capable of expressing the fully human antibody.

Generally, a VELOCIMMUNE® mouse is challenged with the antigen of interest, and lymphatic cells (such as B-cells) are recovered from the mice that express antibodies. The lymphatic cells may be fused with a myeloma cell line to prepare immortal hybridoma cell lines, and such hybridoma cell lines are screened and selected to identify hybridoma cell lines that produce antibodies specific to the antigen of interest. DNA encoding the variable regions of the heavy chain and light chain may be isolated and linked to desirable isotypic constant regions of the heavy chain and light chain. Such an antibody protein may be produced in a cell, such as a CHO cell. Alternatively, DNA encoding the antigen-specific chimeric antibodies, or the variable domains of the light and heavy chains may be isolated directly from antigen-specific lymphocytes.

Initially, high affinity chimeric antibodies are isolated having a human variable region and a mouse constant region. As in the experimental section below, the antibodies are characterized and selected for desirable characteristics, including affinity, selectivity, epitope, etc. The mouse constant regions are replaced with a desired human constant region to generate the fully human antibody of the disclosure, for example wild-type or modified IgG1 or IgG4. While the constant region selected may vary according to specific use, high affinity antigen-binding and target specificity characteristics reside in the variable region.

In some embodiments, antibodies and antigen-binding fragments, disclosed herein may also be produced in an E. coli/T7 expression system. By way of a non-limiting example, nucleic acids encoding the anti-spike glycoprotein antibody immunoglobulin molecules may be inserted into a pET-based plasmid and expressed in the E. coli/T7 system. For example, the present disclosure includes methods for expressing an antibody or antigen-binding fragment thereof or immunoglobulin chain thereof in a host cell (e.g., bacterial host cell such as E. coli such as BL21 or BL21DE3) comprising expressing T7 RNA polymerase in the cell which also includes a polynucleotide encoding an immunoglobulin chain that is operably linked to a T7 promoter. For example, in an embodiment of the disclosure, a bacterial host cell, such as an E. coli, includes a polynucleotide encoding the T7 RNA polymerase gene operably linked to a lac promoter and expression of the polymerase and the chain is induced by incubation of the host cell with IPTG (isopropyl-beta-D-thiogalactopyranoside).

### Kits

The present disclosure further comprises a kit which may comprise any of various compositions of the present disclosure, including but not limited to, the antibodies, antigens, vaccines, nucleic acid molecules, vectors, lipid nanoparticles, or cells of the disclosure. In certain embodiments, such kits may include components that preserve or maintain, e.g., the nucleic acid molecules contained therein, such as reagents that protect against nucleic acid degradation. Such components may be nuclease or RNase- or DNase-free or protect against RNases or DNAses, for example. Any of the compositions or reagents described herein may be components in a kit.

As a non-limiting example, the kit may comprise (i) an antigen or a nucleic acid molecule encoding the antigen, and (ii) one or more antibodies targeting one or more first epitopes of the antigen or one or more nucleic acid molecules encoding the one or more antibodies.

Kits can also include a suitable container, for example, vials, tubes, mini- or microfuge tubes, test tube, flask, bottle, syringe or other container. Where an additional component or agent is provided, the kit can contain one or more additional containers into which this agent or component may be placed. Kits herein will also typically include a means for containing the antigen and/or antibody, or antigen- and/or antibody-based molecules (such as vaccines, complexes, fusion proteins, or conjugates comprising the antigen and/or antibodies, and related nucleic acid molecules, vectors, cells, or binding moieties disclosed herein) and any other reagent containers in close confinement for commercial sale. Such containers may include injection or blow-molded plastic containers into which the desired vials are retained. Optionally, one or more additional active agents may be needed for compositions described.

The present disclosure also provides articles of manufacture comprising any one of the compositions or kits described herein.

### LIST OF NON-LIMITING EMBODIMENTS

The present disclosure also includes the following non-limiting embodiments:
Embodiment 1. A method for redirecting an antibody response in a subject from one or more undesirable epitopes of an antigen towards other epitopes of said antigen, said method comprising administering to the subject an effective amount of one or more antibodies targeting said one or more undesirable epitopes, wherein said one or more antibodies are administered to the subject before or during administering said antigen or a nucleic acid encoding said antigen.
Embodiment 2. The method of embodiment 1, wherein said one or more antibodies are administered before administering said antigen or a nucleic acid encoding said antigen to the subject.
Embodiment 3. The method of embodiment 1 or embodiment 2, further comprising isolating from the subject antibodies which recognize other antigen epitopes that are not undesirable epitopes.
Embodiment 4. The method of embodiment 3, further comprising generating monoclonal antibodies (mAbs) based on the antibodies isolated from the subject.
Embodiment 5. A method for increasing efficacy of a vaccine in a subject, wherein the vaccine comprises an antigen or a nucleic acid encoding said antigen, said method comprising administering to the subject an effective amount of one or more antibodies targeting one or more undesirable epitopes of said antigen, wherein said one or more antibodies are administered to the subject before or during administering said vaccine.
Embodiment 6. The method of embodiment 5, wherein said one or more antibodies are administered before administering said vaccine to the subject.
Embodiment 7. The method of embodiment 5, wherein said vaccine is administered in a prime-boost regimen, and wherein said one or more antibodies are administered after administering prime but before administering boost of said vaccine to the subject.
Embodiment 8. The method of any one of embodiments 1-7, wherein said one or more undesirable epitopes are immunodominant epitopes.
Embodiment 9. The method of embodiment 8, wherein said immunodominant epitopes are less conserved than other epitopes of said antigen between different strains or species of a pathogen from which said antigen is derived.
Embodiment 10. The method of any one of embodiments 1-9, wherein the antigen is a protein antigen.
Embodiment 11. The method of any one of embodiments 1-10, wherein the antigen is derived from a Class I pathogen.
Embodiment 12. The method of any one of embodiments 1-10, wherein the antigen is derived from a Class II pathogen.
Embodiment 13. The method of embodiment 12, wherein said pathogen is a virus.
Embodiment 14. The method of embodiment 13, wherein said virus is a coronavirus.
Embodiment 15. The method of embodiment 14, wherein said coronavirus is SARS-CoV-2.
Embodiment 16. The method of embodiment 15, wherein said antigen is SARS-CoV-2 spike glycoprotein and said one or more undesirable epitopes are neutralizing epitopes comprised within receptor binding domain (RBD) of said SARS-CoV-2 spike glycoprotein.
Embodiment 17. A method for shielding one or more undesirable epitopes of an antigen from recognition by the immune system in a subject, said method comprising administering to the subject an effective amount of one or more antibodies targeting said one or more undesirable epitopes.
Embodiment 18. The method of embodiment 17, wherein said antigen is an endogenous molecule of a subject.
Embodiment 19. The method of embodiment 18, wherein said antigen is targeted by an immune response in an autoimmune disease.
Embodiment 20. The method of any one of embodiments 1-19, wherein said one or more antibodies are monoclonal antibodies (mAbs).
Embodiment 21. A composition comprising two or more monoclonal antibodies (mAbs) targeting undesirable epitopes of an antigen.
Embodiment 22. The composition of embodiment 21, wherein said undesirable epitopes are immunodominant epitopes.
Embodiment 23. The composition of embodiment 22, wherein said immunodominant epitopes are less conserved than other epitopes of said antigen between different strains or species of a pathogen from which said antigen is derived.
Embodiment 24. The composition of any one of embodiments 21-23, wherein the antigen is a protein antigen.
Embodiment 25. The composition of any one of embodiments 21-24, wherein the antigen is derived from a Class I pathogen.
Embodiment 26. The composition of any one of embodiments 21-24, wherein the antigen is derived from a Class II pathogen.
Embodiment 27. The composition of embodiment 26, wherein said pathogen is a virus.
Embodiment 28. The composition of embodiment 27, wherein said virus is a coronavirus.
Embodiment 29. The composition of embodiment 28, wherein said coronavirus is SARS-CoV-2.
Embodiment 30. The composition of embodiment 29, wherein said antigen is SARS-CoV-2 spike glycoprotein and said undesirable epitopes are neutralizing epitopes comprised within receptor binding domain (RBD) of said SARS-CoV-2 spike glycoprotein.
Embodiment 31. The composition of embodiment 21, wherein said antigen is a molecule targeted by an immune response in an autoimmune disease.

### EXAMPLES

The present disclosure is also described and demonstrated by way of the following examples. However, the use of this and other examples anywhere in the specification is illustrative only and in no way limits the scope and meaning of the disclosure or of any exemplified term. Likewise, the disclosure is not limited to any particular preferred embodiments described here. Indeed, many modifications and variations of the disclosure may be apparent to those skilled in the art upon reading this specification, and such variations can be made without departing from the disclosure in spirit or in scope. The disclosure is therefore to be limited only by the terms of the appended claims along with the full scope of equivalents to which those claims are entitled.

### Material and Methods

### Proteins for Immunization and Immunoassays:

SARS-CoV-2 spike trimer (E11047) amino acids 14-1211 and spike RBD (E10621) amino acids 319-541 from Wuhan-Hu-1 (accession number MN908947.3) were expressed and purified for use as protein immunogens and proteins for SARS-CoV-2 spike antibody detection in SARS-CoV-spike immunized mice. SARS-CoV-2 spike N-terminal domain (NTD), S1, and S2 regions; all from Wuhan-Hu-1 sequence, MN908947.3) as well as SARS-CoV-2 nucleocapsid protein was obtained commercially.

### Immunization of Mice

Female C57BL/6 mice were treated with anti-SARS CoV-2 spike RBD mAb antibodies, E10933 and E10987 that target neutralizing epitopes that overlap with ACE2 binding, at 10 mg/kg-0.001 mg/kg intravenously at either day −3 or day 18. A subset of mice received no mAb treatment. Mice were then immunized with SARS-CoV-2 spike trimer, SARS-CoV-2 RBD or PBS at 5 μg with 50 μg poly(I:C) HMW subcutaneously at day 0, and then boosted at day 21. Mice were euthanized at day 42 and serum was obtained for serological analysis of SARS-CoV-2 antibody responses.

### Cell Lines

African green monkey (C. aethiops) kidney epithelial cells, American Type Culture Collection (ATCC®)-CCL81 were cultured in T225 flasks in complete Dulbecco's Modified Eagle Medium (DMEM) containing 10% fetal bovine serum, 1% penicillin-streptomycin-glutamine and 1% sodium pyruvate.

### Pseudoviral Production

Non-replicating pseudo particles VSV-SARS-CoV-2-Spike virus were generated as previously described (Baum A, Fulton B O, Wloga E, Copin R, Pascal K E, Russo V, et al. Antibody cocktail to SARS-CoV-2 Spike Protein prevents rapid mutational escape seen with individual antibodies. Science 2020b; 369(6506):1014-8). Briefly, pseudoparticles were generated using a VSVΔG system in which the VSV glycoprotein was deleted from the genome and in which the VSV was engineered to express firefly luciferase (Fluc) fluorescent reporter. Pseudoparticles were pseudotyped with WT SARS-CoV-2 S protein (aa 14-1255; Wuhan-Hu-1, accession number MN908947.3 containing D614G substitution) by cloning the synthesized SARS-CoV-2 Spike Protein into an expression plasmid.

### Multiplex Luminex Assay to Detect Ag-Specific IgG Responses in Mouse Serum

SARS-CoV-2 specific antibody responses were measured through a non-GLP modified multiplexed Luminex immunoassay. SARS-CoV-2 spike recombinant antigens (full length SARS-CoV-2 spike trimerized, N-terminal domain (NTD), RBD, S1, and S2 regions; all from Wuhan-Hu-1 sequence, MN908947.3) as well as SARS-CoV-2 nucleocapsid protein were coupled to fluorescently barcoded microspheres. Chemical coupling of proteins to microspheres were performed as previously described (Blauvelt A, Simpson E L, Tyring S K, et al. Dupilumab does not affect correlates of vaccine-induced immunity: A randomized, placebo-controlled trial in adults with moderate-to-severe atopic dermatitis. J Am Acad Dermatol 2019; 80: 158-167.) Serum samples were diluted 1:50 and anti-E10933+E10987 idiotype antibodies (E13269 and E13261, respectively) at 54 μg/ml were included to block circulating E10933+E10987. Diluted serum (at 1:50 or 1:1250) and mAb mixture were then added to the Ag-coupled bead mixture and incubated overnight at 4° C. Antibody bound beads detected via PE conjugated anti-mouse IgG (Columbia Bio, Cat: D5-112-Fc). Antibody levels for each antigen-coated bead are represented as the median fluorescence intensity (MFI) at a given serum dilution.

### Pseudoviral Neutralization Assay

Neutralization serum neutralization titers against SARS-CoV-2 were measured by utilizing a non-replicative recombinant vesicular stomatitis virus (VSV) encoding firefly luciferase (Fluc) and complemented with SARS-CoV-2 spike (aa 14-1255, Wuhan-Hu-1 sequence, MN908947.3) instead of the native VSV viral glycoprotein (G). To assess neutralization antibodies in mouse serum, pseudotyped viral particles (pVSVLuc-SARS-CoV-2 spike) were incubated with serially diluted serum treated with anti-E10933+E10987 idiotype antibodies at 135 μg/ml at a starting 1:20 plasma dilution (10× molar excess of expected E10933+E10987 plasma concentration at Cₘₐₓ) to block E10933+E10987 mediated neutralization. To assess neutralization titers from monoclonal mAbs obtained from SARS-CoV-2 RBD immunized mice either pre-dosed with anti-spike mAbs E14315+E15160, E15160, E14315, or E10987+E10933 or not mAb pre-dosed; SARS-CoV-2 spike pseudotyped virus mixed with either mouse serum or obtained anti-SARS-CoV-2 mAbs were overlayed onto Vero cells and infectivity was detected by utilizing expression of Fluc reporter using the SpectraMax i3 plate reader with MiniMax imaging cytometer. Neutralization observed with media alone or virus alone was defined as 100% or 0% neutralization, respectively. Percentage of neutralization was calculated by 1 minus the difference between the experimental condition and cell culture media alone, divided by the difference between the virus alone and cell culture media alone, multiplied by 100:

$$Neutralization ( \% ) - [ 1 - \frac{( Experimental condition - media alone )}{( Virus alone - media alone )} ] \times 100$$

For final serum neutralization titers, IC₅₀ and HillSlope values were calculated from an assay performed in duplicate wells using GraphPad prism. Limit of detection is based on the starting plasma dilution (1:20 diluted plasma mixed equal volume with pseudotyped viral particles, equaling 1:40 dilution).

### Determination of Anti-SARS-CoV-2 Antibodies Binding to Recombinant SARS-CoV-2 Proteins.

Binding of samples containing anti-SARS-CoV-2 monoclonal antibodies to SARS-CoV-2 recombinant proteins was determined using a bead-based multiplex immunoassay. Briefly, anti-SARS-CoV-2 antibody samples were incubated with an array of beads coated with individual SARS-CoV-2 spike ectodomain recombinant proteins, and the binding signals of the bound antibodies were detected with fluorophore-labeled anti-human kappa or anti-human lambda antibody and binding signals recorded using a Luminex instrument.

To generate the antigen bead array, SARS-CoV-2 spike ectodomain recombinant proteins (Table 3) and neutravidin (ThermoFisher, Cat. No. 31050) were covalently coupled to paramagnetic Luminex beads (MagPlex microspheres, Luminex Corp.). Each protein was coupled at 10 μg/12.5×10⁶ beads. Biotinylated proteins were captured at 10 μg/12.5×10⁶ neutravidin coupled beads. For the binding assay, a mixture of the bead array was prepared in blocking buffer (PBS containing 2% BSA and 0.05% Na Azide), by adding 2,700 beads of each antigen in a final volume of 75 μL/well on a 96-well ProcartaPlex plate followed by addition of 25 μL of the antibody samples. After two hours incubation at 25° C., the beads were washed twice with 200 μL of wash buffer (DPBS with 0.05% Tween 20). To detect bound antibody levels on the beads, 100 μL of 2.5 μg/mL R-Phycoerythrin conjugated goat anti-human kappa F(ab′)2 (SouthernBiotech, Cat. No: 2063-09) in blocking buffer or 100 μL of 1.25 μg/mL R-Phycoerythrin Goat Anti-Human Lambda (SouthernBiotech, Cat. No: 2070-09) in blocking buffer was added. After 30 minutes incubation, the beads were washed twice and resuspended in 150 μL of wash buffer. The plates were then read in a Luminex FlexMap 3D instrument with Luminex xPonent software version 4.3, fluorescence intensity of each bead was recorded as median fluorescence intensity (MFI).

TABLE 3
SARS-CoV-2 recombinant proteins used to
determine anti-SARS-CoV-2 mAb binding

| | | Vendor/Cat. |
|---|---|---|
| Strain | Description | No. |
| WT spike | SARS-CoV-2 Spike ecto (aa 14-1211, | 11047-L6 |
| trimer | R682G, R683S, R685S, K986P, V987P). | |
| | foldon Trimer domain.GS.Thrombin.mmH | |
| Omicron | SARS-CoV-2 Spike Trimer, His Tag | Acrobiosystems, |
| BA.1 | | SPN-C52Hz |
| Omicron | SARS-CoV-2 Spike Trimer, His Tag | Acrobiosystems, |
| BA.2 | (BA.2/Omicron) | SPN-C5223 |
| Omicron | SARS-CoV-2 Spike Trimer, His Tag | Acrobiosystems, |
| BA.3 | | SPN-C5225 |
| Alpha | SARS-CoV-2 (2019-nCoV) Spike S1 | Sinobiologicals, |
| | (HV69-70 deletion, Y144 deletion, | 40591-V08H12 |
| | N501Y, A570D, D614G, P681H)-His | |
| | Tag | |
| Beta | SARS-CoV-2 (2019-nCoV) Spike S1 | Sinobiologicals, |
| | (K417N, E484K, N501Y, D614G)-His | 40591-V08H10 |
| | Tag | |
| Delta | SARS CoV2 S1 (T95I, G142D, E154K, | Acrobiosystems, |
| | L452R, E484Q, D614G, P681R) His | S1N-C52Ht |
| | Tag | |
| Gamma | Bt-SARS CoV2 protein RBD (K417T, | Acrobiosystems, |
| | E484K, N501Y) His Tag | SPD-C82E6 |

### Example 1. Administration of mAbs E10933 and E10987 that Target Neutralizing Epitopes Prior to or at the Same Time as Administration of an Immunogen can Change the Properties of the Resulting Antibody Response

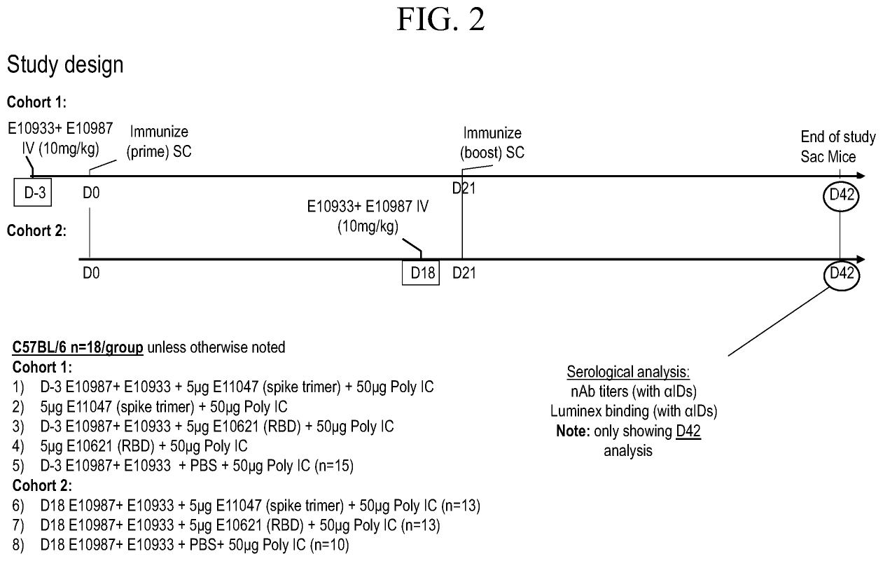

The effect of pre-treatment of mice with αSARS-CoV-2 RBD mAbs (E10933 and E10987) that target neutralizing epitopes before priming or boosting doses of SARS-CoV-2 spike or receptor binding domain (RBD) vaccination on overall IgG binding levels across SARS-CoV-2 spike regions was assessed. The study design is shown in FIG. 2.

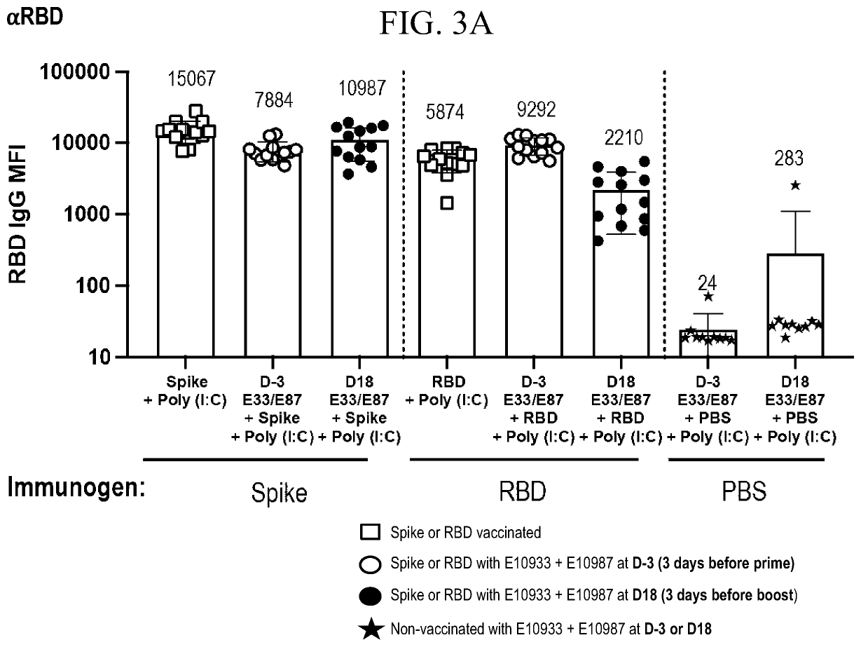

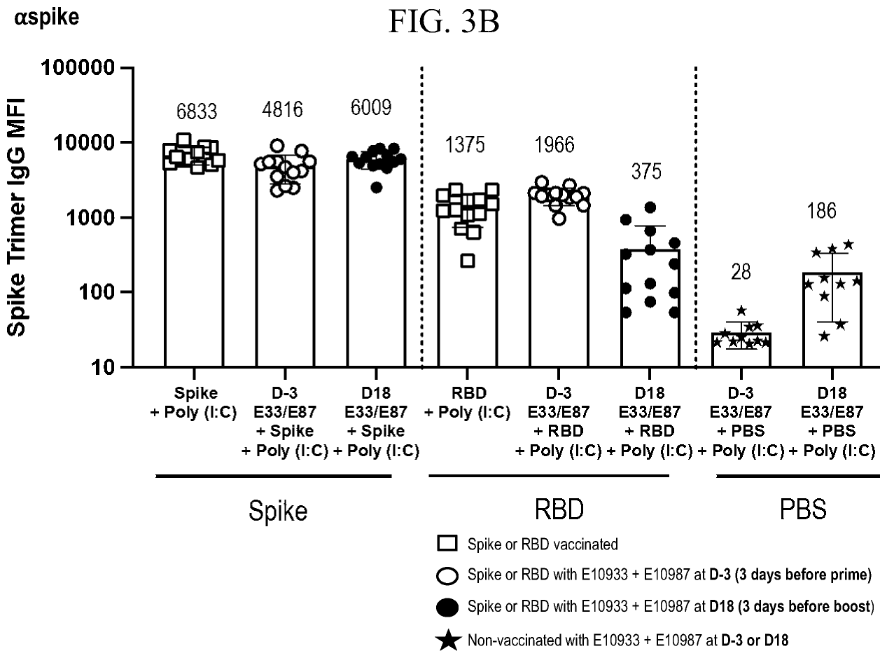

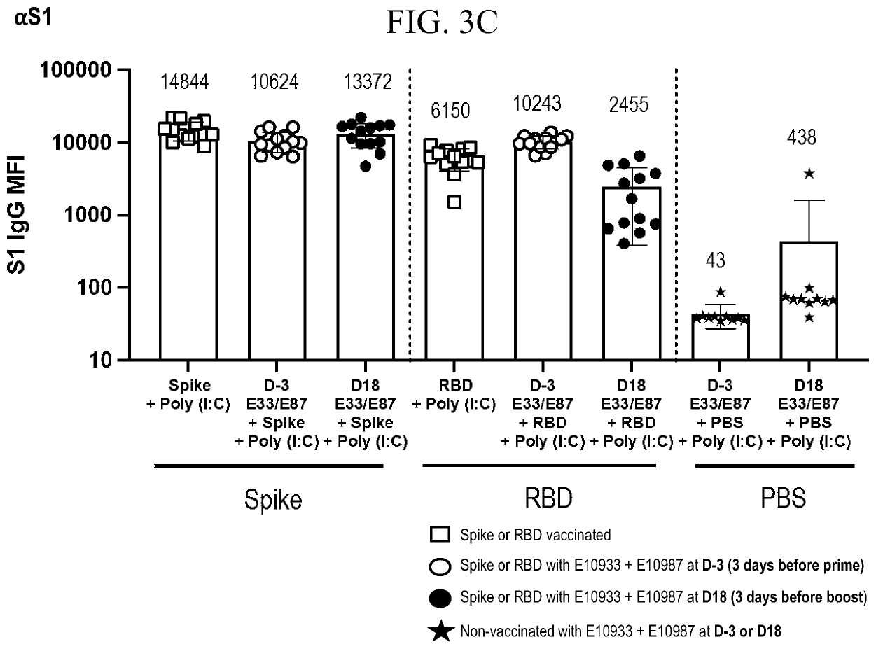

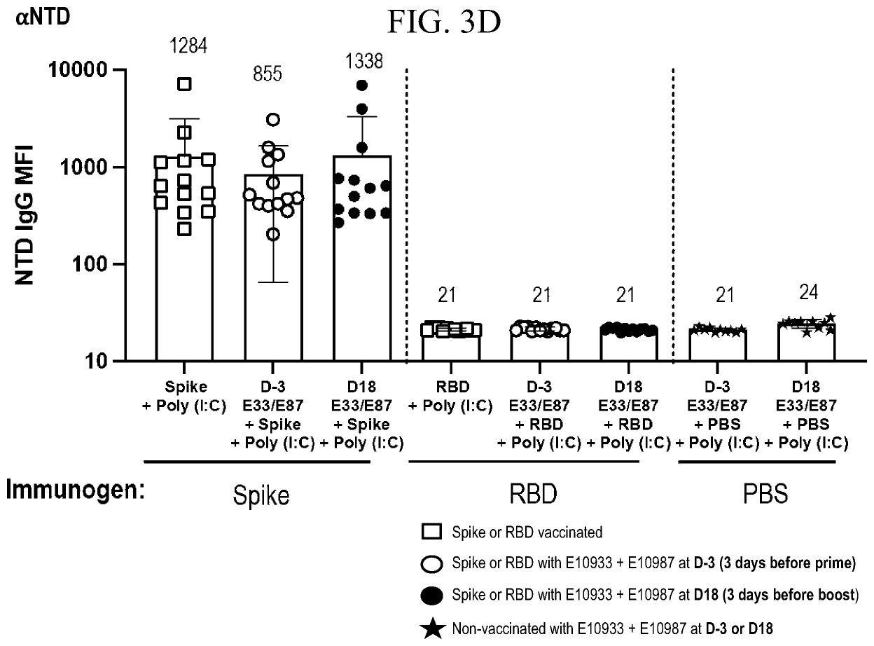

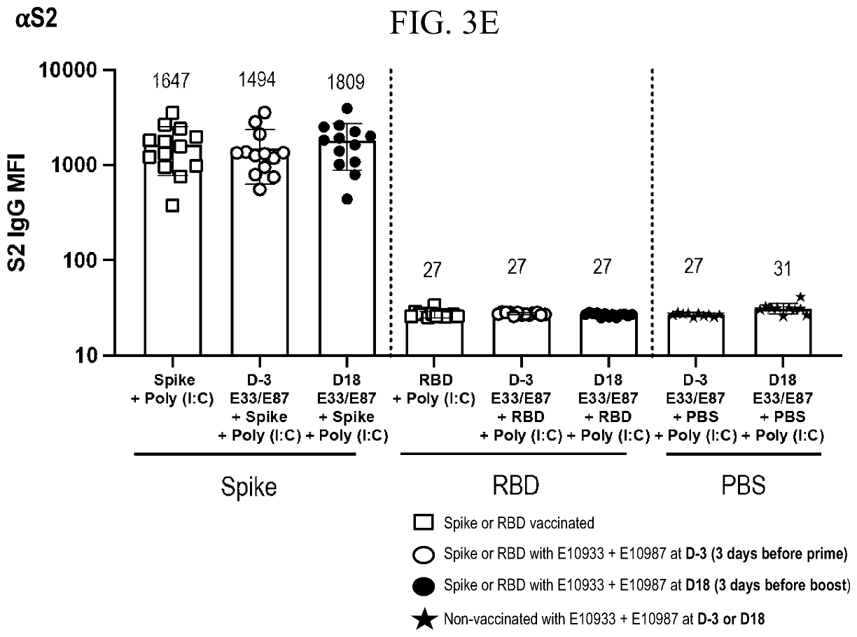

Pre-treatment with αSARS-CoV-2 RBD mAbs (E10933 and E10987) that target neutralizing epitopes before priming or boosting doses of SARS-CoV-2 spike or RBD vaccination was assessed to determine the impact on overall IgG binding levels across SARS-CoV-2 spike regions. As shown in FIGS. 3, E10933 and E10987 mAb treated mice were observed to elicit high αspike IgG levels across all spike regions by day 42, albeit a slight decrease in spike IgG levels was observed in some spike regions, compared to non-mAb treated mice. This suggested that there was not a difference in overall magnitude to different spike regions when mice received αSARS-CoV-2 RBD mAbs prior to vaccination.

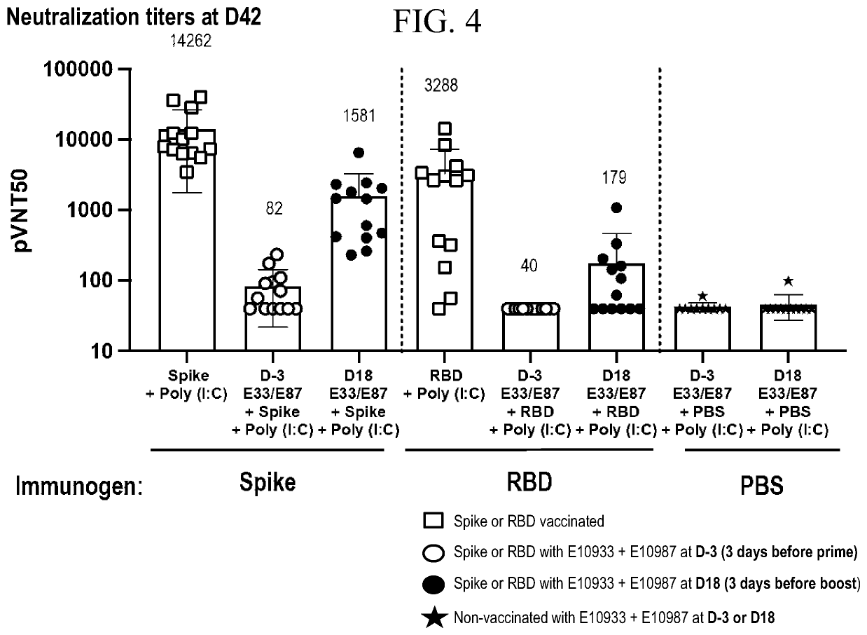

Mice were further evaluated for functional antibody responses by looking at αSARS-CoV-2 spike pseudoviral neutralization titers. Mice pre-treated with αSARS-CoV-2 RBD mAbs (E10933 and E10987) before spike or RBD immunizations elicited a significant decrease in neutralization titers compared to non-mAb treated mice. As shown in FIG. 4, the most substantial difference was seen in mice pre-treated with αSARS-CoV-2 RBD mAbs before spike priming immunization (mean pVNT50 of 82) compared to non-mAb treated mice (mean pVNT50 14262).

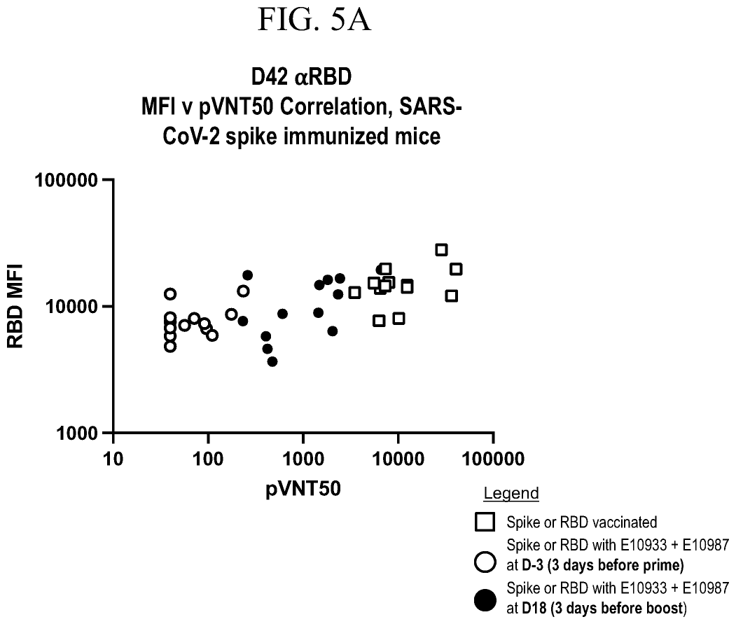

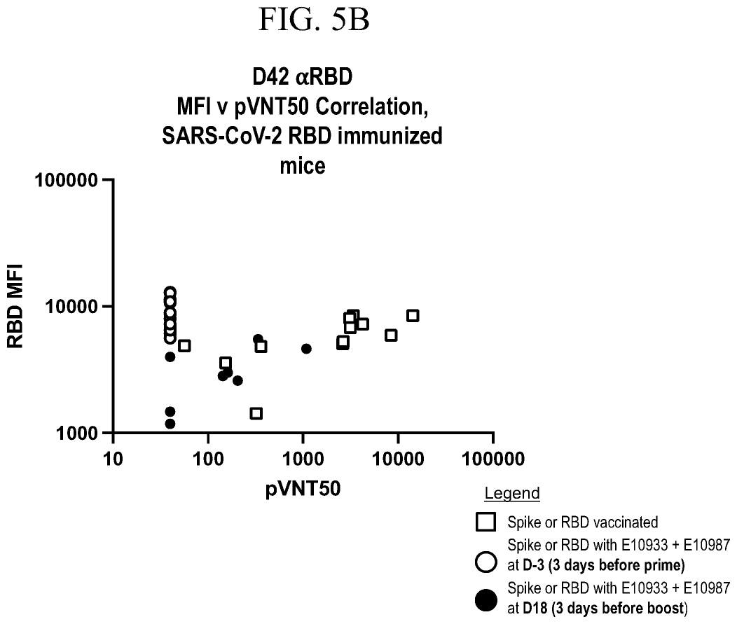

To further understand if the αSARS-CoV-2 RBD mAbs that target neutralizing epitopes skew responses away from epitopes during immunization, the correlation of RBD antibody levels to pVNT50 titers was evaluated. As shown in FIG. 5, in non-mAb treated mice, immunization with spike or RBD elicited high pVNT50 titers that correlated with high RBD IgG levels. However, in mice pre-treated with αSARS-CoV-2 RBD mAbs before spike or RBD prime or boosting immunizations, high RBD IgG antibodies were elicited that did not correlate with high pVNT50 titers. This suggested that when giving αSARS-CoV-2 RBD mAbs targeting neutralizing epitopes prior to SARS-CoV-2 spike or RBD immunizations, the antibody responses were skewed away from these highly potent neutralizing epitopes to weakly or non-neutralizing epitopes on the RBD or spike protein. This is evident as mice still elicited high RBD antibodies that had low neutralization capacity unlike non-mAb treated mice.

The above studies demonstrate that administration of mAbs targeting select epitopes on an immunogen, prior or at the same time as the administration of an immunogen (which can be, e.g., protein based or can be mRNA or DNA that encodes the protein) can profoundly change the properties of the resulting antibody response. This technology can be applied to any vaccine platform that delivers or encodes an antigen that encompasses multiple antibody epitopes. This technology could also be applied to shielding epitopes on endogenous molecules or molecules present on/in pathogens from recognition by the immune system.

### Example 2. Mice Pre-Dosed with SARS-CoV-2 mAbs can Block the Dominant Epitopes During RBD Immunization, and can be Used to Obtain Anti-SARS-CoV-2 mAbs with Different Antigenic Recognition of SARS-CoV-2 Spike than Non-mAb Treated, RBD Immunized Mice

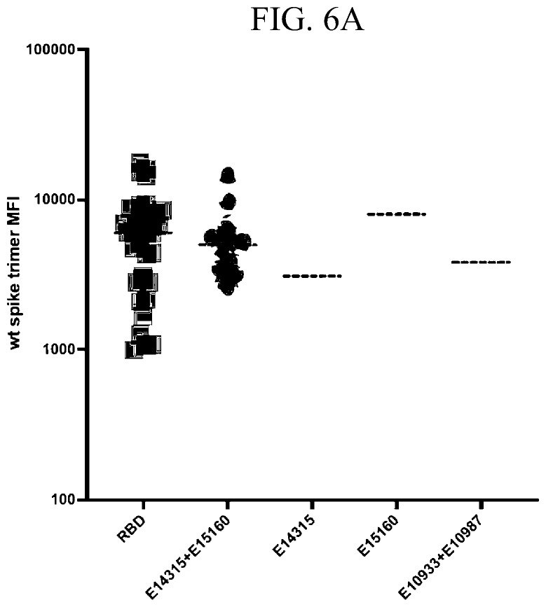

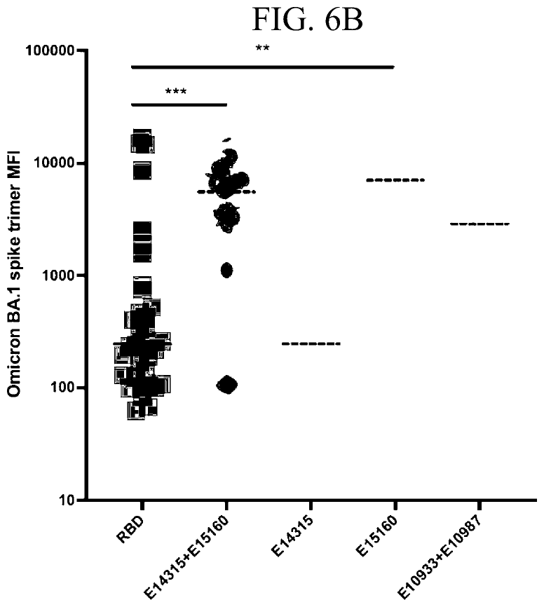

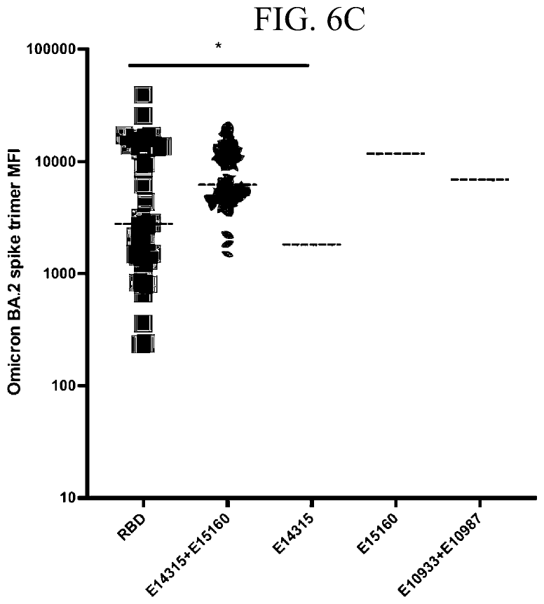

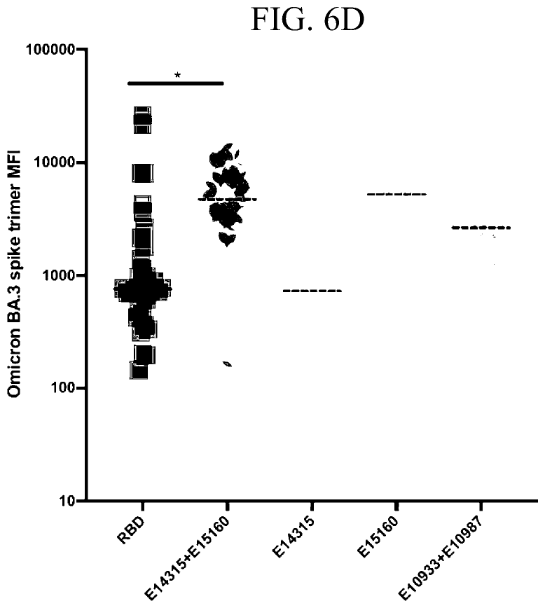

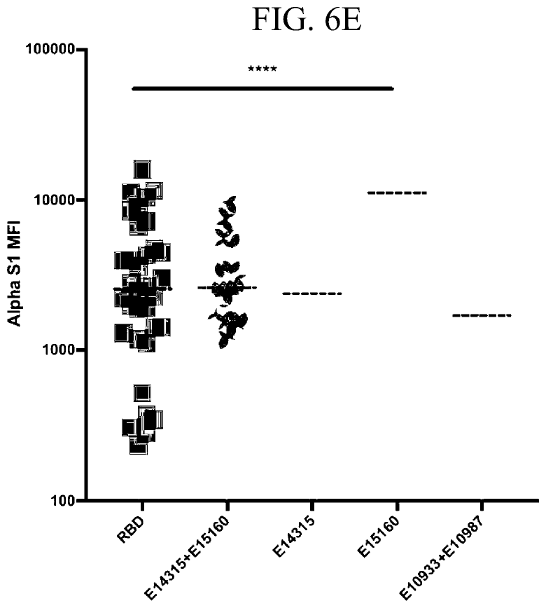

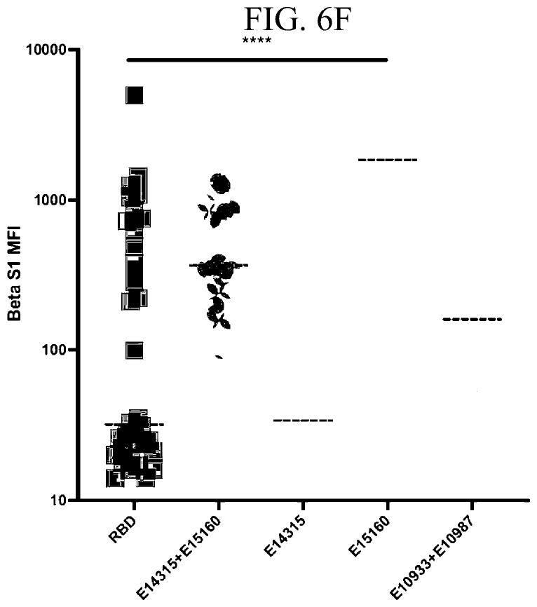

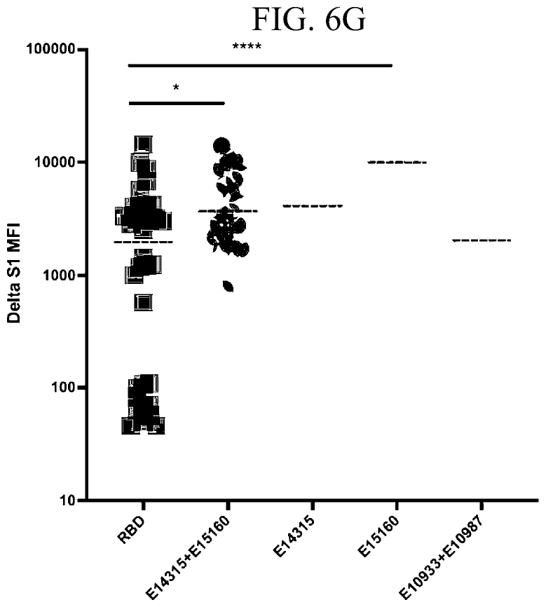

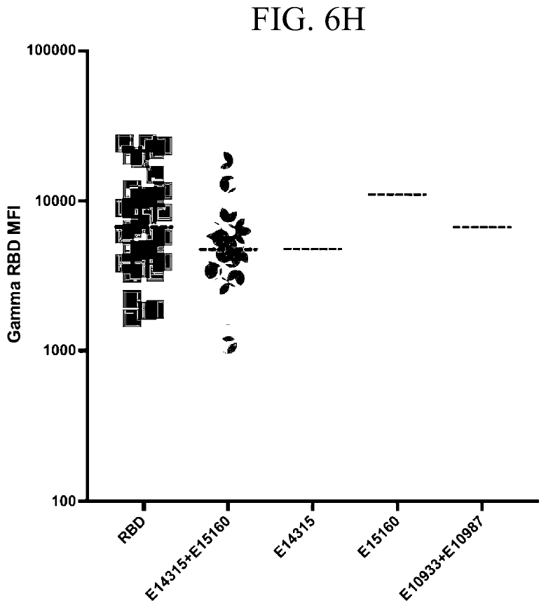

The present Example investigated binding patterns of SARS-CoV-2 monoclonal antibodies (mAbs) obtained from animals pre-dosed with anti-spike mAbs across Variants of Concern (VOC), in particular, VOCs Omicron BA.1, Omicron BA.2, Omicron BA.3, Alpha, Beta, Delta and, Gamma. The SARS-CoV-2 mAbs tested for pre-dosing animals were: E14315+E15160, E14315, E15160, and E10987+E10933. The results showed monoclonal antibodies obtained from animals pre-dosed with anti-spike mAbs E14315+E15160, E14315, E15160, or E10987+E10933 subsequently immunized with RBD displayed differential binding patterns across VOCs, but not against wt recombinant spike proteins compared to RBD immunized, non-mAb pre-treated mice (see, e.g., FIGS. 6A-6H). Most notable was the higher binding to Omicron BA.1 and Delta VOCs spike protein in E14315+E15160, E15160 pre-treated mice. This suggests utilizing mice pre-dosed with SARS-CoV-2 mAbs can block the dominant epitopes during RBD immunization and can be used to obtain anti-SARS-CoV-2 mAbs with different antigenic recognition of SARS-CoV-2 spike than non-mAb treated, RBD immunized mice.

### Example 3. Assessment of E10933 and E10987 Dose Titration on Skewing Antibody Responses to SARS-CoV-2 Spike Immunization

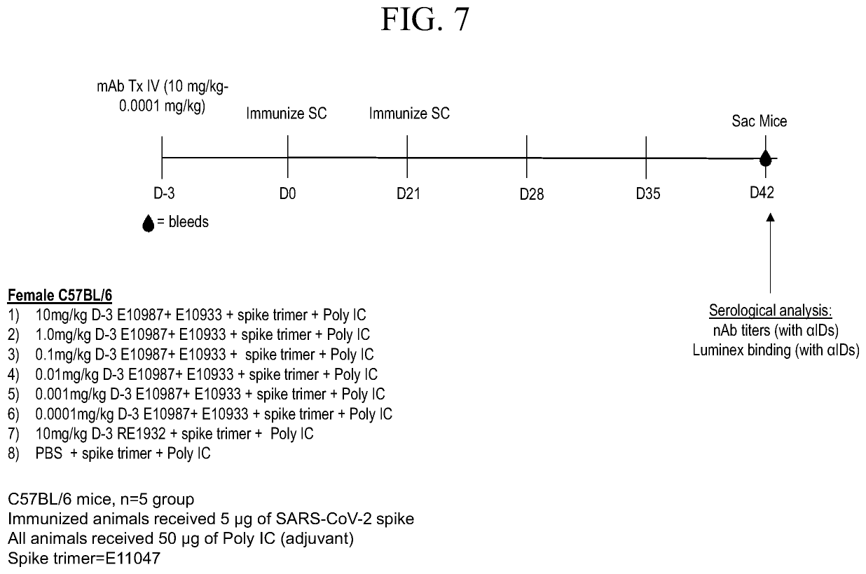

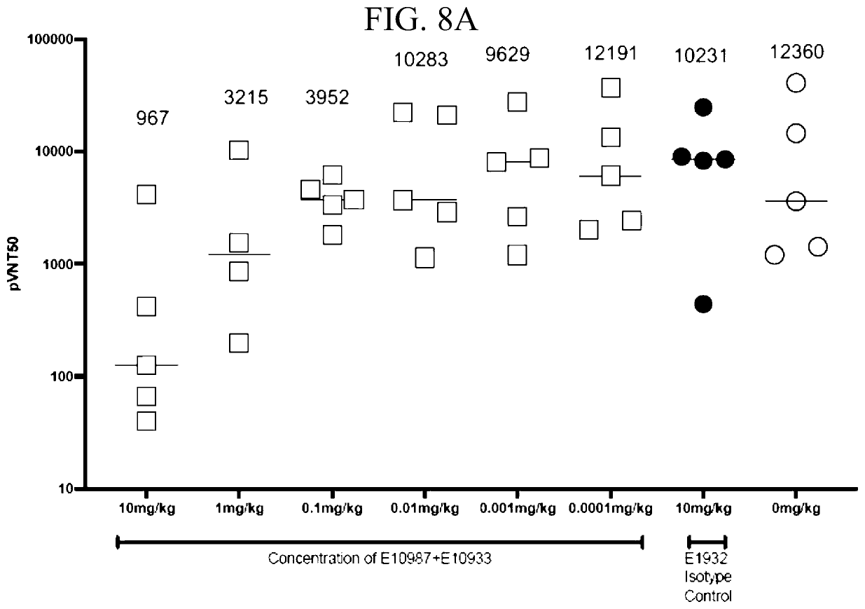

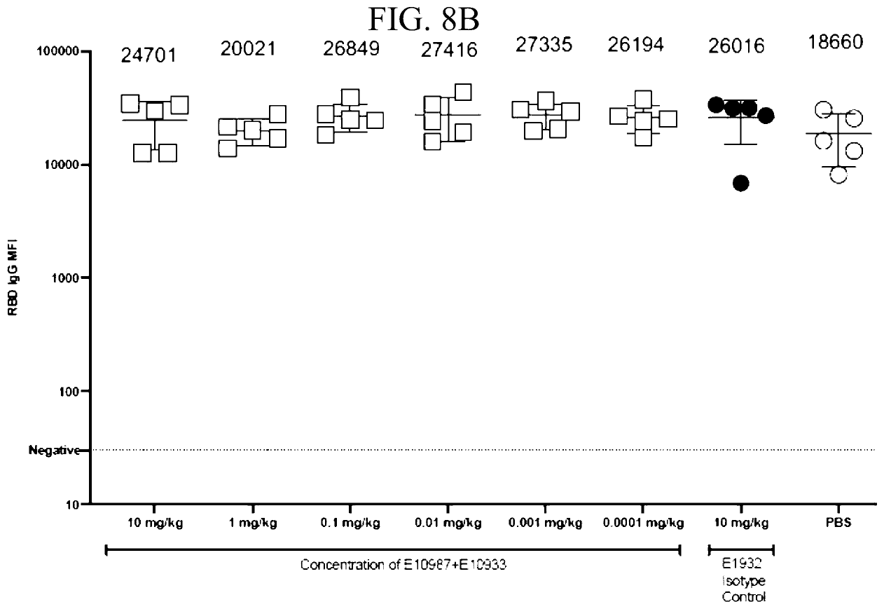

A study design to assess E10933 and E10987 dose titration on skewing antibody responses to SARS-CoV-2 spike immunization is shown in FIG. 7. To assess whether there was a mAb concentration threshold for the skewing effect seen from dosing mice with αSARS-CoV-2 RBD mAbs, pVNT50 titers and RBD binding from mice pre-treated at 10 mg/kg down to 0.0001 mg/kg of αSARS-CoV-2 RBD mAbs before immunizing against SARS-CoV-2 spike were measured (FIGS. 8A-8B). The result showed that mice started to shift back to higher neutralization titers at 0.1 mg/kg dosing, with full neutralization seen at 0.01 mg/kg when compared to non-mAb treated, SARS-CoV-2 spike immunized mice. All groups had similar RBD binding titers demonstrating a skew in antibody responses to different RBD epitopes, and this effect is titratable.

### Example 4. Immunization of VelocImmune (VI) Mice Pre-Treated with Immunogen-Specific Anti-SARS-CoV2 Monoclonal Antibodies (mAbs) and Analysis of Serum Antibody Responses to the Immunogen

The description below relates to immunization of VelocImmune (VI) mice which were pre-treated with immunogen specific anti-SARS-CoV2 monoclonal antibodies (mAbs) and analysis of serum antibody responses to the immunogen.

### Immunization

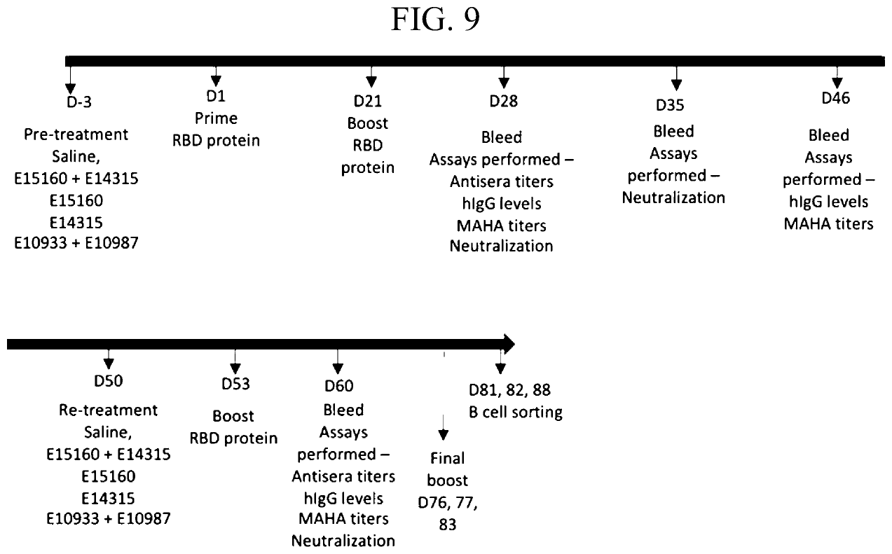

VelocImmune (VI) mice (see, e.g., U.S. Pat. No. 6,596,541, Regeneron Pharmaceuticals, VELCOIMMUNE®, incorporated herein by reference in its entirety for all intended purposes) were immunized with a protein immunogen (Day 1) containing SARS-CoV-2 Spike Protein Receptor Binding Domain (RBD) fused to a C-terminal mFc tag following standard immunization protocols. Three days prior to RBD protein priming injection and 50 days after, mice were pre-treated with 4 different anti-SARS-CoV-2 spike human mAbs in four different combinations, at a dose of 10 mg/kg of each antibody, and a cohort without antibody pre-treatment (saline only) was also included, as shown in the immunization scheme displayed in FIG. 9. Mice were pre-bled prior to the mAbs pre-treatment, post immunogen boosts at days 28, 35, 46 and 60, and prior to euthanizing mice for antibody isolation. Serum from bleeds were subjected to titer analysis on SARS-CoV-2 Spike Protein RBD domain fused to a C-terminal myc-myc-histidine tag (referred to as SARS-CoV-2 Spike Protein (RBD).mmH), and against human mAbs dosed in the pre-treatment. Bleeds were also subjected to human IgG quantification analysis on anti-SARS-CoV-2 spike human mAb for pre-treatment.

### Anti-SARS-CoV-2 Spike Protein Serum Titer Determination

Antibody titers in serum (with and without depleting pre-treated anti-SARS-CoV2 human mAbs using anti-human IgG antibody) against SARS-CoV-2 Spike Protein (RBD) were determined by solid-phase enzyme-linked immunoassay (ELISA). Ninety-six-well microtiter plates were coated with SARS-CoV-2 Spike Protein (RBD).mmH at 2 μg/ml in phosphate-buffered saline (PBS, Irvine Scientific) overnight at 4° C. Plates were washed with PBS containing 0.05% Tween-20 (PBS-T) and blocked with 250 μL of 1% bovine serum albumin (BSA) in PBS for 1 hour at room temperature (RT). The plates were washed with PBS-T. Pre-immune and immune anti-sera were serially diluted three-fold in 1% BSA-PBS and added to the plates for 1 hour at RT. The plates were washed, and goat anti-mouse IgG Horseradish Peroxidase (HRP) conjugated secondary antibodies (Jackson ImmunoResearch) were added at 1:5000 dilution to the plates and incubated for 1 hour at RT. Plates were washed and developed using TMB/H₂O₂ (Tetramethyl benzidine/Hydrogen Peroxide) as substrate (BD) by incubating for 15-20 min. The reaction was stopped with acid and plates read on a spectrophotometer (EnVision, Perkin Elmer) and absorbance at 450 nm was recorded. Antibody titers were computed using Graphpad PRISM software. The titer was defined as interpolated serum dilution factor of which the binding signal is 2-fold over background.

### Anti-Human IgG Serum Titer Determination

To determine whether the tested mice also mounted an immune response against the human IgG included in the pretreatment, the aforementioned protocol was applied to detect immune response against the anti-SARS-CoV-2 Spike human mAbs included in the pre-treatment (mouse anti-human antibody, MAHA), except that the microtiter plates were coated with the individual anti-SARS-CoV-2 Spike mAbs.

### Total Human IgG Quantification

Levels of the total amount of the dosed anti-RBD mAbs in anti-sera were also quantitated with an immunoassay similar to the ELISA described above. The pre-immune and immune anti-sera were serially diluted three-fold, added to microtiter plates coated with the RBD recombinant protein, and goat anti-human IgG-Fc-HRP conjugated secondary antibodies (Jackson ImmunoResearch) were used as detection. The antibody concentrations in the sera were calculated using Graphpad PRISM software using a calibration curve of respective anti-SARS-CoV-2 Spike mAbs included in the pre-treatment.

### Results

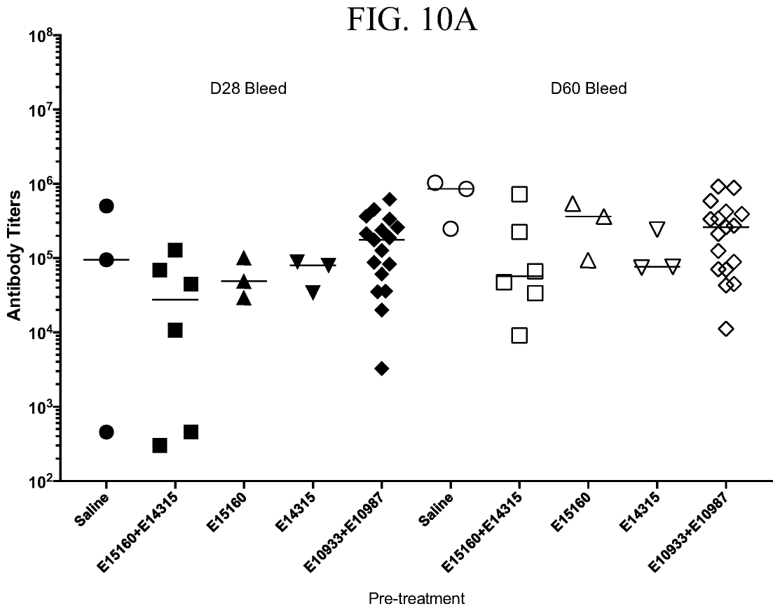

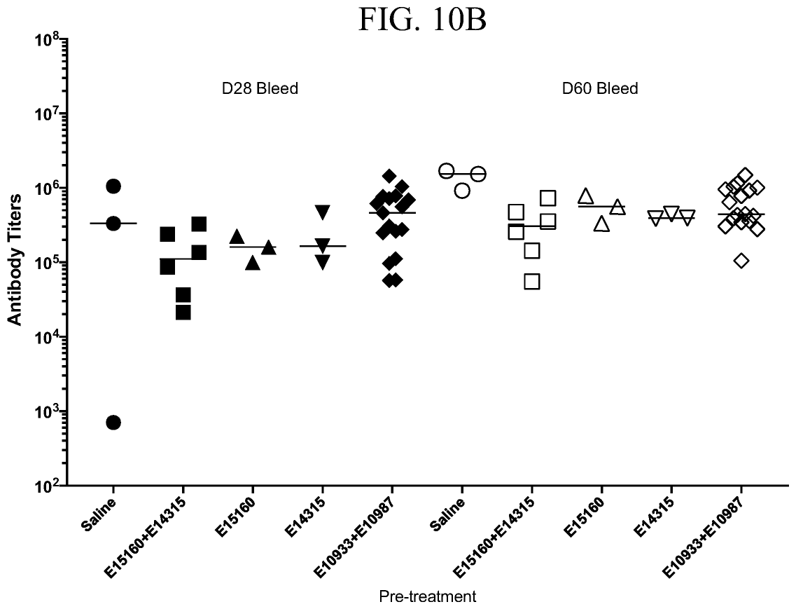

The humoral immune response in VI mice was determined using recombinant SARS-CoV-2 Spike protein (RBD) post-immunization. A portion of the sera sample was depleted of the pre-treated human anti-SARS-CoV-2 mAbs by immunoprecipitation using anti-human IgG beads. Briefly, 0.23 mg of anti-human IgG beads (AbraMag, Cat:544061) were incubated with 25 μL of mouse sera for 30 mins. The bead and mouse serum mixture were added to a magnetic separator and mouse serum supernatants were gathered. This process was repeated twice more to completely remove any interfering human IgG mAbs for mouse antibody analysis. High titers against SARS-CoV-2 Spike RBD were observed with median titers >100,000 without human mAb depletion in all cohorts of mice on day 28 (FIG. 10B), while the median titers ranged from about ˜27,000-176,000 after human mAb in the sera samples were removed. Comparing to the titers on day 28, increase in titers were observed on day 60 post immunization with median titers >300,000 (without human mAb depletion) and ˜56,000-˜ 855,000 (with human mAb depletion) against SARS-CoV-2 Spike RBD (FIGS. 10A-10B).

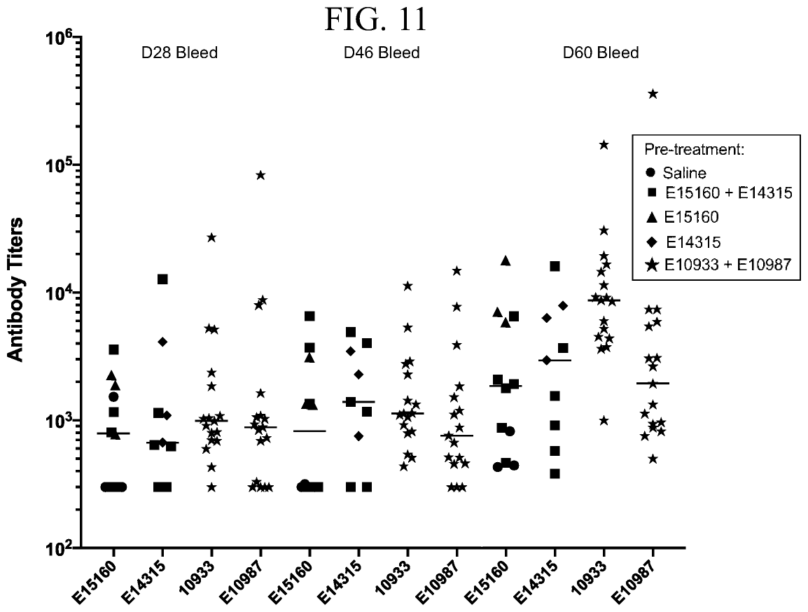

Mouse anti-human IgG antibody (MAHA) titers were detected in sera from anti-SARS-CoV-2 Spike human mAb treated mice. Antibody titers on plate coated anti-SARS-CoV-2 Spike mAbs ranged in median from ˜668-989, 758-1,395 and 1,851-8,671 on days 28, 46 and 60, respectively (FIG. 11).

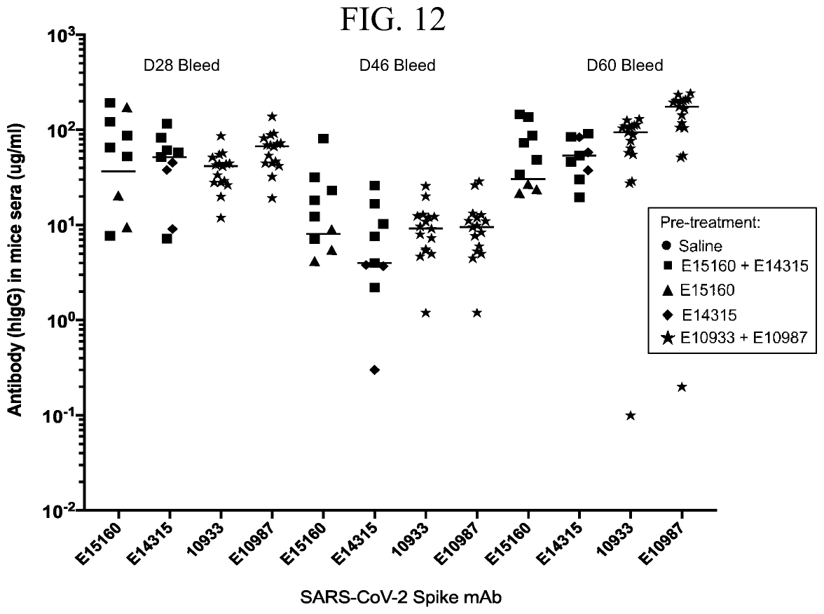

An average level of total human mAb was determined to be 57.7 μg/mL, 12 μg/mL, and 98.7 μg/mL at days 28, 46, and 60 respectively (FIG. 12). The higher level at day 60 is a consequence of re-dosing of the mAbs at day 50. There were no detectable levels of human mAb in sera with hIgG depleted by anti-human IgG beads. Low (<0.5 μg/ml) or BDL (below detection limit) of SARS-CoV-2 Spike RBD specific human mAb were observed before human IgG removal from mouse with an outstanding MAHA titers (>27,000 and 83,000 titers on day 28 from cohort pre-dosed with E10933+E10987, FIG. 11).

The results presented herein demonstrate that mice pre-dosed with anti-SARS-CoV-mAbs followed by RBD immunization still mount a detectable and strong antibody response to the RBD immunogen similar to the control non-mAb pre-dosed mice.

### Example 5. Octet Cross-Competition Between 773 Anti-COVID19 Monoclonal Primary Supernatant from CHOt

Binding competition between the test anti-SARS-CoV-2 monoclonal antibodies (total of 773 mAbs) and each of the four antibodies included in pre-treatment of immunization was determined using a real-time, label-free bio-layer interferometry (BLI) assay on an Octet HTX biosensor (ForteBio Corp., A Division of Sartorius). Table 5 describes the test anti-SARS-CoV-2 monoclonal antibodies. Table 6 describes the number of anti-SARS-CoV-2 mAbs tested per immunization group. Table 7 describes the reagents used in the present Example. MW, molecular weight.

TABLE 5
Test anti-SARS-CoV-2 monoclonal antibodies
List of transiently expressed anti-SARS-CoV-2 mAbs

| | Sample ID | Aliquot | mAb | |
|---|---|---|---|---|
| Clone ID | (ws/“0”) | ID | # | Immunization group |
| 1120-1-61A10 | 1120-61-A10 | 46410800 | 1 | RBD + E14315 + E15160 |
| 1120-1-61A11 | 1120-61-A11 | 46410806 | 2 | RBD + E14315 + E15160 |
| 1120-1-61A12 | 1120-61-A12 | 46410813 | 3 | RBD + E14315 + E15160 |
| 1120-1-61A2 | 1120-61-A02 | 46410740 | 4 | RBD + E14315 + E15160 |
| 1120-1-61A3 | 1120-61-A03 | 46410748 | 5 | RBD + E14315 + E15160 |
| 1120-1-61A4 | 1120-61-A04 | 46410755 | 6 | RBD + E14315 + E15160 |
| 1120-1-61A5 | 1120-61-A05 | 46410762 | 7 | RBD + E14315 + E15160 |
| 1120-1-61A6 | 1120-61-A06 | 46410770 | 8 | RBD + E14315 + E15160 |
| 1120-1-61A7 | 1120-61-A07 | 46410776 | 9 | RBD + E14315 + E15160 |
| 1120-1-61A8 | 1120-61-A08 | 46410784 | 10 | RBD + E14315 + E15160 |
| 1120-1-61A9 | 1120-61-A09 | 46410792 | 11 | RBD + E14315 + E15160 |
| 1120-1-61B10 | 1120-61-B10 | 46410801 | 12 | RBD + E14315 + E15160 |
| 1120-1-61B11 | 1120-61-B11 | 46410807 | 13 | RBD + E14315 + E15160 |
| 1120-1-61B12 | 1120-61-B12 | 46410814 | 14 | RBD + E14315 + E15160 |
| 1120-1-61B2 | 1120-61-B02 | 46410741 | 15 | RBD + E14315 + E15160 |
| 1120-1-61B3 | 1120-61-B03 | 46410749 | 16 | RBD + E14315 + E15160 |
| 1120-1-61B4 | 1120-61-B04 | 46410756 | 17 | RBD + E14315 + E15160 |
| 1120-1-61B5 | 1120-61-B05 | 46410763 | 18 | RBD + E14315 + E15160 |
| 1120-1-61B6 | 1120-61-B06 | 46410771 | 19 | RBD + E14315 + E15160 |
| 1120-1-61B7 | 1120-61-B07 | 46410777 | 20 | RBD + E14315 + E15160 |
| 1120-1-61B8 | 1120-61-B08 | 46410785 | 21 | RBD + E14315 + E15160 |
| 1120-1-61B9 | 1120-61-B09 | 46410793 | 22 | RBD + E14315 + E15160 |
| 1120-1-61C10 | 1120-61-C10 | 46410802 | 23 | RBD + E14315 + E15160 |
| 1120-1-61C12 | 1120-61-C12 | 46410815 | 24 | RBD + E14315 + E15160 |
| 1120-1-61C2 | 1120-61-C02 | 46410742 | 25 | RBD + E14315 + E15160 |
| 1120-1-61C3 | 1120-61-C03 | 46410750 | 26 | RBD + E14315 + E15160 |
| 1120-1-61C4 | 1120-61-C04 | 46410757 | 27 | RBD + E14315 + E15160 |
| 1120-1-61C5 | 1120-61-C05 | 46410764 | 28 | RBD + E14315 + E15160 |
| 1120-1-61C6 | 1120-61-C06 | 46410772 | 29 | RBD + E14315 + E15160 |
| 1120-1-61C7 | 1120-61-C07 | 46410778 | 30 | RBD + E14315 + E15160 |
| 1120-1-61C8 | 1120-61-C08 | 46410786 | 31 | RBD + E14315 + E15160 |
| 1120-1-61C9 | 1120-61-C09 | 46410794 | 32 | RBD + E14315 + E15160 |
| 1120-1-61D10 | 1120-61-D10 | 46410803 | 33 | RBD + E14315 + E15160 |
| 1120-1-61D11 | 1120-61-D11 | 46410808 | 34 | RBD + E14315 + E15160 |
| 1120-1-61D12 | 1120-61-D12 | 46410816 | 35 | RBD + E14315 + E15160 |
| 1120-1-61D2 | 1120-61-D02 | 46410743 | 36 | RBD + E14315 + E15160 |
| 1120-1-61D3 | 1120-61-D03 | 46410751 | 37 | RBD + E14315 + E15160 |
| 1120-1-61D4 | 1120-61-D04 | 46410758 | 38 | RBD + E14315 + E15160 |
| 1120-1-61D5 | 1120-61-D05 | 46410765 | 39 | RBD + E14315 + E15160 |
| 1120-1-61D6 | 1120-61-D06 | 46410773 | 40 | RBD + E14315 + E15160 |
| 1120-1-61D7 | 1120-61-D07 | 46410779 | 41 | RBD + E14315 + E15160 |
| 1120-1-61D8 | 1120-61-D08 | 46410787 | 42 | RBD + E14315 + E15160 |
| 1120-1-61D9 | 1120-61-D09 | 46410795 | 43 | RBD + E14315 + E15160 |
| 1120-1-61E10 | 1120-61-E10 | 46410804 | 44 | RBD + E14315 + E15160 |
| 1120-1-61E11 | 1120-61-E11 | 46410809 | 45 | RBD + E14315 + E15160 |
| 1120-1-61E2 | 1120-61-E02 | 46410744 | 46 | RBD + E14315 + E15160 |
| 1120-1-61E3 | 1120-61-E03 | 46410752 | 47 | RBD + E14315 + E15160 |
| 1120-1-61E4 | 1120-61-E04 | 46410759 | 48 | RBD + E14315 + E15160 |
| 1120-1-61E5 | 1120-61-E05 | 46410766 | 49 | RBD + E14315 + E15160 |
| 1120-1-61E6 | 1120-61-E06 | 46410774 | 50 | RBD + E14315 + E15160 |
| 1120-1-61E7 | 1120-61-E07 | 46410780 | 51 | RBD + E14315 + E15160 |
| 1120-1-61E8 | 1120-61-E08 | 46410788 | 52 | RBD + E14315 + E15160 |
| 1120-1-61E9 | 1120-61-E09 | 46410796 | 53 | RBD + E14315 + E15160 |
| 1120-1-61F11 | 1120-61-F11 | 46410810 | 54 | RBD + E14315 + E15160 |
| 1120-1-61F12 | 1120-61-F12 | 46410817 | 55 | RBD + E14315 + E15160 |
| 1120-1-61F2 | 1120-61-F02 | 46410745 | 56 | RBD + E14315 + E15160 |
| 1120-1-61F4 | 1120-61-F04 | 46410760 | 57 | RBD + E14315 + E15160 |
| 1120-1-61F5 | 1120-61-F05 | 46410767 | 58 | RBD + E14315 + E15160 |
| 1120-1-61F7 | 1120-61-F07 | 46410781 | 59 | RBD + E14315 + E15160 |
| 1120-1-61F8 | 1120-61-F08 | 46410789 | 60 | RBD + E14315 + E15160 |
| 1120-1-61F9 | 1120-61-F09 | 46410797 | 61 | RBD + E14315 + E15160 |
| 1120-1-61G10 | 1120-61-G10 | 46410805 | 62 | RBD + E14315 + E15160 |
| 1120-1-61G11 | 1120-61-G11 | 46410811 | 63 | RBD + E14315 + E15160 |
| 1120-1-61G12 | 1120-61-G12 | 46410818 | 64 | RBD + E14315 + E15160 |
| 1120-1-61G2 | 1120-61-G02 | 46410746 | 65 | RBD + E14315 + E15160 |
| 1120-1-61G3 | 1120-61-G03 | 46410753 | 66 | RBD + E14315 + E15160 |
| 1120-1-61G4 | 1120-61-G04 | 46410761 | 67 | RBD + E14315 + E15160 |
| 1120-1-61G5 | 1120-61-G05 | 46410768 | 68 | RBD + E14315 + E15160 |
| 1120-1-61G7 | 1120-61-G07 | 46410782 | 69 | RBD + E14315 + E15160 |
| 1120-1-61G8 | 1120-61-G08 | 46410790 | 70 | RBD + E14315 + E15160 |
| 1120-1-61G9 | 1120-61-G09 | 46410798 | 71 | RBD + E14315 + E15160 |
| 1120-1-61H11 | 1120-61-H11 | 46410812 | 72 | RBD + E14315 + E15160 |
| 1120-1-61H12 | 1120-61-H12 | 46410819 | 73 | RBD + E14315 + E15160 |
| 1120-1-61H2 | 1120-61-H02 | 46410747 | 74 | RBD + E14315 + E15160 |
| 1120-1-61H3 | 1120-61-H03 | 46410754 | 75 | RBD + E14315 + E15160 |
| 1120-1-61H5 | 1120-61-H05 | 46410769 | 76 | RBD + E14315 + E15160 |
| 1120-1-61H6 | 1120-61-H06 | 46410775 | 77 | RBD + E14315 + E15160 |
| 1120-1-61H7 | 1120-61-H07 | 46410783 | 78 | RBD + E14315 + E15160 |
| 1120-1-61H8 | 1120-61-H08 | 46410791 | 79 | RBD + E14315 + E15160 |
| 1120-1-61H9 | 1120-61-H09 | 46410799 | 80 | RBD + E14315 + E15160 |
| 1120-1-62A2 | 1120-62-A02 | 46410820 | 81 | RBD + E14315 + E15160 |
| 1120-1-62B2 | 1120-62-B02 | 46410821 | 82 | RBD + E14315 + E15160 |
| 1120-1-62C2 | 1120-62-C02 | 46410822 | 83 | RBD + E14315 + E15160 |
| 1120-5-63A11 | 1120-63-A11 | 46410942 | 84 | RBD + E14315 |
| 1120-5-63 A4 | 1120-63-A04 | 46410906 | 85 | RBD + E14315 |
| 1120-5-63A5 | 1120-63-A05 | 46410910 | 86 | RBD + E14315 |
| 1120-5-63A6 | 1120-63-A06 | 46410914 | 87 | RBD + E14315 |
| 1120-5-63A8 | 1120-63-A08 | 46410926 | 88 | RBD + E14315 |
| 1120-5-63B11 | 1120-63-B11 | 46410943 | 89 | RBD + E14315 |
| 1120-5-63B4 | 1120-63-B04 | 46410907 | 90 | RBD + E14315 |
| 1120-5-63B6 | 1120-63-B06 | 46410915 | 91 | RBD + E14315 |
| 1120-5-63B8 | 1120-63-B08 | 46410927 | 92 | RBD + E14315 |
| 1120-5-63C10 | 1120-63-C10 | 46410937 | 93 | RBD + E14315 |
| 1120-5-63C11 | 1120-63-C11 | 46410944 | 94 | RBD + E14315 |
| 1120-5-63C12 | 1120-63-C12 | 46410948 | 95 | RBD + E14315 |
| 1120-5-63C6 | 1120-63-C06 | 46410916 | 96 | RBD + E14315 |
| 1120-5-63C7 | 1120-63-C07 | 46410922 | 97 | RBD + E14315 |
| 1120-5-63C8 | 1120-63-C08 | 46410928 | 98 | RBD + E14315 |
| 1120-5-63C9 | 1120-63-C09 | 46410933 | 99 | RBD + E14315 |
| 1120-5-63D10 | 1120-63-D10 | 46410938 | 100 | RBD + E14315 |
| 1120-5-63D12 | 1120-63-D12 | 46410949 | 101 | RBD + E14315 |
| 1120-5-63D5 | 1120-63-D05 | 46410911 | 102 | RBD + E14315 |
| 1120-5-63D6 | 1120-63-D06 | 46410917 | 103 | RBD + E14315 |
| 1120-5-63D7 | 1120-63-D07 | 46410923 | 104 | RBD + E14315 |
| 1120-5-63D8 | 1120-63-D08 | 46410929 | 105 | RBD + E14315 |
| 1120-5-63D9 | 1120-63-D09 | 46410934 | 106 | RBD + E14315 |
| 1120-5-63E10 | 1120-63-E10 | 46410939 | 107 | RBD + E14315 |
| 1120-5-63E11 | 1120-63-E11 | 46410945 | 108 | RBD + E14315 |
| 1120-5-63E3 | 1120-63-E03 | 46410904 | 109 | RBD + E14315 |
| 1120-5-63E4 | 1120-63-E04 | 46410908 | 110 | RBD + E14315 |
| 1120-5-63E5 | 1120-63-E05 | 46410912 | 111 | RBD + E14315 |
| 1120-5-63E6 | 1120-63-E06 | 46410918 | 112 | RBD + E14315 |
| 1120-5-63E7 | 1120-63-E07 | 46410924 | 113 | RBD + E14315 |
| 1120-5-63E8 | 1120-63-E08 | 46410930 | 114 | RBD + E14315 |
| 1120-5-63E9 | 1120-63-E09 | 46410935 | 115 | RBD + E14315 |
| 1120-5-63F10 | 1120-63-F10 | 46410940 | 116 | RBD + E14315 |
| 1120-5-63F11 | 1120-63-F11 | 46410946 | 117 | RBD + E14315 |
| 1120-5-63F12 | 1120-63-F12 | 46410950 | 118 | RBD + E14315 |
| 1120-5-63F3 | 1120-63-F03 | 46410905 | 119 | RBD + E14315 |
| 1120-5-63F5 | 1120-63-F05 | 46410913 | 120 | RBD + E14315 |
| 1120-5-63F6 | 1120-63-F06 | 46410919 | 121 | RBD + E14315 |
| 1120-5-63F8 | 1120-63-F08 | 46410931 | 122 | RBD + E14315 |
| 1120-5-63F9 | 1120-63-F09 | 46410936 | 123 | RBD + E14315 |
| 1120-5-63G10 | 1120-63-G10 | 46410941 | 124 | RBD + E14315 |
| 1120-5-63G11 | 1120-63-G11 | 46410947 | 125 | RBD + E14315 |
| 1120-5-63G4 | 1120-63-G04 | 46410909 | 126 | RBD + E14315 |
| 1120-5-63G6 | 1120-63-G06 | 46410920 | 127 | RBD + E14315 |
| 1120-5-63G7 | 1120-63-G07 | 46410925 | 128 | RBD + E14315 |
| 1120-5-63G8 | 1120-63-G08 | 46410932 | 129 | RBD + E14315 |
| 1120-5-63H12 | 1120-63-H12 | 46410951 | 130 | RBD + E14315 |
| 1120-5-63H6 | 1120-63-H06 | 46410921 | 131 | RBD + E14315 |
| 1120-5-64A4 | 1120-64-A04 | 46410961 | 132 | RBD + E14315 |
| 1120-5-64B2 | 1120-64-B02 | 46410952 | 133 | RBD + E14315 |
| 1120-5-64B3 | 1120-64-B03 | 46410955 | 134 | RBD + E14315 |
| 1120-5-64B4 | 1120-64-B04 | 46410962 | 135 | RBD + E14315 |
| 1120-5-64C3 | 1120-64-C03 | 46410956 | 136 | RBD + E14315 |
| 1120-5-64D3 | 1120-64-D03 | 46410957 | 137 | RBD + E14315 |
| 1120-5-64F3 | 1120-64-F03 | 46410958 | 138 | RBD + E14315 |
| 1120-5-64G2 | 1120-64-G02 | 46410953 | 139 | RBD + E14315 |
| 1120-5-64G3 | 1120-64-G03 | 46410959 | 140 | RBD + E14315 |
| 1120-5-64H2 | 1120-64-H02 | 46410954 | 141 | RBD + E14315 |
| 1120-5-64H3 | 1120-64-H03 | 46410960 | 142 | RBD + E14315 |
| 1120-7-66A11 | 1120-66-A11 | 46411119 | 143 | RBD + E14315 |
| 1120-7-66A12 | 1120-66-A12 | 46411126 | 144 | RBD + E14315 |
| 1120-7-66A6 | 1120-66-A06 | 46411096 | 145 | RBD + E14315 |
| 1120-7-66A7 | 1120-66-A07 | 46411102 | 146 | RBD + E14315 |
| 1120-7-66A9 | 1120-66-A09 | 46411112 | 147 | RBD + E14315 |
| 1120-7-66B11 | 1120-66-B11 | 46411120 | 148 | RBD + E14315 |
| 1120-7-66B12 | 1120-66-B12 | 46411127 | 149 | RBD + E14315 |
| 1120-7-66B6 | 1120-66-B06 | 46411097 | 150 | RBD + E14315 |
| 1120-7-66B7 | 1120-66-B07 | 46411103 | 151 | RBD + E14315 |
| 1120-7-66B8 | 1120-66-B08 | 46411108 | 152 | RBD + E14315 |
| 1120-7-66B9 | 1120-66-B09 | 46411113 | 153 | RBD + E14315 |
| 1120-7-66C12 | 1120-66-C12 | 46411128 | 154 | RBD + E14315 |
| 1120-7-66C6 | 1120-66-C06 | 46411098 | 155 | RBD + E14315 |
| 1120-7-66C7 | 1120-66-C07 | 46411104 | 156 | RBD + E14315 |
| 1120-7-66C8 | 1120-66-C08 | 46411109 | 157 | RBD + E14315 |
| 1120-7-66D10 | 1120-66-D10 | 46411116 | 158 | RBD + E14315 |
| 1120-7-66D11 | 1120-66-D11 | 46411121 | 159 | RBD + E14315 |
| 1120-7-66D12 | 1120-66-D12 | 46411129 | 160 | RBD + E14315 |
| 1120-7-66D8 | 1120-66-D08 | 46411110 | 161 | RBD + E14315 |
| 1120-7-66D9 | 1120-66-D09 | 46411114 | 162 | RBD + E14315 |
| 1120-7-66E11 | 1120-66-E11 | 46411122 | 163 | RBD + E14315 |
| 1120-7-66E12 | 1120-66-E12 | 46411130 | 164 | RBD + E14315 |
| 1120-7-66E6 | 1120-66-E06 | 46411099 | 165 | RBD + E14315 |
| 1120-7-66E7 | 1120-66-E07 | 46411105 | 166 | RBD + E14315 |
| 1120-7-66E8 | 1120-66-E08 | 46411111 | 167 | RBD + E14315 |
| 1120-7-66F10 | 1120-66-F10 | 46411117 | 168 | RBD + E14315 |
| 1120-7-66F11 | 1120-66-F11 | 46411123 | 169 | RBD + E14315 |
| 1120-7-66F5 | 1120-66-F05 | 46411093 | 170 | RBD + E14315 |
| 1120-7-66F6 | 1120-66-F06 | 46411100 | 171 | RBD + E14315 |
| 1120-7-66F7 | 1120-66-F07 | 46411106 | 172 | RBD + E14315 |
| 1120-7-66G11 | 1120-66-G11 | 46411124 | 173 | RBD + E14315 |
| 1120-7-66G12 | 1120-66-G12 | 46411131 | 174 | RBD + E14315 |
| 1120-7-66G5 | 1120-66-G05 | 46411094 | 175 | RBD + E14315 |
| 1120-7-66G6 | 1120-66-G06 | 46411101 | 176 | RBD + E14315 |
| 1120-7-66G9 | 1120-66-G09 | 46411115 | 177 | RBD + E14315 |
| 1120-7-66H10 | 1120-66-H10 | 46411118 | 178 | RBD + E14315 |
| 1120-7-66H11 | 1120-66-H11 | 46411125 | 179 | RBD + E14315 |
| 1120-7-66H5 | 1120-66-H05 | 46411095 | 180 | RBD + E14315 |
| 1120-7-66H7 | 1120-66-H07 | 46411107 | 181 | RBD + E14315 |
| 1120-7-67A2 | 1120-67-A02 | 46411132 | 182 | RBD + E14315 |
| 1120-7-67A5 | 1120-67-A05 | 46411153 | 183 | RBD + E14315 |
| 1120-7-67A6 | 1120-67-A06 | 46411161 | 184 | RBD + E14315 |
| 1120-7-67B2 | 1120-67-B02 | 46411133 | 185 | RBD + E14315 |
| 1120-7-67B3 | 1120-67-B03 | 46411140 | 186 | RBD + E14315 |
| 1120-7-67B4 | 1120-67-B04 | 46411146 | 187 | RBD + E14315 |
| 1120-7-67B5 | 1120-67-B05 | 46411154 | 188 | RBD + E14315 |
| 1120-7-67B6 | 1120-67-B06 | 46411162 | 189 | RBD + E14315 |
| 1120-7-67C2 | 1120-67-C02 | 46411134 | 190 | RBD + E14315 |
| 1120-7-67C3 | 1120-67-C03 | 46411141 | 191 | RBD + E14315 |
| 1120-7-67C4 | 1120-67-C04 | 46411147 | 192 | RBD + E14315 |
| 1120-7-67C5 | 1120-67-C05 | 46411155 | 193 | RBD + E14315 |
| 1120-7-67C6 | 1120-67-C06 | 46411163 | 194 | RBD + E14315 |
| 1120-7-67D2 | 1120-67-D02 | 46411135 | 195 | RBD + E14315 |
| 1120-7-67D4 | 1120-67-D04 | 46411148 | 196 | RBD + E14315 |
| 1120-7-67D5 | 1120-67-D05 | 46411156 | 197 | RBD + E14315 |
| 1120-7-67E2 | 1120-67-E02 | 46411136 | 198 | RBD + E14315 |
| 1120-7-67E3 | 1120-67-E03 | 46411142 | 199 | RBD + E14315 |
| 1120-7-67E4 | 1120-67-E04 | 46411149 | 200 | RBD + E14315 |
| 1120-7-67E5 | 1120-67-E05 | 46411157 | 201 | RBD + E14315 |
| 1120-7-67F2 | 1120-67-F02 | 46411137 | 202 | RBD + E14315 |
| 1120-7-67F3 | 1120-67-F03 | 46411143 | 203 | RBD + E14315 |
| 1120-7-67F4 | 1120-67-F04 | 46411150 | 204 | RBD + E14315 |
| 1120-7-67F5 | 1120-67-F05 | 46411158 | 205 | RBD + E14315 |
| 1120-7-67G2 | 1120-67-G02 | 46411138 | 206 | RBD + E14315 |
| 1120-7-67G3 | 1120-67-G03 | 46411144 | 207 | RBD + E14315 |
| 1120-7-67G4 | 1120-67-G04 | 46411151 | 208 | RBD + E14315 |
| 1120-7-67G5 | 1120-67-G05 | 46411159 | 209 | RBD + E14315 |
| 1120-7-67H2 | 1120-67-H02 | 46411139 | 210 | RBD + E14315 |
| 1120-7-67H3 | 1120-67-H03 | 46411145 | 211 | RBD + E14315 |
| 1120-7-67H4 | 1120-67-H04 | 46411152 | 212 | RBD + E14315 |
| 1120-7-67H5 | 1120-67-H05 | 46411160 | 213 | RBD + E14315 |
| 1120-3-62A10 | 1120-62-A10 | 46410873 | 214 | RBD + E15160 |
| 1120-3-62A11 | 1120-62-A11 | 46410881 | 215 | RBD + E15160 |
| 1120-3-62A12 | 1120-62-A12 | 46410888 | 216 | RBD + E15160 |
| 1120-3-62A3 | 1120-62-A03 | 46410823 | 217 | RBD + E15160 |
| 1120-3-62A4 | 1120-62-A04 | 46410830 | 218 | RBD + E15160 |
| 1120-3-62A5 | 1120-62-A05 | 46410838 | 219 | RBD + E15160 |
| 1120-3-62A6 | 1120-62-A06 | 46410846 | 220 | RBD + E15160 |
| 1120-3-62A7 | 1120-62-A07 | 46410852 | 221 | RBD + E15160 |
| 1120-3-62A8 | 1120-62-A08 | 46410860 | 222 | RBD + E15160 |
| 1120-3-62A9 | 1120-62-A09 | 46410865 | 223 | RBD + E15160 |
| 1120-3-62B10 | 1120-62-B10 | 46410874 | 224 | RBD + E15160 |
| 1120-3-62B11 | 1120-62-B11 | 46410882 | 225 | RBD + E15160 |
| 1120-3-62B12 | 1120-62-B12 | 46410889 | 226 | RBD + E15160 |
| 1120-3-62B3 | 1120-62-B03 | 46410824 | 227 | RBD + E15160 |
| 1120-3-62B4 | 1120-62-B04 | 46410831 | 228 | RBD + E15160 |
| 1120-3-62B5 | 1120-62-B05 | 46410839 | 229 | RBD + E15160 |
| 1120-3-62B6 | 1120-62-B06 | 46410847 | 230 | RBD + E15160 |
| 1120-3-62B7 | 1120-62-B07 | 46410853 | 231 | RBD + E15160 |
| 1120-3-62B8 | 1120-62-B08 | 46410861 | 232 | RBD + E15160 |
| 1120-3-62B9 | 1120-62-B09 | 46410866 | 233 | RBD + E15160 |
| 1120-3-62C10 | 1120-62-C10 | 46410875 | 234 | RBD + E15160 |
| 1120-3-62C11 | 1120-62-C11 | 46410883 | 235 | RBD + E15160 |
| 1120-3-62C12 | 1120-62-C12 | 46410890 | 236 | RBD + E15160 |
| 1120-3-62C3 | 1120-62-C03 | 46410825 | 237 | RBD + E15160 |
| 1120-3-62C4 | 1120-62-C04 | 46410832 | 238 | RBD + E15160 |
| 1120-3-62C5 | 1120-62-C05 | 46410840 | 239 | RBD + E15160 |
| 1120-3-62C6 | 1120-62-C06 | 46410848 | 240 | RBD + E15160 |
| 1120-3-62C7 | 1120-62-C07 | 46410854 | 241 | RBD + E15160 |
| 1120-3-62C8 | 1120-62-C08 | 46410862 | 242 | RBD + E15160 |
| 1120-3-62C9 | 1120-62-C09 | 46410867 | 243 | RBD + E15160 |
| 1120-3-62D10 | 1120-62-D10 | 46410876 | 244 | RBD + E15160 |
| 1120-3-62D11 | 1120-62-D11 | 46410884 | 245 | RBD + E15160 |
| 1120-3-62D12 | 1120-62-D12 | 46410891 | 246 | RBD + E15160 |
| 1120-3-62D3 | 1120-62-D03 | 46410826 | 247 | RBD + E15160 |
| 1120-3-62D4 | 1120-62-D04 | 46410833 | 248 | RBD + E15160 |
| 1120-3-62D5 | 1120-62-D05 | 46410841 | 249 | RBD + E15160 |
| 1120-3-62D6 | 1120-62-D06 | 46410849 | 250 | RBD + E15160 |
| 1120-3-62D7 | 1120-62-D07 | 46410855 | 251 | RBD + E15160 |
| 1120-3-62D9 | 1120-62-D09 | 46410868 | 252 | RBD + E15160 |
| 1120-3-62E10 | 1120-62-E10 | 46410877 | 253 | RBD + E15160 |
| 1120-3-62E11 | 1120-62-E11 | 46410885 | 254 | RBD + E15160 |
| 1120-3-62E12 | 1120-62-E12 | 46410892 | 255 | RBD + E15160 |
| 1120-3-62E3 | 1120-62-E03 | 46410827 | 256 | RBD + E15160 |
| 1120-3-62E4 | 1120-62-E04 | 46410834 | 257 | RBD + E15160 |
| 1120-3-62E5 | 1120-62-E05 | 46410842 | 258 | RBD + E15160 |
| 1120-3-62E6 | 1120-62-E06 | 46410850 | 259 | RBD + E15160 |
| 1120-3-62E7 | 1120-62-E07 | 46410856 | 260 | RBD + E15160 |
| 1120-3-62E8 | 1120-62-E08 | 46410863 | 261 | RBD + E15160 |
| 1120-3-62E9 | 1120-62-E09 | 46410869 | 262 | RBD + E15160 |
| 1120-3-62F10 | 1120-62-F10 | 46410878 | 263 | RBD + E15160 |
| 1120-3-62F11 | 1120-62-F11 | 46410886 | 264 | RBD + E15160 |
| 1120-3-62F12 | 1120-62-F12 | 46410893 | 265 | RBD + E15160 |
| 1120-3-62F4 | 1120-62-F04 | 46410835 | 266 | RBD + E15160 |
| 1120-3-62F5 | 1120-62-F05 | 46410843 | 267 | RBD + E15160 |
| 1120-3-62F6 | 1120-62-F06 | 46410851 | 268 | RBD + E15160 |
| 1120-3-62F7 | 1120-62-F07 | 46410857 | 269 | RBD + E15160 |
| 1120-3-62F9 | 1120-62-F09 | 46410870 | 270 | RBD + E15160 |
| 1120-3-62G10 | 1120-62-G10 | 46410879 | 271 | RBD + E15160 |
| 1120-3-62G12 | 1120-62-G12 | 46410894 | 272 | RBD + E15160 |
| 1120-3-62G3 | 1120-62-G03 | 46410828 | 273 | RBD + E15160 |
| 1120-3-62G4 | 1120-62-G04 | 46410836 | 274 | RBD + E15160 |
| 1120-3-62G5 | 1120-62-G05 | 46410844 | 275 | RBD + E15160 |
| 1120-3-62G7 | 1120-62-G07 | 46410858 | 276 | RBD + E15160 |
| 1120-3-62G8 | 1120-62-G08 | 46410864 | 277 | RBD + E15160 |
| 1120-3-62G9 | 1120-62-G09 | 46410871 | 278 | RBD + E15160 |
| 1120-3-62H10 | 1120-62-H10 | 46410880 | 279 | RBD + E15160 |
| 1120-3-62H11 | 1120-62-H11 | 46410887 | 280 | RBD + E15160 |
| 1120-3-62H12 | 1120-62-H12 | 46410895 | 281 | RBD + E15160 |
| 1120-3-62H3 | 1120-62-H03 | 46410829 | 282 | RBD + E15160 |
| 1120-3-62H4 | 1120-62-H04 | 46410837 | 283 | RBD + E15160 |
| 1120-3-62H5 | 1120-62-H05 | 46410845 | 284 | RBD + E15160 |
| 1120-3-62H7 | 1120-62-H07 | 46410859 | 285 | RBD + E15160 |
| 1120-3-62H9 | 1120-62-H09 | 46410872 | 286 | RBD + E15160 |
| 1120-3-63A2 | 1120-63-A02 | 46410896 | 287 | RBD + E15160 |
| 1120-3-63B2 | 1120-63-B02 | 46410897 | 288 | RBD + E15160 |
| 1120-3-63B3 | 1120-63-B03 | 46410901 | 289 | RBD + E15160 |
| 1120-3-63C2 | 1120-63-C02 | 46410898 | 290 | RBD + E15160 |
| 1120-3-63C3 | 1120-63-C03 | 46410902 | 291 | RBD + E15160 |
| 1120-3-63D2 | 1120-63-D02 | 46410899 | 292 | RBD + E15160 |
| 1120-3-63D3 | 1120-63-D03 | 46410903 | 293 | RBD + E15160 |
| 1120-3-63E2 | 1120-63-E02 | 46410900 | 294 | RBD + E15160 |
| 1120-10-64A11 | 1120-64-A11 | 46411005 | 295 | RBD only |
| 1120-10-64A12 | 1120-64-A12 | 46411011 | 296 | RBD only |
| 1120-10-64A5 | 1120-64-A05 | 46410967 | 297 | RBD only |
| 1120-10-64A7 | 1120-64-A07 | 46410981 | 298 | RBD only |
| 1120-10-64A8 | 1120-64-A08 | 46410986 | 299 | RBD only |
| 1120-10-64B11 | 1120-64-B11 | 46411006 | 300 | RBD only |
| 1120-10-64B12 | 1120-64-B12 | 46411012 | 301 | RBD only |
| 1120-10-64B5 | 1120-64-B05 | 46410968 | 302 | RBD only |
| 1120-10-64B6 | 1120-64-B06 | 46410974 | 303 | RBD only |
| 1120-10-64B8 | 1120-64-B08 | 46410987 | 304 | RBD only |
| 1120-10-64B9 | 1120-64-B09 | 46410994 | 305 | RBD only |
| 1120-10-64C10 | 1120-64-C10 | 46411000 | 306 | RBD only |
| 1120-10-64C11 | 1120-64-C11 | 46411007 | 307 | RBD only |
| 1120-10-64C12 | 1120-64-C12 | 46411013 | 308 | RBD only |
| 1120-10-64C4 | 1120-64-C04 | 46410963 | 309 | RBD only |
| 1120-10-64C5 | 1120-64-C05 | 46410969 | 310 | RBD only |
| 1120-10-64C6 | 1120-64-C06 | 46410975 | 311 | RBD only |
| 1120-10-64C8 | 1120-64-C08 | 46410988 | 312 | RBD only |
| 1120-10-64C9 | 1120-64-C09 | 46410995 | 313 | RBD only |
| 1120-10-64D10 | 1120-64-D10 | 46411001 | 314 | RBD only |
| 1120-10-64D12 | 1120-64-D12 | 46411014 | 315 | RBD only |
| 1120-10-64D4 | 1120-64-D04 | 46410964 | 316 | RBD only |
| 1120-10-64D5 | 1120-64-D05 | 46410970 | 317 | RBD only |
| 1120-10-64D6 | 1120-64-D06 | 46410976 | 318 | RBD only |
| 1120-10-64D7 | 1120-64-D07 | 46410982 | 319 | RBD only |
| 1120-10-64D8 | 1120-64-D08 | 46410989 | 320 | RBD only |
| 1120-10-64E10 | 1120-64-E10 | 46411002 | 321 | RBD only |
| 1120-10-64E11 | 1120-64-E11 | 46411008 | 322 | RBD only |
| 1120-10-64E12 | 1120-64-E12 | 46411015 | 323 | RBD only |
| 1120-10-64E4 | 1120-64-E04 | 46410965 | 324 | RBD only |
| 1120-10-64E5 | 1120-64-E05 | 46410971 | 325 | RBD only |
| 1120-10-64E6 | 1120-64-E06 | 46410977 | 326 | RBD only |
| 1120-10-64E8 | 1120-64-E08 | 46410990 | 327 | RBD only |
| 1120-10-64E9 | 1120-64-E09 | 46410996 | 328 | RBD only |
| 1120-10-64F10 | 1120-64-F10 | 46411003 | 329 | RBD only |
| 1120-10-64F11 | 1120-64-F11 | 46411009 | 330 | RBD only |
| 1120-10-64F12 | 1120-64-F12 | 46411016 | 331 | RBD only |
| 1120-10-64F5 | 1120-64-F05 | 46410972 | 332 | RBD only |
| 1120-10-64F6 | 1120-64-F06 | 46410978 | 333 | RBD only |
| 1120-10-64F7 | 1120-64-F07 | 46410983 | 334 | RBD only |
| 1120-10-64F8 | 1120-64-F08 | 46410991 | 335 | RBD only |
| 1120-10-64F9 | 1120-64-F09 | 46410997 | 336 | RBD only |
| 1120-10-64G11 | 1120-64-G11 | 46411010 | 337 | RBD only |
| 1120-10-64G12 | 1120-64-G12 | 46411017 | 338 | RBD only |
| 1120-10-64G6 | 1120-64-G06 | 46410979 | 339 | RBD only |
| 1120-10-64G7 | 1120-64-G07 | 46410984 | 340 | RBD only |
| 1120-10-64G8 | 1120-64-G08 | 46410992 | 341 | RBD only |
| 1120-10-64G9 | 1120-64-G09 | 46410998 | 342 | RBD only |
| 1120-10-64H10 | 1120-64-H10 | 46411004 | 343 | RBD only |
| 1120-10-64H12 | 1120-64-H12 | 46411018 | 344 | RBD only |
| 1120-10-64H4 | 1120-64-H04 | 46410966 | 345 | RBD only |
| 1120-10-64H5 | 1120-64-H05 | 46410973 | 346 | RBD only |
| 1120-10-64H6 | 1120-64-H06 | 46410980 | 347 | RBD only |
| 1120-10-64H7 | 1120-64-H07 | 46410985 | 348 | RBD only |
| 1120-10-64H8 | 1120-64-H08 | 46410993 | 349 | RBD only |
| 1120-10-64H9 | 1120-64-H09 | 46410999 | 350 | RBD only |
| 1120-10-65B4 | 1120-65-B04 | 46411030 | 351 | RBD only |
| 1120-10-65C3 | 1120-65-C03 | 46411024 | 352 | RBD only |
| 1120-10-65C4 | 1120-65-C04 | 46411031 | 353 | RBD only |
| 1120-10-65D2 | 1120-65-D02 | 46411019 | 354 | RBD only |
| 1120-10-65D3 | 1120-65-D03 | 46411025 | 355 | RBD only |
| 1120-10-65D4 | 1120-65-D04 | 46411032 | 356 | RBD only |
| 1120-10-65E2 | 1120-65-E02 | 46411020 | 357 | RBD only |
| 1120-10-65E3 | 1120-65-E03 | 46411026 | 358 | RBD only |
| 1120-10-65E4 | 1120-65-E04 | 46411033 | 359 | RBD only |
| 1120-10-65F2 | 1120-65-F02 | 46411021 | 360 | RBD only |
| 1120-10-65F3 | 1120-65-F03 | 46411027 | 361 | RBD only |
| 1120-10-65G2 | 1120-65-G02 | 46411022 | 362 | RBD only |
| 1120-10-65G3 | 1120-65-G03 | 46411028 | 363 | RBD only |
| 1120-10-65H2 | 1120-65-H02 | 46411023 | 364 | RBD only |
| 1120-10-65H3 | 1120-65-H03 | 46411029 | 365 | RBD only |
| 1120-11-70A10 | 1120-70-A10 | 46411385 | 366 | RBD only |
| 1120-11-70B10 | 1120-70-B10 | 46411386 | 367 | RBD only |
| 1120-11-70B11 | 1120-70-B11 | 46411393 | 368 | RBD only |
| 1120-11-70B12 | 1120-70-B12 | 46411400 | 369 | RBD only |
| 1120-11-70B8 | 1120-70-B08 | 46411374 | 370 | RBD only |
| 1120-11-70C10 | 1120-70-C10 | 46411387 | 371 | RBD only |
| 1120-11-70C11 | 1120-70-C11 | 46411394 | 372 | RBD only |
| 1120-11-70C12 | 1120-70-C12 | 46411401 | 373 | RBD only |
| 1120-11-70D10 | 1120-70-D10 | 46411388 | 374 | RBD only |
| 1120-11-70D11 | 1120-70-D11 | 46411395 | 375 | RBD only |
| 1120-11-70D12 | 1120-70-D12 | 46411402 | 376 | RBD only |
| 1120-11-70D8 | 1120-70-D08 | 46411375 | 377 | RBD only |
| 1120-11-70E10 | 1120-70-E10 | 46411389 | 378 | RBD only |
| 1120-11-70E11 | 1120-70-E11 | 46411396 | 379 | RBD only |
| 1120-11-70E12 | 1120-70-E12 | 46411403 | 380 | RBD only |
| 1120-11-70E8 | 1120-70-E08 | 46411376 | 381 | RBD only |
| 1120-11-70F10 | 1120-70-F10 | 46411390 | 382 | RBD only |
| 1120-11-70F11 | 1120-70-F11 | 46411397 | 383 | RBD only |
| 1120-11-70F12 | 1120-70-F12 | 46411404 | 384 | RBD only |
| 1120-11-70G10 | 1120-70-G10 | 46411391 | 385 | RBD only |
| 1120-11-70G11 | 1120-70-G11 | 46411398 | 386 | RBD only |
| 1120-11-70G12 | 1120-70-G12 | 46411405 | 387 | RBD only |
| 1120-11-70H10 | 1120-70-H10 | 46411392 | 388 | RBD only |
| 1120-11-70H11 | 1120-70-H11 | 46411399 | 389 | RBD only |
| 1120-11-70H12 | 1120-70-H12 | 46411406 | 390 | RBD only |
| 1120-11-70H9 | 1120-70-H09 | 46411384 | 391 | RBD only |
| 1120-11-71A2 | 1120-71-A02 | 46411407 | 392 | RBD only |
| 1120-11-71A3 | 1120-71-A03 | 46411412 | 393 | RBD only |
| 1120-11-71A4 | 1120-71-A04 | 46411419 | 394 | RBD only |
| 1120-11-71A5 | 1120-71-A05 | 46411427 | 395 | RBD only |
| 1120-11-71A6 | 1120-71-A06 | 46411434 | 396 | RBD only |
| 1120-11-71A7 | 1120-71-A07 | 46411440 | 397 | RBD only |
| 1120-11-71A8 | 1120-71-A08 | 46411447 | 398 | RBD only |
| 1120-11-71A9 | 1120-71-A09 | 46411455 | 399 | RBD only |
| 1120-11-71B3 | 1120-71-B03 | 46411413 | 400 | RBD only |
| 1120-11-71B4 | 1120-71-B04 | 46411420 | 401 | RBD only |
| 1120-11-71B5 | 1120-71-B05 | 46411428 | 402 | RBD only |
| 1120-11-71B6 | 1120-71-B06 | 46411435 | 403 | RBD only |
| 1120-11-71B7 | 1120-71-B07 | 46411441 | 404 | RBD only |
| 1120-11-71B8 | 1120-71-B08 | 46411448 | 405 | RBD only |
| 1120-11-71B9 | 1120-71-B09 | 46411456 | 406 | RBD only |
| 1120-11-71C2 | 1120-71-C02 | 46411408 | 407 | RBD only |
| 1120-11-71C3 | 1120-71-C03 | 46411414 | 408 | RBD only |
| 1120-11-71C4 | 1120-71-C04 | 46411421 | 409 | RBD only |
| 1120-11-71C5 | 1120-71-C05 | 46411429 | 410 | RBD only |
| 1120-11-71C6 | 1120-71-C06 | 46411436 | 411 | RBD only |
| 1120-11-71C7 | 1120-71-C07 | 46411442 | 412 | RBD only |
| 1120-11-71C8 | 1120-71-C08 | 46411449 | 413 | RBD only |
| 1120-11-71C9 | 1120-71-C09 | 46411457 | 414 | RBD only |
| 1120-11-71D2 | 1120-71-D02 | 46411409 | 415 | RBD only |
| 1120-11-71D3 | 1120-71-D03 | 46411415 | 416 | RBD only |
| 1120-11-71D4 | 1120-71-D04 | 46411422 | 417 | RBD only |
| 1120-11-71D5 | 1120-71-D05 | 46411430 | 418 | RBD only |
| 1120-11-71D6 | 1120-71-D06 | 46411437 | 419 | RBD only |
| 1120-11-71D7 | 1120-71-D07 | 46411443 | 420 | RBD only |
| 1120-11-71D8 | 1120-71-D08 | 46411450 | 421 | RBD only |
| 1120-11-71D9 | 1120-71-D09 | 46411458 | 422 | RBD only |
| 1120-11-71E2 | 1120-71-E02 | 46411410 | 423 | RBD only |
| 1120-11-71E3 | 1120-71-E03 | 46411416 | 424 | RBD only |
| 1120-11-71E4 | 1120-71-E04 | 46411423 | 425 | RBD only |
| 1120-11-71E8 | 1120-71-E08 | 46411451 | 426 | RBD only |
| 1120-11-71E9 | 1120-71-E09 | 46411459 | 427 | RBD only |
| 1120-11-71F2 | 1120-71-F02 | 46411411 | 428 | RBD only |
| 1120-11-71F3 | 1120-71-F03 | 46411417 | 429 | RBD only |
| 1120-11-71F4 | 1120-71-F04 | 46411424 | 430 | RBD only |
| 1120-11-71F5 | 1120-71-F05 | 46411431 | 431 | RBD only |
| 1120-11-71F6 | 1120-71-F06 | 46411438 | 432 | RBD only |
| 1120-11-71F7 | 1120-71-F07 | 46411444 | 433 | RBD only |
| 1120-11-71F8 | 1120-71-F08 | 46411452 | 434 | RBD only |
| 1120-11-71F9 | 1120-71-F09 | 46411460 | 435 | RBD only |
| 1120-11-71G3 | 1120-71-G03 | 46411418 | 436 | RBD only |
| 1120-11-71G4 | 1120-71-G04 | 46411425 | 437 | RBD only |
| 1120-11-71G5 | 1120-71-G05 | 46411432 | 438 | RBD only |
| 1120-11-71G7 | 1120-71-G07 | 46411445 | 439 | RBD only |
| 1120-11-71G8 | 1120-71-G08 | 46411453 | 440 | RBD only |
| 1120-11-71G9 | 1120-71-G09 | 46411461 | 441 | RBD only |
| 1120-11-71H4 | 1120-71-H04 | 46411426 | 442 | RBD only |
| 1120-11-71H5 | 1120-71-H05 | 46411433 | 443 | RBD only |
| 1120-11-71H6 | 1120-71-H06 | 46411439 | 444 | RBD only |
| 1120-11-71H7 | 1120-71-H07 | 46411446 | 445 | RBD only |
| 1120-11-71H8 | 1120-71-H08 | 46411454 | 446 | RBD only |
| 1120-13-67A10 | 1120-67-A10 | 46411192 | 447 | RBD only |
| 1120-13-67A11 | 1120-67-A11 | 46411200 | 448 | RBD only |
| 1120-13-67A12 | 1120-67-A12 | 46411208 | 449 | RBD only |
| 1120-13-67A7 | 1120-67-A07 | 46411169 | 450 | RBD only |
| 1120-13-67A8 | 1120-67-A08 | 46411176 | 451 | RBD only |
| 1120-13-67A9 | 1120-67-A09 | 46411184 | 452 | RBD only |
| 1120-13-67B10 | 1120-67-B10 | 46411193 | 453 | RBD only |
| 1120-13-67B11 | 1120-67-B11 | 46411201 | 454 | RBD only |
| 1120-13-67B12 | 1120-67-B12 | 46411209 | 455 | RBD only |
| 1120-13-67B8 | 1120-67-B08 | 46411177 | 456 | RBD only |
| 1120-13-67B9 | 1120-67-B09 | 46411185 | 457 | RBD only |
| 1120-13-67C10 | 1120-67-C10 | 46411194 | 458 | RBD only |
| 1120-13-67C11 | 1120-67-C11 | 46411202 | 459 | RBD only |
| 1120-13-67C12 | 1120-67-C12 | 46411210 | 460 | RBD only |
| 1120-13-67C7 | 1120-67-C07 | 46411170 | 461 | RBD only |
| 1120-13-67C8 | 1120-67-C08 | 46411178 | 462 | RBD only |
| 1120-13-67C9 | 1120-67-C09 | 46411186 | 463 | RBD only |
| 1120-13-67D10 | 1120-67-D10 | 46411195 | 464 | RBD only |
| 1120-13-67D11 | 1120-67-D11 | 46411203 | 465 | RBD only |
| 1120-13-67D6 | 1120-67-D06 | 46411164 | 466 | RBD only |
| 1120-13-67D7 | 1120-67-D07 | 46411171 | 467 | RBD only |
| 1120-13-67D8 | 1120-67-D08 | 46411179 | 468 | RBD only |
| 1120-13-67D9 | 1120-67-D09 | 46411187 | 469 | RBD only |
| 1120-13-67E10 | 1120-67-E10 | 46411196 | 470 | RBD only |
| 1120-13-67E11 | 1120-67-E11 | 46411204 | 471 | RBD only |
| 1120-13-67E12 | 1120-67-E12 | 46411211 | 472 | RBD only |
| 1120-13-67E6 | 1120-67-E06 | 46411165 | 473 | RBD only |
| 1120-13-67E7 | 1120-67-E07 | 46411172 | 474 | RBD only |
| 1120-13-67E8 | 1120-67-E08 | 46411180 | 475 | RBD only |
| 1120-13-67E9 | 1120-67-E09 | 46411188 | 476 | RBD only |
| 1120-13-67F10 | 1120-67-F10 | 46411197 | 477 | RBD only |
| 1120-13-67F11 | 1120-67-F11 | 46411205 | 478 | RBD only |
| 1120-13-67F12 | 1120-67-F12 | 46411212 | 479 | RBD only |
| 1120-13-67F6 | 1120-67-F06 | 46411166 | 480 | RBD only |
| 1120-13-67F7 | 1120-67-F07 | 46411173 | 481 | RBD only |
| 1120-13-67F8 | 1120-67-F08 | 46411181 | 482 | RBD only |
| 1120-13-67F9 | 1120-67-F09 | 46411189 | 483 | RBD only |
| 1120-13-67G10 | 1120-67-G10 | 46411198 | 484 | RBD only |
| 1120-13-67G11 | 1120-67-G11 | 46411206 | 485 | RBD only |
| 1120-13-67G12 | 1120-67-G12 | 46411213 | 486 | RBD only |
| 1120-13-67G6 | 1120-67-G06 | 46411167 | 487 | RBD only |
| 1120-13-67G7 | 1120-67-G07 | 46411174 | 488 | RBD only |
| 1120-13-67G8 | 1120-67-G08 | 46411182 | 489 | RBD only |
| 1120-13-67G9 | 1120-67-G09 | 46411190 | 490 | RBD only |
| 1120-13-67H10 | 1120-67-H10 | 46411199 | 491 | RBD only |
| 1120-13-67H11 | 1120-67-H11 | 46411207 | 492 | RBD only |
| 1120-13-67H12 | 1120-67-H12 | 46411214 | 493 | RBD only |
| 1120-13-67H6 | 1120-67-H06 | 46411168 | 494 | RBD only |
| 1120-13-67H7 | 1120-67-H07 | 46411175 | 495 | RBD only |
| 1120-13-67H8 | 1120-67-H08 | 46411183 | 496 | RBD only |
| 1120-13-67H9 | 1120-67-H09 | 46411191 | 497 | RBD only |
| 1120-13-68A2 | 1120-68-A02 | 46411215 | 498 | RBD only |
| 1120-13-68A3 | 1120-68-A03 | 46411221 | 499 | RBD only |
| 1120-13-68A4 | 1120-68-A04 | 46411229 | 500 | RBD only |
| 1120-13-68B2 | 1120-68-B02 | 46411216 | 501 | RBD only |
| 1120-13-68B3 | 1120-68-B03 | 46411222 | 502 | RBD only |
| 1120-13-68B4 | 1120-68-B04 | 46411230 | 503 | RBD only |
| 1120-13-68C2 | 1120-68-C02 | 46411217 | 504 | RBD only |
| 1120-13-68C3 | 1120-68-C03 | 46411223 | 505 | RBD only |
| 1120-13-68C4 | 1120-68-C04 | 46411231 | 506 | RBD only |
| 1120-13-68C5 | 1120-68-C05 | 46411235 | 507 | RBD only |
| 1120-13-68C6 | 1120-68-C06 | 46411241 | 508 | RBD only |
| 1120-13-68D3 | 1120-68-D03 | 46411224 | 509 | RBD only |
| 1120-13-68D4 | 1120-68-D04 | 46411232 | 510 | RBD only |
| 1120-13-68D5 | 1120-68-D05 | 46411236 | 511 | RBD only |
| 1120-13-68D6 | 1120-68-D06 | 46411242 | 512 | RBD only |
| 1120-13-68E2 | 1120-68-E02 | 46411218 | 513 | RBD only |
| 1120-13-68E3 | 1120-68-E03 | 46411225 | 514 | RBD only |
| 1120-13-68E4 | 1120-68-E04 | 46411233 | 515 | RBD only |
| 1120-13-68E5 | 1120-68-E05 | 46411237 | 516 | RBD only |
| 1120-13-68E6 | 1120-68-E06 | 46411243 | 517 | RBD only |
| 1120-13-68F2 | 1120-68-F02 | 46411219 | 518 | RBD only |
| 1120-13-68F3 | 1120-68-F03 | 46411226 | 519 | RBD only |
| 1120-13-68F5 | 1120-68-F05 | 46411238 | 520 | RBD only |
| 1120-13-68F6 | 1120-68-F06 | 46411244 | 521 | RBD only |
| 1120-13-68G3 | 1120-68-G03 | 46411227 | 522 | RBD only |
| 1120-13-68G4 | 1120-68-G04 | 46411234 | 523 | RBD only |
| 1120-13-68G5 | 1120-68-G05 | 46411239 | 524 | RBD only |
| 1120-13-68G6 | 1120-68-G06 | 46411245 | 525 | RBD only |
| 1120-13-68H2 | 1120-68-H02 | 46411220 | 526 | RBD only |
| 1120-13-68H3 | 1120-68-H03 | 46411228 | 527 | RBD only |
| 1120-13-68H5 | 1120-68-H05 | 46411240 | 528 | RBD only |
| 1120-13-68H6 | 1120-68-H06 | 46411246 | 529 | RBD only |
| 1120-14-69A9 | 1120-69-A09 | 46411321 | 530 | RBD only |
| 1120-14-69B8 | 1120-69-B08 | 46411314 | 531 | RBD only |
| 1120-14-69B9 | 1120-69-B09 | 46411322 | 532 | RBD only |
| 1120-14-69C10 | 1120-69-C10 | 46411328 | 533 | RBD only |
| 1120-14-69C8 | 1120-69-C08 | 46411315 | 534 | RBD only |
| 1120-14-69D10 | 1120-69-D10 | 46411329 | 535 | RBD only |
| 1120-14-69D8 | 1120-69-D08 | 46411316 | 536 | RBD only |
| 1120-14-69D9 | 1120-69-D09 | 46411323 | 537 | RBD only |
| 1120-14-69E10 | 1120-69-E10 | 46411330 | 538 | RBD only |
| 1120-14-69E8 | 1120-69-E08 | 46411317 | 539 | RBD only |
| 1120-14-69E9 | 1120-69-E09 | 46411324 | 540 | RBD only |
| 1120-14-69F10 | 1120-69-F10 | 46411331 | 541 | RBD only |
| 1120-14-69F8 | 1120-69-F08 | 46411318 | 542 | RBD only |
| 1120-14-69F9 | 1120-69-F09 | 46411325 | 543 | RBD only |
| 1120-14-69G10 | 1120-69-G10 | 46411332 | 544 | RBD only |
| 1120-14-69G8 | 1120-69-G08 | 46411319 | 545 | RBD only |
| 1120-14-69G9 | 1120-69-G09 | 46411326 | 546 | RBD only |
| 1120-14-69H7 | 1120-69-H07 | 46411313 | 547 | RBD only |
| 1120-14-69H8 | 1120-69-H08 | 46411320 | 548 | RBD only |
| 1120-14-69H9 | 1120-69-H09 | 46411327 | 549 | RBD only |
| 1120-18-71A11 | 1120-71-A11 | 46411468 | 550 | 33 + 87 + RBD |
| 1120-18-71B10 | 1120-71-B10 | 46411462 | 551 | 33 + 87 + RBD |
| 1120-18-71B11 | 1120-71-B11 | 46411469 | 552 | 33 + 87 + RBD |
| 1120-18-71B12 | 1120-71-B12 | 46411474 | 553 | 33 + 87 + RBD |
| 1120-18-71C10 | 1120-71-C10 | 46411463 | 554 | 33 + 87 + RBD |
| 1120-18-71C11 | 1120-71-C11 | 46411470 | 555 | 33 + 87 + RBD |
| 1120-18-71C12 | 1120-71-C12 | 46411475 | 556 | 33 + 87 + RBD |
| 1120-18-71D10 | 1120-71-D10 | 46411464 | 557 | 33 + 87 + RBD |
| 1120-18-71D12 | 1120-71-D12 | 46411476 | 558 | 33 + 87 + RBD |
| 1120-18-71E10 | 1120-71-E10 | 46411465 | 559 | 33 + 87 + RBD |
| 1120-18-71E11 | 1120-71-E11 | 46411471 | 560 | 33 + 87 + RBD |
| 1120-18-71E12 | 1120-71-E12 | 46411477 | 561 | 33 + 87 + RBD |
| 1120-18-71F12 | 1120-71-F12 | 46411478 | 562 | 33 + 87 + RBD |
| 1120-18-71G10 | 1120-71-G10 | 46411466 | 563 | 33 + 87 + RBD |
| 1120-18-71G11 | 1120-71-G11 | 46411472 | 564 | 33 + 87 + RBD |
| 1120-18-71G12 | 1120-71-G12 | 46411479 | 565 | 33 + 87 + RBD |
| 1120-18-71H10 | 1120-71-H10 | 46411467 | 566 | 33 + 87 + RBD |
| 1120-18-71H11 | 1120-71-H11 | 46411473 | 567 | 33 + 87 + RBD |
| 1120-18-72A2 | 1120-72-A02 | 46411480 | 568 | 33 + 87 + RBD |
| 1120-18-72A3 | 1120-72-A03 | 46411486 | 569 | 33 + 87 + RBD |
| 1120-18-72A4 | 1120-72-A04 | 46411494 | 570 | 33 + 87 + RBD |
| 1120-18-72A5 | 1120-72-A05 | 46411501 | 571 | 33 + 87 + RBD |
| 1120-18-72A6 | 1120-72-A06 | 46411509 | 572 | 33 + 87 + RBD |
| 1120-18-72A7 | 1120-72-A07 | 46411517 | 573 | 33 + 87 + RBD |
| 1120-18-72A8 | 1120-72-A08 | 46411524 | 574 | 33 + 87 + RBD |
| 1120-18-72A9 | 1120-72-A09 | 46411530 | 575 | 33 + 87 + RBD |
| 1120-18-72B10 | 1120-72-B10 | 46411538 | 576 | 33 + 87 + RBD |
| 1120-18-72B2 | 1120-72-B02 | 46411481 | 577 | 33 + 87 + RBD |
| 1120-18-72B3 | 1120-72-B03 | 46411487 | 578 | 33 + 87 + RBD |
| 1120-18-72B4 | 1120-72-B04 | 46411495 | 579 | 33 + 87 + RBD |
| 1120-18-72B5 | 1120-72-B05 | 46411502 | 580 | 33 + 87 + RBD |
| 1120-18-72B6 | 1120-72-B06 | 46411510 | 581 | 33 + 87 + RBD |
| 1120-18-72B7 | 1120-72-B07 | 46411518 | 582 | 33 + 87 + RBD |
| 1120-18-72B8 | 1120-72-B08 | 46411525 | 583 | 33 + 87 + RBD |
| 1120-18-72B9 | 1120-72-B09 | 46411531 | 584 | 33 + 87 + RBD |
| 1120-18-72C10 | 1120-72-C10 | 46411539 | 585 | 33 + 87 + RBD |
| 1120-18-72C2 | 1120-72-C02 | 46411482 | 586 | 33 + 87 + RBD |
| 1120-18-72C3 | 1120-72-C03 | 46411488 | 587 | 33 + 87 + RBD |
| 1120-18-72C4 | 1120-72-C04 | 46411496 | 588 | 33 + 87 + RBD |
| 1120-18-72C5 | 1120-72-C05 | 46411503 | 589 | 33 + 87 + RBD |
| 1120-18-72C6 | 1120-72-C06 | 46411511 | 590 | 33 + 87 + RBD |
| 1120-18-72C7 | 1120-72-C07 | 46411519 | 591 | 33 + 87 + RBD |
| 1120-18-72C8 | 1120-72-C08 | 46411526 | 592 | 33 + 87 + RBD |
| 1120-18-72C9 | 1120-72-C09 | 46411532 | 593 | 33 + 87 + RBD |
| 1120-18-72D10 | 1120-72-D10 | 46411540 | 594 | 33 + 87 + RBD |
| 1120-18-72D2 | 1120-72-D02 | 46411483 | 595 | 33 + 87 + RBD |
| 1120-18-72D3 | 1120-72-D03 | 46411489 | 596 | 33 + 87 + RBD |
| 1120-18-72D4 | 1120-72-D04 | 46411497 | 597 | 33 + 87 + RBD |
| 1120-18-72D5 | 1120-72-D05 | 46411504 | 598 | 33 + 87 + RBD |
| 1120-18-72D6 | 1120-72-D06 | 46411512 | 599 | 33 + 87 + RBD |
| 1120-18-72D7 | 1120-72-D07 | 46411520 | 600 | 33 + 87 + RBD |
| 1120-18-72D8 | 1120-72-D08 | 46411527 | 601 | 33 + 87 + RBD |
| 1120-18-72D9 | 1120-72-D09 | 46411533 | 602 | 33 + 87 + RBD |
| 1120-18-72E10 | 1120-72-E10 | 46411541 | 603 | 33 + 87 + RBD |
| 1120-18-72E2 | 1120-72-E02 | 46411484 | 604 | 33 + 87 + RBD |
| 1120-18-72E3 | 1120-72-E03 | 46411490 | 605 | 33 + 87 + RBD |
| 1120-18-72E4 | 1120-72-E04 | 46411498 | 606 | 33 + 87 + RBD |
| 1120-18-72E5 | 1120-72-E05 | 46411505 | 607 | 33 + 87 + RBD |
| 1120-18-72E6 | 1120-72-E06 | 46411513 | 608 | 33 + 87 + RBD |
| 1120-18-72E7 | 1120-72-E07 | 46411521 | 609 | 33 + 87 + RBD |
| 1120-18-72E8 | 1120-72-E08 | 46411528 | 610 | 33 + 87 + RBD |
| 1120-18-72E9 | 1120-72-E09 | 46411534 | 611 | 33 + 87 + RBD |
| 1120-18-72F10 | 1120-72-F10 | 46411542 | 612 | 33 + 87 + RBD |
| 1120-18-72F2 | 1120-72-F02 | 46411485 | 613 | 33 + 87 + RBD |
| 1120-18-72F3 | 1120-72-F03 | 46411491 | 614 | 33 + 87 + RBD |
| 1120-18-72F5 | 1120-72-F05 | 46411506 | 615 | 33 + 87 + RBD |
| 1120-18-72F6 | 1120-72-F06 | 46411514 | 616 | 33 + 87 + RBD |
| 1120-18-72F7 | 1120-72-F07 | 46411522 | 617 | 33 + 87 + RBD |
| 1120-18-72F9 | 1120-72-F09 | 46411535 | 618 | 33 + 87 + RBD |
| 1120-18-72G3 | 1120-72-G03 | 46411492 | 619 | 33 + 87 + RBD |
| 1120-18-72G4 | 1120-72-G04 | 46411499 | 620 | 33 + 87 + RBD |
| 1120-18-72G5 | 1120-72-G05 | 46411507 | 621 | 33 + 87 + RBD |
| 1120-18-72G6 | 1120-72-G06 | 46411515 | 622 | 33 + 87 + RBD |
| 1120-18-72G7 | 1120-72-G07 | 46411523 | 623 | 33 + 87 + RBD |
| 1120-18-72G9 | 1120-72-G09 | 46411536 | 624 | 33 + 87 + RBD |
| 1120-18-72H3 | 1120-72-H03 | 46411493 | 625 | 33 + 87 + RBD |
| 1120-18-72H4 | 1120-72-H04 | 46411500 | 626 | 33 + 87 + RBD |
| 1120-18-72H5 | 1120-72-H05 | 46411508 | 627 | 33 + 87 + RBD |
| 1120-18-72H6 | 1120-72-H06 | 46411516 | 628 | 33 + 87 + RBD |
| 1120-18-72H8 | 1120-72-H08 | 46411529 | 629 | 33 + 87 + RBD |
| 1120-18-72H9 | 1120-72-H09 | 46411537 | 630 | 33 + 87 + RBD |
| 1120-20-75A12 | 1120-75-A12 | 46411728 | 631 | 33 + 87 + RBD |
| 1120-20-75B12 | 1120-75-B12 | 46411729 | 632 | 33 + 87 + RBD |
| 1120-20-75C12 | 1120-75-C12 | 46411730 | 633 | 33 + 87 + RBD |
| 1120-20-75D12 | 1120-75-D12 | 46411731 | 634 | 33 + 87 + RBD |
| 1120-20-75E12 | 1120-75-E12 | 46411732 | 635 | 33 + 87 + RBD |
| 1120-20-75F11 | 1120-75-F11 | 46411725 | 636 | 33 + 87 + RBD |
| 1120-20-75F12 | 1120-75-F12 | 46411733 | 637 | 33 + 87 + RBD |
| 1120-20-75G11 | 1120-75-G11 | 46411726 | 638 | 33 + 87 + RBD |
| 1120-20-75G12 | 1120-75-G12 | 46411734 | 639 | 33 + 87 + RBD |
| 1120-20-75H11 | 1120-75-H11 | 46411727 | 640 | 33 + 87 + RBD |
| 1120-20-75H12 | 1120-75-H12 | 46411735 | 641 | 33 + 87 + RBD |
| 1120-20-76A10 | 1120-76-A10 | 46411795 | 642 | 33 + 87 + RBD |
| 1120-20-76A11 | 1120-76-A11 | 46411803 | 643 | 33 + 87 + RBD |
| 1120-20-76A12 | 1120-76-A12 | 46411811 | 644 | 33 + 87 + RBD |
| 1120-20-76A2 | 1120-76-A02 | 46411736 | 645 | 33 + 87 + RBD |
| 1120-20-76A3 | 1120-76-A03 | 46411743 | 646 | 33 + 87 + RBD |
| 1120-20-76A4 | 1120-76-A04 | 46411750 | 647 | 33 + 87 + RBD |
| 1120-20-76A5 | 1120-76-A05 | 46411757 | 648 | 33 + 87 + RBD |
| 1120-20-76A6 | 1120-76-A06 | 46411765 | 649 | 33 + 87 + RBD |
| 1120-20-76A7 | 1120-76-A07 | 46411773 | 650 | 33 + 87 + RBD |
| 1120-20-76A8 | 1120-76-A08 | 46411780 | 651 | 33 + 87 + RBD |
| 1120-20-76A9 | 1120-76-A09 | 46411787 | 652 | 33 + 87 + RBD |
| 1120-20-76B10 | 1120-76-B10 | 46411796 | 653 | 33 + 87 + RBD |
| 1120-20-76B11 | 1120-76-B11 | 46411804 | 654 | 33 + 87 + RBD |
| 1120-20-76B12 | 1120-76-B12 | 46411812 | 655 | 33 + 87 + RBD |
| 1120-20-76B2 | 1120-76-B02 | 46411737 | 656 | 33 + 87 + RBD |
| 1120-20-76B3 | 1120-76-B03 | 46411744 | 657 | 33 + 87 + RBD |
| 1120-20-76B4 | 1120-76-B04 | 46411751 | 658 | 33 + 87 + RBD |
| 1120-20-76B5 | 1120-76-B05 | 46411758 | 659 | 33 + 87 + RBD |
| 1120-20-76B6 | 1120-76-B06 | 46411766 | 660 | 33 + 87 + RBD |
| 1120-20-76B7 | 1120-76-B07 | 46411774 | 661 | 33 + 87 + RBD |
| 1120-20-76B8 | 1120-76-B08 | 46411781 | 662 | 33 + 87 + RBD |
| 1120-20-76B9 | 1120-76-B09 | 46411788 | 663 | 33 + 87 + RBD |
| 1120-20-76C10 | 1120-76-C10 | 46411797 | 664 | 33 + 87 + RBD |
| 1120-20-76C11 | 1120-76-C11 | 46411805 | 665 | 33 + 87 + RBD |
| 1120-20-76C2 | 1120-76-C02 | 46411738 | 666 | 33 + 87 + RBD |
| 1120-20-76C3 | 1120-76-C03 | 46411745 | 667 | 33 + 87 + RBD |
| 1120-20-76C4 | 1120-76-C04 | 46411752 | 668 | 33 + 87 + RBD |
| 1120-20-76C5 | 1120-76-C05 | 46411759 | 669 | 33 + 87 + RBD |
| 1120-20-76C6 | 1120-76-C06 | 46411767 | 670 | 33 + 87 + RBD |
| 1120-20-76C7 | 1120-76-C07 | 46411775 | 671 | 33 + 87 + RBD |
| 1120-20-76C8 | 1120-76-C08 | 46411782 | 672 | 33 + 87 + RBD |
| 1120-20-76C9 | 1120-76-C09 | 46411789 | 673 | 33 + 87 + RBD |
| 1120-20-76D10 | 1120-76-D10 | 46411798 | 674 | 33 + 87 + RBD |
| 1120-20-76D11 | 1120-76-D11 | 46411806 | 675 | 33 + 87 + RBD |
| 1120-20-76D2 | 1120-76-D02 | 46411739 | 676 | 33 + 87 + RBD |
| 1120-20-76D3 | 1120-76-D03 | 46411746 | 677 | 33 + 87 + RBD |
| 1120-20-76D4 | 1120-76-D04 | 46411753 | 678 | 33 + 87 + RBD |
| 1120-20-76D5 | 1120-76-D05 | 46411760 | 679 | 33 + 87 + RBD |
| 1120-20-76D6 | 1120-76-D06 | 46411768 | 680 | 33 + 87 + RBD |
| 1120-20-76D7 | 1120-76-D07 | 46411776 | 681 | 33 + 87 + RBD |
| 1120-20-76D9 | 1120-76-D09 | 46411790 | 682 | 33 + 87 + RBD |
| 1120-20-76E10 | 1120-76-E10 | 46411799 | 683 | 33 + 87 + RBD |
| 1120-20-76E11 | 1120-76-E11 | 46411807 | 684 | 33 + 87 + RBD |
| 1120-20-76E2 | 1120-76-E02 | 46411740 | 685 | 33 + 87 + RBD |
| 1120-20-76E3 | 1120-76-E03 | 46411747 | 686 | 33 + 87 + RBD |
| 1120-20-76E4 | 1120-76-E04 | 46411754 | 687 | 33 + 87 + RBD |
| 1120-20-76E5 | 1120-76-E05 | 46411761 | 688 | 33 + 87 + RBD |
| 1120-20-76E6 | 1120-76-E06 | 46411769 | 689 | 33 + 87 + RBD |
| 1120-20-76E8 | 1120-76-E08 | 46411783 | 690 | 33 + 87 + RBD |
| 1120-20-76E9 | 1120-76-E09 | 46411791 | 691 | 33 + 87 + RBD |
| 1120-20-76F10 | 1120-76-F10 | 46411800 | 692 | 33 + 87 + RBD |
| 1120-20-76F11 | 1120-76-F11 | 46411808 | 693 | 33 + 87 + RBD |
| 1120-20-76F2 | 1120-76-F02 | 46411741 | 694 | 33 + 87 + RBD |
| 1120-20-76F3 | 1120-76-F03 | 46411748 | 695 | 33 + 87 + RBD |
| 1120-20-76F4 | 1120-76-F04 | 46411755 | 696 | 33 + 87 + RBD |
| 1120-20-76F5 | 1120-76-F05 | 46411762 | 697 | 33 + 87 + RBD |
| 1120-20-76F6 | 1120-76-F06 | 46411770 | 698 | 33 + 87 + RBD |
| 1120-20-76F7 | 1120-76-F07 | 46411777 | 699 | 33 + 87 + RBD |
| 1120-20-76F8 | 1120-76-F08 | 46411784 | 700 | 33 + 87 + RBD |
| 1120-20-76F9 | 1120-76-F09 | 46411792 | 701 | 33 + 87 + RBD |
| 1120-20-76G10 | 1120-76-G10 | 46411801 | 702 | 33 + 87 + RBD |
| 1120-20-76G11 | 1120-76-G11 | 46411809 | 703 | 33 + 87 + RBD |
| 1120-20-76G2 | 1120-76-G02 | 46411742 | 704 | 33 + 87 + RBD |
| 1120-20-76G4 | 1120-76-G04 | 46411756 | 705 | 33 + 87 + RBD |
| 1120-20-76G5 | 1120-76-G05 | 46411763 | 706 | 33 + 87 + RBD |
| 1120-20-76G6 | 1120-76-G06 | 46411771 | 707 | 33 + 87 + RBD |
| 1120-20-76G7 | 1120-76-G07 | 46411778 | 708 | 33 + 87 + RBD |
| 1120-20-76G8 | 1120-76-G08 | 46411785 | 709 | 33 + 87 + RBD |
| 1120-20-76G9 | 1120-76-G09 | 46411793 | 710 | 33 + 87 + RBD |
| 1120-20-76H10 | 1120-76-H10 | 46411802 | 711 | 33 + 87 + RBD |
| 1120-20-76H11 | 1120-76-H11 | 46411810 | 712 | 33 + 87 + RBD |
| 1120-20-76H3 | 1120-76-H03 | 46411749 | 713 | 33 + 87 + RBD |
| 1120-20-76H5 | 1120-76-H05 | 46411764 | 714 | 33 + 87 + RBD |
| 1120-20-76H6 | 1120-76-H06 | 46411772 | 715 | 33 + 87 + RBD |
| 1120-20-76H7 | 1120-76-H07 | 46411779 | 716 | 33 + 87 + RBD |
| 1120-20-76H8 | 1120-76-H08 | 46411786 | 717 | 33 + 87 + RBD |
| 1120-20-76H9 | 1120-76-H09 | 46411794 | 718 | 33 + 87 + RBD |
| 1120-22-73A12 | 1120-73-A12 | 46411606 | 719 | 33 + 87 + RBD |
| 1120-22-73B12 | 1120-73-B12 | 46411607 | 720 | 33 + 87 + RBD |
| 1120-22-73C12 | 1120-73-C12 | 46411608 | 721 | 33 + 87 + RBD |
| 1120-22-73D11 | 1120-73-D11 | 46411601 | 722 | 33 + 87 + RBD |
| 1120-22-73D12 | 1120-73-D12 | 46411609 | 723 | 33 + 87 + RBD |
| 1120-22-73E11 | 1120-73-E11 | 46411602 | 724 | 33 + 87 + RBD |
| 1120-22-73E12 | 1120-73-E12 | 46411610 | 725 | 33 + 87 + RBD |
| 1120-22-73F11 | 1120-73-F11 | 46411603 | 726 | 33 + 87 + RBD |
| 1120-22-73G11 | 1120-73-G11 | 46411604 | 727 | 33 + 87 + RBD |
| 1120-22-73G12 | 1120-73-G12 | 46411611 | 728 | 33 + 87 + RBD |
| 1120-22-73H11 | 1120-73-H11 | 46411605 | 729 | 33 + 87 + RBD |
| 1120-22-73H12 | 1120-73-H12 | 46411612 | 730 | 33 + 87 + RBD |
| 1120-22-74A10 | 1120-74-A10 | 46411650 | 731 | 33 + 87 + RBD |
| 1120-22-74A5 | 1120-74-A05 | 46411623 | 732 | 33 + 87 + RBD |
| 1120-22-74A7 | 1120-74-A07 | 46411633 | 733 | 33 + 87 + RBD |
| 1120-22-74A9 | 1120-74-A09 | 46411643 | 734 | 33 + 87 + RBD |
| 1120-22-74B10 | 1120-74-B10 | 46411651 | 735 | 33 + 87 + RBD |
| 1120-22-74B5 | 1120-74-B05 | 46411624 | 736 | 33 + 87 + RBD |
| 1120-22-74B6 | 1120-74-B06 | 46411629 | 737 | 33 + 87 + RBD |
| 1120-22-74B7 | 1120-74-B07 | 46411634 | 738 | 33 + 87 + RBD |
| 1120-22-74B8 | 1120-74-B08 | 46411640 | 739 | 33 + 87 + RBD |
| 1120-22-74B9 | 1120-74-B09 | 46411644 | 740 | 33 + 87 + RBD |
| 1120-22-74C10 | 1120-74-C10 | 46411652 | 741 | 33 + 87 + RBD |
| 1120-22-74C4 | 1120-74-C04 | 46411620 | 742 | 33 + 87 + RBD |
| 1120-22-74C5 | 1120-74-C05 | 46411625 | 743 | 33 + 87 + RBD |
| 1120-22-74C6 | 1120-74-C06 | 46411630 | 744 | 33 + 87 + RBD |
| 1120-22-74C7 | 1120-74-C07 | 46411635 | 745 | 33 + 87 + RBD |
| 1120-22-74C9 | 1120-74-C09 | 46411645 | 746 | 33 + 87 + RBD |
| 1120-22-74D2 | 1120-74-D02 | 46411613 | 747 | 33 + 87 + RBD |
| 1120-22-74D4 | 1120-74-D04 | 46411621 | 748 | 33 + 87 + RBD |
| 1120-22-74D5 | 1120-74-D05 | 46411626 | 749 | 33 + 87 + RBD |
| 1120-22-74D7 | 1120-74-D07 | 46411636 | 750 | 33 + 87 + RBD |
| 1120-22-74D9 | 1120-74-D09 | 46411646 | 751 | 33 + 87 + RBD |
| 1120-22-74E10 | 1120-74-E10 | 46411653 | 752 | 33 + 87 + RBD |
| 1120-22-74E2 | 1120-74-E02 | 46411614 | 753 | 33 + 87 + RBD |
| 1120-22-74E3 | 1120-74-E03 | 46411616 | 754 | 33 + 87 + RBD |
| 1120-22-74E7 | 1120-74-E07 | 46411637 | 755 | 33 + 87 + RBD |
| 1120-22-74E8 | 1120-74-E08 | 46411641 | 756 | 33 + 87 + RBD |
| 1120-22-74E9 | 1120-74-E09 | 46411647 | 757 | 33 + 87 + RBD |
| 1120-22-74F10 | 1120-74-F10 | 46411654 | 758 | 33 + 87 + RBD |
| 1120-22-74F2 | 1120-74-F02 | 46411615 | 759 | 33 + 87 + RBD |
| 1120-22-74F3 | 1120-74-F03 | 46411617 | 760 | 33 + 87 + RBD |
| 1120-22-74F6 | 1120-74-F06 | 46411631 | 761 | 33 + 87 + RBD |
| 1120-22-74F7 | 1120-74-F07 | 46411638 | 762 | 33 + 87 + RBD |
| 1120-22-74F8 | 1120-74-F08 | 46411642 | 763 | 33 + 87 + RBD |
| 1120-22-74F9 | 1120-74-F09 | 46411648 | 764 | 33 + 87 + RBD |
| 1120-22-74G3 | 1120-74-G03 | 46411618 | 765 | 33 + 87 + RBD |
| 1120-22-74G5 | 1120-74-G05 | 46411627 | 766 | 33 + 87 + RBD |
| 1120-22-74H10 | 1120-74-H10 | 46411655 | 767 | 33 + 87 + RBD |
| 1120-22-74H3 | 1120-74-H03 | 46411619 | 768 | 33 + 87 + RBD |
| 1120-22-74H4 | 1120-74-H04 | 46411622 | 769 | 33 + 87 + RBD |
| 1120-22-74H5 | 1120-74-H05 | 46411628 | 770 | 33 + 87 + RBD |
| 1120-22-74H6 | 1120-74-H06 | 46411632 | 771 | 33 + 87 + RBD |
| 1120-22-74H7 | 1120-74-H07 | 46411639 | 772 | 33 + 87 + RBD |
| 1120-22-74H9 | 1120-74-H09 | 46411649 | 773 | 33 + 87 + RBD |

TABLE 6
Number of anti-SARS-CoV-2 mAbs tested per immunization group

| | Pretreatment during immunization | | | | |
|---|---|---|---|---|---|
| | | E14315 + | | | E10933 + |
| | Saline | E15160 | E14315 | E15160 | E10987 |
| Total # of | 255 | 83 | 130 | 81 | 224 |
| mAb tested | | | | | |

TABLE 7
Reagents used and lot numbers:

| | | | Monomer |
|---|---|---|---|
| Reagent | | | MW |
| ID | Lot # | Common Name | (g/mol) |
| E10620 | E10620-L4 | SARS-CoV-2 RBD- | 28410 |
| | | MMH | |
| E10933 | 9046400001 | Anti-SARS-CoV-2 mAb | 145280 |
| E10987 | 9046900001 | Anti-SARS-CoV-2 mAb | 144180 |
| E14315 | 9050800001 | Anti-SARS-CoV-2 mAb | 145840 |
| E15160 | 9052800001 | Anti-SARS-CoV-2 mAb | 145460 |
| E1932 | E1932-L181 | Anti-FELD1 mAb | 145720 |
| | | (iso-type control) | |

The entire experiment was performed at 25° C. in buffer containing 0.01 M HEPES pH 7.4, 0.15 M NaCl, 3 mM EDTA, 0.05% v/v Surfactant P20, 0.1 mg/mL BSA (Octet HBS-EP buffer) with the plate shaking at a speed of 1,000 rpm. To assess whether two antibodies were able to compete with one another for binding to their respective epitopes on SARS-CoV-2 RBD extracellular domain expressed with a C-terminal myc-myc-hexahistidine (SARS-CoV-2 RBD-MMH), approximately 0.33 nM of SARS-CoV-2 RBD.mmH was first captured onto anti-his antibody coated Octet biosensors (HIS1K; Fortebio Inc, #18-5120) by submerging the biosensors for 1 minute into wells containing a 10 μg/mL solution of SARS-CoV-2 RBD.mmH. The antigen-captured biosensors were then saturated with the first anti-SARS-CoV-2 monoclonal antibodies (subsequently referred to as mAb-1) by immersion into wells containing a 50 μg/mL solution of mAb-1 (E10933, E10987, E15160, E14315, or E1932 (isotype control)) for 3 minutes. Subsequently, the biosensor tips were dipped into wells of CHOt conditioned media, each containing one of the test anti-SARS-CoV-2 monoclonal antibodies (mAb-2), for 3 minutes. All the biosensors were washed with HBS-EP buffer between steps of the experiment. The real-time binding response was monitored and the binding response at the end of every step was recorded. The responses of mAb-2 binding to SARS-CoV-2 RBD.mmH pre-complexed with E10933, E10987, E15160, E14315, or the isotype control were compared. The percentage inhibition by the prebound E10933, E10987, E15160, E14315 was calculated using the formula below.

$$Percent inhibition = 100 \% * ( 1 - ratio of test mAb on \\ mAb - 1 pre - bound sensor to isotype control pre - bound sensor )$$

### Results

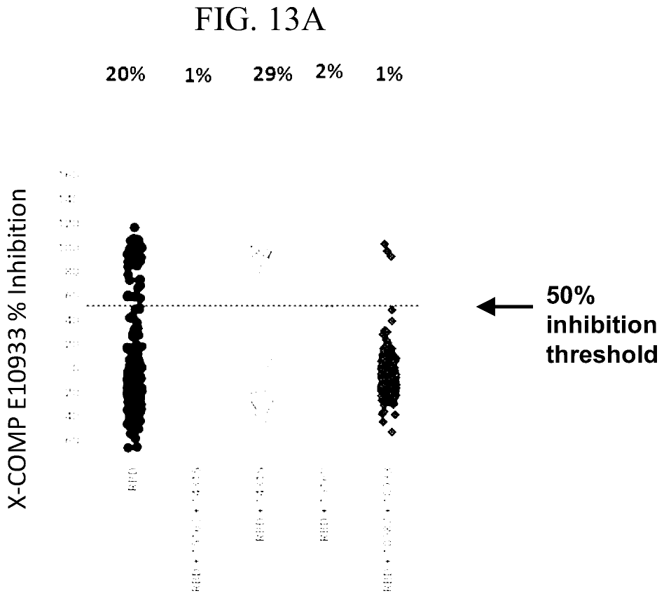

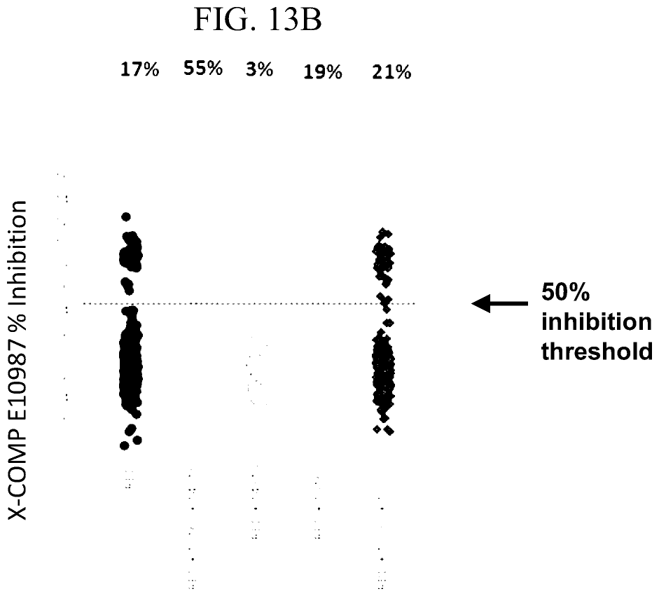

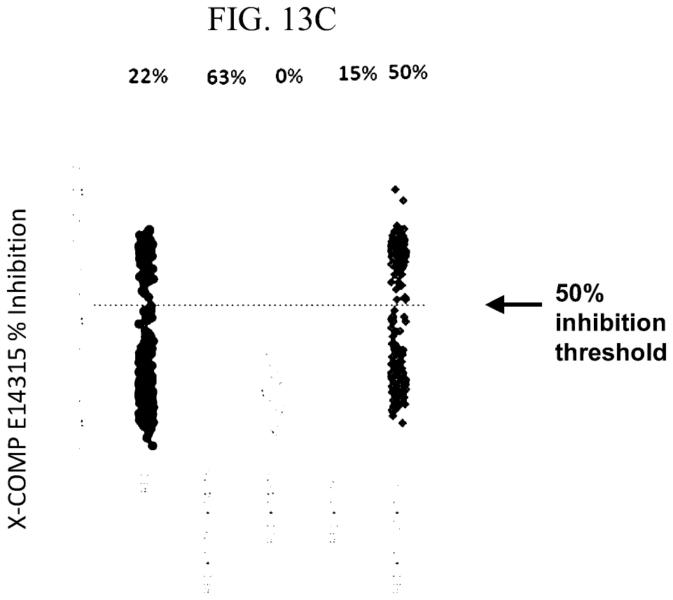

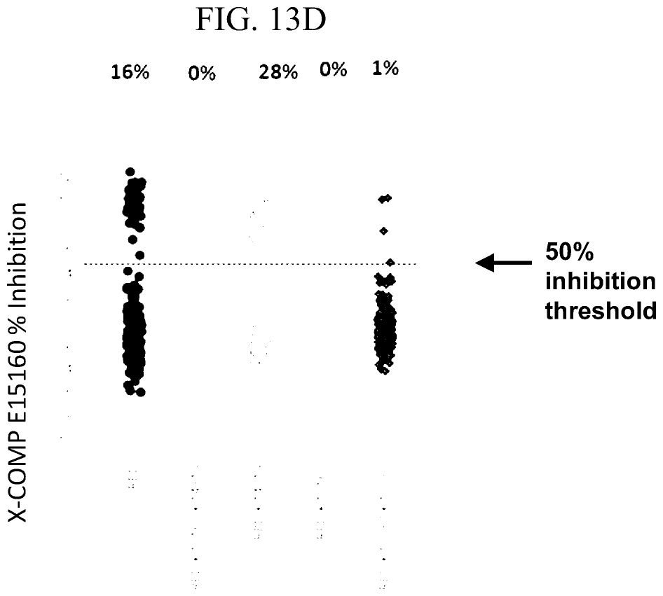

A panel of 773 anti-SARS-CoV-2 mAbs in CHOt conditioned media was assessed for cross competition against 4 selected anti-SARS CoV-2 mAbs that were included in the pre-treatments of the mice during SARS-CoV-2 RBD immunization. These 4 mAbs were prebound to anti-His captured SARS-Cov-2 RBD.mmh to determine whether the test mAbs share the same binding epitopes on RBD.mmh. Percent inhibition of prebound E10933, E10987, E15160, and E14315 on the test mAbs binding to anti-SARS-CoV-2 RBD.mmh were calculated. The test mAbs were grouped by the respective pre-treatment conditions of the mice from which the mAbs were isolated. FIGS. 13A-13D display the calculated percentage inhibition of prebound E10933 (FIG. 13A), E10987 (FIG. 13B), E15160 (FIG. 13C), and E14315 (FIG. 13D) on individual mAbs of each pre-treatment conditions (saline, E15160+E14315, E15160, E14315, and E10933+E10987). In addition, the effect of the pre-treatment conditions on the generation of antibodies that showed a greater than 50% reduction in SARS-CoV2 RBD.mmh binding as a result of prebound E10933, E10987, E15160, E14315 are summarized in Table 8. Strat, Strategy; Sal, Saline.

TABLE 8
Summary of Cross-competition Between mAb-1 anti-SARS-CoV-2 mAbs and E10933, E10987, E14315, or E15160

| | | | | | | | Percentage of the total mAbs tested | | | | |
|---|---|---|---|---|---|---|---|---|---|---|---|
| | # of Samples showing >50% | | | | | | that blocked more than 50% between | | | | |
| | inhibition to anti-SARS-CoV-2 mAb | | | | | | mAb-1 (% >50% inhibition) | | | | |
| | | RBD + | | | RBD + | Anti-SARS- | | RBD + | | | RBD + |
| Immunization | RBD + | 15160 + | RBD + | RBD + | 10987 + | CoV-2 mAb | RBD + | 15160 + | RBD + | RBD + | 10987 + |
| Strat | Sal | 14315 | 14315 | 15160 | 10933 | x-comp | Sal | 14315 | 14315 | 15160 | 10933 |
| E10933 | 52 | 1 | 38 | 2 | 3 | E10933 | 20 | 1 | 29 | 2 | 1 |
| E10987 | 43 | 46 | 4 | 15 | 48 | E10987 | 17 | 55 | 3 | 19 | 21 |
| E14315 | 55 | 52 | 0 | 12 | 111 | E14315 | 22 | 63 | 0 | 15 | 50 |
| E15160 | 42 | 0 | 37 | 0 | 3 | E15160 | 16 | 0 | 28 | 0 | 1 |
| Grand Total | 255 | 83 | 130 | 81 | 224 | | | | | | |
| (773 Samples) | | | | | | | | | | | |

In RBD immunized mice without pretreatment (saline only cohort), the percentage of antibodies that displayed >50% reduction in binding to RBD-prebound with E10933, E10987, E14315, or E15160 were 20%, 17%, 22%, and 16%, respectively. Pretreatment with E15160+E14315 reduced blockers of E15160 to 0% compared to 16% of the saline arm; pretreatment with E14315 reduced blockers of E14315 to 0% from 22% of the saline arm; pretreatment with E15160 reduced blockers of E15160 to 0% from 16% of the saline arm; and pretreatment with E10933+E10987 reduced E10933 blockers to 1% from 20% of the saline arm.

Pretreatment with E15160+E14315 and E10933+10987 showed a reduction in the percentage of mAbs that were blocked by only one of the antibodies included in the pretreatment.

Anti-SARS-CoV-2 mAbs obtained from E10933+E10987, E15160, E14315, and/or E15160+E14315 pre-dosed mice showed reduced or complete loss of competition against E10933, E15160, E14315 and E15160. Conversely, we were able to detect anti-SARS-CoV-2 mAbs obtained from RBD immunized, non-mAb pre-dosed mice that compete against E10933, E10987, E15160 and/or E14315 on RBD. These results show that utilization of mAbs that block dominant epitopes during immunization can result in generating an immune response away from those blocked epitopes as demonstrated via loss of binding competition.

### Example 6. Identification of Selected mAbs that have Anti-SARS-CoV-2 Neutralization Activity and do not Compete with mAbs Used During Immunization

A binding competition assay using the Octet HTX biosensor platform is used to identify selected mAbs that have anti-SARS-CoV-2 neutralization activity and do not compete with E10933+E10987 or E15160+E14315. As an example, percent inhibition representing the amount of E10933, E10987, E15160, E14315 that is inhibited or competed off from anti-SARS-CoV-2 mAbs obtained from mAb-pre-dosed or non-pre-dosed RBD immunized mice is calculated. Identification of anti-SARS-CoV-2 mAbs that do not compete with the mAbs used during immunization to block certain epitopes but still afford neutralization activity is achieved via a pVSV-SARS-CoV-2 spike neutralization assay described herein. Neutralizing mAbs during immunization are used to find additional desirable mAbs that do not compete with the potent neutralizing mAbs and could be included in a mAb cocktail drug product. This example determines, in particular, mAbs that can be included with E10987+E10933 or with E15160+E14315 for triple mAb cocktail.

### Example 7. Modulation of Influenza Hemagglutinin (HA) Antibody Responses in Mice Pre-Dosed with Monoclonal Antibodies with Specificity to the HA Head

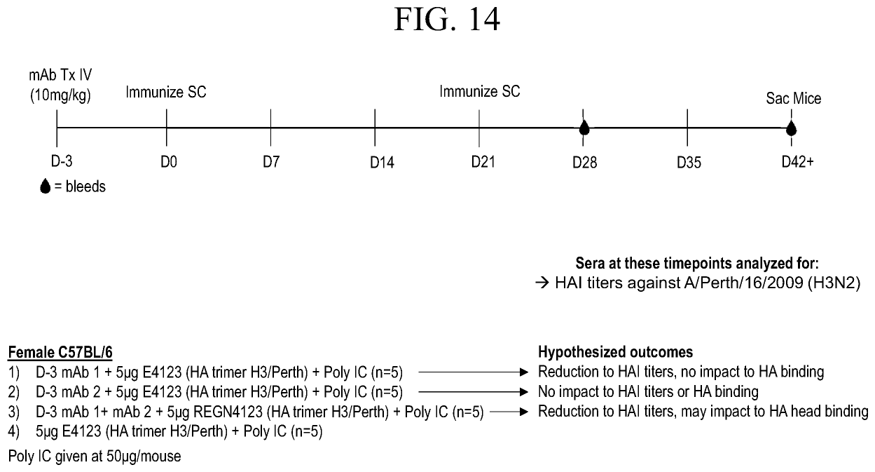

This study investigates modulation of influenza hemagglutinin (HA) antibody responses in which mice are pre-dosed with a first monoclonal antibody (mAb 1) which has specificity to sialic-acid, receptor binding site (RBS) on the HA head, and/or with a second monoclonal antibody (mAb 2) which also binds the HA head but outside of the RBS. In accordance with the study design displayed in FIG. 14, mice are immunized with a protein immunogen (Day 0) comprised of an HA trimeric protein of H3 serotype from A/Perth/16/2009 (H3N2). Three days prior to protein injection, mice were pre-treated with the above-described monoclonal antibodies, or combinations thereof, or control conditions (no antibody). Mice were pre-bled prior to the mAbs pre-treatment, and post immunogen boosts at days 28 and 42, and prior to euthanizing mice for antibody isolation. At end of study hemagglutinin inhibition serum titers (HAI) from immunized mice are assessed (i.e., serum antibodies that bind to the RBS on HA from influenza and inhibit agglutination of red blood cells). Mice dosed with mAb 1 or combination of mAb 1 and mAb 2 are expected to not elicit HAI serum titers due to mAb 1 blocking the RBS site during immunization and thus inhibiting antibodies specific to that site.

### LIST OF SEQUENCES

Amino Acid Sequence of SARS-COV-2 full-length S glycoprotein (SEQ ID NO: 1; NCBI
Reference Sequence: YP_009724390.1; S1 subunit is underlined; RBD site is shown in
bold)
MFVFLVLLPLVSSQCVNLTTRTQLPPAYTNSFTRGVYYPDKVFRSSVLHSTQDLFLPFF
SNVTWFHAIHVSGTNGTKRFDNPVLPENDGVYFASTEKSNIIRGWIFGTTLDSKTQSLL
IVNNATNVVIKVCEFQFCNDPFLGVYYHKNNKSWMESEFRVYSSANNCTFEYVSQPFLM
DLEGKQGNFKNLREFVFKNIDGYFKIYSKHTPINLVRDLPQGFSALEPLVDLPIGINIT
RFQTLLALHRSYLTPGDSSSGWTAGAAAYYVGYLQPRTELLKYNENGTITDAVDCALDP
LSETKCTLKSFTVEKGIYQTSNFRVQPTESIVRFPNITNLCPFGEVENATRFASVYAWN
RKRISNCVADYSVLYNSASFSTFKCYGVSPTKLNDLCFTNVYADSFVIRGDEVRQIAPG
QTGKIADYNYKLPDDFTGCVIAWNSNNLDSKVGGNYNYLYRLFRKSNLKPFERDISTEI
YQAGSTPCNGVEGENCYFPLQSYGFQPTNGVGYQPYRVVVLSFELLHAPATVCGPKKST
NLVKNKCVNFNFNGLTGTGVLTESNKKELPFQQFGRDIADTTDAVRDPQTLEILDITPC
SFGGVSVITPGTNTSNOVAVLYQDVNCTEVPVAIHADQLTPTWRVYSTGSNVFQTRAGC
LIGAEHVNNSYECDIPIGAGICASYQTQTNSPRRARSVASQSIIAYTMSLGAENSVAYS
NNSIAIPTNFTISVTTEILPVSMTKTSVDCTMYICGDSTECSNLLLQYGSFCTQLNRAL
TGIAVEQDKNTQEVFAQVKQIYKTPPIKDFGGENFSQILPDPSKPSKRSFIEDLLENKV
TLADAGFIKQYGDCLGDIAARDLICAQKENGLTVLPPLLTDEMIAQYTSALLAGTITSG
WTFGAGAALQIPFAMQMAYRENGIGVTQNVLYENQKLIANQFNSAIGKIQDSLSSTASA
LGKLQDVVNQNAQALNTLVKQLSSNFGAISSVLNDILSRLDKVEAEVOIDRLITGRLOS
LQTYVTQQLIRAAEIRASANLAATKMSECVLGQSKRVDFCGKGYHLMSFPQSAPHGVVE
LHVTYVPAQEKNFTTAPAICHDGKAHFPREGVFVSNGTHWFVTORNFYEPQIITTDNTF
VSGNCDVVIGIVNNTVYDPLQPELDSFKEELDKYFKNHTSPDVDLGDISGINASVVNIQ
KEIDRLNEVAKNLNESLIDLQELGKYEQYIKWPWYIWLGFIAGLIAIVMVTIMLCCMTS
CCSCLKGCCSCGSCCKFDEDDSEPVLKGVKLHYT
Amino Acid Sequence of SARS-COV-2 Δ19CT S glycoprotein (SEQ ID NO: 2; S1
subunit is underlined; RBD site is shown in bold)
MFVFLVLLPLVSSQCVNLTTRTQLPPAYTNSFTRGVYYPDKVFRSSVLHSTQDLFLPFF
SNVTWFHAIHVSGTNGTKRFDNPVLPFNDGVYFASTEKSNIIRGWIFGTTLDSKTQSLL
IVNNATNVVIKVCEFQFCNDPFLGVYYHKNNKSWMESEFRVYSSANNCTFEYVSQPFLM
DLEGKQGNFKNLREFVFKNIDGYFKIYSKHTPINLVRDLPQGFSALEPLVDLPIGINIT
RFQTLLALHRSYLTPGDSSSGWTAGAAAYYVGYLOPRTFLLKYNENGTITDAVDCALDP
LSETKCTLKSFTVEKGIYQTSNFRVQPTESIVRFPNITNLCPFGEVENATRFASVYAWN
RKRISNCVADYSVLYNSASFSTFKCYGVSPTKLNDLCFTNVYADSFVIRGDEVRQIAPG
QTGKIADYNYKLPDDFTGCVIAWNSNNLDSKVGGNYNYLYRLFRKSNLKPFERDISTEI
YQAGSTPCNGVEGENCYFPLQSYGFQPTNGVGYQPYRVVVLSFELLHAPATVCGPKKST
NLVKNKCVNFNFNGLTGTGVLTESNKKELPFQQFGRDIADTTDAVRDPQTLEILDITPC
SFGGVSVITPGTNTSNOVAVLYQDVNCTEVPVAIHADQLTPTWRVYSTGSNVFQTRAGC
LIGAEHVNNSYECDIPIGAGICASYQTQTNSPRRARSVASQSIIAYTMSLGAENSVAYS
NNSIAIPTNFTISVTTEILPVSMTKTSVDCTMYICGDSTECSNLLLQYGSFCTQLNRAL
TGIAVEQDKNTQEVFAQVKQIYKTPPIKDFGGFNFSQILPDPSKPSKRSFIEDLLENKV
TLADAGFIKQYGDCLGDIAARDLICAQKENGLTVLPPLLTDEMIAQYTSALLAGTITSG
WTFGAGAALQIPFAMQMAYRENGIGVTQNVLYENQKLIANQFNSAIGKIQDSLSSTASA
LGKLQDVVNQNAQALNTLVKQLSSNFGAISSVLNDILSRLDKVEAEVQIDRLITGRLOS
LQTYVTQQLIRAAEIRASANLAATKMSECVLGQSKRVDFCGKGYHLMSFPQSAPHGVVF
LHVTYVPAQEKNFTTAPAICHDGKAHFPREGVFVSNGTHWFVTORNFYEPQIITTDNTF
VSGNCDVVIGIVNNTVYDPLQPELDSFKEELDKYFKNHTSPDVDLGDISGINASVVNIQ
KEIDRLNEVAKNLNESLIDLQELGKYEQYIKWPWYIWLGFIAGLIAIVMVTIMLCCMTS
CCSCLKGCCSCGSCC
SARS-COV-2 Spike D614G VLP DNA 1-1242 (amino acids 13-1255) (SEQ ID NO: 3)
QCVNLTTRTQLPPAYTNSFTRGVYYPDKVFRSSVLHSTQDLFLPFFSNVTWFHAIHVSG
TNGTKRFDNPVLPFNDGVYFASTEKSNIIRGWIFGTTLDSKTQSLLIVNNATNVVIKVC
EFQFCNDPFLGVYYHKNNKSWMESEFRVYSSANNCTFEYVSQPFLMDLEGKQGNEKNLR
EFVFKNIDGYFKIYSKHTPINLVRDLPQGFSALEPLVDLPIGINITRFQTLLALHRSYL
TPGDSSSGWTAGAAAYYVGYLQPRTELLKYNENGTITDAVDCALDPLSETKCTLKSFTV
EKGIYQTSNFRVQPTESIVRFPNITNLCPFGEVENATRFASVYAWNRKRISNCVADYSV
LYNSASFSTFKCYGVSPTKLNDLCFTNVYADSFVIRGDEVRQIAPGQTGKIADYNYKLP
DDFTGCVIAWNSNNLDSKVGGNYNYLYRLERKSNLKPFERDISTEIYQAGSTPCNGVEG
FNCYFPLQSYGFQPTNGVGYQPYRVVVLSFELLHAPATVCGPKKSTNLVKNKCVNFNEN
GLTGTGVLTESNKKFLPFQQFGRDIADTTDAVRDPQTLEILDITPCSFGGVSVITPGIN
TSNQVAVLYQGVNCTEVPVAIHADQLTPTWRVYSTGSNVFQTRAGCLIGAEHVNNSYEC
DIPIGAGICASYQTQTNSPRRARSVASQSIIAYTMSLGAENSVAYSNNSIAIPTNFTIS
VTTEILPVSMTKTSVDCTMYICGDSTECSNLLLQYGSFCTQLNRALTGIAVEQDKNTQE
VFAQVKQIYKTPPIKDFGGFNFSQILPDPSKPSKRSFIEDLLENKVTLADAGFIKQYGD
CLGDIAARDLICAQKENGLTVLPPLLTDEMIAQYTSALLAGTITSGWTFGAGAALQIPF
AMQMAYRFNGIGVTQNVLYENQKLIANQFNSAIGKIQDSLSSTASALGKLQDVVNONAQ
ALNTLVKQLSSNFGAISSVLNDILSRLDKVEAEVOIDRLITGRLOSLOTYVTQQLIRAA
EIRASANLAATKMSECVLGQSKRVDFCGKGYHLMSFPQSAPHGVVFLHVTYVPAQEKNF
TTAPAICHDGKAHFPREGVFVSNGTHWFVTORNFYEPQIITTDNTFVSGNCDVVIGIVN
NTVYDPLQPELDSFKEELDKYFKNHTSPDVDLGDISGINASVVNIQKEIDRLNEVAKNL
NESLIDLQELGKYEQYIKWPWYIWLGFIAGLIAIVMVTIMLCCMTSCCSCLKGCCSCGS
CCK
RBD (R319-F541).mFc (E10621):
Amino acids: SARS-COV-2 RBD mFc (R319-F541) 1-223 (amino acids 319 through 541
of GenBank Accession No. MN908947.3)
Amino acids: 224-456 mFc-(mouse Fc tag)
(SEQ ID NO: 4)
RVQPTESIVRFPNITNLCPFGEVENATRFASVYAWNRKRISNCVADYSVLYNSASESTE
KCYGVSPTKLNDLCFTNVYADSFVIRGDEVRQIAPGQTGKIADYNYKLPDDETGCVIAW
NSNNLDSKVGGNYNYLYRLFRKSNLKPFERDISTEIYQAGSTPCNGVEGENCYFPLQSY
GFQPTNGVGYQPYRVVVLSFELLHAPATVCGPKKSTNLVKNKCVNFEPRGPTIKPCPPC
KCPAPNLLGGPSVFIFPPKIKDVLMISLSPIVTCVVVDVSEDDPDVQISWFVNNVEVHT
AQTQTHREDYNSTLRVVSALPIQHQDWMSGKEFKCKVNNKDLPAPIERTISKPKGSVRA
PQVYVLPPPEEEMTKKQVTLTCMVTDEMPEDIYVEWTNNGKTELNYKNTEPVLDSDGSY
FMYSKLRVEKKNWVERNSYSCSVVHEGLHNHHTTKSFSRTPGK
SARS-COV-2 Spike Ecto (aa 14-1211, R682G, R683S, R685S, K986P, V987P)
foldon Trimer domain.GS.Thrombin.mmH (E11047):
Amino acids: SARS-COV-2 Spike Ecto (aa 14-1211, R682G, R683S, R685S, K986P,
V987P)_ foldon Trimer domain.GS.Thrombin.mmH 1-223 (amino acids 319 through
541 of GenBank Accession No. MN908947.3)
Spike ecto: 1-1198
Fold on trimer: 1199-1225
GS: 1226-1227
Thrombin: 1228-1233
Amino acids: myc-myc-hexahistidine tag: 1234-1261
(SEQ ID NO: 5)
QCVNLTTRTQLPPAYTNSFTRGVYYPDKVFRSSVLHSTQDLFLPFFSNVTWFHAIHVSG
TNGTKRFDNPVLPENDGVYFASTEKSNIIRGWIFGTTLDSKTQSLLIVNNATNVVIKVC
EFQFCNDPFLGVYYHKNNKSWMESEFRVYSSANNCTFEYVSQPFLMDLEGKQGNFKNLR
EFVFKNIDGYFKIYSKHTPINLVRDLPQGFSALEPLVDLPIGINITRFQTLLALHRSYL
TPGDSSSGWTAGAAAYYVGYLOPRTFLLKYNENGTITDAVDCALDPLSETKCTLKSFTV
EKGIYQTSNERVOPTESIVRFPNITNLCPFGEVENATRFASVYAWNRKRISNCVADYSV
LYNSASFSTFKCYGVSPTKLNDLCFTNVYADSFVIRGDEVROIAPGQTGKIADYNYKLP
DDFTGCVIAWNSNNLDSKVGGNYNYLYRLFRKSNLKPFERDISTEIYQAGSTPCNGVEG
FNCYFPLQSYGFQPTNGVGYQPYRVVVLSFELLHAPATVCGPKKSTNLVKNKCVNENEN
GLTGTGVLTESNKKELPFQQFGRDIADTTDAVRDPQTLEILDITPCSFGGVSVITPGTN
TSNQVAVLYQDVNCTEVPVAIHADQLTPTWRVYSTGSNVFQTRAGCLIGAEHVNNSYEC
DIPIGAGICASYQTQTNSPGSASSVASQSIIAYTMSLGAENSVAYSNNSIAIPTNFTIS
VTTEILPVSMTKTSVDCTMYICGDSTECSNLLLQYGSFCTQLNRALTGIAVEQDKNTQE
VFAQVKQIYKTPPIKDFGGFNFSQILPDPSKPSKRSFIEDLLENKVTLADAGFIKQYGD
CLGDIAARDLICAQKFNGLTVLPPLLTDEMIAQYTSALLAGTITSGWTFGAGAALQIPF
AMQMAYRFNGIGVTQNVLYENQKLIANQFNSAIGKIQDSLSSTASALGKLQDVVNQNAQ
ALNTLVKQLSSNFGAISSVLNDILSRLDPPEAEVOIDRLITGRLQSLQTYVTQQLIRAA
EIRASANLAATKMSECVLGQSKRVDFCGKGYHLMSFPQSAPHGVVFLHVTYVPAQEKNF
TTAPAICHDGKAHFPREGVFVSNGTHWFVTORNFYEPQIITTDNTFVSGNCDVVIGIVN
NTVYDPLQPELDSFKEELDKYFKNHTSPDVDLGDISGINASVVNIQKEIDRLNEVAKNL
NESLIDLQELGKYEQYIKGYIPEAPRDGQAYVRKDGEWVLLSTFLGSLVPRGSEQKLIS
EEDLGGEQKLISEEDLHHHHHH
SARS-COV-2 Spike DNA Immunogen (amino acids 1-1273 of GenBank Accession No.
MN908947.3) (SEQ ID NO: 6)
MFVFLVLLPLVSSQCVNLTTRTQLPPAYTNSFTRGVYYPDKVFRSSVLHSTQDLFLPFF
SNVTWFHAIHVSGTNGTKRFDNPVLPENDGVYFASTEKSNIIRGWIFGTTLDSKTQSLL
IVNNATNVVIKVCEFQFCNDPFLGVYYHKNNKSWMESEFRVYSSANNCTFEYVSQPFLM
DLEGKQGNFKNLREFVFKNIDGYFKIYSKHTPINLVRDLPQGFSALEPLVDLPIGINIT
RFQTLLALHRSYLTPGDSSSGWTAGAAAYYVGYLOPRTFLLKYNENGTITDAVDCALDP
LSETKCTLKSFTVEKGIYQTSNERVOPTESIVRFPNITNLCPFGEVENATRFASVYAWN
RKRISNCVADYSVLYNSASFSTFKCYGVSPTKLNDLCFTNVYADSFVIRGDEVRQIAPG
QTGKIADYNYKLPDDFTGCVIAWNSNNLDSKVGGNYNYLYRLFRKSNLKPFERDISTEI
YQAGSTPCNGVEGENCYFPLQSYGFQPTNGVGYQPYRVVVLSFELLHAPATVCGPKKST
NLVKNKCVNFNFNGLTGTGVLTESNKKELPFQQFGRDIADTTDAVRDPQTLEILDITPC
SFGGVSVITPGTNTSNOVAVLYQDVNCTEVPVAIHADQLTPTWRVYSTGSNVFQTRAGC
LIGAEHVNNSYECDIPIGAGICASYQTQTNSPRRARSVASQSIIAYTMSLGAENSVAYS
NNSIAIPTNFTISVTTEILPVSMTKTSVDCTMYICGDSTECSNLLLQYGSFCTQLNRAL
TGIAVEQDKNTQEVFAQVKQIYKTPPIKDFGGFNFSQILPDPSKPSKRSFIEDLLENKV
TLADAGFIKQYGDCLGDIAARDLICAQKENGLTVLPPLLTDEMIAQYTSALLAGTITSG
WTFGAGAALQIPFAMQMAYRENGIGVTQNVLYENQKLIANQFNSAIGKIQDSLSSTASA
LGKLQDVVNQNAQALNTLVKQLSSNFGAISSVLNDILSRLDKVEAEVOIDRLITGRLOS
LQTYVTQQLIRAAEIRASANLAATKMSECVLGQSKRVDFCGKGYHLMSFPQSAPHGVVF
LHVTYVPAQEKNFTTAPAICHDGKAHFPREGVFVSNGTHWFVTORNFYEPQIITTDNTF
VSGNCDVVIGIVNNTVYDPLQPELDSFKEELDKYFKNHTSPDVDLGDISGINASVVNIQ
KEIDRLNEVAKNLNESLIDLQELGKYEQYIKWPWYIWLGFIAGLIAIVMVTIMLCCMTS
CCSCLKGCCSCGSCCKFDEDDSEPVLKGVKLHYT
Ab13261 Heavy Chain:
Variable Heavy Chain
mIgG2a
(SEQ ID NO: 7)
QVQLQQSGAELMKPGASVKISCKATGYTFSSYWIEWVKORPGHGLEWIGEILPGSGSTN
YNEKFKGKATFTADTSSKTAYMQLSSLTSEDSAVYYCARRNYRNDGFTYWGQGTLVTVS
SAKTTAPSVYPLAPVCGDTTGSSVTLGCLVKGYFPEPVTLTWNSGSLSSGVHTFPAVLQ
SDLYTLSSSVTVTSSTWPSQSITCNVAHPASSTKVDKKIEPRGPTIKPCPPCKCPAPNL
LGGPSVFIFPPKIKDVLMISLSPIVTCVVVDVSEDDPDVQISWFVNNVEVHTAQTQTHR
EDYNSTLRVVSALPIQHQDWMSGKEFKCKVNNKDLPAPIERTISKPKGSVRAPQVYVLP
PPEEEMTKKQVTLTCMVTDFMPEDIYVEWTNNGKTELNYKNTEPVLDSDGSYFMYSKLR
VEKKNWVERNSYSCSVVHEGLHNHHTTKSFSRTPGK
Ab13261 Light Chain:
Variable Light Chain
Constant Light Chain
(SEQ ID NO: 8)
DIVMTQSPAALSVAPGDRVSLSCRASQSISDYLHWYQQKSHESPRLLIKYASQSISGIP
SRFSGSGSGSDFTLSINSVEPEDVGVYYCONGHSFPWTFGGGTKLEIKRADAAPTVSIF
PPSSEQLTSGGASVVCFLNNFYPKDINVKWKIDGSERQNGVLNSWTDQDSKDSTYSMSS
TLTLTKDEYERHNSYTCEATHKTSTSPIVKSFNRGEC
Ab13269 Heavy Chain:
Variable Heavy Chain
mIgG2a
(SEQ ID NO: 9)
EVOLVESGGGLVKPGGSLKLSCAASGFTFSDYAMSWVROTPEKRLEWVATISSGGPFTY
YPDSVKGRFIISRDNAKNTLYLOMSSLRSEDTAMFFCARGYGNYRYFDVWGAGTTVTVS
SAKTTAPSVYPLAPVCGDTTGSSVTLGCLVKGYFPEPVTLTWNSGSLSSGVHTFPAVLQ
SDLYTLSSSVTVTSSTWPSQSITCNVAHPASSTKVDKKIEPRGPTIKPCPPCKCPAPNL
LGGPSVFIFPPKIKDVLMISLSPIVTCVVVDVSEDDPDVQISWFVNNVEVHTAQTOTHR
EDYNSTLRVVSALPIQHQDWMSGKEFKCKVNNKDLPAPIERTISKPKGSVRAPQVYVLP
PPEEEMTKKQVTLTCMVTDFMPEDIYVEWTNNGKTELNYKNTEPVLDSDGSYFMYSKLR
VEKKNWVERNSYSCSVVHEGLHNHHTTKSFSRTPGK
Ab13269 Light Chain:
Variable Light Chain
Constant Light Chain
(SEQ ID NO: 10)
DILLTQSPAILSVSPGERVTFSCRASQSIGTSIHWFQQRINGSPRLLIKYASESISGIP
SRFSGSGSGTDFTLTINSVESEDIADYYCQQSNSWPLTFGAGTKLELKRADAAPTVSIF
PPSSEQLTSGGASVVCFLNNFYPKDINVKWKIDGSERQNGVLNSWTDQDSKDSTYSMSS
TLTLTKDEYERHNSYTCEATHKTSTSPIVKSFNRGEC
Amino Acid Sequence of SARS-COV-1 Spike (S) protein (UniProtKB/Swiss-Prot:
P59594.1)
(SEQ ID NO: 11)
MFIFLLFLTLTSGSDLDRCTTFDDVQAPNYTQHTSSMRGVYYPDEIFRSDTLYLTQDLF
LPFYSNVTGFHTINHTFGNPVIPFKDGIYFAATEKSNVVRGWVFGSTMNNKSQSVIIIN
NSTNVVIRACNFELCDNPFFAVSKPMGTQTHTMIFDNAFNCTFEYISDAFSLDVSEKSG
NFKHLREFVFKNKDGFLYVYKGYQPIDVVRDLPSGENTLKPIFKLPLGINITNFRAILT
AFSPAQDIWGTSAAAYFVGYLKPTTEMLKYDENGTITDAVDCSONPLAELKCSVKSFEI
DKGIYQTSNFRVVPSGDVVRFPNITNLCPFGEVENATKFPSVYAWERKKISNCVADYSV
LYNSTFFSTFKCYGVSATKLNDLCFSNVYADSFVVKGDDVRQIAPGQTGVIADYNYKLP
DDFMGCVLAWNTRNIDATSTGNYNYKYRYLRHGKLRPFERDISNVPFSPDGKPCTPPAL
NCYWPLNDYGFYTTTGIGYQPYRVVVLSFELLNAPATVCGPKLSTDLIKNQCVNENENG
LTGTGVLTPSSKRFQPFQQFGRDVSDFTDSVRDPKTSEILDISPCSFGGVSVITPGTNA
SSEVAVLYQDVNCTDVSTAIHADQLTPAWRIYSTGNNVNFSISITTEVMPVSMAKTSVD
CNMYICGDSTECANLLLQYGSFCTQLNRALSGIAAEQDRNTREVFAQVKQMYKTPTLKY
FGGFNFSQILPDPLKPTKRSFIEDLLENKVTLADAGEMKQYGECLGDINARDLICAQKF
NGLTVLPPLLTDDMIAAYTAALVSGTATAGWTFGAGAALQIPFAMQMAYRENGIGVTON
VLYENQKQIANQFNKAISQIQESLTTTSTALGKLQDVVNQNAQALNTLVKOLSSNFGAI
SSVLNDILSRLDKVEAEVQIDRLITGRLQSLQTYVTQQLIRAAEIRASANLAATKMSEC
VLGQSKRVDFCGKGYHLMSFPQAAPHGVVFLHVTYVPSQERNFTTAPAICHEGKAYFPR
EGVFVFNGTSWFITQRNFFSPQIITTDNTFVSGNCDVVIGIINNTVYDPLQPELDSFKE
ELDKYFKNHTSPDVDLGDISGINASVVNIQKEIDRLNEVAKNLNESLIDLQELGKYEQY
IKWPWYVWLGFIAGLIAIVMVTILLCCMTSCCSCLKGACSCGSCCKFDEDDSEPVLKGV
KLHYT
Wild-type SARS-COV-2 spike glycoprotein peptide sequence
(SEQ ID NO: 12)
QTNSPRRARSV
Mutant SARS-COV-2 S glycoprotein peptide sequence
(SEQ ID NO: 13)
QTILRSV
Mutant SARS-COV-2 S glycoprotein peptide sequence
(SEQ ID NO: 14)
QTNSPGSASSV
2A sequence
(SEQ ID NO: 15)
PVKQLLNFDLLKLAGDVESNPGP
2A sequence
(SEQ ID NO: 16)
QCTNYALLKLAGDVESNPGP
2A sequence
(SEQ ID NO: 17)
ATNFSLLKQAGDVEENPGP
2A sequence
(SEQ ID NO: 18)
EGRGSLLTCGDVESNPGP
Influenza A/Perth Y98F-foldon-BirA-6xHis (E4123)
Amino acids 1-573 (amino acids 1-520; Y114F of accession number ACS71642)
(SEQ ID NO: 19)
Signal sequence: 1-16
Influenza H1N1 New Caledonia ecto (1-520; Y98F): 17-520
Linker: 521-522
Foldon: 523-549
Linker: 550-551
BirA: 552-566
Linker: 567
6xhis: 568-573

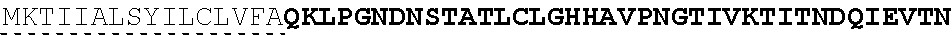

ATELVQSSSTGEICDSPHQILDGKNCTLIDALLGDPQCDGFQNKKWDLFVER
SKAYSNCFPYDVPDYASLRSLVASSGTLEFNNESFNWTGVTQNGTSSACIRRS
KNSFFSRLNWLTHLNFKYPALNVTMPNNEQFDKLYIWGVHHPGTDKDQIFL
YAQASGRITVSTKRSQQTVSPNIGSRPRVRNIPSRISIYWTIVKPGDILLINSTG
NLIAPRGYFKIRSGKSSIMRSDAPIGKCNSECITPNGSIPNDKPFQNVNRITYG
ACPRYVKQNTLKLATGMRNVPEKQTRGIFGAIAGFIENGWEGMVDGWYGF
RHQNSEGRGQAADLKSTQAAIDQINGKLNRLIGKTNEKFHQIEKEFSEVEGR
IQDLEKYVEDTKIDLWSYNAELLVALENQHTIDLTDSEMNKLFEKTKKQLR
ENAEDMGNGCFKIYHKCDNACIGSIRNGTYDHDVYRDEALNNRFQIKGGSG

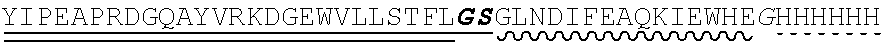

E10987
E10987 Heavy Chain
Variable heavy chain; Constant heavy chain
(SEQ ID NO: 20)
QVQLVESGGGVVQPGRSLRLSCAASGFTFSNYAMYWVRQAPGKGLEWVAVISY
DGSNKYYADSVKGRFTISRDNSKNTLYLQMNSLRTEDTAVYYCASGSDYGDYL
LVYWGQGTLVTVSSASTKGPSVFPLAPSSKSTSGGTAALGCLVKDYFPEPVTV
SWNSGALTSGVHTFPAVLQSSGLYSLSSVVTVPSSSLGTQTYICNVNHKPSNT
KVDKKVEPKSCDKTHTCPPCPAPELLGGPSVFLFPPKPKDTLMISRTPEVTC
VVVDVSHEDPEVKFNWYVDGVEVHNAKTKPREEQYNSTYRVVSVLTVLHQ
DWLNGKEYKCKVSNKALPAPIEKTISKAKGQPREPQVYTLPPSRDELTKNQ
VSLTCLVKGFYPSDIAVEWESNGQPENNYKTTPPVLDSDGSFFLYSKLTVDK
SRWQQGNVFSCSVMHEALHNHYTQKSLSLSPGK
E10987 Light Chain (Lambda):
Variable light chain; Constant light chain
(SEQ ID NO: 21)
QSALTQPASVSGSPGQSITISCTGTSSDVGGYNYVSWYQQHPGKAPKLMIYDVSK
RPSGVSNRFSGSKSGNTASLTISGLQSEDEADYYCNSLTSISTWVFGGGTKLTVLG
QPKAAPSVTLFPPSSEELQANKATLVCLISDFYPGAVTVAWKADSSPVKAGV
ETTTPSKQSNNKYAASSYLSLTPEQWKSHRSYSCQVTHEGSTVEKTVAPTEC
S
E10933
E10933 Heavy Chain:
Variable heavy chain; Constant heavy chain
(SEQ ID NO: 22)
QVQLVESGGGLVKPGGSLRLSCAASGFTFSDYYMSWIRQAPGKGLEWVSYITYS
GSTIYYADSVKGRFTISRDNAKSSLYLQMNSLRAEDTAVYYCARDRGTTMVPFD
YWGQGTLVTVSSASTKGPSVFPLAPSSKSTSGGTAALGCLVKDYFPEPVTVS
WNSGALTSGVHTFPAVLQSSGLYSLSSVVTVPSSSLGTQTYICNVNHKPSNTK
VDKKVEPKSCDKTHTCPPCPAPELLGGPSVFLFPPKPKDTLMISRTPEVTCV
VVDVSHEDPEVKFNWYVDGVEVHNAKTKPREEQYNSTYRVVSVLTVLHQD
WLNGKEYKCKVSNKALPAPIEKTISKAKGQPREPQVYTLPPSRDELTKNQVS
LTCLVKGFYPSDIAVEWESNGQPENNYKTTPPVLDSDGSFFLYSKLTVDKSR
WQQGNVFSCSVMHEALHNHYTQKSLSLSPGK
E10933 Light Chain (kappa):
Variable light chain; Constant light chain
(SEQ ID NO: 23)
DIQMTQSPSSLSASVGDRVTITCQASQDITNYLNWYQQKPGKAPKLLIYAASNLE
TGVPSRFSGSGSGTDFTFTISGLQPEDIATYYCQQYDNLPLTFGGGTKVEIKRTVA
APSVFIFPPSDEQLKSGTASVVCLLNNFYPREAKVQWKVDNALQSGNSQESV
TEQDSKDSTYSLSSTLTLSKADYEKHKVYACEVTHQGLSSPVTKSFNRGEC
E14315
E14315 Heavy Chain:
Variable heavy chain; Constant heavy chain
(SEQ ID NO: 24)
QVQLVQSGAEVKKPGSSVKVSCKASGDTFSTYAINWVRQAPGQGLEWMGRFIHI
FGTANYAQKFQGRVTITADESTSTAYMELRSLRSEDTAVYYCARDGVDYGDYR
PDYWGQGTLVTVSSASTKGPSVFPLAPSSKSTSGGTAALGCLVKDYFPEPVTV
SWNSGALTSGVHTFPAVLQSSGLYSLSSVVTVPSSSLGTQTYICNVNHKPSNT
KVDKKVEPKSCDKTHTCPPCPAPELLGGPSVFLFPPKPKDTLMISRTPEVTC
VVVDVSHEDPEVKFNWYVDGVEVHNAKTKPREEQYNSTYRVVSVLTVLHQ
DWLNGKEYKCKVSNKALPAPIEKTISKAKGQPREPQVYTLPPSRDELTKNQ
VSLTCLVKGFYPSDIAVEWESNGQPENNYKTTPPVLDSDGSFFLYSKLTVDK
SRWQQGNVFSCSVMHEALHNHYTQKSLSLSPGK
E14315 Light Chain (kappa):
Variable light chain; Constant light chain
(SEQ ID NO: 25)
EIVLTQSPGTLSLSPGERATLSCRASQSVSSNYLAWYQQKPGQAPRLLIYGASSRA
TGIPERFSGSGSGTDFTLTISRLEPEDFAVYYCQQYGSSLYTFGQGTKLEIKRTVA
APSVFIFPPSDEQLKSGTASVVCLLNNFYPREAKVQWKVDNALQSGNSQESV
TEQDSKDSTYSLSSTLTLSKADYEKHKVYACEVTHQGLSSPVTKSFNRGEC
E15160
E15160 Heavy Chain:
Variable heavy chain; Constant heavy chain
(SEQ ID NO: 26)
EVOLVESGGGLVQPGGSLRLSCSASGFTFSRYAMYWVRQAPGKGLEYVSAISSD
GGSTYDADSVKGRFTISRANSKNTLYLQMSSLRAEDTAVYYCVKGLRELLYYY
YGMDVWGQGTTVTVSSASTKGPSVFPLAPSSKSTSGGTAALGCLVKDYFPEP
VTVSWNSGALTSGVHTFPAVLQSSGLYSLSSVVTVPSSSLGTQTYICNVNHKP
SNTKVDKKVEPKSCDKTHTCPPCPAPELLGGPSVFLFPPKPKDTLMISRTPE
VTCVVVDVSHEDPEVKFNWYVDGVEVHNAKTKPREEQYNSTYRVVSVLTV
LHQDWLNGKEYKCKVSNKALPAPIEKTISKAKGQPREPQVYTLPPSRDELT
KNQVSLTCLVKGFYPSDIAVEWESNGQPENNYKTTPPVLDSDGSFFLYSKLT
VDKSRWQQGNVFSCSVMHEALHNHYTQKSLSLSPGK
E15160 Light Chain (kappa):
Variable light chain; Constant light chain
(SEQ ID NO: 27)
DIQMTQSPSSLSASVGDRVTITCRAGQSISSFLNWYQQKPGKAPKLLIYAASSLQS
GVPSRFSGSGSGTDFTLTISSLQPEDFATYYCQQSYITPFTFGPGTKVDIKRTVAAP
SVFIFPPSDEQLKSGTASVVCLLNNFYPREAKVQWKVDNALQSGNSQESVTE
QDSKDSTYSLSSTLTLSKADYEKHKVYACEVTHQGLSSPVTKSFNRGEC

The claimed subject matter is not to be limited in scope by the specific embodiments described herein. Indeed, various modifications of the claimed subject matter in addition to those described herein will become apparent to those skilled in the art from the foregoing description. Such modifications are intended to fall within the scope of the appended claims.

All patents, applications, publications, test methods, literature, and other materials cited herein are hereby incorporated by reference in their entirety as if physically present in this specification.

1. A method for redirecting an antibody response in a subject from one or more first epitopes of an antigen towards one or more second epitopes of said antigen, said method comprising administering to the subject (i) said antigen or a nucleic acid molecule encoding said antigen and (ii) one or more antibodies targeting said one or more first epitopes of said antigen or one or more nucleic acid molecules encoding said one or more antibodies, wherein said antigen or a nucleic acid molecule encoding said antigen and said one or more antibodies or one or more nucleic acid molecules encoding said one or more antibodies are administered to the subject in amounts effective for generating antibodies to one or more second epitopes of said antigen.

2. A method for shielding one or more first epitopes of an antigen from recognition by the immune system of a subject, said method comprising administering to the subject (i) said antigen or a nucleic acid molecule encoding said antigen and (ii) one or more antibodies targeting said one or more first epitopes of said antigen or one or more nucleic acid molecules encoding said one or more antibodies, wherein said one or more antibodies or one or more nucleic acid molecules encoding said one or more antibodies are administered to the subject in an amount effective to shield one or more first epitopes of said antigen from recognition by the immune system of the subject.

3. A method for generating one or more antibodies targeting a second epitope of an antigen, said method comprising administering to a subject (i) said antigen or a nucleic acid molecule encoding said antigen and (ii) one or more antibodies targeting one or more first epitopes of said antigen or one or more nucleic acid molecules encoding said one or more antibodies, wherein said antigen or a nucleic acid molecule encoding said antigen and said one or more antibodies or one or more nucleic acid molecules encoding said one or more antibodies are administered to the subject in amounts effective for generating antibodies to one or more second epitopes of said antigen.

4. The method of claim 3, further comprising isolating from the subject one or more antibodies which target said antigen or isolating cells producing antibodies which target said antigen.

5. The method of claim 4, wherein said isolating comprises binding of said antibodies or cells producing said antibodies to said antigen, wherein said antigen comprises a detectable label.

6. The method of claim 4 or claim 5, wherein the cells producing antibodies are B cells.

7. The method of any one of claims 4-6, further comprising generating a monoclonal antibody (mAb) based on the antibody isolated from the subject or an antigen-binding fragment thereof.

8. The method of claim 7, wherein the monoclonal antibody (mAb) is a human antibody.

9. The method of claim 7, wherein the monoclonal antibody (mAb) is a humanized antibody.

10. A method for increasing efficacy of a vaccine in a subject in need thereof, wherein the vaccine comprises an antigen or a nucleic acid molecule encoding said antigen, said method comprising administering to the subject (i) said vaccine and (ii) one or more antibodies or one or more nucleic acid molecules encoding said one or more antibodies targeting one or more first epitopes of said antigen, wherein said vaccine and said one or more antibodies or one or more nucleic acid molecules encoding said one or more antibodies are administered to the subject in amounts effective for increasing efficacy of said vaccine.

11. The method of claim 10, wherein said vaccine is administered to the subject in a prime-boost regimen, and wherein said prime-boost regimen comprises administering said one or more antibodies or one or more nucleic acid molecules encoding said one or more antibodies to the subject after administering a prime dose of said vaccine to the subject but before administering a boost dose of said vaccine to the subject.

12. The method of any one of claims 1-11, wherein said one or more antibodies or one or more nucleic acid molecules encoding said one or more antibodies are administered to the subject before administering said antigen or said nucleic acid molecule encoding said antigen.

13. The method of claim 12, wherein said one or more antibodies or one or more nucleic acid molecules encoding said one or more antibodies are administered to the subject up to three weeks before administering said antigen or said nucleic acid molecule encoding said antigen.

14. The method of claim 13, wherein said one or more antibodies or one or more nucleic acid molecules encoding said one or more antibodies are administered to the subject up to three days before administering said antigen or said nucleic acid molecule encoding said antigen.

15. The method of any one of claims 1-11, wherein said one or more antibodies or one or more nucleic acid molecules encoding said one or more antibodies are administered to the subject after administering said antigen or said nucleic acid molecule encoding said antigen.

16. The method of claim 15, wherein said one or more antibodies or one or more nucleic acid molecules encoding said one or more antibodies are administered to the subject up to three weeks after administering said antigen or said nucleic acid molecule encoding said antigen.

17. The method of any one of claims 1-11, wherein said one or more antibodies or one or more nucleic acid molecules encoding said one or more antibodies are administered to the subject during administering said antigen or said nucleic acid molecule encoding said antigen.

18. The method of claim 17, wherein (i) said one or more antibodies or one or more nucleic acid molecules encoding said one or more antibodies and (ii) said antigen or said nucleic acid molecule encoding said antigen are administered as different formulations.

19. The method of claim 17, wherein (i) said one or more antibodies or one or more nucleic acid molecules encoding said one or more antibodies and (ii) said antigen or said nucleic acid molecule encoding said antigen are administered in the same formulation.

20. The method of claim 19, wherein the method comprises administering to the subject a nucleic acid molecule encoding (i) said one or more antibodies and (ii) said antigen.

21. The method of claim 20, wherein said nucleic acid molecule is an RNA molecule

22. The method of claim 21, wherein said RNA molecule is an mRNA molecule.

23. The method of claim 20, wherein said nucleic acid molecule is a DNA molecule.

24. The method of any one of claims 20-23, wherein said nucleic acid molecule is chemically modified.

25. The method of any one of claims 20-24, wherein said nucleic acid molecule comprises at least one regulatory element operably linked to a nucleotide sequence encoding said antigen and/or a nucleotide sequence encoding said one or more antibodies.

26. The method of claim 25, wherein the regulatory element is a promoter.

27. The vector of any one of claims 20-26, wherein the nucleic acid molecule is comprised within a vector.

28. The method of claim 27, wherein the vector is a viral vector.

29. The method of claim 28, wherein the viral vector is a retroviral vector, an adenoviral vector, an adeno-associated virus vector, an alphaviral vector, a herpes virus vector, a baculovirus vector, or a vaccinia virus vector.

30. The method of claim 29, wherein the retroviral vector is a lentiviral vector.

31. The method of claim 27, wherein the vector is a non-viral vector.

32. The method of claim 31, wherein the non-viral vector is a minicircle plasmid, a Sleeping Beauty transposon, a piggyBac transposon, or a single- or double-stranded DNA molecule that is used as a template for homology directed repair (HDR) based gene editing.

33. The method of any one of claims 1-32, wherein said one or more first epitopes are immunodominant epitopes.

34. The method of claim 33, wherein said immunodominant epitopes are less conserved than other epitopes of said antigen between different strains or species of a pathogen from which said antigen is derived.

35. The method of any one of claims 1-34, wherein said antigen is a protein antigen.

36. The method of any one of claims 1-34, wherein said antigen is a non-protein antigen.

37. The method of any one of claims 1-36, wherein said antigen is derived from a Class I pathogen.

38. The method of any one of claims 1-36, wherein said antigen is derived from a Class II pathogen.

39. The method of claim 38, wherein said pathogen is a virus.

40. The method of claim 39, wherein said virus is a coronavirus.

41. The method of claim 40, wherein said coronavirus is SARS-CoV-2.

42. The method of claim 41, wherein said antigen is SARS-CoV-2 spike glycoprotein and said one or more first epitopes are neutralizing epitopes comprised within receptor binding domain (RBD) of said SARS-CoV-2 spike glycoprotein.

43. The method of claim 39, wherein said virus is an influenza virus.

44. The method of claim 43, wherein said antigen is influenza hemagglutinin (HA) and said one or more first epitopes are comprised within sialic-acid, receptor binding site (RBS) on the HA head.

45. The method of any one of claims 1-36, wherein said antigen is an endogenous molecule of the subject.

46. The method of claim 45, wherein said antigen is targeted by an immune response in an autoimmune disease.

47. The method of any one of claims 1-46, wherein said one or more antibodies are monoclonal antibodies (mAbs).

48. The method of any one of claims 1-47, wherein the subject is a mammal.

49. The method of claim 48, wherein the subject is a human.

50. The method of claim 48, wherein the subject is an experimental animal.

51. The method of claim 50, wherein the subject is a mouse.

52. A nucleic acid molecule encoding an antigen and one or more antibodies targeting one or more first epitopes of said antigen.

53. The nucleic acid molecule of claim 52, wherein the nucleic acid molecule is an RNA molecule

54. The nucleic acid molecule of claim 53, wherein the RNA molecule is an mRNA molecule.

55. The nucleic acid molecule of claim 52, wherein the nucleic acid molecule is a DNA molecule.

56. The nucleic acid molecule of any one of claims 52-55, wherein the nucleic acid molecule is chemically modified.

57. The nucleic acid molecule of any one of claims 52-56, wherein the nucleic acid molecule comprises at least one regulatory element operably linked to a nucleotide sequence encoding said antigen and/or a nucleotide sequence encoding said one or more antibodies.

58. The nucleic acid molecule of claim 57, wherein the regulatory element is a promoter.

59. A vector comprising the nucleic acid molecule of any one of claims 52-58.

60. The vector of claim 59, wherein the vector is a viral vector.

61. The vector of claim 60, wherein the viral vector is a retroviral vector, an adenoviral vector, an adeno-associated virus vector, an alphaviral vector, a herpes virus vector, a baculovirus vector, or a vaccinia virus vector.

62. The vector of claim 61, wherein the retroviral vector is a lentiviral vector.

63. The vector of claim 59, wherein the vector is a non-viral vector.

64. The vector of claim 63, wherein the non-viral vector is a minicircle plasmid, a Sleeping Beauty transposon, a piggyBac transposon, or a single or double stranded DNA molecule that is used as a template for homology directed repair (HDR) based gene editing.

65. An isolated host cell comprising a nucleic acid molecule of any one of claims 52-58, or a vector or any one of claims 59-64.

66. The isolated host cell of claim 65, wherein the host cell is a mammalian cell.

67. A lipid nanoparticle comprising the nucleic acid of any one of claims 52-58 or the vector of any one of claims 59-64.

68. A formulation comprising the nucleic acid molecule of any one of claims 52-58, the vector of any one of claims 59-64 or the lipid nanoparticle of claim 67.

69. A formulation comprising an antigen or a nucleic acid molecule encoding said antigen, and one or more antibodies targeting one or more first epitopes of said antigen or one or more nucleic acid molecules encoding said one or more antibodies.

70. A formulation comprising two or more monoclonal antibodies (mAbs) targeting one or more first epitopes of an antigen.

71. The formulation of claim 70, wherein said first epitopes are immunodominant epitopes.

72. The formulation of claim 71, wherein said immunodominant epitopes are less conserved than other epitopes of said antigen between different strains or species of a pathogen from which said antigen is derived.

73. The formulation of any one of claims 70-72, wherein the antigen is a protein antigen.

74. The formulation of any one of claims 70-72, wherein the antigen is a non-protein antigen.

75. The formulation of any one of claims 70-74, wherein the antigen is derived from a Class I pathogen.

76. The formulation of any one of claims 70-74, wherein the antigen is derived from a Class II pathogen.

77. The formulation of claim 76, wherein said pathogen is a virus.

78. The formulation of claim 77, wherein said virus is a coronavirus.

79. The formulation of claim 78, wherein said coronavirus is SARS-CoV-2.

80. The formulation of claim 79, wherein said antigen is SARS-CoV-2 spike glycoprotein and said first epitopes are neutralizing epitopes comprised within receptor binding domain (RBD) of said SARS-CoV-2 spike glycoprotein.

81. The formulation of claim 77, wherein said virus is an influenza virus.

82. The formulation of claim 81, wherein said antigen is influenza hemagglutinin (HA) and said one or more first epitopes are comprised within sialic-acid, receptor binding site (RBS) on the HA head.

83. The formulation of any one of claims 70-74, wherein said antigen is a molecule targeted by an immune response in an autoimmune disease.

84. A kit comprising (i) an antigen or a nucleic acid molecule encoding said antigen, and (ii) one or more antibodies targeting one or more first epitopes of said antigen or one or more nucleic acid molecules encoding said one or more antibodies.


SEQUENCE LISTING (28 sequences)

SEQ ID NO: 1 - protein, 1273 aa, Severe acute respiratory syndrome coronavirus 2
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 2 - protein, 1254 aa, Severe acute respiratory syndrome coronavirus 2
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCC

SEQ ID NO: 3 - protein, 1242 aa, Severe acute respiratory syndrome coronavirus 2
QCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCK

SEQ ID NO: 4 - protein, 456 aa, synthetic construct
RVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 5 - protein, 1261 aa, synthetic construct
QCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPGSASSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKGYIPEAPRDGQAYVRKDGEWVLLSTFLGSLVPRGSEQKLISEEDLGGEQKLISEEDLHHHHHH

SEQ ID NO: 6 - protein, 1273 aa, Severe acute respiratory syndrome coronavirus 2; identical to SEQ ID NO: 1

SEQ ID NO: 7 - protein, 449 aa, synthetic construct
QVQLQQSGAELMKPGASVKISCKATGYTFSSYWIEWVKQRPGHGLEWIGEILPGSGSTNYNEKFKGKATFTADTSSKTAYMQLSSLTSEDSAVYYCARRNYRNDGFTYWGQGTLVTVSSAKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 8 - protein, 214 aa, synthetic construct
DIVMTQSPAALSVAPGDRVSLSCRASQSISDYLHWYQQKSHESPRLLIKYASQSISGIPSRFSGSGSGSDFTLSINSVEPEDVGVYYCQNGHSFPWTFGGGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRGEC

SEQ ID NO: 9 - protein, 449 aa, synthetic construct
EVQLVESGGGLVKPGGSLKLSCAASGFTFSDYAMSWVRQTPEKRLEWVATISSGGPFTYYPDSVKGRFIISRDNAKNTLYLQMSSLRSEDTAMFFCARGYGNYRYFDVWGAGTTVTVSSAKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 10 - protein, 214 aa, synthetic construct
DILLTQSPAILSVSPGERVTFSCRASQSIGTSIHWFQQRTNGSPRLLIKYASESISGIPSRFSGSGSGTDFTLTINSVESEDIADYYCQQSNSWPLTFGAGTKLELKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRGEC

SEQ ID NO: 11 - protein, 1185 aa, Severe acute respiratory syndrome coronavirus 1
MFIFLLFLTLTSGSDLDRCTTFDDVQAPNYTQHTSSMRGVYYPDEIFRSDTLYLTQDLFLPFYSNVTGFHTINHTFGNPVIPFKDGIYFAATEKSNVVRGWVFGSTMNNKSQSVIIINNSTNVVIRACNFELCDNPFFAVSKPMGTQTHTMIFDNAFNCTFEYISDAFSLDVSEKSGNFKHLREFVFKNKDGFLYVYKGYQPIDVVRDLPSGFNTLKPIFKLPLGINITNFRAILTAFSPAQDIWGTSAAAYFVGYLKPTTFMLKYDENGTITDAVDCSQNPLAELKCSVKSFEIDKGIYQTSNFRVVPSGDVVRFPNITNLCPFGEVFNATKFPSVYAWERKKISNCVADYSVLYNSTFFSTFKCYGVSATKLNDLCFSNVYADSFVVKGDDVRQIAPGQTGVIADYNYKLPDDFMGCVLAWNTRNIDATSTGNYNYKYRYLRHGKLRPFERDISNVPFSPDGKPCTPPALNCYWPLNDYGFYTTTGIGYQPYRVVVLSFELLNAPATVCGPKLSTDLIKNQCVNFNFNGLTGTGVLTPSSKRFQPFQQFGRDVSDFTDSVRDPKTSEILDISPCSFGGVSVITPGTNASSEVAVLYQDVNCTDVSTAIHADQLTPAWRIYSTGNNVNFSISITTEVMPVSMAKTSVDCNMYICGDSTECANLLLQYGSFCTQLNRALSGIAAEQDRNTREVFAQVKQMYKTPTLKYFGGFNFSQILPDPLKPTKRSFIEDLLFNKVTLADAGFMKQYGECLGDINARDLICAQKFNGLTVLPPLLTDDMIAAYTAALVSGTATAGWTFGAGAALQIPFAMQMAYRFNGIGVTQNVLYENQKQIANQFNKAISQIQESLTTTSTALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQAAPHGVVFLHVTYVPSQERNFTTAPAICHEGKAYFPREGVFVFNGTSWFITQRNFFSPQIITTDNTFVSGNCDVVIGIINNTVYDPLQPELDSFKEELDKYFKNHTSPDVDLGDISGINASVVNIQKEIDRLNEVAKNLNESLIDLQELGKYEQYIKWPWYVWLGFIAGLIAIVMVTILLCCMTSCCSCLKGACSCGSCCKFDEDDSEPVLKGVKLHYT

SEQ ID NO: 12 - protein, 11 aa, Severe acute respiratory syndrome coronavirus 2
QTNSPRRARSV

SEQ ID NO: 13 - protein, 7 aa, synthetic construct
QTILRSV

SEQ ID NO: 14 - protein, 11 aa, synthetic construct
QTNSPGSASSV

SEQ ID NO: 15 - protein, 23 aa, synthetic construct
PVKQLLNFDLLKLAGDVESNPGP

SEQ ID NO: 16 - protein, 20 aa, synthetic construct
QCTNYALLKLAGDVESNPGP

SEQ ID NO: 17 - protein, 19 aa, synthetic construct
ATNFSLLKQAGDVEENPGP

SEQ ID NO: 18 - protein, 18 aa, synthetic construct
EGRGSLLTCGDVESNPGP

SEQ ID NO: 19 - protein, 573 aa, synthetic construct
MKTIIALSYILCLVFAQKLPGNDNSTATLCLGHHAVPNGTIVKTITNDQIEVTNATELVQSSSTGEICDSPHQILDGKNCTLIDALLGDPQCDGFQNKKWDLFVERSKAYSNCFPYDVPDYASLRSLVASSGTLEFNNESFNWTGVTQNGTSSACIRRSKNSFFSRLNWLTHLNFKYPALNVTMPNNEQFDKLYIWGVHHPGTDKDQIFLYAQASGRITVSTKRSQQTVSPNIGSRPRVRNIPSRISIYWTIVKPGDILLINSTGNLIAPRGYFKIRSGKSSIMRSDAPIGKCNSECITPNGSIPNDKPFQNVNRITYGACPRYVKQNTLKLATGMRNVPEKQTRGIFGAIAGFIENGWEGMVDGWYGFRHQNSEGRGQAADLKSTQAAIDQINGKLNRLIGKTNEKFHQIEKEFSEVEGRIQDLEKYVEDTKIDLWSYNAELLVALENQHTIDLTDSEMNKLFEKTKKQLRENAEDMGNGCFKIYHKCDNACIGSIRNGTYDHDVYRDEALNNRFQIKGGSGYIPEAPRDGQAYVRKDGEWVLLSTFLGSGLNDIFEAQKIEWHEGHHHHHH

SEQ ID NO: 20 - protein, 450 aa, synthetic construct
QVQLVESGGGVVQPGRSLRLSCAASGFTFSNYAMYWVRQAPGKGLEWVAVISYDGSNKYYADSVKGRFTISRDNSKNTLYLQMNSLRTEDTAVYYCASGSDYGDYLLVYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 21 - protein, 216 aa, synthetic construct
QSALTQPASVSGSPGQSITISCTGTSSDVGGYNYVSWYQQHPGKAPKLMIYDVSKRPSGVSNRFSGSKSGNTASLTISGLQSEDEADYYCNSLTSISTWVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 22 - protein, 450 aa, synthetic construct
QVQLVESGGGLVKPGGSLRLSCAASGFTFSDYYMSWIRQAPGKGLEWVSYITYSGSTIYYADSVKGRFTISRDNAKSSLYLQMNSLRAEDTAVYYCARDRGTTMVPFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 23 - protein, 214 aa, synthetic construct
DIQMTQSPSSLSASVGDRVTITCQASQDITNYLNWYQQKPGKAPKLLIYAASNLETGVPSRFSGSGSGTDFTFTISGLQPEDIATYYCQQYDNLPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 24 - protein, 451 aa, synthetic construct
QVQLVQSGAEVKKPGSSVKVSCKASGDTFSTYAINWVRQAPGQGLEWMGRFIHIFGTANYAQKFQGRVTITADESTSTAYMELRSLRSEDTAVYYCARDGVDYGDYRPDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 25 - protein, 215 aa, synthetic construct
EIVLTQSPGTLSLSPGERATLSCRASQSVSSNYLAWYQQKPGQAPRLLIYGASSRATGIPERFSGSGSGTDFTLTISRLEPEDFAVYYCQQYGSSLYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 26 - protein, 453 aa, synthetic construct
EVQLVESGGGLVQPGGSLRLSCSASGFTFSRYAMYWVRQAPGKGLEYVSAISSDGGSTYDADSVKGRFTISRANSKNTLYLQMSSLRAEDTAVYYCVKGLRELLYYYYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 27 - protein, 214 aa, synthetic construct
DIQMTQSPSSLSASVGDRVTITCRAGQSISSFLNWYQQKPGKAPKLLIYAASSLQSGVPSRFSGSGSGTDFTLTISSLQPEDFATYYCQQSYITPFTFGPGTKVDIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 28 - protein, 6 aa, synthetic construct
HHHHHH